(19) United States
(12) Patent Application Publication
(10) Pub. No.: US 2021/0401008 A1
(43) Pub. Date: Dec. 30, 2021

(54) COMPOSITIONS AND METHODS FOR INCORPORATING HEME FROM ALGAE IN EDIBLE PRODUCTS

(71) Applicant: Triton Algae Innovations, Inc., San Diego, CA (US)

(72) Inventors: Miller Tran, San Diego, CA (US); John Deaton, San Diego, CA (US); Brock Adams, San Diego, CA (US); Michael Mayfield, San Diego, CA (US); Amanda Longo, San Diego, CA (US); Oscar Gonzalez, San Diego, CA (US); Jon Hansen, San Diego, CA (US); Xun Wang, San Diego, CA (US); David Schroeder, San Diego, CA (US)

(21) Appl. No.: 17/291,583

(22) PCT Filed: Nov. 7, 2019
(86) PCT No.: PCT/US2019/060315

§ 371 Date: May 5, 2021

Related U.S. Application Data
(60) Provisional application No. 62/757,534, filed on Nov. 8, 2018.
(60) Provisional application No. 62/850,227, filed on May 20, 2019.
(60) Provisional application No. 62/865,800, filed on Jun. 24, 2019.

(51) Int. Cl. A23L 17/60; A23J 3/20; A23J 1/00; A23J 3/22; A23L 13/40; A23L 5/46

(52) U.S. Cl. CPC A23L 17/60; A23J 3/20; A23L 5/46; A23J 3/227; A23L 13/428; A23J 1/009

(57) ABSTRACT

Provided herein are compositions and processes for producing compositions from an algae to provide heme and a red or red-like color to edible compositions including ingredients and finished food products. Also provided are methods of growing heme-producing algae, methods of producing algae preparations therefrom and methods of making ingredients and food products with algae preparations. Also provided are compositions, including edible compositions that include heme and other nutrient components produced from algae.

### CROSS-REFERENCE TO RELATED APPLICATION(S)

This application claims the benefit of priority under 35 U.S.C. § 119(e) of U.S. Provisional Application No. 62/865,800, filed Jun. 24, 2019, of U.S. Provisional Application No. 62/850,227, filed May 20, 2019, and of U.S. Provisional Application No. 62/757,534, filed Nov. 8, 2018, the entire content of each of which is hereby incorporated by reference.

### SEQUENCE LISTING

The instant application contains a Sequence Listing which has been submitted electronically in ASCII format and is hereby incorporated by reference in its entirety. Said ASCII copy, created on Nov. 6, 2019, is named 20498-202379_SL.txt and is 208 kilobytes in size.

### BACKGROUND

With the advent of industrialized animal agriculture, the consumption of animal meat has continued to rise. Animal agriculture requires a significant amount of land use and fresh water, finite resources that are becoming increasingly difficult to access.

To address the sustainability and ethical concerns over animal meat consumption, the food industry has been aggressively trying to develop plant-based alternatives that taste, touch and smell like meat products. However, many of the current plant-based alternatives have not been able to penetrate the larger food and consumer markets. To improve the sustainability of the food ecosystem it is imperative that products are developed that appeal to consumers who currently prefer meat.

Recent advances made have demonstrated the potential of using heme-containing proteins, purified from a host organism, to make the flavor and aroma profile of a product closer to that of meat. It is thought that the heme from heme-containing proteins are responsible for imparting a “meaty” flavor and aroma to meat products. However, the available sources of heme-containing proteins are expensive and technically intensive limiting their utility. In addition to poor economics, the product is genetically modified making it less appealing to many consumers who have chosen to consume foods that are not a result of genetic engineering. Additionally, there is a trend to products with increased nutrition benefit and a balance of caloric intake. A number of the current meat alternatives cannot fully satisfy these demands while maintain the taste, texture and visual appeal desired by consumers. Thus, a need exists for edible products incorporating heme-containing proteins as set forth herein.

### SUMMARY OF THE INVENTION

To address both the economic and consumer concerns associated with the current approaches of incorporating heme into a product, provided herein are compositions and processes for producing such compositions that provide flavorful and nutritious alternatives to meat. In particular, provided herein are compositions and methods of producing such compositions that incorporate heme from algae, along with other nutrition components. Algae can be incorporated into finished products without the costly process of purification.

The present disclosure includes compositions of engineered algae overexpressing or accumulating heme and methods of using such engineered algae for food products. Thus, one aspect of the disclosure includes an engineered algae having a genetic modifications, where the genetic modification results in an accumulation of heme in the algae as compared to an algae lacking the genetic modification. In some embodiments, the engineered algae has reduced or absence of chlorophyll production. In some embodiments, the algae has red or red-like color. In some embodiments, the algae is capable of growth on glucose as the sole carbon source.

Preferably, the genetic modification comprises a genetic alteration to chlorophyll synthesis pathway, protoporphyrinogen IX synthesis pathway or heme synthesis pathway. In some embodiments, the genetic modification is associated with a deficiency in the expression of magnesium chelatase. Alternatively and/or additionally, the genetic modification comprises an alteration in one or more of CHLD, CHLI1, CHLI2 or CHLH1. Alternatively and/or additionally, the genetic modification comprises an alteration in an upstream regulatory region, a downstream regulatory region, an exon, an intron or any combination thereof. In some embodiments, the genetic modification comprises an insertion, a deletion, a point mutation, an inversion, a duplication, a frameshift or any combination thereof.

In some embodiments, the engineered algae has a heme content greater than the chlorophyll content. Alternatively and/or additionally, the engineered algae has a protoporphyrin IX content greater than the chlorophyll content. Alternatively and/or additionally, the engineered algae has reduced production of one or more fatty acids.

In some embodiments, the engineered algae further comprises a genetic modification that reduces or eliminates the expression of light independent protochlorophyllide oxidoreductase. In such embodiments, it is contemplated that the genetic modification comprises a mutation or deletion in one or more of ChlB, ChlL or ChlN. In some embodiments, the engineered algae has upregulated expression of ferrocheletase and/or upregulated expression of protoporphyrinogen IX oxidase. Optionally, the algae contain a recombinant or heterologous nucleic acid. In some embodiments, the engineered algae comprises a Chlamydomonas sp. Alternatively and/or additionally, the Chlamydomonas sp. is Chlamydomonas reinhardtii.

Another aspect of the disclosure includes an edible composition comprising an algae preparation, wherein the algae preparation comprises an engineered algae as described above or a portion thereof. In some embodiments, the edible composition comprises heme derived from the engineered algae. In some embodiments, the algae preparation comprises algae cells. In some embodiments, the algae preparation is a fractionated algae preparation. In some embodiments, the algae preparation is red or red-like in color.

In some embodiments, the edible composition has a red or red-like color derived from the algae preparation. Alternatively and/or additionally, the algae preparation confers a meat or meat-like flavor to the edible composition. Alternatively and/or additionally, the edible composition has a meat or meat-like texture derived from the algae preparation. In such embodiment, it is contemplated that the meat or meat-like texture comprises a beef or beef-like texture, a fish or fish-like texture, a chicken or chicken-like texture, a pork or pork-like texture or a texture of a meat replica.

In some embodiments, the edible composition is a finished product selected from the group consisting of a beef-like food product, a fish-like product, a chicken-like product, a pork-like product and a meat replica. Alternatively and/or additionally, the edible composition is vegan, vegetarian or gluten-free. Alternatively and/or additionally, the edible composition has an appearance of blood derived from the algae preparation.

Alternatively and/or additionally, the algae preparation has a heme content greater than the chlorophyll content. Alternatively and/or additionally, the algae preparation has a protoporphyrin IX content greater than the chlorophyll content. In some embodiments, the algae preparation provides at least about 5%, 10%, 15%, 20%, 25%, 30%, 35%, 40%, 45%, 50%, 55%, 60%, 65%, 70%, 75%, 80%, 85%, 90%, 95%, 99% or 100% of the total protein content to the edible composition. Alternatively and/or additionally, the algae preparation provides vitamin A, beta carotene or a combination thereof to the composition. Optionally, the vitamin A, the beta carotene or the combination thereof is at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended requirement. Alternatively and/or additionally, the algae preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the edible composition. Alternatively and/or additionally, the algae preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in a finished product comprising the edible composition. Alternatively and/or additionally, the algae preparation provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the edible composition. Alternatively and/or additionally, the algae preparation has reduced fatty acid content.

In some embodiments, the edible product is combined with a protein source, a fat source, a carbohydrate, a starch, a thickener, a vitamin, a mineral, or any combination thereof. In such embodiments, it is preferred that the protein source is selected from the group consisting of textured wheat protein, textured soy protein and textured pea protein, fungal protein or algal protein. Alternatively and/or additionally, the fat source comprises at least one of refined coconut oil or sunflower oil. In some embodiments, the edible component further comprises at least one of potato starch, methylcellulose, water, and a flavor, wherein the flavor is selected at least one of yeast extract, garlic powder, onion powder, and salt.

In some embodiments, the edible product is an ingredient for a burger, a sausage, a kebab, a filet, a fish-alternative, a ground meat-like product or a meatball. In some embodiments, the burger comprises about 5% of the algae preparation, about 20% textured soy protein and about 20% refined coconut oil. Optionally, the burger further comprises about 3% sunflower oil, about 2% potato starch, about 1% methylcellulose, about 45% water and about 4-9% flavors. Alternatively and/or additionally, the burger further comprises about 0.5% Kojac gum, about 0.5% Xanthan gum, about 45% water and about 4-9% flavors. In some embodiments, fish-alternative comprises 20% textured soy protein, about 5% of algae preparation, about 65% water and about 10% flavors. In some embodiments, the edible composition is free of animal proteins.

In some embodiments, the algae preparation comprises an algae having an increase in protoporphyrinogen IX synthesis or accumulation. Alternatively and/or additionally, the algae preparation comprises an algae that exhibits a red or red-like color when grown in the dark conditions. In some embodiments, the algae comprised in the algae preparation are recombinant or genetically modified algae. In some embodiments, the algae preparation comprises a Chlamydomonas sp. Optionally, the Chlamydomonas sp. is Chlamydomonas reinhardtii.

Another aspect of the disclosure includes a method for the production of an edible composition. The method includes steps of (a) culturing an engineered algae as described above in a condition where the engineered algae exhibits a red or red-like color and wherein the engineered algae produces heme, (b) collecting the cultured engineered algae to produce an algae preparation, and (c) combining the algae preparation with at least one edible ingredient to produce an edible composition. In some embodiments, the condition comprises a fermentation condition. Alternatively and/or additionally, the condition comprises acetate as a reduced carbon source for growth of the engineered algae. Alternatively and/or additionally, the condition comprises sugar as a reduced carbon source for growth of the engineered algae. Alternatively and/or additionally, the condition comprises dark or limited light condition. Alternatively and/or additionally, the condition further comprises iron supplements.

In some embodiments, the method further comprises fractionating the cultured algae to produce the algae preparation. In some embodiments, the algae preparation has a heme content that is greater than the chlorophyll content. Alternatively and/or additionally, algae preparation has a protoporphyrin IX content that is greater than the chlorophyll content. In some embodiments, the engineered algae is a Chlamydomonas sp. Optionally, the engineered algae is a Chlamydomonas reinhardtii.

In some embodiments, the edible composition has at least one of the features selected from the group consisting of a meat or meat-like flavor, a meat or meat-like texture, a blood-like appearance and a meat or meat-like color, where the at least one of the features is derived from the algae preparation. In some embodiments, the method further comprises producing a finished product comprising the edible composition and wherein the finished product is a beef-like food product, a fish-like product, a chicken-like product, a pork-like product or a meat replica. In some embodiments, the edible composition is free of animal proteins. In some embodiments, the algae preparation is fractionated to remove one or more of starch, protein, PPIX, fatty acids and chlorophyll.

Another aspect of the disclosure includes a method of making an engineered algae enriched in heme content. The method includes steps of (a) subjecting an algae strain to a process that produces genetic modification to create a first algae population, and (b) from the first algae population, selecting a second algae population that is enriched in heme content, and optionally, PPIX content. In some embodiments, the process comprises at least one of a random UV mutagenesis, a random chemical mutagenesis, a recombinant genetic engineering, a gene editing, or a gene silencing. In some embodiments, the method further comprises a step of culturing the first algae population in a fermentation condition. In some embodiments, the fermentation condition comprises a media having sugar as a sole carbon source. In such embodiments, it is preferred that the sugar is selected from glucose, dextrose, fructose, maltose, galactose, sucrose, and ribose. Alternatively and/or additionally, the fermentation condition comprises a brightness of less than 500 lux.

In some embodiments, the selecting the second algae population step comprises sorting or identifying algae cells having a red or red-like color. Alternatively and/or additionally, the second algae population step is performed by FACS. In some embodiments, the second algae population is selected with its capability to grow in the fermentation condition.

### BRIEF DESCRIPTION OF THE DRAWINGS

FIG. 1 is a pictorial diagram showing an exemplary pathway for the production of heme in algae. This exemplary pathway can be used by wildtype algae to produce chlorophyll, but it can also be used to generate heme.

FIGS. 2A and 2B show the composition of an exemplary algae growth media (FIG. 2A) and selection process (FIG. 2B).

FIG. 3 is a pictorial diagram showing algae growth in complete dark condition with dextrose as the only carbon source.

FIG. 4 is a pictorial diagram showing an exemplary fractionation of algae overexpressing heme, showing the separation into a protein and heme-enriched biomass, which is separated from the starch and carotenoid fractions.

FIG. 5 is a pictorial diagram showing extraction process of PPIX and/or heme from the red algae.

FIG. 6 is a graphical diagram showing an exemplary growth curve (dry cell weight) of a heme-overproducing strain when grown in aerobic fermentation conditions.

FIG. 7 is a graphical diagram showing increased dry cell weight of Chlamydomonas sp. in a glucose-containing media.

FIG. 8 is a graphical diagram showing the fractionated components of the red algae preparation before and after hexane extraction.

FIG. 9 shows a portion of sequence alignments of a wild type green algae and a red-algae with a mutation in CHLH gene (upper sequence (Seq_1) is a partial nucleic acid sequence (residues 1621-1679 of SEQ ID NO: 27) and a partial amino acid sequence (residues 451-460 of SEQ ID NO: 28) of CHLH gene of green algae, and lower sequence (Seq_2) is a partial nucleic acid sequence (residues 1621-1680 of SEQ ID NO: 129) and partial amino acid sequence (residues 451-460 of SEQ ID NO: 152) of CHLH gene of red algae has a mutation (asterisk)). As shown, the wild-type CHLH nucleic acid sequence (SEQ ID NO: 27) has an insertion of a thiamine at position 1678 resulting in a change of the wild-type CHLH amino acid sequence of SEQ ID NO: 28 of a proline to a serine at amino acid position 560.

FIG. 10 is a pictorial diagram showing burgers created with 0.01 g, 0.1 g, 1.0 g, and 5.0 g of the heme enriched algae.

FIG. 11 is a pictorial diagram showing ingredient mixes of the plant-based burger ingredients with no heme-enriched algae, with the addition of heme-enriched algae, or the ingredients with the addition of heme-enriched algae shaped into a burger before and after cooking.

FIG. 12 is a pictorial diagram showing an example of heme-enriched meatless “tuna”.

### DETAILED DESCRIPTION

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, “a deficiency in” or the “lack of”, or “reduction of”, one or more genes and/or enzymes include, for example, mutation or deletion of the gene sequence, a reduction in or lack in the expression from a gene (RNA and/or protein) and/or a lack of accumulation or stability of a gene product (RNA and/or protein).

As used herein, “overexpresses” and “overexpression” of an enzyme or gene include, for example, an increase in expression from a gene (RNA and/or protein) and/or an increase in accumulation or stability of a gene product (RNA and/or protein). Such overexpression can include alterations to the regulatory region(s) and/or to the gene sequence, as well as copy number, genomic position and post-translational modifications.

As used herein, the term “engineered algae” is used to refer to an algae that contains one or more genetic modifications. In some cases, an engineered algae is also a recombinantly modified organism when it incorporates heterologous nucleic acid into its genome through recombinant technology. In other cases, an engineered algae is not a recombinantly modified organism (for example when it is modified through UV, chemical or radiation mutagenesis). In some cases an algae that is not a recombinantly modified organism is referred to as non-GMO, and components from such algae can be referred to as non-GMO components.

As used herein, the term “genetic modification” is used to refer to any manipulation of an organism's genetic material in a way that does not occur under natural conditions. A genetic modification can include modifications that are made through mutagenesis (such as with UV light, X-rays, gamma irradiation and chemical exposure). A genetic modification can include gene editing. In some cases, genetic modifications can be made through recombinant technology. As used herein, “recombinantly modified organism” is used to refer to an organism that incorporates heterologous nucleic acid (e.g., recombinant nucleic acid) into its genome through recombinant technology. Methods of performing such manipulations are known to those of ordinary skill in the art and include, but are not limited to, techniques that make use of vectors for transforming cells with a nucleic acid sequence of interest. Included in the definition are various forms of gene editing in which DNA is inserted, deleted or replaced in the genome of a living organism using engineered nucleases, or “molecular scissors.” These nucleases create site-specific double-strand breaks (DSBs) at desired locations in the genome. The induced double-strand breaks are repaired through nonhomologous end-joining (NHEJ) or homologous recombination (HR), resulting in targeted mutations (i.e., edits).

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Provided herein are compositions and methods to provide heme and other nutrition components from algae. Algae are known for producing many compounds that result in these aquatic organisms being various colors. These compounds include, but are not limited to, chlorophyll which makes algae green, beta-carotene which makes algae appear yellow or orange, astaxanthin which makes algae appear red or other various pigments such as phycocyanin which make algae blue. While each of these previously mentioned compounds has been added to food products, there are to date no products that incorporate an algae over-producing heme to impart a red color and/or a meaty taste and smell.

Provided herein are strains, methods and compositions that employ algae overproducing heme. In some embodiments, the algae strain when grown is red or red-like in color. As used herein, in some embodiments, red-like color can be any color with a wavelength between 590 nm to 750 nm or any mixture of the color. Alternatively and/or additionally, in some embodiments, red-like color can be defined as any color in RGB (r.g.b) having r value between 255 and 80 with g or b values between 0 and 80. In some embodiments, a preparation made from the algae culture overproducing heme, imparts a pink or red color when incorporated into food and other edible products. In some embodiments, a preparation made from the algae culture overproducing heme, imparts a “meaty” flavor, smell and/or texture when incorporated into food and other edible products. In some embodiments, a preparation made from the algae culture overproducing heme, imparts a desired color, taste and/or smell, as well as one or more additional nutrition components such as omega-3 fatty acids, saturated fats, protein, vitamin A, beta-carotene or any combination thereof.

### Algae Producing and Over-Producing Heme

Provided herein are algae strains that over-produce heme and strains that produce or accumulate heme and/or protoporphyrin IX (PPIX) content greater than chlorophyll content and that can be used to produce edible compositions and ingredients. Also provided herein are methods of making such strains and ingredients and compositions therefrom. and use with the methods herein to make such compositions. Such strains are created by modifying one or more steps in the biochemical pathways that produce heme, PPIX and chlorophyll.

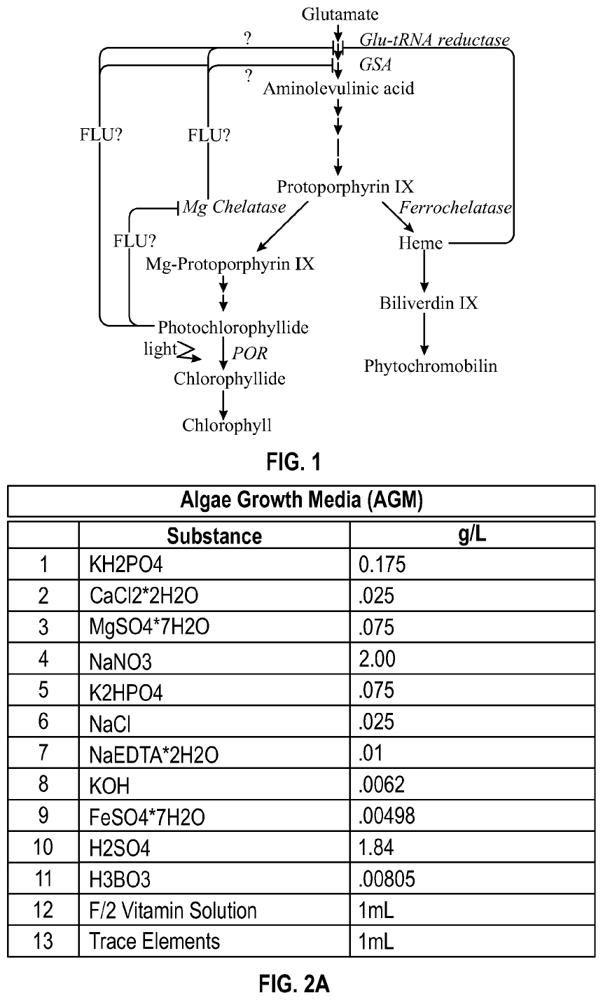

Without being bound by theory, the heme pathway is a biochemical pathway that branches from the chlorophyll biochemical pathway, as shown in FIG. 1. In short, this pathway starts with a glutamate tRNA which is converted to 5-aminolaevulinic acid (ALA) by a GlutRNA reductase and a GSA amino transferase. Next, ALA is converted to porphobilinogen by ALA dehydrase. Next, porophobilinogen is converted to hydroxymethylbilane by pophobilinogen deaminase. Next, hydroxymethylbilane is converted to uroporphyrinogen III by UPG III synthase. Next, uroporphyrinogen III is converted to coprophyrinogen by UPG III decarboxylase. Next, coprophyrinogen is converted to protoporphyrinogen IX by CPG oxidase. Next, protoporphyrinogen IX is converted to protoporphyrin IX by PPG oxidase. Protoporphyrin IX can be shuttled to the chlorophyll production pathway or towards heme B. Finally, protoporphyrin IX is converted to heme B by the enzyme ferrochelatase which attaches iron to protoporphyrin IX.

By reducing metabolic flux towards chlorophyll, it is possible to increase metabolic flux towards heme B. In some embodiments herein, the algae strains used in the methods and compositions produced therewith are reduced in metabolic flux towards chlorophyll and increased metabolic flux towards heme B (also referred to herein as “heme”). In some embodiments, the algae strain is one where chlorophyll and carotenoid synthesis is decreased and heme synthesis or accumulation is increased. In some embodiments, the algae strain is deficient or reduced in the amount of chlorophyll. In some embodiments, the algae strain is red or red-like in color.

In some embodiments, the algae strain is deficient for one or more enzymes in the chlorophyll biosynthesis pathway. Such deficiencies include, but are not limited to, gene deletions, mutations and other alterations that result in a lack expression of the enzyme or a deficiency in the functionality of the enzyme. In some embodiments, the algae strain is deficient in magnesium chelatase which is the first step in converting protoporphyrin IX to chlorophyll. In some embodiments, the algae strain is deficient for light dependent protochlorophyllide which converts protochlorophyllide to chlorophyllide. In some embodiments, the algae strain is deficient for a light independent protochlorophyllide which converts protochlorophyllide to chlorophyllide in the dark. In some embodiments, the algae strain is deficient for one or more of ChlB, ChlL, or ChlN gene products which are encoded in the chloroplast genome and are subunits of light independent protochlorophyllide oxidoreductase (LIPOR) that coverts protochlorophyllide to chlorophyllide. This enzyme, when expressed, can allow algae such as Chlamydomonas to produce chlorophyll and remain green even when the algae is not provided with illumination. When one or more of these genes are knocked out, the algae strain has a yellow color under dark growing conditions.

In some embodiments, the algae strain is lacking or reduced in one or more of magnesium chelatase, magnesium protoporphyrinogen IX, protochlorophyllide, chlorophyllide, and chlorophyll.

In some embodiments, the algae strain is deficient for one or more of the magnesium chelatase subunits CHLD, CHLH and CHLI. These subunits are also referred to by the gene names, CHLD1 (alternatively written as CH1D1), corresponding to the CHLD subunit, CHLH1 (alternatively written as CH1H1), corresponding to the CHLH subunit, and CHLI1 and CHLI2, corresponding to the CHLI subunit, encoded by two genes, CHLI1 and CHLI2 (alternatively written as CH1I1 and CH1I2).

In some embodiments, a heme-enriched algae strain is deficient in one or more of a nuclearly encoded subunit of magnesium chelatase, for example in one or more of the subunits encoded by the genes for the subunits CHLD, CHLH and CHLI. A deficiency in one or more of these subunits reduces or eliminates chlorophyll expression. In some embodiments, the gene encoding a subunit can be modified, such as by one or more point mutations that change a codon to a stop codon, resulting in a truncated coding region. In some embodiments, the gene encoding a subunit can be modified by a deletion that removed some of or all of the gene encoding the subunit. In some embodiments, the gene encoding a subunit can be modified by a frameshift mutation, such as caused by a deletion or insertion of one or more bases into the coding region, resulting in a non-functional and/or truncated protein. In some embodiments, the gene encoding a subunit can be modified by an insertion into the coding region that creates a non-functional protein, such as by adding one or more amino acids internally or at the N or C terminus of the protein that creates a non-functional subunit or reduces the activity or stability of the subunit or enzyme.

In some embodiments, a heme-enriched algae has at least one modification in the nucleotide sequence encoding CHLD, CHLI1, CHLI2 or CHLH1 (e.g., a modification in SEQ ID NOs: 23, 25, 27, 153) including the intron, exon, regulatory regions, or full gene sequences. In some embodiments, a heme-enriched algae has at least one modification in the amino acid sequence of CHLD, CHLI1, CHLI2 or CHLH1 (e.g., a modification in SEQ ID NOs: 24, 26, 28, 151). In some embodiments, a heme-enriched algae strain contains at least one modification (point mutation, deletion, or insertion) in an exon encoding a portion of CHLD, CHLI1, CHLI2 or CHLH1. In some embodiments, a heme-enriched algae strain contains at least one modification to a wildtype sequence of such exons, such as a modification in any of SEQ ID NOs: 47-58, 72-80, 91-102, and 132-141.

In some embodiments, a heme-enriched algae strain contains at least one modification (point mutation, deletion, or insertion) in an untranslated region of CHLD, CHLI1, CHLI2 or CHLH1, such as in the 5′ untranslated region or the 3′ untranslated region. In some embodiments, a heme-enriched algae strain contains at least one modification to a wildtype sequence of such untranslated regions, such as a modification in any of SEQ ID NOs: 45, 46, 70, 71, 89, 90, 130 or 131.

In some embodiments, the regulation of expression of one or more subunit of Mg-chelatase is altered to create a strain that has reduced amounts of chlorophyll. The regulatory regions of one or more of CHLD, CHLI1, CHLI2 and CHLH1 can be modified to reduce expression, such as by an insertion, deletion or one or more point mutations. Such alterations may modify, for example, transcription factor binding sites, enhancer sites, RNA polymerase interactions and transcriptional start sites in a manner the reduces or eliminates the transcription of a subunit gene.

In some embodiments, the expression of one or more subunits is altered by modifying the splicing of an intron with the gene of a subunit, such as a mutation, insertion or deletion that eliminates or alters a splicing donor or acceptor site or that otherwise alters the efficiency or accuracy of the gene splicing. In some embodiments, a heme-enriched algae strain contains at least one modification (point mutation, deletion, or insertion) in an intron of CHLD, CHLI1, CHLI2 or CHLH1. In some embodiments, a heme-enriched algae strain contains at least one modification to a wildtype sequence of such introns, such as a modification in any of SEQ ID NOs: 59-69, 81-88, 103-113, 142-150.

In some embodiments, the algae strain overexpresses one or more enzymes such that the balance of pathways favors heme production. In some embodiments, the algae strain overexpresses one or more of glutamyl-tRNA reductase, glutamyl-1-semialdehyde aminotransferase, ALA dehydrongenase, porphobilinogen deaminase, UPG III synthase, UPG III decarboxylase, CPG oxidase, PPG oxidase, and ferrochelatase. In some embodiments, the algae strain is improved for its ability to produce ALA, a rate limiting precursor of heme B synthesis. In some embodiments, the algae strain is improved for its ability to produce a functional ferrochelatase gene, the enzyme responsible for the conversion of protoporphyrin IX to heme B. In some embodiments, the algae strain is improved for its ability to produce UPG III synthase, UPG III decarboxylase, CPG oxidase, or PPG oxidase. In some embodiments, the algae strain has an increased amount of one or more of heme, a heme-containing protein, protoporphyrinogen IX, biliverdin IX, photochromobilin, and ferrocheletase, as compared to a wildtype strain.

In some embodiments, the algae strain produces carotenoids or precursors of carotenoids. Without being bound by theory, carotenoids confer color and can have an impact on the visual appearance of a plant-based alternative. Exemplary carotenoids include, but are not limited to, gamma-carotene, beta-carotene, beta cryptoxanthin, zeaxanthin, autheraxanthin, lutein, prolycopene and lycopene.

In some embodiments, the algae strain is deficient for carotenoids or precursors of carotenoids. Deficiencies in carotenoid biosynthesis can occur due to mutations, such as mutations that impact carotenoid biosynthesis, for example, mutations in the phytoene synthase gene.

In some embodiments herein, algae used in the compositions and methods herein is non-GMO, does not contain heterologous nucleic acid and/or is not created using recombinant technology. In some embodiments, algae used in the compositions and methods herein is selected based on its color, heme content, rate of heme synthesis, accumulation of heme, or protoporphyrin IX content, rate of synthesis or accumulation. In some embodiments, the algae have reduced levels of chlorophyll and/or levels of chlorophyll that are less than the levels of heme and/or protoporphyrin IX. In some embodiments, algae used in the compositions and methods herein does not contain a heterologous gene for one more genes involved in heme biosynthesis or accumulation, e.g., the algae does not contain a bacterial, fungal, plant or animal-derived gene or nucleic acid that is involved in heme biosynthesis, heme accumulation, protoporphyrin IX biosynthesis, or protoporphyrin IX accumulation.

In some embodiments, algae are modified in expression of one or more genes contributing to an increase in heme synthesis or accumulation, a decrease in chlorophyll synthesis or accumulation or a combination thereof. Such modifications can be created through mutagenesis such as by exposure to UV light, radiation or chemicals.

In some embodiments, modifications can be created through gene editing such as precisely engineered nuclease targeting to alter the expression of one or more components, such as by CRISPR-CAS nucleases. Such nucleases can be used to create insertions, deletions, mutations and replacements of one or more nucleotides or regions of nucleotides to modify the expression of one or more pathway enzymes in the pathway to reduce chlorophyll and/or to increase the production of heme. Subsequent to the creation of the modification, the algae strain can be grown and/or mated such that the nuclease and associated guide nucleic acids are removed, and the algae strain that remains does not retain the nuclease and associated editing system. In some embodiments, a nuclease such as the CRISPR-CAS nuclease can be used to make a modification to a component of the chlorophyll pathway such that chlorophyll expression and/or accumulation is reduced or abrogated. In some embodiments, a nuclease such as the CRISPR-CAS nuclease can be used to make a modification to a component of the chlorophyll pathway such that heme expression and/or accumulation is increased. In some embodiments, a nuclease such as the CRISPR-CAS nuclease is used to make a modification in one or more of CHLD, CHLI1, CHLI2 or CHLH1 resulting in a heme-enriched algae strain. Such modifications can be made by designing guide RNAs with modifications to one or more of SEQ ID NOs:45-113, 130-150 and/or 153 to include one or more point mutations, insertions, deletions or combinations thereof.

There are several families of engineered nucleases that can be used for gene editing described herein, for example, but not limited to, meganucleases, zinc finger nucleases (ZFNs), transcription activator-like effector-based nucleases (TALEN), the CRISPR-Cas system, and ARCUS. However, it should be understood that any known gene editing system utilizing engineered nucleases may be used in the methods described herein. Thus, in some embodiments, the algae strain overproducing heme can be created by using techniques such as a CRISPR-Cas system (e.g., CRISPR-CAS9) or by the use of zinc-finger nucleases.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with elements including a Cas gene and specifically designed CRISPRs, nucleic acid sequences can be cut and modified at any desired location. Methods of preparing compositions for use in genome editing using the CRISPR/Cas systems are described in detail in US Pub. No. 2016/0340661, US Pub. No. 2016/0340662, US Pub. No. 2016/0354487, US Pub. No. 2016/0355796, US Pub. No. 2016/0355797, and WO 2014/018423, which are specifically incorporated by reference herein in their entireties.

Zinc-finger nucleases (ZFNs) are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. The most common cleavage domain is the Type IIS enzyme Fok1. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b), all of which are incorporated herein by reference. One or more of these enzymes (or enzymatically functional fragments thereof) can be used as a source of cleavage domains.

In some embodiments, a heme-enriched algae is created by genetically modifying a strain to modify the chlorophyll and/or heme pathways. Introduction of recombinant nucleic acids such as those that interfere with, inhibit or down-regulate expression of an endogenous gene (e.g., one or more of CHLD, CHLI1, CHLI2 or CHLH1) can alter the flux through the pathway. Such genetic modifications can include the integration of recombinant DNA in a regulatory region, exon or intron for an endogenous gene, as well as the gene silencing (e.g., introduction of antisense or siRNA for down regulating or silencing the expression of one or more endogenous genes). In some embodiments, expression of genes within the pathway can be unregulated such that the pathway produced more PPIX that can be converted to heme, or upregulates the expression or activity of ferrochelatase to produce more heme in the algae. Nucleic acids for modification of ferrochelatase can include the regulatory regions, such as those of SEQ ID NOs: 114, 115, exons, such as those of SEQ ID NOs: 116-122, and introns, such as those of SEQ ID NOs: 123-128. In some embodiments, a heme enriched algae may include an increased copy number of ferrocheletase or the provision of a construct to overexpress ferrocheletase (such as those provided by nucleic acid sequence SEQ ID NO: 7, and protein sequence SEQ ID NO: 8). In some embodiments, genetic modifications include modifications to or expression of one or more genes in the chloroplast. In some embodiments, modifications are made to nuclear encoded genes or expression of such genes.

### Algae Genus and Species for Use in the Compositions and Methods

In the compositions and methods provided herein for producing heme and heme-containing compositions, algae strains that have a heme biosynthesis pathway are employed. In some embodiments, the algae strain for providing heme is a Chlorophyta (green algae). In some embodiments, the green algae is selected from the group consisting of Chlamydomonas, Dunaliella, Haematococcus, Chlorella, and Scenedesmaceae. In some embodiments, the Chlamydomonas is a Chlamydomonas reinhardtii. In varying embodiments, the green algae can be a Chlorophycean, a Chlamydomonas, C. reinhardtii, C. reinhardtii 137c, or a psbA deficient C. reinhardtii strain. In some embodiments, the selected host is Chlamydomonas reinhardtii, such as in Rasala and Mayfield, Bioeng Bugs. (2011) 2(1):50-4; Rasala, et al., Plant Biotechnol J. (2011) May 2, PMID 21535358; Coragliotti, et al., Mol Biotechnol. (2011) 48(1):60-75; Specht, et al., Biotechnol Lett. (2010) 32(10):1373-83; Rasala, et al., Plant Biotechnol J. (2010) 8(6):719-33; Mulo, et al., Biochim Biophys Acta. (2011) May 2, PMID:21565160; and Bonente, et al., Photosynth Res. (2011) May 6, PMID:21547493; US Pub. No. 2012/0309939; US Pub. No. 2010/0129394; and Intl. Pub. No. WO 2012/170125. All of the foregoing references are incorporated herein by reference in their entirety for all purposes.

In some embodiments, the algae strain for providing heme is a single-celled algae. Illustrative and additional microalgae species of interest include without limitation, Achnanthes orientalis, Agmenellum, Amphiprora hyaline, Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp., Anabaena, Ankistrodesmus, Ankistrodesmus falcatus, Boekelovia hooglandii, Borodinella sp., Botryococcus braunii, Botryococcus sudeticus, Carteria, Chaetoceros gracilis, Chaetoceros muelleri, Chaetoceros muelleri subsalsum, Chaetoceros sp., Chlamydomonas sp., Chlamydomonas reinhardtii, Chlorella anitrata, Chlorella Antarctica, Chlorella aureoviridis, Chlorella candida, Chlorella capsulate, Chlorella desiccate, Chlorella Chlorella emersonii, Chlorella fusca, Chlorella fusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella parva, Chlorella photophila, Chlorella pringsheimii, Chlorella protothecoides, Chlorella protothecoides var. acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgaris, Chlorella vulgaris f. tertia, Chlorella vulgaris var. autotrophica, Chlorella vulgaris var. viridis, Chlorella vulgaris var. vulgaris, Chlorella vulgaris var. vulgaris f. tertia, Chlorella vulgaris var. vulgaris f. viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Dunaliella tertiolecta, Eremosphaera viridis, Eremosphaera sp., Ellipsoidon sp., Euglena, Franceia sp., Fragilaria crotonensis, Fragilaria sp., Gleocapsa sp., Gloeothamnion sp., Hymenomonas sp., Isochrysis aff galbana, Isochrysis galbana, Lepocinclis, Micractinium, Micractinium (UTEX LB 2614), Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia communis, Nitzschia dissipata, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Pascheria acidophila, Pavlova sp., Phagus, Phormidium, Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pyramimonas sp., Pyrobotrys, Sarcinoid chrysophyte, Scenedesmus armatus, Schizochytrium, Spirogyra, Spirulina platensis, Stichococcus sp., Synechococcus sp., Tetraedron, Tetraselmis sp., Tetraselmis suecica, Thalassiosira weissflogii, and Viridiella fridericiana. In some embodiments, the algae is a Chlamydomonas species. In some embodiments, the algae is a Chlamydomonas reinhardtii. In some embodiments, the algae is a derivative of a green Chlamydomonas strain made by mutagenesis, by screening, by selection or by mating with another algae strain.

In some embodiments, the algae strain for use in the methods herein and for making heme-containing compositions is selected or identified based on one or more phenotypes and/or genotypes. In some embodiments, the algae strain for overproducing heme can be created through mating processes. In some embodiments, the algae strain for overproducing heme can be created through mutagenesis, such as ultra violet mutagenesis. In some embodiments, the algae strain for overproducing heme can be generated through chemical mutagenesis with a compound that results in DNA alterations.

Methods for selection of algae include, but are not limited to, genetic screening or phenotypic screening for deficiencies, mutations and changes in the chlorophyll biosynthesis pathway and/or chlorophyll accumulation, and by genetic screening or phenotypic screening for increased expression and/or accumulation of heme, heme biosynthesis intermediates and heme biosynthesis enzymes. In some embodiments, the algae strain for use in the methods herein and for making heme-containing compositions is selected or identified based on its spectral profile and/or its red or red-like color. In some embodiments, the algae for use in the methods herein and for making heme-containing compositions is selected or identified based on its growth rate in dark conditions. In some embodiments, the selection is based on growth rate in dark conditions and the appearance or enhancement of a red or red-like color when grown in dark conditions. In some embodiments, an algae strain is selected which is deficient in or reduced in the amount of carotenoids produced or accumulated.

In some embodiments, algae strains are mated to combine or enhance characteristics that contribute to heme production, heme accumulation, reduction in chlorophyll and/or reduction in carotenoids. In some embodiments, an algae strain that has fast growth under dark conditions (e.g., faster than a wildtype strain) is mated with an algae strain that exhibits a red or red-like color. In some embodiments, an algae strain deficient for carotenoid production or accumulation is mated with an algae strain exhibiting a red or red-like color.

In some embodiments, an algae strain is mutagenized and then a new strain is selected or identified that exhibits one or more characteristics of increased heme production, heme accumulation, reduction in chlorophyll and/or reduction in carotenoids. In some embodiments, an algae strain is generated by mutagenesis of a first starting strain and selection of a second strain that grows faster in the dark than the first starting strain. In some embodiments, an algae strain is generated by mutagenesis of a first starting strain and selection of a second strain that lacks one or more carotenoids. In some embodiments, the strain includes further modifications, such as a modification that decreases omega oils (e.g., omega-3 fatty acids) and/or a modification that allows the strain to grow on a particular carbon source such as glucose, dextrose, sucrose, etc.

In some embodiments, the algae is a Chlamydomonas species, such as Chlamydomonas reinhardtii and the strain has a visible red or reddish-brown appearance. In some embodiments, the strain also exhibits growth on glucose. In some embodiments, the strain has a genetic modification in the chlorophyll synthetic pathway, such as in a nuclearly encoded subunit of Mg-chelatase, such as in a gene encoding CHLD, CHLI1, CHLI2 or CHLH1, or in an intron or regulatory region thereof, whereby the strain overexpresses or is enriched in heme content. In some embodiments, the strain is also enriched in PPIX content. In some embodiments, the strain is capable of growing to high culture density under fermentation conditions.

### Culture Methods for Overproducing Heme Strains

Methods for growing algae in liquid media include a wide variety of options including ponds, aqueducts, small scale laboratory systems, and closed and partially closed bioreactor systems. Algae can also be grown directly in water, for example, in an ocean, sea, lake, river, reservoir, etc.

In some embodiments, the heme overproducing algae useful in the methods and compositions provided herein are grown in a controlled culture system, such as a small scale laboratory systems, large scale systems and closed systems and partially closed bioreactor systems. Small scale laboratory systems refer to cultures in volumes of less than about 6 liters, and can range from about 1 milliliter or less up to about 6 liters. Large scale cultures refer to growth of cultures in volumes of greater than about 6 liters, and can range from about 6 liters to about 200 liters, and even larger scale systems covering 5 to 2500 square meters in area, or greater. Large scale culture systems can include liquid culture systems from about 10,000 to about 20,000 liters and up to about 1,000,000 liters.

The culture systems for use with the methods for producing the compositions herein include closed structures such as bioreactors, where the environment is under stricter control than in open systems or semi-closed systems. A photobioreactor is a bioreactor which incorporates some type of light source to provide photonic energy input into the reactor. The term bioreactor can refer to a system closed to the environment and having no direct exchange of gases and contaminants with the environment. A bioreactor can be described as an enclosed, and in the case of a photobioreactor, illuminated, culture vessel designed for controlled biomass production of liquid cell suspension cultures.

In some embodiments, the algae used in the methods and for the compositions provided herein are grown in fermentation vessels. In some embodiments, the vessel is a stainless steel fermentation vessel. In some embodiments, the algae are grown in heterotrophic conditions whereby one or more carbon sources is provided to the culture. In some embodiments, the algae are grown in aerobic and heterotrophic conditions. In some embodiments, the algae are grown to a density greater than or about 10 g/L, about 20 g/L, about 30 g/L, about 40 g/L, about 50 g/L, about 75 g/L, about 100 g/L, about 125 g/L, or about 150 g/L.

In some embodiments, the algae are inoculated from a seed tank to a starting density of greater than about 0.1 g/L, about 1.0 g/L, about 5.0 g/L, about 10.0 g/L, about 20.0 g/L, about 50 g/L, about 80 g/L, or about 100 g/L. Once inoculated, the algae are grown heterotrophically using an aerobic fermentation process. During this process, the algae are fed nutrients to maintain their growth. In some embodiments, these nutrients include a reduced carbon source. Exemplary aerobic fermentation process and/or reduced carbon sources include, but are not limited to, acetate, glucose, sucrose, fructose, glycerol and other types of sugars (e.g., dextrose, maltose, galactose, sucrose, ribose, etc.). In some embodiments, the algae culture is supplemented with iron.

In some embodiments, the algae are cultured under dark conditions. Preferably, the dark condition has a brightness of less than 1000 lux, less than 750 lux, less than 500 lux, less than 400 lux, less than 300 lux, less than 200 lux, less than 100 lux. In some embodiments, the algae cultured under dark conditions lack or are reduced in chlorophyll production at least 10%, at least 20%, at least 30%, at least 40%, at least 50%, at least 60%, at least 70%, or at least 80% compared to the algae cultured under dark conditions. In some embodiments, the algae grown under dark conditions are supplemented with one or more nutrients. In some embodiments, the algae grown under dark conditions are grown in the presence of a reduced carbon source, such as acetate, glucose, sucrose, fructose, glycerol or other types of sugars (e.g., dextrose, maltose, galactose, sucrose, ribose, etc.). In some embodiments, the algae grown under dark conditions are grown in the presence of iron or otherwise supplemented with iron.

In some embodiments, the heme-enriched strains herein are grown in dark or limited light conditions such that the pathway flux to biliverdin IX and photochromobilin are decreased, and the amount of heme in such strains is increased. In some embodiments, the heme-enriched strains herein are grown in dark or limited light condition and utilize a carbon source such as glucose.

### Edible Food Products and Ingredients

Provided herein are edible products for human and animal consumption that contain heme from algae. In some embodiments, the edible product is a beef-like product, a fish-like product or a meat replica. In some embodiments, the edible product contains whole cell algae, where the algae provides heme to the composition. In some embodiments, the heme is imparted to the edible product by a whole cell algae component where the algae overproduce heme. In some embodiments, the heme is imparted to the edible product by an algae having a heme content greater than the chlorophyll content of the algae. In some embodiments, the heme is imparted to the edible product by an algae having a protoporphyrin content greater than chlorophyll content by at least 5%, at least 10%, at least 20%, at least 30%, at least 40%, or at least 50%.

In some embodiments, the edible product is a beef-like product, a fish-like product or a meat replica and the heme is provided by fractionated algae. For example, whole cell alga producing or overproducing heme can be subjected to fractionation methods to separate some or a substantial amount of biomass from the heme-containing fraction. The fractionation may remove one or more components of the algae biomass while leaving other components such as omega-3 fatty acids, fats, protein, vitamin A, beta-carotene or any combination thereof associated with the heme-containing fraction. In some embodiments, the heme can be separated from one or more of the omega-3 fatty acids, saturated fats, protein, vitamin A, and/or beta-carotene of the algae. Extraction with solvents and buffers or a combination thereof can be used to provide a heme-enriched fraction. For example, an alga biomass or a fractions thereof can be enriched for heme through hexane extraction.

In some embodiments, the biomass is fractionated or otherwise treated to separate heme content and optionally, PPIX. Such fractionation can include separation of PPIX from heme. For example, heme-binding proteins and heme associated with proteins can be separated from PPIX which is not a protein-conjugated or protein-associated compound. Both free heme and protein-associated heme can be separated from PPIX based on heme's association with iron. PPIX does not contain an iron moiety and as such, this feature can be used to separate PPIX from a heme-containing fraction. In some embodiments, an algae biomass herein is fractionated or otherwise treated such that the heme is separated from other components, including PPIX.

In some embodiments, the heme-containing fraction has a heme content greater than the chlorophyll content of the fraction by at least 5%, at least 10%, at least 20%, at least 30%, at least 40%, or at least 50%. In some embodiments, the heme-containing fraction has a protoporphyrin IX content greater than chlorophyll content of the fraction by at least 5%, at least 10%, at least 20%, at least 30%, at least 40%, or at least 50%. In some embodiments, the heme-containing fraction contains no chlorophyll or substantially no chlorophyll. In some embodiments, the heme-containing fraction has no chlorophyll or substantially no chlorophyll and has about 4.5% protoporphyrin IX content (on a weight per total weight basis, e.g., 45 mg protoporphyrin IX in a 1 gram sample). In some embodiments, the heme-containing fraction has no chlorophyll or substantially no chlorophyll and has about 0.5% heme content (on a weight per total weight basis, e.g., 5 mg heme in a 1 gram sample). In some embodiments, the heme-containing fraction has no chlorophyll or substantially no chlorophyll and has about 4.5% protoporphyrin IX content and has about 0.5% heme content (on a weight per total weight basis).

In some embodiments, a whole algae preparation used in the preparation of an edible composition has a heme content greater than the chlorophyll content of the fraction. In some embodiments, the whole algae preparation has a protoporphyrin IX content greater than chlorophyll content of the fraction. In some embodiments, the whole algae preparation contains no chlorophyll or substantially no chlorophyll. In some embodiments, the whole algae preparation has no chlorophyll or substantially no chlorophyll and has about 4.5% protoporphyrin IX content (on a weight per total weight basis, e.g., 45 mg protoporphyrin IX in a 1 gram sample). In some embodiments, the whole algae preparation has no chlorophyll or substantially no chlorophyll and has about 0.5% heme content (on a weight per total weight basis, e.g., 5 mg heme in a 1 gram sample). In some embodiments, the whole algae preparation has no chlorophyll or substantially no chlorophyll and has about 4.5% protoporphyrin IX content and has about 0.5% heme content (on a weight per total weight basis).

In some embodiments, the whole algae preparation or fractionated algae preparation has no chlorophyll or substantially no chlorophyll and is made from an algae strain that does not make or accumulate chlorophyll. In some embodiments, the whole algae preparation or fractionated algae preparation has no chlorophyll or substantially no chlorophyll and is made from an algae strain that has one or more mutations in the chlorophyll synthesis pathway and/or has one or more mutations in the pathways that impact the accumulation or turnover of chlorophyll, for example, having a modification in one or more subunits of magnese chelatase such as a modification in one or more of CHLD, CHLI1, CHLI2 or CHLH1.

In some embodiments, the whole algae preparation or fractionated algae preparation contains heme at about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.5% or more than 2.5% on a weight per total weight basis. In some embodiments, the whole algae preparation or fractionated algae preparation contains protoporphyrin IX at about 0.5%, 1.0%, 1.5%, 2.0%, 2.5%, 3.0%, 3.5%, 4.5%, 5.0%, 5.5%, 6.0%, 6.5%, 7.0%, 7.5%, 8.0%, 8.5%, 9.0%, 9.5%, 10.0% or more than 10% on a weight per total weight basis. In some embodiments, the heme in the whole algae preparation or fractionated algae preparation is free heme. In some embodiments, the heme in the whole algae preparation or fractionated algae preparation is complexed with one or more proteins, for example complexed to one or more truncated hemoglobins. In some embodiments, the heme in the whole algae preparation or fractionated algae preparation is a mixture of free heme and heme complexed with protein.

In some embodiments, the whole cell or fractionated algae provides protein to the edible composition as well as providing heme. In some embodiments, the algae provides at least about 1%, 2%, 3%, 4%, 5%, 6%, 7%, 8%, 9% or 10% of the protein to the edible composition. In some embodiments, the algae provides greater than about 5%, 10%, 15%, 20%, 25%, 30%, 35%, 40%, 45%, 50%, 55%, 60%, 65%, 70%, 75%, 80%, 85%, 90%, 95%, 99% or 100% of the protein in the edible product. In some embodiments, the whole cell or fractionated algae provides protein to the edible composition and the edible composition also contains protein from one or more additional sources, such as a plant-based source. In some embodiments, an alga fraction is enriched for protein as compared to the starting biomass. hexane extraction or an equivalent solvent can be used to enrich the protein content of the fraction. In some embodiments, carbohydrates and/or fatty acids are removed or reduced in amount through such extraction(s), while enriching for protein and/or enriching for heme.

In some embodiments, the whole cell or fractionated algae provides omega-3 fatty acids to the edible composition as well as providing heme. In some embodiments, the algae provides a daily recommended dosage of omega-3 fatty acids or a portion thereof to the edible product. For example, the whole cell or fractionated algae provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg, or 500 mg of omega-3 fatty acids to the edible composition.

In some embodiments, omega oils such as omega-3 fatty acids are removed from the alga biomass or a fractionated alga sample. Such oil removal can modify the aroma and taste of the alga biomass or faction, such as by decreasing or removing a “fishy” aroma or taste that can be present in an alga-derived product. In some embodiments, hexane or a similar solvent such as isohexane, heptane, butane or other alcohol, is used in the preparation of the alga biomass or fractionation to modify the aroma and taste. In some cases, hexane or similar solvent extraction removes or decreases the amount of oils, as well as enriches for heme and/or enriches for protein in the resulting product.

In some embodiments, algae biomass or fractionate algae are made using a strain deficient in one or more omega oils. Such strains can be combined with a heme-enriched strain, such as through mating to produce a heme-enriched strain that produces less omega oils.

In some embodiments, the whole cell or fractionated algae provides vitamin A to the edible composition as well as providing heme. In some embodiments, the algae provides a daily recommended dosage of vitamin A or a portion thereof to the edible product. For example, the whole cell or fractionated algae provides at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended dosage of vitamin A or at least about 20 μg, 50 μg, 100 μg, 200 μg, 300 μg, 400 μg, 500 μg, 600 μg, 700 μg, 800 μg, 900 μg or 1000 μg of retinol activity equivalents (RAE) for vitamin A. In some embodiments, the whole cell or fractionated algae provides no more than about 2,000 μg, 2,500 μg or 3,000 μg of retinol activity equivalents (RAE) for vitamin A.

In some embodiments, the whole cell or fractionated algae provides beta-carotene to the edible composition as well as providing heme. In some embodiments, the algae provides a daily recommended dosage of beta-carotene or a portion thereof to the edible product. For example, the whole cell or fractionated algae provides at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended dosage of beta-carotene. In some embodiments, the algae provides about 0.25 mg, 0.5 mg, 1 mg, 1.5 mg, 2 mg, 2.5 mg, 3 mg, 4 mg, 5, mg, 6 mg, 9 mg, 10 mg, 12 mg, or 15 mg of beta-carotene.

In some embodiments, the whole cell or fractionated algae that provides heme contains saturated fat. In some embodiments, the algae provides less than daily recommended limit for saturated fat or a portion thereof to the edible product. For example, the whole cell or fractionated algae provides no more than about 1%, 5%, 10%, 15%, 20%, 25%, 30%, 35%, 40%, 45%, 50%, 55%, 60%, 65%, 70%, 75%, 80%, 85%, 90%, 95%, or 100% of the daily recommended dosage of saturated fat. In some embodiments, the algae provides no more than 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the edible composition or in the finished product made from the edible composition.

In some embodiments herein, the heme-containing whole algae or algae fraction is used to create an edible composition that is then used as an ingredient in a finished product. The ingredient may provide heme as well as omega-3 fatty acids, fats, protein, vitamin A, beta-carotene or any combination thereof to the ingredient. Such ingredient may be a colorant, texturant, binder, nutrient source, taste or flavor enhancer, or a filler.

In some embodiments, the heme-containing whole algae or algae fraction is used to create an edible composition that is a finished product. For example, the finished product may be a meat-like product such as a burger, a patty, a cake, a ground “meat,” a sausage, a kebab, a steak, cubed “meat,” a “meatball,” a filet, a drumstick, a “chicken finger,” or a “chicken nugget.” The finished product may be a meat-like product made to resemble beef, chicken, pork, wild game, turkey or other consumable meat product. The finished product may be a fish-like product made to resemble a fish filet, a fish patty or cake, a fish ball, a fish salad, ground fish, a fish nugget, a fish burger or the like, such as a tuna product, a spicy tuna product or a salmon product.

The whole algae or algae fraction may provide omega-3 fatty acids, saturated fats, protein, vitamin A, beta-carotene or any combination thereof to the finished product. In some embodiments, the whole algae or algae fraction can be reduced in omega oils and used for the finished product. Meat-like products can be made with a whole algae or algae fraction from a heme-enriched algae that is as described herein, by processing or by strain type, reduced in the amount of omega oils.

In some embodiments, the finished product comprising the whole algae or algae fraction is a cooked product. In some embodiments, the finished product comprising the whole algae or algae fraction is a uncooked product or raw product. In some embodiments, the finished product comprising the whole algae or algae fraction is a partially-cooked product.

### Heme-Containing Preparations and Products

Algae strains and cultures overproducing heme such as described herein can be used in various forms and preparations. In some embodiments, a heme-containing composition is prepared from an algae culture overproducing heme, where the composition is red or red-like in color.

In some embodiments, the heme-containing composition is prepared from a biomass isolated from cultured algae. In some embodiments, the biomass is further fractionated to remove one or more components. In some embodiments, the biomass is fractionated to remove starch. In some embodiments, the biomass is fractionated to remove protein. In some embodiments, the biomass is fractionated or otherwise treated to remove carotenoids. In some embodiments, the biomass is fractionated or otherwise treated to enrich for certain components. In some embodiments, the fractionated or treated biomass is enriched in heme. In some embodiments, the fractionated or treated biomass is enriched in protein or in protein and heme. In some embodiments, the fractionation or treatment enhances the red or red-like color of the preparation. The fractionated or treated biomass can be enriched for protein content such that the composition is about 10% protein, greater than about 10% protein, or greater than about 20%, about 30%, about 40%, or about 50% protein.

In some embodiments, the heme-containing composition is a heme-containing liquid prepared from the culture media of the cultured algae. In some embodiments, the heme-containing composition is prepared from heme found extracellularly in the algae culture. In some embodiments, the algae culture is lysed or otherwise treated to release heme from the cells. In some embodiments, the heme-containing liquid is further fractionated to remove one or more components. In some embodiments, the heme-containing liquid is fractionated to remove starch. In some embodiments, the heme-containing liquid is fractionated to remove protein. In some embodiments, the heme-containing liquid is fractionated or otherwise treated to remove carotenoids. In some embodiments, the heme-containing liquid is fractionated or otherwise treated to enrich for certain components. In some embodiments, the fractionated or treated heme-containing liquid is enriched in heme. In some embodiments, the fractionation or treatment enhances the red or red-like color of the preparation.

The heme-containing compositions, including biomass, liquid and fractionated preparations can be further processed. Such processing can include concentrating, drying, lyophilizing, and freezing. In various embodiments, the heme-containing compositions can be combined with additional components and ingredients. In some embodiments, the heme-containing composition is combined with additional ingredients to create an edible product. In some embodiments, the heme-containing composition confers a red or red-like color to the edible product. In some embodiments, the heme-containing composition confers a meat-like characteristic such as a meat-like taste, meat-like flavor aroma and/or texture to the edible product. In some embodiments, the heme-containing composition provides the appearance of blood to an edible product, such as to a meat replica, a beef-like product, a chicken-like product or the like. Alternatively, at least one of the features of meat or meat-like flavor or aroma, a meat or meat-like texture, a blood-like appearance, a meat or meat-like color are derived from the algae preparation.

In some embodiments, heme-containing compositions are combined with additional ingredients to create a meat-like product. Such meat-like products can include clean meat or cultured meat (made from animal cells grown in the laboratory or otherwise outside of an animal), plant-based and non-animal based meats (made from plant ingredients and/or ingredients not from animal sources). In some embodiments, a heme-containing composition made from an over-producing algae is combined with additional ingredients to create a meat-like product whereby the addition of the heme-containing composition confers a red or red-like color, a meat-like aroma, a meat-like taste and/or a meat-like texture to the meat-like product. In some embodiments, the meat-like features conferred by the heme-containing composition are conferred to the raw or uncooked product. In some embodiments, the meat-like features conferred by the heme-containing composition is conferred to the cooked product.

In some embodiments, whole algae or fractionated algae is combined with an additional protein source in an edible composition. For example, the protein source is wheat protein, such as wheat protein textured wheat protein, pea protein, textured pea protein, soy protein, textured soy protein, potato protein, whey protein, yeast extract, or other plant-based protein source or any combination thereof. In some embodiments, whole algae or fractionated algae is combined with an oil or source of fat in an edible composition. For example, the oil or fat source is coconut oil, canola oil, sunflower oil, safflower oil, corn oil, olive oil, avocado oil, nut oil or other plant-based oil or fat source or any combination thereof. In some embodiments, whole algae or fractionated algae is combined with a starch or other carbohydrate source such as from potato, chickpea, wheat, soy, beans, corn or other plant-based starch or carbohydrate or any combination thereof. In some embodiments, whole algae or fractionated algae is combined with a thickener in an edible composition. For example, starches as arrowroot, cornstarch, katakuri starch, potato starch, sago, tapioca and their starch derivatives may be used as a thickener; microbial and vegetable gums used as food thickeners include alginin, guar gum, locust bean gum, konjac and xanthan gum; and proteins such as collagen and egg whites may be used as thickeners; and sugar polymers for use as thickeners include agar, methylcellulose, carboxymethyl cellulose, pectin and carrageenan. In some embodiments, whole algae or an algae fraction may be combined with vitamins and minerals in an edible composition, such as vitamin E, vitamin C, thiamine (vitamin B1), zinc, niacin, vitamin B6, riboflavin (vitamin B2), and vitamin B12.

In some embodiments, whole algae or an algae fraction may be combined with additional ingredients such that the edible composition and/or finished product is vegetarian, vegan or gluten-free and therefore may conform to the dietary guidelines of Jewish kosher practitioners, and halal practitioners. Thus, in some embodiments, the edible composition and/or finished product may be suitable for consumption by vegetarians, vegans, gluten-free populations, Jewish kosher practitioners, and halal practitioners. In some embodiments, whole algae or an algae fraction may be combined with additional ingredients such that the edible composition and/or finished product is GMO-free and/or does not contain any ingredients derived from genetically engineered organisms or cells.

### EXEMPLARY NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. An engineered algae having a genetic modifications, where the genetic modification results in an accumulation of heme in the algae as compared to an algae lacking the genetic modification. 2. The engineered algae of embodiment 1, wherein the engineered algae has reduced or absence of chlorophyll production. 3. The engineered algae of embodiment 1 or embodiment 2, wherein the algae has red or red-like color. 4. The engineered algae according to any of embodiments 1-3, wherein the algae is capable of growth on glucose as the sole carbon source. 5. The engineered algae according to any of embodiments 1-4, wherein the genetic modification comprises a genetic alteration to chlorophyll synthesis pathway, protoporphyrinogen IX synthesis pathway or heme synthesis pathway. 6. The engineered algae according to any of embodiments 1-5, wherein the genetic modification is associated with a deficiency in the expression of magnesium chelatase. 7. The engineered algae according to any of embodiments 1-6, wherein the genetic modification comprises an alteration in one or more of CHLD, CHLI1, CHLI2 or CHLH1. 8. The engineered algae of embodiment 7, wherein the genetic modification comprises an alteration in an upstream regulatory region, a downstream regulatory region, an exon, an intron or any combination thereof 9. The engineered algae according to any of embodiments 5-8, wherein the genetic modification comprises an insertion, a deletion, a point mutation, an inversion, a duplication, a frameshift or any combination thereof 10. The engineered algae according to any of embodiments 1-9, wherein the engineered algae has a heme content greater than the chlorophyll content. 11. The engineered algae according to any of embodiments 1-10, wherein the engineered algae has a protoporphyrin IX content greater than the chlorophyll content. 12. The engineered algae according to any of embodiments 1-11, wherein the engineered algae has reduced production of one or more fatty acids. 13. The engineered algae according to any of embodiments 1-12, wherein the engineered algae further comprises a genetic modification that reduces or eliminates the expression of light independent protochlorophyllide oxidoreductase. 14. The engineered algae of embodiment 13, wherein the genetic modification comprises a mutation or deletion in one or more of ChlB, ChlL or ChlN. 15. The engineered algae according to any of embodiments 1-14, wherein the engineered algae has upregulated expression of ferrocheletase. 16. The engineered algae according to any of embodiments 1-15, wherein the engineered algae has upregulated expression of protoporphyrinogen IX oxidase. 17. The engineered algae according to any of embodiments 1-16, wherein the algae contain a recombinant or heterologous nucleic acid. 18. The engineered algae according to any of embodiments 1-17, wherein the engineered algae comprises a Chlamydomonas sp. 19. The engineered algae of embodiment 18, wherein the Chlamydomonas sp. is Chlamydomonas reinhardtii.

Embodiment 20. An edible composition comprising an algae preparation, wherein the algae preparation comprises an engineered algae of any of embodiments 1-19 or a portion thereof 21. The edible composition of embodiment 20, wherein the edible composition comprises heme derived from the engineered algae. 22. The edible composition of embodiment 20, wherein the algae preparation comprises algae cells. 23. The edible composition of embodiment 20, wherein the algae preparation is a fractionated algae preparation. 24. The edible composition according to any of embodiments 20-23, wherein the algae preparation is red or red-like in color. 25. The edible composition according to any of embodiments 20-24, wherein the edible composition has a red or red-like color derived from the algae preparation. 26. The edible composition according to any of embodiments 20-25, wherein the algae preparation confers a meat or meat-like flavor to the edible composition. 27. The edible composition according to any of embodiments 20-26, wherein the edible composition has a meat or meat-like texture derived from the algae preparation. 28. The edible composition according to embodiment 27, wherein the meat or meat-like texture comprises a beef or beef-like texture, a fish or fish-like texture, a chicken or chicken-like texture, a pork or pork-like texture or a texture of a meat replica. 29. The edible composition according to any of embodiments 20-28, wherein the edible composition is a finished product selected from the group consisting of a beef-like food product, a fish-like product, a chicken-like product, a pork-like product and a meat replica. 30. The edible composition according to any of embodiments 20-29, wherein the edible composition is vegan, vegetarian or gluten-free. 31. The edible composition according to any of embodiments 20-30, wherein the edible composition has an appearance of blood derived from the algae preparation. 32. The edible composition according to any of embodiments 20-31, wherein the algae preparation has a heme content greater than the chlorophyll content. 33. The edible composition according to any of embodiments 20-32, wherein the algae preparation has a protoporphyrin IX content greater than the chlorophyll content. 34. The edible composition according to any of embodiments 20-33, wherein the algae preparation provides at least 5%, 10%, 15%, 20%, 25%, 30%, 35%, 40%, 45%, 50%, 55%, 60%, 65%, 70%, 75%, 80%, 85%, 90%, 95%, 99% or 100% of the total protein content to the edible composition. 35. The edible composition according to any of embodiments 20-34, wherein the algae preparation provides vitamin A, beta carotene or a combination thereof to the composition. 36. The edible composition of embodiment 35, wherein the vitamin A, the beta carotene or the combination thereof is at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended requirement. 37. The edible composition according to any of embodiments 20-36, wherein the algae preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the edible composition. 38. The edible composition according to any of embodiments 20-37, wherein the algae preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in a finished product comprising the edible composition. 39. The edible composition according to any of embodiments 20-38, wherein the algae preparation provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the edible composition. 40. The edible composition according to any of embodiments 20-39, wherein the algae preparation has reduced fatty acid content. 41. The edible composition according to any of embodiments 20-40, wherein the edible product is combined with a protein source, a fat source, a carbohydrate, a starch, a thickener, a vitamin, a mineral, or any combination thereof 42. The edible composition of embodiment 41, wherein the protein source is selected from the group consisting of textured wheat protein, textured soy protein and textured pea protein, fungal protein or algal protein. 43. The edible composition of embodiment 41, wherein the fat source comprises at least one of refined coconut oil or sunflower oil. 44. The edible composition of any of embodiments 41-43, further comprising at least one of potato starch, methylcellulose, water, and a flavor, wherein the flavor is selected at least one of yeast extract, garlic powder, onion powder, and salt. 45. The edible composition of any of embodiments 41-44, wherein the edible product is an ingredient for a burger, a sausage, a kebab, a filet, a fish-alternative, a ground meat-like product or a meatball. 46. The edible composition of embodiment 45, wherein the burger comprises about 5% of the algae preparation, about 20% textured soy protein and about 20% refined coconut oil. 47. The edible composition of embodiment 46, further comprising about 3% sunflower oil, about 2% potato starch, about 1% methylcellulose, about 45% water and about 4-9% flavors. 48. The edible composition of embodiment 46, further comprising about 0.5% Kojac gum, about 0.5% Xanthan gum, about 45% water and about 4-9% flavors. 49. The edible composition of embodiment 45, wherein the fish-alternative comprises 20% textured soy protein, about 5% of algae preparation, about 65% water and about 10% flavors. 50. The edible composition according to any of embodiments 20-49, wherein the edible composition is free of animal proteins. 51. The edible composition according to any of embodiments 20-50, wherein the algae preparation comprises an algae having an increase in protoporphyrinogen IX synthesis or accumulation. 52. The edible composition according to any of embodiments 20-51, wherein the algae preparation comprises an algae that exhibits a red or red-like color when grown in the dark conditions. 53. The edible composition according to any of embodiments 20-52, wherein the algae comprised in the algae preparation are recombinant or genetically modified algae. 54. The edible composition according to any of embodiments 20-53, wherein the algae preparation comprises a Chlamydomonas sp. 55. The edible composition of embodiment 54, wherein the Chlamydomonas sp. is Chlamydomonas reinhardtii.

Embodiment 56. A method for the production of an edible composition comprising: (a) culturing an engineered algae according to any of embodiments 1-19 in a condition where the engineered algae exhibits a red or red-like color and wherein the engineered algae produces heme; (b) collecting the cultured engineered algae to produce an algae preparation; and (c) combining the algae preparation with at least one edible ingredient to produce an edible composition. 57. The method of embodiment 56, wherein the condition comprises a fermentation condition. 58. The method according to any of embodiments 56-57, wherein the condition comprises acetate as a reduced carbon source for growth of the engineered algae. 59. The method according to any of embodiments 56-58, wherein the condition comprises sugar as a reduced carbon source for growth of the engineered algae. 60. The method according to any of embodiments 56-59, wherein the condition comprises dark or limited light conditions. 61. The method according to any of embodiments 56-60, wherein the method further comprises fractionating the cultured algae to produce the algae preparation. 62. The method according to any of embodiments 56-61, wherein the algae preparation has a heme content that is greater than the chlorophyll content. 63. The method according to any of embodiments 56-62, wherein the algae preparation has a protoporphyrin IX content that is greater than the chlorophyll content. 64. The method according to any of embodiments 56-63, wherein the condition further comprises iron supplements. 65. The method according to any of embodiments 56-64, wherein the engineered algae is a Chlamydomonas sp. 66. The method of embodiment 65, wherein the engineered algae is a Chlamydomonas reinhardtii. 67. The method according to any of embodiments 56-66, wherein the edible composition has at least one of the features selected from the group consisting of a meat or meat-like flavor, a meat or meat-like texture, a blood-like appearance and a meat or meat-like color, where the at least one of the features is derived from the algae preparation. 68. The method according to any of embodiments 56-67, wherein the method further comprises producing a finished product comprising the edible composition and wherein the finished product is a beef-like food product, a fish-like product, a chicken-like product, a pork-like product or a meat replica. 69. The method according to any of embodiments 56-68, wherein the edible composition is free of animal proteins. 70. The method according to any of embodiments 56-69, wherein the algae preparation is fractionated to remove one or more of starch, protein, PPIX, fatty acids and chlorophyll.

Embodiment 71. A method of making an engineered algae enriched in heme content, comprising: (a) subjecting an algae strain to a process that produces genetic modification to create a first algae population; and (b) from the first algae population, selecting a second algae population that is enriched in heme content, and optionally, PPIX content. 72. The method according to embodiment 71, wherein the process comprises at least one of a random UV mutagenesis, a random chemical mutagenesis, a recombinant genetic engineering, a gene editing, or a gene silencing. 73. The method according to embodiment 71 or embodiment 72, further comprising culturing the first algae population in a fermentation condition. 74. The method according to embodiment 73, wherein the fermentation condition comprises a media having sugar as a sole carbon source. 75. The method according to embodiment 74, wherein the sugar is selected from glucose, dextrose, fructose, maltose, galactose, sucrose, and ribose. 76. The method according to any of embodiments 73-75, wherein the fermentation condition comprises a brightness of less than 500 lux. 77. The method of any of embodiments 73-76, wherein the selecting the second algae population comprises sorting or identifying algae cells having a red or red-like color. 78. The method of any of embodiments 73-77, wherein the selecting is performed by FACS. 79. The method according to any of embodiments 73-78, the second algae population is selected with its capability to grow in the fermentation condition.

### EXAMPLES

### Example 1: Mutagenesis of Algae and Selection of Strains

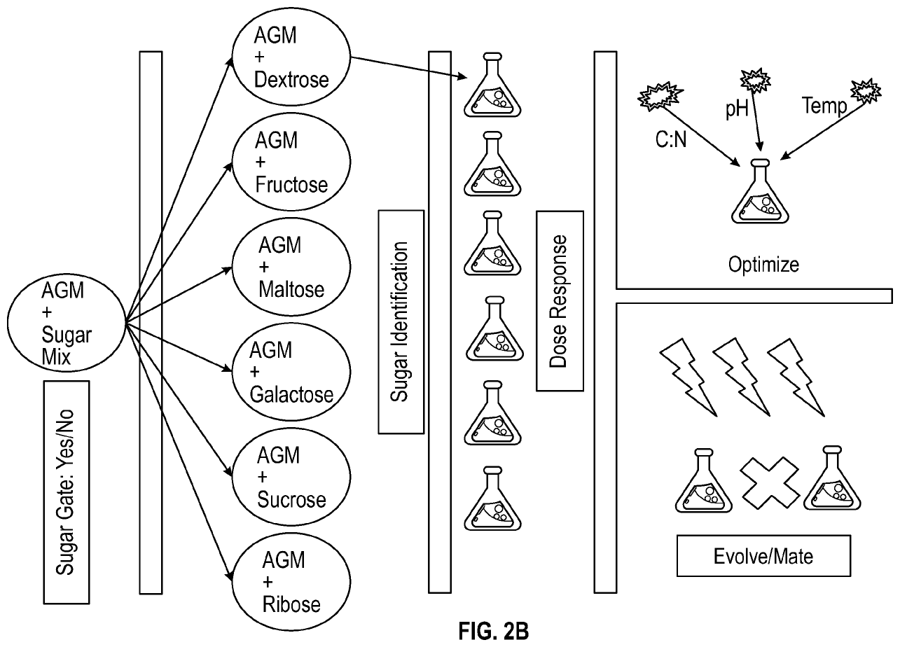

A wildtype strain of algae (Chlamydomonas sp.) was subjected to UV irradiation with an excitation wavelength of 420 nm and an emission of 635 nm. Strains were first selected for their ability to grow on alternatives carbon sources such as glucose. One of these selected strains was further mutagenized using similar conditions to select and/or identify for red-colored strains using fluorescence screening (e.g., Fluorescence-activated cell sorting (FACS)) or magnetic or bead-based cell sorting. These selections are illustrated in FIG. 2 and as further detailed below.

Strains of algae (Chlamydomonas reinhardtii) overexpressing heme were identified by their inability to produce chlorophyll. Additionally, these strains exhibited red, brown, orange or some variation of the listed color. The identified strains exhibit light sensitivity and cannot be grown in direct light greater than 10 μE m⁻² s⁻¹ for extended periods of time.

To generate strains of algae overexpressing heme, green parental strains of Chlamydomonas reinhardtii were placed in a UV-light cross linker and exposed to 25-300 mJ/cm² of UV-light to induce random mutations. Following the exposure to UV-light strains were recovered on agar plates and placed into the dark. Once recovered, the strains were pulled into a flask with growth media and grown placed in a shaker in the dark to limit their potential for exposure to light which could cause many of the heme rich strains to be lost. Flask for cultured for a week in the dark and then applied to a flow cytometer. Cells were excited with a 420 nm light and excitation was measured at 595±15 nm and 635±15 nm. Cells that had a high excitation signal at 595±15 nm were avoided as this the fluorescent signal for Mg-protoporphyrin, a precursor to the formation of chlorophyll. Cells that had a high fluorescent excitation signal at 635±15 nm were sorted into a pulled population as this fluorescent signal is indicative of high protoporphyrin IX. Once pulled, cells were spread on a plate an individual colonies grown and their individual fluorescent characteristics determined by a 96-well plate reader. This process resulted in the identification of 50 strains that had elevated levels of protoporphyrin IX and heme.

One of these red strains was subjected to genomic sequencing at the loci involved in chlorophyll and heme biosynthesis. Sequencing indicated that the genetic modification occurred in the CHLH locus. The sequence of CHLH of the red strain is provided in SEQ ID NO: 129 (nucleotide sequence) and SEQ ID NO: 152 (amino acid sequence). The modification deletes a single base pair in CHLH as compared to a green strain, causing a frameshift in the CHLH open reading frame and/or generate a stop codon such that the protein is translated into a truncated form. The sequence comparison is shown in FIG. 9 (upper sequence (Seq_1) is a partial nucleic acid sequence (residues 1621-1679 of SEQ ID NO: 27) and a partial amino acid sequence (residues 451-460 of SEQ ID NO: 28) of CHLH gene of green algae, and lower sequence (Seq_2) is a partial nucleic acid sequence (residues 1621-1680 of SEQ ID NO: 129) and partial amino acid sequence (residues 451-460 of SEQ ID NO: 152) of CHLH gene of red algae has a mutation (asterisk)). The nucleic acid sequences of additional genes that may be altered in such algae strains are provided herein.

### Example 1A: Identification of Heme Rich Chlamydomonas sp. that Grow on Sugar as their Sole Reduced Carbon Source

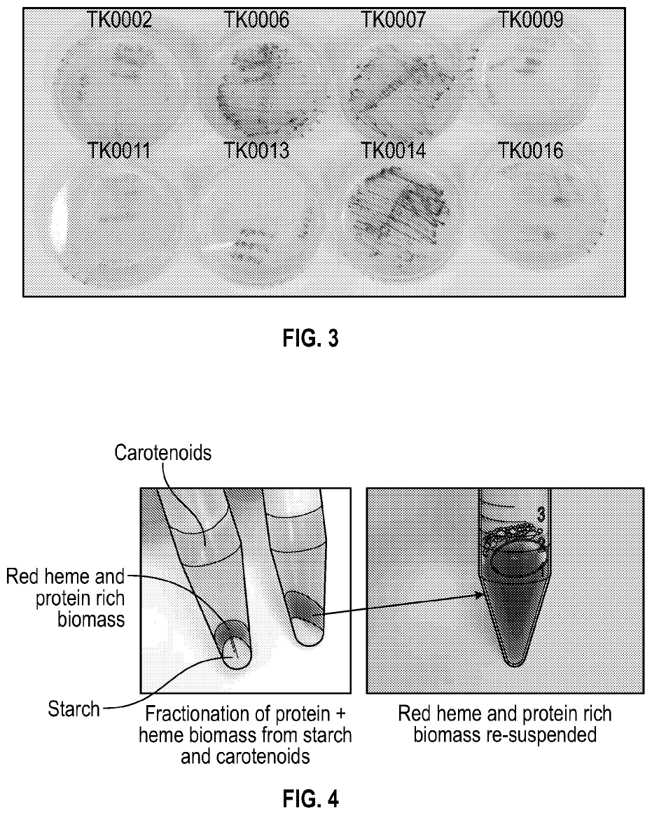

The use of sugar as a carbon source versus acetate has an economic benefit to the cost of production Chlamydomonas algae. To date, no strains of Chlamydomonas reinhardtii have been identified that grow on sugar as a carbon source. Typically, as shown in FIG. 3, Chlamydomonas reinhardtii requires acetate or sunlight and carbon dioxide to grow. Strains of algae from the wild or various culture collection centers were plated on agar growth media with dextrose added at 25 g/L. The plates were then placed in the dark to ensure that photosynthesis could not occur. Cultures were allowed to grow for 2 weeks. At the end of two weeks cultures were studied for their ability to grow in conditions devoid of light. Strains that were capable of growing in the dark with dextrose as their primary carbon source were then placed into shake flasks with growth medium and dextrose at 25 g/L as the primary carbon source and growth for a week in the dark. Culture density and sugar concentration in the media was monitored daily to determine if dextrose was being metabolized by the strains.

Following their identification, Chlamydomonas sp. strains that grew on dextrose as a carbon source were mutagenized using a UV-crosslinker. Cultures were exposed to 25-300 mJ/cm² of UV-light to induce mutations. Following the exposure to UV-light strains were recovered on agar plates and placed into the dark. Once recovered, the strains were pulled into a flask with growth media and grown placed in a shaker in the dark to limit their potential for exposure to light which could cause many of the heme rich strains to be lost. Flask for cultured for a week in the dark and then applied to a flow cytometer. Cells were excited with a 420 nm light and excitation was measured at 595±15 nm and 635±15 nm. Cells that had a high excitation signal at 595±15 nm were avoided as this the fluorescent signal for Mg-protoporphyrin, a precursor to the formation of chlorophyll. Cells that had a high fluorescent excitation signal at 635±15 nm were sorted into a pulled population as this fluorescent signal is indicative of high protoporphyrin IX. Once pulled, cells were spread on a plate an individual colonies grown and their individual fluorescent characteristics determined by a 96-well plate reader. This process resulted in the identification of 20 strains that had elevated levels of protoporphyrin IX and heme and that were still able to grow on dextrose.

Tables 1-5 show characteristic analysis of one exemplary, identified red heme algae (Strain number: TAI114, Species name: Chlamydomonas reinhardtii).

TABLE 1
MICROBIAL ANALYSIS

| Quality Measure | Specification | Result | Units | Method | Conclusion |
|---|---|---|---|---|---|
| Aerobic Plate Count | ≤10,000 | 7,250 | CFU · g−1 | AOAC 990.12 | Specification Met |
| E. coli (Generic) | Negative | Negative | CFU · g−1 | AOAC 991.14 | Specification Met |
| Total coliforms | ≤1,000 | Negative | CFU · g−1 | AOAC 991.14 | Specification Met |
| Salmonella | Negative | Negative | ORG · 25 g | AOAC 030301 | Specification Met |
| Staphylococcus | Negative | Negative | CFU · g−1 | AOAC2003.07 | Specification Met |
| aureus | | | | | |
| Pseudomonas | Negative | Negative | CFU · g−1 | USP | Specification Met |
| aeruginosa | | | | | |

TABLE 2
HEAVY METAL ANALYSIS

| Quality Measure | Specification | | Result | Units | Method | Conclusion |
|---|---|---|---|---|---|---|
| Arsenic | ≤0.01 | ppm | ≤0.01 ppm | ppm | MET-CH-030 | Specification Met |
| Cadmium | ≤0.1 | ppm | ≤0.01 ppm | ppm | MET-CH-030 | Specification Met |
| Lead | ≤0.01 | ppm | ≤0.01 ppm | ppm | MET-CH-030 | Specification Met |
| Mercury | ≤0.005 | ppm | ≤0.01 ppm | ppm | MET-CH-030 | Specification Met |
| Sulfite | ≤10 | ppm | ≤0.01 ppm | ppm | MET-NHP-018 | Specification Met |

TABLE 3
BIOMASS ANALYSIS

| Quality Measure | Result | Unit |
|---|---|---|
| Moisture | 10.66 | Percent of biomass |
| Ash | 3.19 | Percent of biomass |
| Protein | 26.00 | Percent of biomass |
| Fat | 4.77 | Percent of biomass |
| Starch | 39.5 | Percent of biomass |
| Soluble Dietary Fiber | 8.85 | Percent of biomass |
| Insoluble Dietary Fiber | 1.15 | Percent of biomass |

TABLE 4
Porphyrin (Heme) ANALYSIS

| Quality Measure | Result | Unit |
|---|---|---|
| Heme | 0.60 | Percent |
| protoporphyrin IX | 4.60 | Percent |

TABLE 5
AMINO ACID COMPOSITION

| Amino Acid | Result | Unit |
|---|---|---|
| Alanine | 2.25 | Percent of biomass |
| Arginine | 2.03 | Percent of biomass |
| Asparagine/Aspartic Acid | 2.38 | Percent of biomass |
| Glycine | 1.49 | Percent of biomass |
| Cysteine | 0.48 | Percent of biomass |
| Glutamine/glutamic acid | 2.83 | Percent of biomass |
| Proline | 1.63 | Percent of biomass |
| Serine | 1.25 | Percent of biomass |
| Tyrosine | 1.05 | Percent of biomass |
| Histidine | 0.51 | Percent of biomass |
| Isoleucine | 1.04 | Percent of biomass |
| Leucine | 2.38 | Percent of biomass |
| Lysine | 1.78 | Percent of biomass |
| Methionine | 0.63 | Percent of biomass |
| Phenylalanine | 1.15 | Percent of biomass |
| Threonine | 0.83 | Percent of biomass |
| Tryptophan | 0.55 | Percent of biomass |
| Valine | 1.88 | Percent of biomass |
| Percent Non-Essential Amino Acids | 51.1 | Percent of protein |
| Percent Amino Acids | 48.9 | Percent of protein |

### Example 1B: Identification of Heme-Overproducing Algae

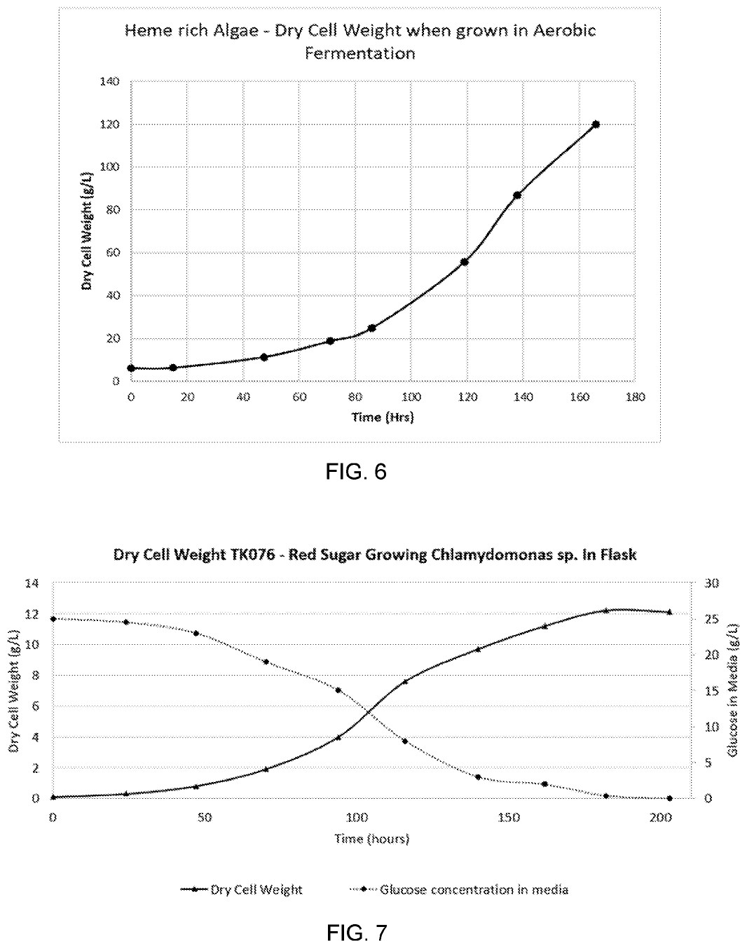

One of the identified strains was grown under fed-batch aerobic fermentation conditions where acetate is used as a reduced carbon source of nutrition for the culture. The strain was grown in a fermenter where minimal light can reach the culture. The strain was grown to a density that is greater than 120 g/L and harvested via centrifugation. The harvested strain is red in color and can be added to compositions, such as food products, to confer a red, orange or brown color. FIG. 6 is a graph showing the cell weight of the heme overproducer strain grown in aerobic fermentation conditions.

### Example 1C: High Density Growth of Heme-Overproducing Algae

Strains of Chlamydomonas that were previously selected for their ability to overexpress heme were grown to high density. To do this, a basal media containing media components that would allow the culture to achieve 120 grams per liter was developed. The strains are fresh water algae as such media components when solubilized with water were made not to exceed 10 mS/cm. Cultures were then grown using an aerobic fed-batch fermentation process. Cultures were fed with a media containing acetate as a carbon source, ammonium hydroxide as a nitrogen source, and phosphoric acid as a phosphate source. Cultures were fed using a one sided acid pH-stat to maintain the pH at 6.8. As shown in FIG. 6, cultures were allowed to grow for 7 day and titers of 120 g/L of biomass were achieved. Heme and protoporphyrin IX was quantified by using a heme quantification assay (Abnova KA1617). Heme and protoporphyrin were found to be greater than 5% of the biomass by weight. Titers of greater than 1 g/L of heme and protoporphyrin IX were achieved. In short, heme/protoporphyrin IX were extracted from a defined amount of algae culture by mixing the algae culture with a solution of 1.7M HCL and 80% Acetone. The mixture was allowed to sit for 30 minutes. After 30 minutes samples were centrifuge to separate the heme/protoporphyrin IX extract from the algal biomass. The soluble heme/protoporphyrin IX samples were used in the assay from Abnova and compared to a standard curve to determine the amount of heme/protoporphyrin IX in the algal biomass.

### Example 2: Fractionation

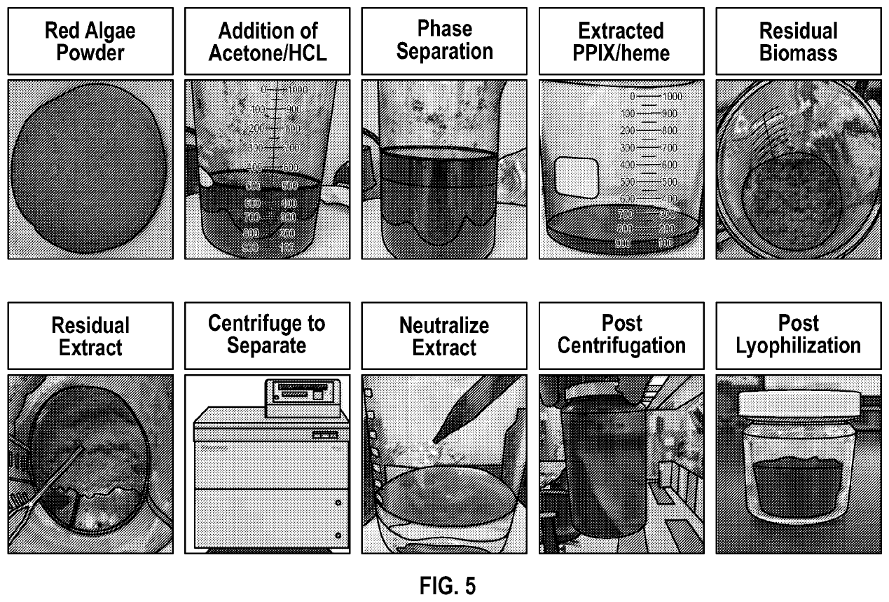

Cells from a heme overproducing strain of Chlamydomonas reinhardtii were harvested from a fermentation culture. The harvested cells were disrupted by sonication and then the samples were separated by centrifugation at 10.000×G. This separated the samples into a carotenoid, starch and protein/heme biomass fractions. The protein/heme biomass was then re-suspended in Phosphate buffered saline pH 7.4. Shown in FIG. 4 is the fractionation following centrifugation (left) and the resuspension of the heme-containing fraction (right). Also shown in FIG. 5 illustrates process of PPIX and heme fractionation process and/or process of generating biomass, extracts, and/or lypophilized products.

### Example 3: Characterization of Heme Production

A number of heme assays can be used to determine the concentration of heme. In one example, the amount of heme can be quantitatively determined by mixing the algae biomass into an aqueous alkaline solution causing the heme to be converted into a uniform color. The intensity of the color can be measured by the absorbance at 400 nm which is directly proportional to the heme concentration in the sample. These measurements can then be compared to standards generated by heme at known concentrations to determine the amount of heme in algae samples.

### Example 4: Preparation of a Heme-Enriched “Meatless” Burger

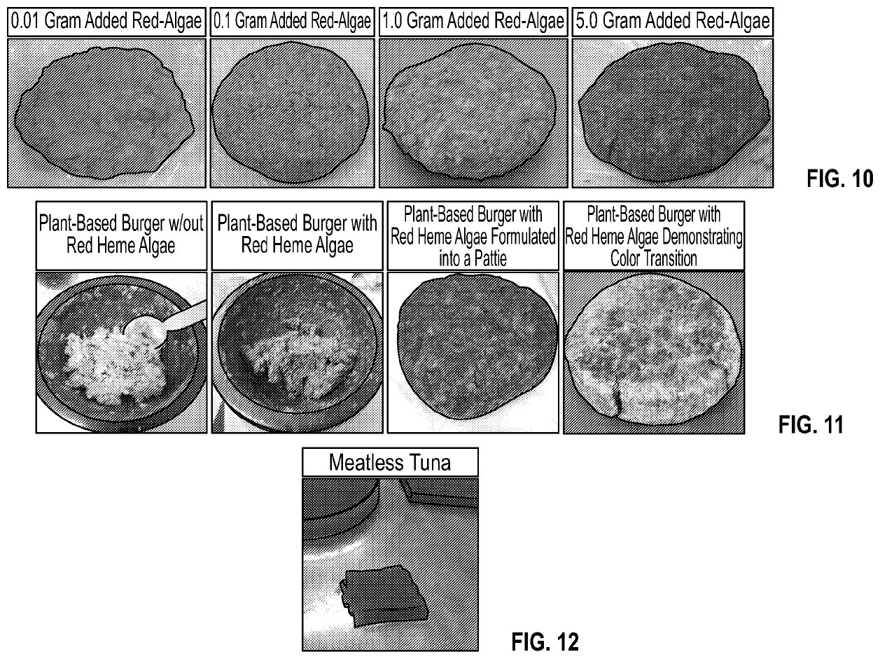

The heme-enriched samples can be used to prepare compositions of meat-like products produced from plant based materials and algae rich in heme. To create a heme-enriched burger, ingredients were mixed in the following proportions and formed into a disc shaped algae-plant based burger: 20% or about 20% Textured wheat protein, 20% or about 20% Refined coconut oil, 3% or about 3% Sunflower oil, 2% or about 2% Potato starch, 0.5% or about 0.5% Kojac gum, 0.5% or about 0.5% Xanthan gum, 45% or about 20% water and 4-9% or about 4-9% Flavors, including yeast extract, garlic powder, onion powder, salt, and heme-enriched (“red”) algae. Shown in FIG. 10 are burgers created with 0.01 g, 0.1 g, 1.0 g, and 5.0 g of the heme enriched algae.

In this example, the composition of the heme-enriched algae was 4.5% protoporphyrin IX, 0.5% heme, 0% chlorophyll, 24.4% protein, 9% dietary fiber, 40% starch, 0.8% omega-3-fatty acids, 3.9% other fats, 7.5% moisture, and 8.4% ash.

### Example 5: Preparation of a Heme-Enriched Plant-Based Burger

The heme-enriched samples can be used to prepare burger compositions from plant based materials and algae rich in heme. To create a heme-enriched plant-based burger, ingredients were mixed in the following proportions and formed into a disc: 20% or about 20% Textured soy protein, 20% or about 20% Refined coconut oil, 3% or about 3% Sunflower oil, 2% or about 2% Potato starch, 1% or about 1% methylcellulose, 45% or about 45% water and 4-9% or about 4-9% Flavors, including yeast extract, garlic powder, onion powder, salt, and heme-enriched (“red”) algae. Shown in FIG. 11 are the ingredient mixes of the plant-based burger ingredients with no heme-enriched algae (far left), with the addition of heme-enriched algae (second from left), the ingredients with the addition of heme-enriched algae shaped into a burger before and after cooking (thirds from left and far right photos, respectively). As shown, the addition of the heme-enriched algae confers a red/red-like color (resembling a burger with animal blood) to the ingredient mix and to the burger, and this color undergoes a transition when cooked.

In this example, the composition of the heme-enriched algae was 4.5% protoporphyrin IX, 0.5% heme, 0% chlorophyll, 24.4% protein, 9% dietary fiber, 40% starch, 0.8% omega-3-fatty acids, 3.9% other fats, 7.5% moisture, and 8.4% ash.

### Example 6: Preparation of a Heme-Enriched Meatless “Tuna”

The heme-enriched samples can be used to prepare fish-like compositions, as shown in FIG. 12. To create a heme-enriched meatless “fish”, ingredients were mixed in the following proportions: 20% or about 20% Textured soy protein, 65% or about 65% water and 10% or about 10% Flavors and 5% or about 5% heme-enriched (“red”) algae. Shown in FIG. 12 is a square portion of the meatless “tuna.”

In this example, the composition of the heme-enriched algae was 4.5% protoporphyrin IX, 0.5% heme, 0% chlorophyll, 24.4% protein, 9% dietary fiber, 40% starch, 0.8% omega-3-fatty acids, 3.9% other fats, 7.5% moisture, and 8.4% ash.

### Example 7: Growth of Heme-Enriched Algae Strain on Glucose

A heme-enriched algae strain was grown in a media with glucose as the sole carbon source. Briefly, as shown in FIG. 2, media was prepared in water, providing per liter of total volume 25 g anhydrous glucose, 5 g KNO₃, 0.5275 g KH₂PO₄, 0.3925 g MgSO₄*7H₂O, 0.031275 g FeSO₄*7 H₂O, 0.007125 g H₃BO₃, 0.002 CuSO₄, 0.002775 g ZnSO₄, 0.002425 g CoSO₄, 0.00325 g MnCl₂*4H₂O, 0.00115 g (NH₄)₆Mo₇O₂₄*4H₂O, and 0.01735 g CaCl. The media was adjusted to pH 7.0, autoclaved and had a final pH between 5.5 to 6.5 The algae strain was inoculated at a density of about 0.1 g/L.

The culture was placed in a dark incubator (devoid of light) and grown at 30° C. on a rotating shaker platform. Culture density (measured by dry cell weight) and residual glucose concentration in the media were measured daily. FIG. 7 shows the increase in dry cell weight over time and a concomitant decrease in residual glucose in the media. Dry cell weight in this experiment reached over 25 g/L dry cell weight.

### Example 8: Extraction of Heme Fraction from Whole Biomass

Using the heme-enriched algae (grown similarly to Example 1), a heme-enriched fraction was prepared. Approximately 100 g of algae biomass was mixed with a 1.0 L of a solution containing 80% acetone and 20% 1.7M HCL for 30 minutes. The biomass was allowed to settle and then the aqueous layer was extracted (containing heme and protoporphyrin IX) away from the solid into new container. Centrifugation was applied to the extracted aqueous layer or in some experiments, the sample was filtered with a filter having a molecular cutoff of 0.4 um. The resulting aqueous fraction was neutralized with 10M NaOH, Then water was added at 100 ml per 100 ml of sample. Following this mixture, the heme and protoporphyrin IX became insoluble and fell out of solution. The solution was then centrifuged to collect the solids (containing the hem and protoporphyrin IX) and dried to form a red powder. FIG. 5 shows the red-like colored fractions (containing the heme and protoporphyrin IX) collected through the steps of the procedure. From 160 g of red algae biomass, 7.7 g of PPIX/heme was extracted.

### Example 9: Removal of Fatty Acids from Algae Biomass to Enrich for Heme

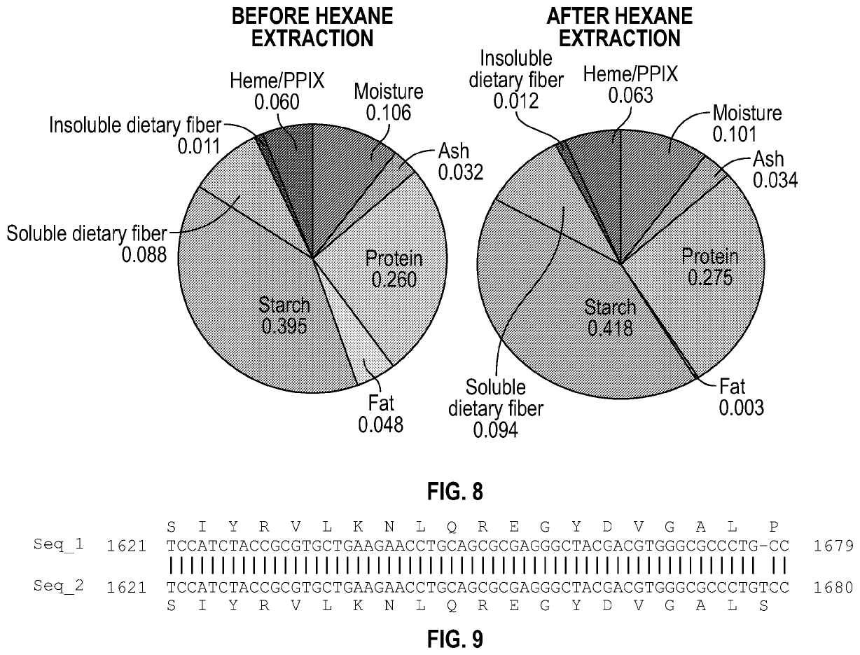

Dry Chlamydomonas cells were mixed together with water ethanol and hexane in a ratio of 6:77:17. Samples were allowed to separate for 4 hours. The aqueous layer containing the fatty acids was then removed. The sample was then centrifuged to full separate the solid biomass layer from any remaining fatty acids. The biomass was then dried prior to further analysis. FIG. 8 shows a biochemical analysis of the algae biomass before and after the fatty acid extraction, demonstrating a greater than 10-fold reduction in fatty acid content after the extraction procedure.

### Example 10: Targeted Modification of Chlorophyll Pathway to Create Heme-Enriched Strains

Guide RNAs (sgRNAs) can be designed against any of the sub-units of the magnesium chelatase gene to cause a deletion or an insertion that renders the protein complex non-functional. Once designed sgRNAs can be combined with the Cas9 protein by incubating them at 37° C. to form ribonuclear proteins (RNPs). These RNPs carrying the sgRNAs to target magnesium chelatase are then electroporated into green algae cultures. 3×10⁸ cells are placed into MAX efficiency transformation buffer reagent for algae (Thermo fisher scientific) and placed into a cuvette with a 0.2 cm gap. The electroporation voltage is set to 250V and the pulse interval is set to 15 ms. Once electroporated cells are recovered in growth media with 40 mM sucrose added to improve recovery efficiency. Cells are then plated on growth media containing agar and grown in the dark due to the photosensitivity of the magnesium chelatase mutants. Once recovered the population can be pulled and struck out for individual colonies. Plates are again placed in the dark for 2 to 3 weeks. Mutants of Mg-chelatase can be identified by eye as they are not green. Mutants are then sequenced to ensure that target mutation was introduced.

### Example 11: Modification of Chlorophyll Pathway to Create Heme-Enriched Strains that are Improved for Different Meat Imitations

Strains of algae that increase the precursors to heme such as aminolevulinic acid can be mated to strains that are overexpressing heme to further increase the amount of heme or protoporphyrin IX that are produced. Mating can be done by identifying strains of Chlamydomonas that are the opposite mating type and then starving them for nitrogen. After nitrogen starvation, strains are re-suspended in water to promote the formation of flagella. The flagella of the different mating types assist in the fusion of algae strains that will result in the formation of a zygote. The mated cultures are then exposed to chloroform to kill strains that did not mate. The chloroform does not kill zygotes. The zygotes are then placed into growth medium and allowed to propagate. Individual colonies are then identified and screened for an increase in heme by measuring for an increase in fluorescence of the precursor protoporphyrin IX or by biochemical assay (Abnova KA1617).

Strains of algae overexpressing heme can also by mated with strains that are under or overproducing omega-3s, omega-6s or omega-9s. For imitation fish, more omega oils in strains of algae overexpressing heme are ideal. For imitation beef-like products, less omega oils in strains of algae overexpressing heme are ideal. As such strains of algae that are mutants for either over or underexpressing omega oils can be mated with strains of algae overexpressing heme to form a more ideal algae for various meat-like products.

SEQUENCES
ALA dehydratase (ALAD) nucleic acid sequence (SEQ ID NO: 1):
atgcagatgatgcagcgcaacgttgtgggccagcgccccgtcgctggctcccgccgctcgctggtggttgccaac
gttgcggaggtgacccgccccgcggtcagcaccaacggcaagcaccggactggtgtgccggagggaactcccatc
gtcacccctcaggacctgccctcgcgccctcgccgcaaccgccgcagcgagagcttccgtgcttccgttcgtgag
gtgaacgtgtcgcccgccaacttcatcctgccgatcttcatccacgaggagagcaaccagaacgtgcccatcgcc
tccatgcctggcatcaaccgcctggcgtatggcaagaacgtgattgactacgttgctgaggctcgctcttacggt
gtcaaccaggtcgtggttttccccaagacgcccgaccacctgaagacgcaaaccgcggaggaggcgttcaacaag
aacggcctcagccagcgcacgatccgcctgctgaaggactctttccctgacctggaggtgtacacggacgtggct
ctggacccctacaactcggacggccacgacggtatcgtgtcggacgccggtgtgatcctgaacgacgagaccatc
gagtacctgtgccgccaggccgtgagccaggccgaggccggtgccgacgtggtgtcgccctctgacatgatggac
ggccgcgtgggcgccatccgccgcgccctggaccgcgagggcttcaccaacgtgtccatcatgtcctacaccgcc
aagtacgcctccgcctactacggccccttccgtgacgccctggcgtccgcgcccaagcccggccaggcgcaccgc
cgcatcccccccaacaagaagacctaccagatggaccccgccaactaccgcgaggccatccgcgaggccaaggcc
gacgaggccgagggcgctgacatcatgatggtcaagcccggcatgccgtacctggacgtggtacgcctgctgcgt
gagaccagcccgctgcccgtggccgtgtaccacgtgtcgggcgagtacgccatgctcaaggcggcggcggagcgc
ggctggctgaacgagaaggatgccgtgcttgaggccatgacctgcttccgccgcgccggcgctgacctcatcctc
acctactacggcattgaggcctccaagtggctggcgggcgagaagtaa
ALA dehydratase (ALAD) amino acid sequence (SEQ ID NO: 2):
MQMMQRNVVGQRPVAGSRRSLVVANVAEVTRPAVSTNGKHRTGVPEGTPIVTPQDLPSRPRRNRRSESFRASVRE
VNVSPANFILPIFIHEESNQNVPIASMPGINRLAYGKNVIDYVAEARSYGVNQVVVFPKTPDHLKTQTAEEAFNK
NGLSQRTIRLLKDSFPDLEVYTDVALDPYNSDGHDGIVSDAGVILNDETIEYLCRQAVSQAEAGADVVSPSDMMD
GRVGAIRRALDREGFTNVSIMSYTAKYASAYYGPFRDALASAPKPGQAHRRIPPNKKTYQMDPANYREAIREAKA
DEAEGADIMMVKPGMPYLDVVRLLRETSPLPVAVYHVSGEYAMLKAAAERGWLNEKDAVLEAMTCFRRAGADLIL
TYYGIEASKWLAGEK
coproporphyrinogen III oxidase (CPX1) nucleic acid sequence (SEQ ID NO: 3):
atggcactgcaagcctcaacccgctcgctccagcagcgccgcgccttctcttcggcccagacctccaagcgtgtg
tctgtgaccaaggtccgcgcgacggctatcgaggcggagaactatgtgaagcaggctccccagtcgctggtccgc
ccgggcatcgacactgaggactctatgcgcgctcgcttcgagaaggtgatccgcaacgcccaggactccatctgc
aatgctatctccgagatcgatggcaagccgttccaccaggacgcctggacccgccccggcggcggtggcggcatc
agccgcgtgctgcaggacggcaacgtgtgggagaaggccggcgtcaacgtgtccgtggtctacggcaccatgccc
cctgaggcctaccgcgctgccactggcaacgccgagaagctgaagaacaagggtgacggtggccgcgtgcccttc
ttcgccgccggcatctcgtcggtgatgcacccccgcaacccccactgccccaccatgcacttcaactaccgctac
ttcgagactgaggagtggaacggcatccccggccagtggtggttcggcggcggcaccgacatcacccccagctat
gtggtgcccgaggacatgaagcacttccacggcacctacaaggcggtgtgcgaccgccacgatcccgcttactac
gagaagttccgcacctggtgcgatgagtacttcctcatcaagcaccgcggcgagcgccgcggcctgggcggcatc
ttcttcgatgacctgaacgaccgcaaccccgaggacatcctgaagttctcgaccgacgccgtgaacaacgtggtg
gaggcatactgccccatcatcaagaagcacatgaacgacccctacacccccgaggagaaggagtggcagcagatc
cgccgcggccgctacgtggagttcaacctggtctatgaccgcggcaccaccttcggcctgaagaccggcggccgc
attgagtcgatcctcatgtccatgccccagaccgcctcatggctgtacgaccaccagcccaaggccggctcgccc
gaggccgagctgctcgacgcctgccgcaacccccgcgtctgggtgtaa
coproporphyrinogen III oxidase (CPX1) amino acid sequence (SEQ ID NO: 4):
MALQASTRSLQQRRAFSSAQTSKRVSVTKVRATAIEAENYVKQAPQSLVRPGIDTEDSMRARFEKVIRNAQDSIC
NAISEIDGKPFHQDAWTRPGGGGGISRVLQDGNVWEKAGVNVSVVYGTMPPEAYRAATGNAEKLKNKGDGGRVPF
FAAGISSVMHPRNPHCPTMHFNYRYFETEEWNGIPGQWWFGGGTDITPSYVVPEDMKHFHGTYKAVCDRHDPAYY
EKERTWCDEYFLIKHRGERRGLGGIFFDDLNDRNPEDILKFSTDAVNNVVEAYCPIIKKHMNDPYTPEEKEWQQI
RRGRYVEFNLVYDRGTTFGLKTGGRIESILMSMPQTASWLYDHQPKAGSPEAELLDACRNPRVWV
coproporphyrinogen III oxidase (CPX2) nucleic acid sequence (SEQ ID NO: 5):
atgctgaggaagcagattggtggatctggccagcagcgggcgggcctccgacgggtgaaccaaggacctgcgcgt
cggcggttggcaccctgccgcgtggcggcccccgtgcaaacctcgtcctccgtcgccacattcaatggcttcgtg
gactacattcacggactccagaagaacattctgagcactgctgaggatctggagaacggcgagcggaagtttgtt
gttgaccgctgggagcgcgacgccagcaaccccaacgccgggtatggcattacgtgcgtgcttgaggacgggaag
gtgctggagaaggccgcagccaatatctcagtggtgcgcgggacgctgtcggcgcagcgcgcagtggccatgagc
tcccgcggccgcagcagcatcgaccccaagggcgggcagccctacgccgcggccgccatgagcctagtgttccac
agcgcgcacccgctcatccccacgctgcgcgcgacgtgcggttgttccaggtgggcgatgaggcgtggtacggcg
gtggctgtgacctgacgcccaactacctagacgtggaggactcgcagtccttccaccgctactggaaggacgtgt
gcggcaagtacaagccgggcctgtacaccgagctcaaggagtggtgcgacaggtacttctacatcccggcccgca
aagagcaccgtggcattggcggcctgttctttgatgacatggccactgcggaggcgggctgcgatgtggaggcgt
ttgtgcgggaagtgggagatggcatcctgccctgctggctgcccatcgtggcgcggcaccgtggccagcccttca
cggagcagcagcggcaatggcagctgctgcgccgcggtcgctacatcgagttcaacctgctgtacgaccgcggca
tcaagttcggtctggacggcggccgcatcgagagcatcatggtgtcggcgccgccgctgatcgcgtggaagtaca
acgtggtgccacagccgggcagccccgaggaggagatgctgaaggtgcttcagcagccccgcgagtgggcctga
coproporphyrinogen III oxidase (CPX2) amino acid sequence (SEQ ID NO: 6):
MLRKQIGGSGQQRAGLRRVNQGPARRRLAPCRVAAPVQTSSSVATFNGFVDYIHGLQKNILSTAEDLENGERKFV
VDRWERDASNPNAGYGITCVLEDGKVLEKAAANISVVRGTLSAQRAVAMSSRGRSSIDPKGGQPYAAAAMSLVFH
SAHPLIPTLRADVRLFQVGDEAWYGGGCDLTPNYLDVEDSQSFHRYWKDVCGKYKPGLYTELKEWCDRYFYIPAR
KEHRGIGGLFFDDMATAEAGCDVEAFVREVGDGILPCWLPIVARHRGQPFTEQQRQWQLLRRGRYIEFNLLYDRG
IKFGLDGGRIESIMVSAPPLIAWKYNVVPQPGSPEEEMLKVLQQPREWA
Ferrochelatase from Chlamydomonas reinhardtii nucleic acid sequence (SEQ ID NO: 7):
atggcgtcgtttggattgatgcaaaggacggtgcactgtccccagcttgtggaggagcggtgttcgccggtcgct
ggctgctctggtcgtggcctgccagttatccagcggcaacggcgtggcgtgtgcagtgccaccaacggtgtccag
cgagggcgtgtgctgcgccggacggccgcttcgaccgacgtggtctccttcgtggaccccaatgacattagaaaa
cccgcagcagcagcagctggccctgcggtggataaggtcggcgttctgctgttaaaccttggcgggcccgaaaag
ctcgacgacgtcaagcctttcctgtataacctattcgccgacccagaaattattcgcctgccagcggcagctcag
ttcctgcagccgctgctcgcgacgatcatctccacgcttcgcgccccgaagagcgcggagggctatgaggccatt
ggcggtggtagcccgttgcgtaggattacagacgagcaggcggaggcgctggcggagtctctgcgcgccaagggc
caacctgcgaacgtgtacgtgggcatgcgctattggcacccctacacggaggaggcgctggagcacattaaggcc
gacggcgtcacgcgcctggtcatcctcccgctgtaccctcagttctccatctctaccagcggctccagccttcga
ctgcttgagtcgctcttcaagagcgacatcgcgctcaagtcgctgcggcacacggtcatcccgtcctggtaccag
cggcggggctacgtgagcgcgatggcggacctgattgtagaggagctgaagaagttccgggacgtgcccagcgtg
gagctgtttttctccgcgcacggcgtgcccaagtcctacgtggaggaggcgggcgacccatacaaggaggagatg
gaggagtgcgtgcggctcattacggacgaggtcaagcggcgcggcttcgccaacacgcacacgctggcctaccag
agccgcgtgggccccgcggaatggctcaagccgtacacggatgagtccatcaaggagctgggcaagcgcggcgtc
aagtcgctgctggcggtgcccatcagctttgtcagcgagcacattgagacgttggaggagatcgacatggagtac
cgcgagctggcggaggagagcggcatccgcaactggggccgcgtgccggcgctgaacaccaacgccgccttcatc
gacgacctggcggacgcggtgatggaggcgctgccctacgtgggctgcctggccgggccgacagactcgctggtg
ccgctgggcgacctggagatgctgctgcaggcctacgaccgcgagcgccgcacgctgccgtcaccggtggtgatg
tgggagtggggctggaccaagagcgcggagacgtggaacggccgcattgccatgattgccatcatcatcatcctg
gcgctggaggcagccagcggccagtccatcctcaaaaacctgttcctggcggagtag
Ferrochelatase from Chlamydomonas reinhardtii amino acid sequence (SEQ ID NO: 8):
MASFGLMQRTVHCPQLVEERCSPVAGCSGRGLPVIQRQRRGVCSATNGVQRGRVLRRTAASTDVVSFVDPNDIRK
PAAAAAGPAVDKVGVLLLNLGGPEKLDDVKPFLYNLFADPEIIRLPAAAQFLQPLLATIISTLRAPKSAEGYEAI
GGGSPLRRITDEQAEALAESLRAKGQPANVYVGMRYWHPYTEEALEHIKADGVTRLVILPLYPQFSISTSGSSLR
LLESLFKSDIALKSLRHTVIPSWYQRRGYVSAMADLIVEELKKFRDVPSVELFFSAHGVPKSYVEEAGDPYKEEM
EECVRLITDEVKRRGFANTHTLAYQSRVGPAEWLKPYTDESIKELGKRGVKSLLAVPISFVSEHIETLEEIDMEY
RELAEESGIRNWGRVPALNTNAAFIDDLADAVMEALPYVGCLAGPTDSLVPLGDLEMLLQAYDRERRTLPSPVVW
EWGWTKSAETWNGRIAMIAIIIILALEAASGQSILKNLFLAE
Glutamate-1-semialdehyde aminotransferase (GSA) nucleic acid sequence (SEQ ID NO:
9):
atgcagatgcagctgaacgccaagaccgtgcagggcgccttcaaggcgcagcgccctcgctctgtccgcggcaac
gtggcggtgcgcgcagtggccgctccccctaagctggtcaccaagcgctccgaggagatcttcaaggaggctcag
gagctgctgcccggtggcgtgaactcgcccgtgcgcgctttccgctcggttggtggcggccccatcgtcttcgac
agggtcaagggtgcctactgctgggacgtcgatggcaacaagtacatcgactacgttggctcttggggccctgcc
atttgcggccacggcaacgacgaggtcaacaacgccctgaaggcgcagatcgacaagggcacctcgttcggtgct
ccctgcgagctggagaacgtgctggccaagatggtgattgaccgcgtgccctcggtggagatggtgcgcttcgtg
tcctcgggcactgaggcgtgcctgtcggtgctgcgcctgatgcgcgcatacaccggccgcgagaaggtgctgaag
ttcaccggctgctaccacggccacgccgactccttcctggtgaaggccggctccggtgtgatcaccctgggcctg
cccgactcgcccggtgtgcccaagagcaccgccgccgccaccctgaccgccacctacaacaacctggactccgtg
cgcgagctgttcgccgccaacaagggcgagattgccggtgtgatcctggagcccgtggtcggcaacagcggcttc
attgtgcccaccaaggagttcctgcagggcctgcgcgagatctgcacggctgagggcgccgtgctgtgcttcgat
gaggtcatgaccggcttccgcattgccaagggctgcgcccaggagcacttcggtatcacccccgacctgaccacc
atgggcaaggtcattggtggcggcatgcctgtgggcgcctacggcggcaagaaggagatcatgaagatggtcgcc
cccgccggccccatgtaccaggccggcaccctttcgggcaaccccatggccatgactgccggcatcaagacgctg
gagatcctgggccgccccggcgcctacgagcacctggagaaggtgaccaagcgcctgatcgacggcatcatggcc
gccgccaaggagcacagccacgagatcaccggcggcaacatcagcggcatgtttggcttcttcttctgcaagggc
cctgtgacctgcttcgaggacgccctggcggccgacactgccaagttcgcgcgcttccaccgcggcatgctggag
gagggcgtctacctggctccctcgcagttcgaggccggcttcacctctctggcccactccgaggcggacgtggat
gccacgatcgccgccgctcgccgcgtgttcgcccgcatctaa
Glutamate-1-semialdehyde aminotransferase (GSA) amino acid sequence (SEQ ID NO:
10):
MQMQLNAKTVQGAFKAQRPRSVRGNVAVRAVAAPPKLVTKRSEEIFKEAQELLPGGVNSPVRAFRSVGGGPIVFD
RVKGAYCWDVDGNKYIDYVGSWGPAICGHGNDEVNNALKAQIDKGTSFGAPCELENVLAKMVIDRVPSVEMVRFV
SSGTEACLSVLRLMRAYTGREKVLKFTGCYHGHADSFLVKAGSGVITLGLPDSPGVPKSTAAATLTATYNNLDSV
RELFAANKGEIAGVILEPVVGNSGFIVPTKEFLQGLREICTAEGAVLCFDEVMTGFRIAKGCAQEHFGITPDLTT
MGKVIGGGMPVGAYGGKKEIMKMVAPAGPMYQAGTLSGNPMAMTAGIKTLEILGRPGAYEHLEKVTKRLIDGIMA
AAKEHSHEITGGNISGMFGEFECKGPVTCFEDALAADTAKFAREHRGMLEEGVYLAPSQFEAGFTSLAHSEADVD
ATIAAARRVFARI
glutamyl-trna reductase (HEMA) nucleic acid sequence (SEQ ID NO: 11):
atgcagaccactatgcagcagcgtctccagggccgtaacgtggccgggcggagcgtcgctccctcggtccctgcc
catcgctccttccactcacaccgggctgccactcaaaccgctacgatcagcgctgctgctagctcaaccaccaag
ctgccagcttcgcatctggagagcagcaagaaggcgctggattcgctgaagcagcaggccgtcaatcgctacgcg
ggtgacaagaagagctccattattgccattggtctcaccattcacaacgcacccgtggagctgcgcgagaagctg
gctgtgcctgaggctgaatggccgcgtgctattgaggagctctgccagttcccgcacatcgaggaggccgcggtg
ctgtcgacgtgcaatcgcatggagctctacgttgtcggtctgtcgtggcaccgcggcgttcgcgaggtggaggag
tggctgtctcgcaccagcggcgtgcctctggatgagctgcgcccctacctgttcctgctgcgcgaccgcgacgcc
acgcaccacctgatgcgcgtgtcgggtggccttgactcgctggttatgggcgagggccagattctcgcccaagtg
cgccaggtctacaaggtcggccagaactgccccggcttcggtcgccacctgaacggcctgttcaagcaggctatc
accgctggcaagcgcgtgcgtgccgagacctccatctccaccggctccgtctccgtctcatccgccgccgtcgag
ctggcgcagctcaagctccccacccacaactggtccgacgctaaggtctgcatcatcggcgctggcaagatgtct
acgctgctggtgaagcacctgcagagcaagggctgcaaggaggtgacggtgctcaaccgctctctgccgcgcgcc
caggcgctggcggaggagttccctgaggtcaagttcaacatccacctgatgcccgacctgctgcagtgcgtggag
gccagcgacgtcatcttcgccgcctccggctctgaggagatcctcatccacaaggagcatgtcgaggccatgtcc
aagccatcggacgttgttggctccaagcgccgcttcgtcgacatctccgtgccccgcaacatcgcccccgccatc
aacgagctggagcacggcatcgtctacaacgtcgacgacctgaaggaggttgtggccgccaacaaggagggccgc
gcgcaggcggccgccgaggccgaggtgctgatccgcgaggagcagcgcgcgttcgaggcctggcgtgactctctg
gagaccgtgcccaccatcaaggcgctgcgctccaaggccgagaccatccgcgccgccgagtttgagaaggccgtg
tctcgcctgggcgaggggctatccaagaagcagctcaaggcggtggaggagctcagcaagggcatcgtcaacaag
ctgctgcacgggcccatgacggcactgcgctgcgacggcaccgatccggatgccgtgggccagaccctcgcgaac
atggaggccctggagcgcatgttccagctctcggaggtggacgtggccgcgctggcgggcaagcagtaa
glutamyl-trna reductase (HEMA) amino acid sequence (SEQ ID NO: 12):
MQTTMQQRLQGRNVAGRSVAPSVPAHRSFHSHRAATQTATISAAASSTTKLPASHLESSKKALDSLKQQAVNRYA
GDKKSSIIAIGLTIHNAPVELREKLAVPEAEWPRAIEELCQFPHIEEAAVLSTCNRMELYVVGLSWHRGVREVEE
WLSRTSGVPLDELRPYLFLLRDRDATHHLMRVSGGLDSLVMGEGQILAQVRQVYKVGQNCPGFGRHLNGLFKQAI
TAGKRVRAETSISTGSVSVSSAAVELAQLKLPTHNWSDAKVCIIGAGKMSTLLVKHLQSKGCKEVTVLNRSLPRA
QALAEEFPEVKFNIHLMPDLLQCVEASDVIFAASGSEEILIHKEHVEAMSKPSDVVGSKRRFVDISVPRNIAPAI
NELEHGIVYNVDDLKEVVAANKEGRAQAAAEAEVLIREEQRAFEAWRDSLETVPTIKALRSKAETIRAAEFEKAV
SRLGEGLSKKQLKAVEELSKGIVNKLLHGPMTALRCDGTDPDAVGQTLANMEALERMFQLSEVDVAALAGKQ
Light independent protochlorophyllide reductase subunit N (ch1N) nucleic acid
sequence (SEQ ID NO: 13):
atgttatactcacaatttaaacattcggtgcctttaggccgtaagtctccccttctttcagggggccccccttct
gggggtcgcccaacaacggctgcctcaggcctaggtcgcaacgtggccgtaagaattgggaccccgttgggcttt
gcccttcgggcccaggtaattatggcagctgcgggcaatactagcggtgcgccgcaccccgtaggggagtcccag
cctgcgttgtcccaggtggattctcaacttgtaattgagtgtgaaacaggaaattaccatactttttgcccaatt
agttgtgtttcttggttataccaaaaaattgaagatagttttttcttagttattggtacaaaaacgtgtgggtat
tttttacaaaatgctttaggggttatgatttttgccgaacctcgttacgctatggcggaattagaagaaagcgat
atttcggcgcaattaaatgattacaaagaattaaaacgtctatgtttacaaattaaacaagaccgtaacccaagt
gttattgtgtggattggcacatgcacaaccgaaattattaaaatggatttagaaggtatggcaccgaaactagaa
gctgaaatcggtattccaattgtggtagcacgcgcaaatggacttgattatgcttttacacaaggtgaagatact
gttttagctgcgatggtccaaaaatgcccggaattaggcgctattccagctattgtacctcagattccttctgac
tctcgtacacttagccaactatctgtagcggcttcggtacccgaaaacagtgcgtctgggccagaaggggagcct
tcactagcccagaagggaatggattctaagttaacaaacaactctccatgccgagtagattctgtctcagaatct
accccggcgtttcctggacgtgctccgcacgtcgggaaaagtactcctcaaaatttagttttatttggttcatta
cctagcacgatggcaaatcaactggagtttgaattaaaacgccaaggtattaatgttactgggtggttacctgcg
gctcgctattcatctttacctgcattaggtgaaaacgtgtatgtttgtgggattaatccatttttaagtcgaact
gctacttctttaatgcgtcgtcgtaaatgcaaattaatttcagctcctttcccaattggtccagatggtacaaaa
gcttgggtcgaaaaaatttgtaatgttttcggtgttacaccaactggtttagaagatcgtgaacgtcttgtttgg
gaaggtttaaaagattatttaaatttcgtaaaagggaaatctgttttctttatgggtgataatctgttagaaatt
tcattagcccgttttttaattcgctgtggtatgaccgtttatgaaatcggtattccgtacatggaccaacgattt
caagctggggaattagaattattaaaaaaaacatgcatggaaatgaacgtgcccctaccgcgtattgttgaaaaa
cctgataattactatcaaattcaacgtattaaagaattacaaccagatttagttattaccggcatggcccatgca
aacccactggaagcgcgcggcattactacgaaatggtccgttgaatttacgtttgcgcaaattcatgggtttggc
aacgcacgtgatatcttagaattagttacaaaaccgttacgtcgtaataaaaatctatctaaatatcaatttccg
ttagatagctgggacaagcctgcttccgtaggcgctcacgaactgtcggcctaa
Light independent protochlorophyllide reductase subunit N (ch1N) amino acid sequence
(SEQ ID NO: 14):
MLYSQFKHSVPLGRKSPLLSGGPPSGGRPTTAASGLGRNVAVRIGTPLGFALRAQVIMAAAGNTSGAPHPVGESQ
PALSQVDSQLVIECETGNYHTFCPISCVSWLYQKIEDSFFLVIGTKTCGYFLQNALGVMIFAEPRYAMAELEESD
ISAQLNDYKELKRLCLQIKQDRNPSVIVWIGTCTTEIIKMDLEGMAPKLEAEIGIPIVVARANGLDYAFTQGEDT
VLAAMVQKCPELGAIPAIVPQIPSDSRTLSQLSVAASVPENSASGPEGEPSLAQKGMDSKLTNNSPCRVDSVSES
TPAFPGRAPHVGKSTPQNLVLFGSLPSTMANQLEFELKRQGINVTGWLPAARYSSLPALGENVYVCGINPFLSRT
ATSLMRRRKCKLISAPFPIGPDGTKAWVEKICNVFGVTPTGLEDRERLVWEGLKDYLNFVKGKSVFFMGDNLLEI
SLARFLIRCGMTVYEIGIPYMDQRFQAGELELLKKTCMEMNVPLPRIVEKPDNYYQIQRIKELQPDLVITGMAHA
NPLEARGITTKWSVEFTFAQIHGFGNARDILELVTKPLRRNKNLSKYQFPLDSWDKPASVGAHELSA
Light Independent protochlorophyllide subunit B (ch1B) nucleic acid sequence (SEQ ID
NO: 15):
atgaaattagcgtattggatgtatgcgggaccggctcatattggaacattacgagttgcaagctcgtttcgaaat
gtgcatgctattatgcatgctcccttaggcgatgattattttaacgtaatgcgttcaatgttagaacgtgaacgt
gattttacgccagtgacggcaagtattgttgatcgtcatgttttagctcgtggttcacaagaaaaagttgttgaa
aacattcaacgaaaagataaagaagaatgtccggatttaattttattaacaccaacatgtacctcaagtattttg
caagaagatttacaaaattttgtaaatcgcgcggccgaagtagcaaagcgttcggatgttttattagctgacgtt
aaccattaccgagtgaatgaattacaagcggctgaccgtacgttagagcaaattgtacgcttttatttagaaaaa
gaagtaaataaacttcacgcggagttaggcggccttaaaaaaccgcttcgctttgcccagcgtacccaaaagccg
tctgccaatattttaggcatgtttacactaggtttccataatcaacatgactgtcgtgaattaaaacgtttatta
aatgatttaggtatcgaagtcaatgaagtgattcctgaaggtagttttgtacatggattaaaaaatttaccaaaa
gcgtggtttaacatcgtcccgtatcgtgaagttggtttaatgacggcaatttatttagaaaaagaatttggcatg
ccttatacctcaatcacgccaatgggcattattgacaccgcggcgtttattcgtgaaattgcggccatttgtagt
caaattagcacttcacaggcatctacaaactcaactgaaggactccagaggggagaaaatgtcagtttaactgaa
actaattcgattatttttaataaagcaaaatatgaacaatacattaatcaacaaacgcattttgtttctcaagca
gcttggttttcacgttctattgactgtcaaaatttaaccggtaaaaaaaccgttgtgtttggtgatgcaactcac
gcggcaagtatgacgaaaattcttgtgcgcgaaatgggtattcatgttgtttgcgcgggcacgtattgtaaacat
gatgcagattggtttagagagcaagtttcaggtttttgtgatcaagttttaattacagatgatcacagccaaatt
gcggaaatcattgctcaaattgaacctgcagccatttttggtacacaaatggaacgtcatgttgggaaaaggtta
gatattccttgtggggttatttctgcaccggtacatattcaaaacttcccactaggctttagaccgtttttaggg
tatgaaggtactaatcaaatttccgatttagtttataattcgtttagtttaggtatggaagatcacttactagaa
attttcaacggtcatgacaataaagaagttattacacgttcgtattcttcagaaactgatttagaatggacaaaa
gaagcattagatgaactagctcgtgttcctggttttgttcgttcaaaagttaaacgtaatactgaaaaatttgcg
cgtacaaataaaaatcaagttattactattgaagttatgtacgcagctaaagaagcggtatcagcgtaa
Light Independent protochlorophyllide subunit B (ch1B) amino acid sequence (SEQ ID
NO: 16):
MKLAYWMYAGPAHIGTLRVASSFRNVHAIMHAPLGDDYFNVMRSMLERERDFTPVTASIVDRHVLARGSQEKVVE
NIQRKDKEECPDLILLTPTCTSSILQEDLQNFVNRAAEVAKRSDVLLADVNHYRVNELQAADRTLEQIVRFYLEK
EVNKLHAELGGLKKPLRFAQRTQKPSANILGMFTLGEHNQHDCRELKRLLNDLGIEVNEVIPEGSFVHGLKNLPK
AWFNIVPYREVGLMTAIYLEKEFGMPYTSITPMGIIDTAAFIREIAAICSQISTSQASTNSTEGLQRGENVSLTE
TNSIIFNKAKYEQYINQQTHFVSQAAWFSRSIDCQNLTGKKTVVFGDATHAASMTKILVREMGIHVVCAGTYCKH
DADWFREQVSGFCDQVLITDDHSQIAEIIAQIEPAAIFGTQMERHVGKRLDIPCGVISAPVHIQNFPLGFRPFLG
YEGTNQISDLVYNSFSLGMEDHLLEIFNGHDNKEVITRSYSSETDLEWTKEALDELARVPGFVRSKVKRNTEKFA
RTNKNQVITIEVMYAAKEAVSA
Light independent protochlorophyllide reductase subunit L (ch1L) nucleic acid
sequence (SEQ ID NO: 17):
atgaaattagcagtttatggcaaaggtggtattggtaaatccacaacaagttgtaacatttcaattgcattagca
aaacgtggcaaaaaagtattacaaattggttgtgatccaaaacacgatagtacttttacattaaccggtttttta
attccaacaattattgatactttacaaagtaaagattatcattacgaagatgtttggccggaagatgttatttac
caaggctacgggagtgtggattgtgttgaagcaggtggcccgccagccggcgccggctgtggtgggtatgttgtt
ggtgaaacagttaaattattaaaagaattaaatgcattttatgaatatgatgttattctgtttgatgttttaggg
gatgttgtatgtggtgggtttgctgcacctttaaattacgccgactattgcattattgtcacagataatggcttt
gatgcgttatttgccgcaaaccgtattgctgcttcagtgcgcgaaaaagcgcgcattcacccattacgtttagct
gggttaattgggaatcgtacagccaaacgcgatttaatcgataaatacgttgaagcgtgcccgatgccagtctta
gaggtattaccgttaattgaagacattcgtgtgtcacgcgtaaaaggtaaaacattatttgaaatggcagaacat
gattcatcattacactacatttgtgacttttatttaaatattgcggatcaattattaactgaaccagaaggtgtt
gttccgcgcgaattagcagaccgtgaattatttactctattatcagatttctatttaaacgctgggactcctagc
cctagtggatctgagttcggctcaggcgcccttagcggaacgagcggcgaaacagctcccggtaatatgggtcag
cacatgagtaacgcagtaaaaacaaacgaacaggaaatgaatttctttcttgtgtaa
Light independent protochlorophyllide reductase subunit L (ch1L) amino acid sequence
(SEQ ID NO: 18):
MKLAVYGKGGIGKSTTSCNISIALAKRGKKVLQIGCDPKHDSTFTLTGFLIPTIIDTLQSKDYHYEDVWPEDVIY
QGYGSVDCVEAGGPPAGAGCGGYVVGETVKLLKELNAFYEYDVILFDVLGDVVCGGFAAPLNYADYCIIVTDNGF
DALFAANRIAASVREKARIHPLRLAGLIGNRTAKRDLIDKYVEACPMPVLEVLPLIEDIRVSRVKGKTLFEMAEH
DSSLHYICDFYLNIADQLLTEPEGVVPRELADRELFTLLSDFYLNAGTPSPSGSEFGSGALSGTSGETAPGNMGQ
HMSNAVKTNEQEMNFFLV
Magnesium Chelatase subunit H (CHLH2) nucleic acid sequence (SEQ ID NO: 19):
atgcggattgtgctggtcagcggcttcgagagctttaacgtgggcctgtacaaggatgcggcggagctgctgaag
cgctccatgcccaacgtcacactccaggtgttctccgaccgcgacctggcctccgacgccacccgctcccggctg
gaggcggctctggggcgcgccgacatcttcttcggatcactgctgttcgactacgaccaggtggagtggctacgg
gcccggctggagcgggtgcctgtgcggctagtgtttgagtcggcgttggagctcatgagctgcaacaaggtgggg
tcgttcatgatgggcggcggcggtcccggcggcggcccgcccggcaaggcgcccggcccgccgcccgcggtgaag
aaggttctctccatgtttggaagcggtcgcgaggaggacaagatgggcggctcctccaatgtggtggccatgttc
agttacctggtggagaccctgatggagccaacgggtgggttatttggtagttggtggttgtgttatggttggccg
tttcggttgggtgatctgggctggtatctacaacccccctcaaccctcacgcctccaggctacgtgccgccgcct
gtggtggagactcccgcactgggctgcctccacccctccgcgcccggccgctacttcgagtcccccgccgagtac
atgaagtggtacgccagggagggcccgctgcgcggcacgggcgccccggtggttggcgtgctgctgtaccgcaag
catgtgatcaccgaccagccgtacatcccgcagctggtcagccagctggaggcggaggggctgctgcccgtgccc
atcttcatcaacggcgtggaggcgcacaccgtggttcgcgacctgctgacctccgtgcacgagcaggatctgctt
gcacgcggcgagacgggcgccatcagccccaccctgaagcgggacgcggtcaaggtggacgcggtggtgagcacc
attggcttcccgctggtgggcggccccgccggcaccatggagggcgggcggcaggcggaggtggccaaggccatc
ctgggcgccaaggacgtgccgtacacggtggcggcgccgctgcttattcaggacatggagagctggagcagggac
ggcgtggcgggtctccagagtgtggtgctgtactcgctgccggagctggacggcgcagtggacacggtgccactg
ggggggctggtgggggacgacatctacctggtgccggagcgggtgaagaagctggcggggcggctcaagtcgtgg
cgtacgacacgcactaagcatgcctctgtttgtgacgtccagcccctcccccccccgtctcccctctccaccctc
cctctcccttcctctcccttcctctcactctccaccctcttccccctccgcccaaacataacgaggcgggggctg
ctgggcgcaagcgggccctggagtacccgctgcgacctagctagtccaactccacccatcccccaatgccgcaat
agctttccggagatgagcacacacacacacacacacacacacacacacacacacacacacacacacacacacaca
cgccacccacgcacacacacacacacacacgctccccccgctcgccacacccccatcccaccccacccgcaggag
ctgctgacgtaccccgcggactggggcccggccgagtggggcccgctgccctacctgcccgaccccgacgtgctg
gttcgccgcatggaggcgcagtggggcgagctgcgagcctaccgcggcctcaacacctcggcgcgcggcatgttc
caggagtacggggctgacgtggtcctgcacttcggcatgcacggcaccgtggagtggttgcctggggcgccgctg
gggaacaacggcctcagctggagcgacgtgctgctcggcgagctgccaaacgtgtacgtgtacgctgccaacaac
ccctccgagtccatcgtggcaaagcggcgcggctacggcaccatcgtcagccacaacgtgccgccgtacgggcgg
gcgggtctgtacaagcagctttccagcctcaaggagacgcttcaggagtaccgcgaggccgcgcaggccgcacgt
gcccgagcaggagccagcagcagcagcggcagtagcagcagtagcagtagcagcggcagtggcagtagcagcagc
agtgtggagctgcgggcggcgttggcaccggtgttcgacgcctacactgaccgcctgtatgcctacctgcagctg
ctggaggggcggctgttcagcgaggggctacacgtactgggagcgccgccggcgccgccgcaggtgggtggtttt
cccgcgagcttccaacggtaccgtaaactgcccaactgcccaacttctccccaaacacaggaggctgtcaagatc
cggaacctgctcatgcagaacacgcaggagctggacgggctgctcaagggcctgggtgggcgttacgtgcttccc
gaggcgggcggcgacctgctgcgggacgggtcgggcgtgctgcccaccggccgcaacatccacgcactggacccc
taccgcatgccctcccccgccgccatggcccgtggggcggcggtggcggcggccattcttgagcagcaccgggcg
gctaacagcggggcgtggcccgagacctgcgccgtcaacctgtgggggctggactccatcaagagcaagggcgag
agtgtgggggtggtgctggcgctggtgggggcggtgccggtgcgcgagggtacgggccgcgtcgcgcgcttccaa
ctggtgccgctgtcagagttgggccggccgcgtgtggacgtgctttgtaacatgagcggcatcttccgcgactcc
ttccagaacgtggtggagctgctcgacgacctgtttgcaagggccgccgccgccgctgacgagccagatgacatg
aacttcatcgccaaacacgcccgagccatggagaagcagggcctgtccgccacctcggcccgcctgttctccaac
ccggctggcgactacgggtcgatggtcaacgagcgagtggggcagggcagctgggccaacggcgacgagctgggt
gacacgtgggcggcccgcaacgccttcagctacggccgaggcaaggagcgaggcacggcgcggcccgaggtgctg
caggcgctgctcaagaccacggaccggatcgtgcagcagatcgacagtgtggagtacggcctgacagacatccag
gagtactacgccaacacgggcgccctcaagagagccgccgaggtggccaaaggcgacccgggccccggtggccgg
cggccgcgcgtggggtgttccattgtggaggcctttggcggcgcgggcgcgggcgcgggcggcgccggtggagcg
ggcgtgccgccgcctcgcgagctggaggaggtgctgcgcctggagtaccgctcgaagctgctcaaccccaagtgg
gcccgggccatggcggcgcagggcagcggcggcgcctacgagatcagtcagcgcatgacggcgttggtgggctgg
ggcgccaccaccgatttcagggagggctgggtgtgggacccaggcgccatggacacgtatgtgggcgatgaggag
atggccagcaagctcaagaagaacaacccgcaggcctttgccaacgtgctgcggcgcatgctggaggcggcgggc
cgcggcatgtggagccccaacaaggaccagctggcacagctcaagtcgctgtacagcgagatggacgaccagctg
gagggggtgacg
Magnesium Chelatase subunit H (CHLH2) amino acid sequence (SEQ ID NO: 20):
MRIVLVSGFESFNVGLYKDAAELLKRSMPNVTLQVFSDRDLASDATRSRLEAALGRADIFFGSLLFDYDQVEWLR
ARLERVPVRLVFESALELMSCNKVGSFMMGGGGPGGGPPGKAPGPPPAVKKVLSMFGSGREEDKMGGSSNVVAMF
SYLVETLMEPTGGLFGSWWLCYGWPFRLGDLGWYLQPPSTLTPPGYVPPPVVETPALGCLHPSAPGRYFESPAEY
MKWYAREGPLRGTGAPVVGVLLYRKHVITDQPYIPQLVSQLEAEGLLPVPIFINGVEAHTVVRDLLTSVHEQDLL
ARGETGAISPTLKRDAVKVDAVVSTIGFPLVGGPAGTMEGGRQAEVAKAILGAKDVPYTVAAPLLIQDMESWSRD
GVAGLQSVVLYSLPELDGAVDTVPLGGLVGDDIYLVPERVKKLAGRLKSWRTTRTKHASVCDVQPLPPPSPLSTL
PLPSSPFLSLSTLFPLRPNITRRGLLGASGPWSTRCDLASPTPPIPQCRNSFPEMSTHTHTHTHTHTHTHTHTHT
RHPRTHTHTHAPPARHTPIPPHPQELLTYPADWGPAEWGPLPYLPDPDVLVRRMEAQWGELRAYRGLNTSARGMF
QEYGADVVLHFGMHGTVEWLPGAPLGNNGLSWSDVLLGELPNVYVYAANNPSESIVAKRRGYGTIVSHNVPPYGR
AGLYKQLSSLKETLQEYREAAQAARARAGASSSSGSSSSSSSSGSGSSSSSVELRAALAPVFDAYTDRLYAYLQL
LEGRLFSEGLHVLGAPPAPPQVGGFPASFQRYRKLPNCPTSPQTQEAVKIRNLLMQNTQELDGLLKGLGGRYVLP
EAGGDLLRDGSGVLPTGRNIHALDPYRMPSPAAMARGAAVAAAILEQHRAANSGAWPETCAVNLWGLDSIKSKGE
SVGVVLALVGAVPVREGTGRVARFQLVPLSELGRPRVDVLCNMSGIFRDSFQNVVELLDDLFARAAAAADEPDDM
NFIAKHARAMEKQGLSATSARLFSNPAGDYGSMVNERVGQGSWANGDELGDTWAARNAFSYGRGKERGTARPEVL
QALLKTTDRIVQQIDSVEYGLTDIQEYYANTGALKRAAEVAKGDPGPGGRRPRVGCSIVEAFGGAGAGAGGAGGA
GVPPPRELEEVLRLEYRSKLLNPKWARAMAAQGSGGAYEISQRMTALVGWGATTDFREGWVWDPGAMDTYVGDEE
MASKLKKNNPQAFANVLRRMLEAAGRGMWSPNKDQLAQLKSLYSEMDDQLEGVT
Magnesium Chelatase subunit 1 (CHLI1) Chlamydomonas reinhardtii nucleic acid
sequence (SEQ ID NO: 21):
atggccctgaacatgcgtgtttcctcttccaaggtcgctgccaagcagcagggccgcatctccgcggtgccggtt
gtgtcgagcaaggtggcctcctccgcccgcgtggcccccttccagggcgctcccgtggccgcgcagcgcgctgct
ctgctggtgcgcgccgctgccgctactgaggtcaaggctgctgagggccgcactgagaaggagctgggccaggcc
cgccccatcttccccttcaccgccatcgtgggccaggatgagatgaagctggcgctgattctgaacgtgatcgac
cccaagatcggtggtgtcatgatcatgggcgaccgtggcactggcaagtccaccaccattcgtgccctggcggat
ctgctgcccgagatgcaggtggttgccaacgacccctttaactcggaccccaccgaccccgagctgatgagcgag
gaggtgcgcaaccgcgtcaaggccggcgagcagctgcccgtgtcttccaagaagattcccatggtggacctgccc
ctgggcgccactgaggaccgcgtgtgcggcaccatcgacatcgagaaggcgctgaccgagggtgtcaaggcgttc
gagcccggcctgctggccaaggccaaccgcggcatcctgtacgtggatgaggtcaacctgctggacgaccacctg
gtcgatgtgctgctggactcggccgcctccggctggaacaccgtggagcgcgagggtatctccatcagccacccc
gcccgcttcatcctggtcggctcgggcaaccccgaggagggtgagctgcgcccccagctgctggatcgcttcggc
atgcacgcccagatcggcaccgtcaaggacccccgcctgcgtgtgcagatcgtgtcgcagcgctcgaccttcgac
gagaaccccgccgccttccgcaaggactacgaggccggccagatggcgctgacccagcgcatcgtggacgcgcgc
aagctgctgaagcagggcgaggtcaactacgacttccgcgtcaagatcagccagatctgctcggacctgaacgtg
gacggcatccgcggcgacatcgtgaccaaccgcgccgccaaggccctggccgccttcgagggccgcaccgaggtg
acccccgaggacatctaccgtgtcattcccctgtgcctgcgccaccgcctccggaaagaccccctggctgagatc
gacgacggtgaccgcgtgcgtgagatcttcaagcaggtgttcggcatggagtaa
Magnesium Chelatase subunit 1 (CHLI1) Chlamydomonas reinhardtii amino acid
sequence (SEQ ID NO: 22):
MALNMRVSSSKVAAKQQGRISAVPVVSSKVASSARVAPFQGAPVAAQRAALLVRAAAATEVKAAEGRTEKELGQA
RPIFPFTAIVGQDEMKLALILNVIDPKIGGVMIMGDRGTGKSTTIRALADLLPEMQVVANDPFNSDPTDPELMSE
EVRNRVKAGEQLPVSSKKIPMVDLPLGATEDRVCGTIDIEKALTEGVKAFEPGLLAKANRGILYVDEVNLLDDHL
VDVLLDSAASGWNTVEREGISISHPARFILVGSGNPEEGELRPQLLDRFGMHAQIGTVKDPRLRVQIVSQRSTFD
ENPAAFRKDYEAGQMALTQRIVDARKLLKQGEVNYDFRVKISQICSDLNVDGIRGDIVTNRAAKALAAFEGRTEV
TPEDIYRVIPLCLRHRLRKDPLAEIDDGDRVREIFKQVFGME
Magnesium Chelatase sunubit1 (CHLI2) Chlamydomonas reinhardtii nucleic acid
sequence (SEQ ID NO: 23):
atgcagagtctccagggtcagcgcgcgttcactgcggtgcgccagggtcgggcgggtcccctgcggactcgcctg
gtcgtgcgctcgtctgttgccttgccatccacgaaagccgcgaagaagccgaacttcccgttcgtcaagattcag
ggccaggaggagatgaagcttgcactgctgctgaacgtggtcgaccccaacatcggcggagtgcttattatgggt
gaccgcggcactgccaagtcggtcgcggtccgcgccctggtggatatgcttcccgacattgacgtggttgagggc
gacgccttcaacagctcccccaccgaccccaagttcatgggccccgacaccctgcagcgcttccgcaacggcgag
aagctgcccaccgtccgcatgcggacccccctggtggagctgcctctgggcgccaccgaggaccgcatctgcggc
accatcgacatcgagaaggcgctgacgcagggcatcaaggcctacgagcccggcctgctggccaaggccaaccgc
ggcatcctgtatgtggacgaggtgaacctgctggatgatggcctggttgatgtcgtgctggactcgtcggctagc
ggcctgaacactgtggagcgtgagggtgtgtccattgtgcaccctgcccgcttcatcatgattggctcaggcaac
ccccaggagggtgagctgcgcccgcagctgctggatcgcttcggcatgagcgtcaacgtggccacgctgcaggac
accaagcagcgcacgcagctggtgctggaccggcttgcgtacgaggcggaccctgacgcatttgtggactcgtgc
aaggccgagcagacggcgctcacggacaagctggaggcggcccgccagcgcctgcggtccgtcaagatcagcgag
gagctgcagatcctgatctcggacatttgctcgcgcctggatgtggatggcctgcgcggtgacattgtgatcaac
cgcgccgccaaggcgcttgtggccttcgagggccgcaccgaggtgaccacgaatgacgtggagcgcgtcatctcg
ggctgcctcaaccaccgcctgcgcaaggacccgctggaccccattgacaacggcaccaaggtggccatcctgttc
aagcgcatgaccgaccccgagatcatgaagcgcgaggaggaggccaagaagaagcgcgaggaggcggccgccaag
gccaaggcggagggcaaggcggaccgccccacgggcgccaaggctggcgcctgggctggcttgccccctcgtcgg
taa
Magnesium Chelatase sunubit1 (CHLI2) Chlamydomonas reinhardtii amino acid
sequence (SEQ ID NO: 24):
MQSLQGQRAFTAVRQGRAGPLRTRLVVRSSVALPSTKAAKKPNFPFVKIQGQEEMKLALLLNVVDPNIGGVLIMG
DRGTAKSVAVRALVDMLPDIDVVEGDAFNSSPTDPKFMGPDTLQRFRNGEKLPTVRMRTPLVELPLGATEDRICG
TIDIEKALTQGIKAYEPGLLAKANRGILYVDEVNLLDDGLVDVVLDSSASGLNTVEREGVSIVHPARFIMIGSGN
PQEGELRPQLLDRFGMSVNVATLQDTKQRTQLVLDRLAYEADPDAFVDSCKAEQTALTDKLEAARQRLRSVKISE
ELQILISDICSRLDVDGLRGDIVINRAAKALVAFEGRTEVTTNDVERVISGCLNHRLRKDPLDPIDNGTKVAILF
KRMTDPEIMKREEEAKKKREEAAAKAKAEGKADRPTGAKAGAWAGLPPRR
Magnesium Chelatase subunit D (CHLD) Chlamydomonas reinhardtii nucleic acid
sequence (SEQ ID NO: 25):
atgaagtctctctgccatgagctcgctggccccagcgttactgggtgcggccggcgaagcctccggaaggctttc
agcggtgccaagattgcgcaggtctctcgccccgctgtgcttaacagcgtgcagcgccaacagcgtctcgcctgt
tctgccgtggccgagctctccgctgctgagctgcgcgccatgaaggtgtctgaggaggactccaagggcttcgat
gcggatgtgtcgacccgcctggcccgctcgtaccctctggcggccgtggtgggccaggacaacatcaagcaggcg
ctgctgctgggcgccgtggacaccgggctgggcggcatcgccatcgccggtcgccgcggtaccgccaagtccatc
atggctcgcggcctgcacgctctgctgccgcccattgaggtggtggagggcagcatctgcaacgccgaccccgag
gacccccgctcctgggaggctggcctggctgagaagtatgcgggcggccctgtgaagaccaagatgcgctcggcg
ccgtttgtgcagatccctctgggtgtgactgaggaccgcttggtgggcactgtggacattgaggcgtccatgaag
gagggcaagactgtgttccagcccggcctgctggctgaggcgcaccgcggcatcctgtacgtggacgagatcaac
ctgctggatgacggcattgccaacctgctgctgtccatcctgtcggacggagtcaacgtggtggagcgcgagggc
atctccatcagccacccctgccggccgctgctgattgccacctacaaccccgaggagggccctctgcgtgagcac
ctgctggaccgcatcgccattggcctcagcgccgacgtccccagcaccagcgacgagcgcgtcaaggccattgac
gcagccatccgcttccaggacaagccgcaggacactattgacgacaccgcggagctcaccgacgccctgcgcacc
tcggtcatcctggctcgcgagtacctgaaggacgtgaccatcgcgccggagcaggtgacctacattgtggaggag
gcgcgccgcggcggagtccaggggcaccgcgcggagctgtacgcggtcaagtgtgccaaggcgtgtgcggctctg
gagggccgtgagcgtgtgaacaaggatgacctgcgccaggccgtgcagctggtcatcctgccgcgcgccaccatc
ctggaccagcccccgcccgagcaggagcagcccccgccgccgcccccgccccctcccccgccgccgccgcaggac
caaatggaggacgaggaccaggaggagaaggaggacgagaaggaggaggaggagaaggagaacgaggaccaggac
gagcccgagatccctcaggagttcatgtttgagtccgagggcgtcatcatggacccctccatcctcatgttcgcg
cagcagcagcagcgcgcgcagggccgctccggccgcgccaagacgctcatcttcagcgacgaccgcggccgctac
atcaagcccatgctgcccaagggtgacaaggtcaagcgcctggcagtggacgccacgcttcgcgccgccgcgccc
taccagaagattcgccggcagcaggccatcagcgagggcaaggtgcagcgcaaggtgtacgtggacaagccagac
atgcgctccaagaagctggcccgcaaggccggtgcgctggtgatttttgttgtggacgcgtccggctccatggct
ctgaaccgcatgagcgccgccaagggcgcctgcatgcgcctgctggctgagtcgtacaccagccgcgaccaggtg
tgcctcatccccttctacggcgacaaggccgaggtgctgctgccgccctccaagtccatcgccatggcccgccgc
cgcctggactcgctgccctgcggcggcggctcgccccttgcgcacggcctgtccacggcggtacgtgtgggcatg
caggccagccaggcgggcgaggtgggccgcgtcatgatggtgctcatcacggacggccgcgccaacgtcagcctg
gccaagtccaacgaggaccccgaggcgctcaagcccgacgcgcccaagcccaccgccgactcgctgaaggacgag
gtgcgcgacatggccaagaaggccgcgtccgccggcatcaacgtgcttgtcattgacacggagaacaagttcgtg
agcaccggctttgcggaggagatctccaaggcagcgcagggcaagtactactacctgcccaacgccagcgacgcc
gccatcgcggcggccgcgtccggcgccatggccgcggccaagggcggctactag
Magnesium Chelatase subunit D (CHLD) Chlamydomonas reinhardtii amino acid
sequence (SEQ ID NO: 26):
MKSLCHELAGPSVTGCGRRSLRKAFSGAKIAQVSRPAVLNSVQRQQRLACSAVAELSAAELRAMKVSEEDSKGFD
ADVSTRLARSYPLAAVVGQDNIKQALLLGAVDTGLGGIAIAGRRGTAKSIMARGLHALLPPIEVVEGSICNADPE
DPRSWEAGLAEKYAGGPVKTKMRSAPFVQIPLGVTEDRLVGTVDIEASMKEGKTVFQPGLLAEAHRGILYVDEIN
LLDDGIANLLLSILSDGVNVVEREGISISHPCRPLLIATYNPEEGPLREHLLDRIAIGLSADVPSTSDERVKAID
AAIRFQDKPQDTIDDTAELTDALRTSVILAREYLKDVTIAPEQVTYIVEEARRGGVQGHRAELYAVKCAKACAAL
EGRERVNKDDLRQAVQLVILPRATILDQPPPEQEQPPPPPPPPPPPPPQDQMEDEDQEEKEDEKEEEEKENEDQD
EPEIPQEFMFESEGVIMDPSILMFAQQQQRAQGRSGRAKTLIFSDDRGRYIKPMLPKGDKVKRLAVDATLRAAAP
YQKIRRQQAISEGKVQRKVYVDKPDMRSKKLARKAGALVIFVVDASGSMALNRMSAAKGACMRLLAESYTSRDQV
CLIPFYGDKAEVLLPPSKSIAMARRRLDSLPCGGGSPLAHGLSTAVRVGMQASQAGEVGRVMMVLITDGRANVSL
AKSNEDPEALKPDAPKPTADSLKDEVRDMAKKAASAGINVLVIDTENKFVSTGFAEEISKAAQGKYYYLPNASDA
AIAAAASGAMAAAKGGY
Magnesium Chelatase subunit H (CHLH1) Chlamydomonas reinhardtii nucleic acid
sequence (SEQ ID NO: 27):
atgcagacttcctcgcttcttggccggcgcacggcccacccggctgcgggcgcgacgcccaagccggttgcgccc
tcgccccgcgtggctagcacccgccaggtcgcgtgcaatgtggcgactggaccccggccgcccatgaccaccttc
accggtggcaacaagggccctgctaagcagcaggtgtcgctggatctgcgcgacgagggcgctggcatgttcacc
agcaccagcccggagatgcgccgtgtcgtccctgacgatgtgaagggtcgcgttaaggtgaaggttgtgtacgtg
gtgctggaggcccagtaccagtcggccatcagcgctgcggtgaagaacatcaacgccaagaactccaaggtgtgc
ttcgaggtggtgggctacctgctggaggagctgcgtgaccagaagaacctcgatatgctcaaggaggatgtggcc
tctgccaacatcttcatcggctcgctcatcttcattgaggagcttgccgagaagattgtggaggcggtgagcccc
ctgcgcgagaagctggacgcgtgcctgatcttcccgtccatgccggcggtcatgaagctgaacaagctgggcacg
ttttcgatggctcagctgggccagtcgaagtcggtgttctcggagttcatcaagtctgctcgcaagaacaacgac
aacttcgaggagggcttgctgaagctggtgcgcaccctgcctaaggtgctgaagtatctgccctcggacaaggcg
caggacgccaagaacttcgtgaacagcctgcagtactggctgggcggtaactcggacaacctggagaacctgctg
ctgaacaccgtcagcaactacgtgcccgctctgaagggcgtggacttcagcgtggctgagcccaccgcctacccc
gatgtgggtatctggcaccctctggcctcgggcatgtacgaggacctgaaggagtacctgaactggtacgacacc
cgcaaggacatggtcttcgccaaggacgcccccgtcattggcctggtgctgcagcgctcgcacctggtgactggc
gatgagggccactacagcggcgtggtcgctgagctggagagccgcggtgctaaggtcatccccgtctttgccggt
ggcctggacttctccgcccccgtcaagaagttcttctacgaccccctgggctctggccgcacgttcgtggacacc
gttgtgtcgctgaccggcttcgcgctggtgggcggccccgcgcgccaggacgcgccgaaggccattgaggcgctg
aagaacctgaacgtgccctacctggtgtcgctgccgctggtgttccagaccactgaggagtggctggacagcgag
ctgggcgtgcaccccgtccaggtggctctgcaggttgccctgcccgagctggatggtgccatggagcccatcgtg
ttcgctggccgtgactcgaacaccggcaagtcgcactcgctgcccgaccgcatcgcttcgctgtgcgctcgcgcc
gtgaactgggccaacctgcgcaagaagcgcaacgccgagaagaagctggccgtcaccgtgttcagcttcccccct
gacaagggcaacgtcggcactgccgcctacctgaacgtgttcggctccatctaccgcgtgctgaagaacctgcag
cgcgagggctacgacgtgggcgccctgccgccctcggaggaggatctgatccagtcggtgctgacccagaaggag
gccaagttcaactcgaccgacctgcacatcgcctacaagatgaaggtggacgagtaccagaagctgtgcccttac
gccgaggcgctggaggagaactggggcaagccccccggcaccctgaacaccaacggccaggagctgctggtgtac
ggccgccagtacggcaacgtcttcatcggcgtgcagcccaccttcggctacgagggcgacccgatgcgcctgctg
ttctcgaagtcggccagcccccaccacggcttcgccgcctactacaccttcctggagaagatcttcaaggccgac
gccgtgctgcacttcggcacccacggctcgctggagttcatgcccggcaagcaggtcggcatgtcgggtgtgtgc
taccccgactcgctgatcggcaccatccccaacctctactactacgccgccaacaacccgtctgaggccaccatc
gccaagcgccgctcgtacgccaacaccatttcgtacctgacgccgcctgccgagaacgccggcctgtacaagggc
ctgaaggagctgaaggagctgatcagctcgtaccagggcatgcgtgagtctggccgcgccgagcagatctgcgcc
accatcattgagaccgccaagctgtgcaacctggaccgcgacgtgaccctgcccgacgctgacgccaaggacctg
accatggacatgcgcgacagcgttgtgggccaggtgtaccgcaagctgatggagattgagtcccgcctgctgccc
tgcggcctgcacgtggtgggctgcccgcccaccgccgaggaggccgtggccaccctggtcaacatcgctgagctg
gaccgcccggacaacaacccccccatcaagggcatgcccggcatcctggcccgcgccattggtcgcgacatcgag
tcgatttacagcggcaacaacaagggcgtcctggctgacgttgaccagctgcagcgcatcaccgaggcctcccgc
acctgcgtgcgcgagttcgtgaaggaccgcaccggcctgaacggccgcatcggcaccaactggatcaccaacctg
ctcaagttcaccggcttctacgtggacccctgggtgcgcggcctgcagaacggcgagttcgccagcgccaaccgc
gaggagctgatcaccctgttcaactacctggagttctgcctgacccaggtggtcaaggacaacgagctgggcgcc
ctggtagaggcgctgaacggccagtacgtcgagcccggccccggcggtgaccccatccgcaaccccaacgtgctg
cccaccggcaagaacatccacgccctggaccctcagtcgattcccactcaggccgcgctgaagagcgcccgcctg
gtggtggaccgcctgctggaccgcgagcgcgacaacaacggcggcaagtaccccgagaccatcgcgctggtgctg
tggggcactgacaacatcaagacctacggcgagtcgctggcccaggtcatgatgatggtcggtgtcaagcccgtg
gccgacgccctgggccgcgtgaacaagctggaggtgatccctctggaggagctgggccgcccccgcgtggacgtg
gttgtcaactgctcgggtgtgttccgcgacctgttcgtgaaccagatgctgctgctggaccgcgccatcaagctg
gcggccgagcaggacgagcccgatgagatgaacttcgtgcgcaagcacgccaagcagcaggcggcggagctgggc
ctgcagagcctgcgcgacgcggccacccgtgtgttctccaacagctcgggctcctactcgtccaacgtcaacctg
gcggtggagaacagcagctggagcgacgagtcgcagctgcaggagatgtacctgaagcgcaagtcgtacgccttc
aactcggaccgccccggcgccggtggcgagatgcagcgcgacgtgttcgagacggccatgaagaccgtggacgtg
accttccagaacctggactcgtccgagatctcgctgaccgatgtgtcgcactacttcgactccgaccccaccaag
ctggtggcgtcgctgcgcaacgacggccgcacccccaacgcctacatcgccgacaccaccaccgccaacgcgcag
gtccgcactctgggtgagaccgtgcgcctggacgcccgcaccaagctgctcaaccccaagtggtacgagggcatg
cttgcctcgggctacgagggcgtgcgcgagatccagaagcgcatgaccaacaccatgggctggtcggccacctcg
ggcatggtggacaactgggtgtacgacgaggccaactcgaccttcatcgaggatgcggccatggccgagcgcctg
atgaacaccaaccccaacagcttccgcaagctggtggccaccttcctggaggccaacggccgcggctactgggac
gccaagcccgagcagctggagcgcctgcgccagctgtacatggacgtggaggacaagattgagggcgtcgaataa
Magnesium Chelatase subunit H (CHLH1) Chlamydomonas reinhardtii amino acid
sequence (SEQ ID NO: 28):
MQTSSLLGRRTAHPAAGATPKPVAPSPRVASTRQVACNVATGPRPPMTTFTGGNKGPAKQQVSLDLRDEGAGMFT
STSPEMRRVVPDDVKGRVKVKVVYVVLEAQYQSAISAAVKNINAKNSKVCFEVVGYLLEELRDQKNLDMLKEDVA
SANTFIGSLIFTEELAEKIVEAVSPLREKLDACLIFPSMPAVMKLNKLGTFSMAQLGQSKSVFSEFIKSARKNND
NFEEGLLKLVRTLPKVLKYLPSDKAQDAKNFVNSLQYWLGGNSDNLENLLLNTVSNYVPALKGVDFSVAEPTAYP
DVGIWHPLASGMYEDLKEYLNWYDTRKDMVFAKDAPVIGLVLQRSHLVTGDEGHYSGVVAELESRGAKVIPVFAG
GLDFSAPVKKFFYDPLGSGRTFVDTVVSLTGFALVGGPARQDAPKAIEALKNLNVPYLVSLPLVFQTTEEWLDSE
LGVHPVQVALQVALPELDGAMEPIVFAGRDSNTGKSHSLPDRIASLCARAVNWANLRKKRNAEKKLAVTVFSFPP
DKGNVGTAAYLNVFGSIYRVLKNLQREGYDVGALPPSEEDLIQSVLTQKEAKFNSTDLHIAYKMKVDEYQKLCPY
AEALEENWGKPPGTLNTNGQELLVYGRQYGNVFIGVQPTFGYEGDPMRLLFSKSASPHHGFAAYYTFLEKIFKAD
AVLHFGTHGSLEFMPGKQVGMSGVCYPDSLIGTIPNLYYYAANNPSEATIAKRRSYANTISYLTPPAENAGLYKG
LKELKELISSYQGMRESGRAEQICATIIETAKLCNLDRDVTLPDADAKDLTMDMRDSVVGQVYRKLMEIESRLLP
CGLHVVGCPPTAEEAVATLVNIAELDRPDNNPPIKGMPGILARAIGRDIESIYSGNNKGVLADVDQLQRITEASR
TCVREFVKDRTGLNGRIGTNWITNLLKFTGFYVDPWVRGLQNGEFASANREELITLFNYLEFCLTQVVKDNELGA
LVEALNGQYVEPGPGGDPIRNPNVLPTGKNIHALDPQSIPTQAALKSARLVVDRLLDRERDNNGGKYPETIALVL
WGTDNIKTYGESLAQVMMMVGVKPVADALGRVNKLEVIPLEELGRPRVDVVVNCSGVFRDLFVNQMLLLDRAIKL
AAEQDEPDEMNFVRKHAKQQAAELGLQSLRDAATRVFSNSSGSYSSNVNLAVENSSWSDESQLQEMYLKRKSYAF
NSDRPGAGGEMQRDVFETAMKTVDVTFQNLDSSEISLTDVSHYFDSDPTKLVASLRNDGRTPNAYIADTTTANAQ
VRTLGETVRLDARTKLLNPKWYEGMLASGYEGVREIQKRMTNTMGWSATSGMVDNWVYDEANSTFIEDAAMAERL
MNTNPNSFRKLVATFLEANGRGYWDAKPEQLERLRQLYMDVEDKIEGVE
Photochlorophyllide reductase subunit B (ch1B) nucleic acid sequence (SEQ ID NO:
29):
atgaaattagcttattggatgtacgcaggtcccgctcatatcggtgtgttgcgtgttagcagctcttttaaaaat
gtacatgccattatgcatgctcctttaggagatgattattttaatgtaatgcgttccatgttagaacgtgaacgt
gattttacaccagtaacagccagtattgtagatcgtcatgttttagcaagaggatcgcaagaaaaagtggttgaa
aatattacgcgaaaaaataaagaagaaactcctgatttaattttattaactcctacttgtacgtcaagcatttta
caagaagatttacacaattttgttgaatcggcattagctaaaccagtacaaatagatgaacatgcagaccataaa
gtaactcaacaaagtgcactttcaagtgtatcccctttactaccgcttgaagaaaatacattaatagtaagtgaa
ctagataagaagcttagcccgtctagcaagttgcatattaatatgcccaatatttgtattcccgaaggagaaggg
gaaggggagcagactaaaaattcaatttttgttaaatctgcaactttaacaaatttgtcagaagaggaactatta
aatcaagaacatcataccaaaacaagaaatcactctgacgttattttagctgatgtaaaccattatcgtgtaaat
gaattacaagctgcagatcgtactcttgaacaaattgtacgttattatatttctcaagcacaaaaacaaaattgt
ttaaacattactaaaacagccaaaccatctgtaaatattattggtatttttactttgggttttcataatcaacat
gattgtcgtgaattaaaacgtttatttaatgatttaggtattcaaatcaatgaaatcatacctgaaggcggaaat
gtacacaacttaaaaaaattaccccaagcttggtttaattttgtgccctaccgtgaaattggcttaatgactgct
atgtatttaaaatccgagtttaatatgccttacgtcgcaattactcctatgggattaattgatacggctgcttgt
attcgttcaatttgtaaaatcattacaactcaattattaaatcagacggctacagtgcaggagccatcaaaattt
atttacccgaaggcgacgtcattagaacaaaccaatattctcgaaacctctcaaaaagaaactattcttaaagac
aatccagatagcggaaataccctttctacaactgtagaagaaattgaaactttatttaataaatatatcgatcaa
caaactcgttttgtttcccaagcagcctggttttcacgttctattgactgtcaaaatttaacaggtaaaaaagcc
gtagttttcggagatgctacacattcagctgccatgacaaaattattagcacgtgaaatgggtattaaggtttca
tgcgctggaacttattgcaaacacgatgcggattggtttagagagcaagttagtgggttttgtgatcaagtttta
attaccgatgatcacacacaagtaggggatatgattgcacaattagaacctgcagccatttttgggacacaaatg
gaacgtcacgttggtaaacgtttagatattccatgtggtgttatatctgctcctgtgcatattcaaaactttccg
ttaggttatcgaccttttttaggttatgaaggtacaaatcaaatagctgatttagtgtataattcatttaatctt
ggaatggaagaccatttattacaaatttttggaggtcatgattcagaaaacaattcgtcaattgcaacgcatttg
aatacaaataacgcaataaatttagcgccaggatatttacctgagggagaaggcagtagtagaacttcaaatgta
gtgtctacaatttctagtgaaaaaaaagccattgtatggtctccagaaggtttagcagaattaaataaagtccca
ggatttgttcgaggaaaagttaaacgtaatacggaaaaatatgctttacaaaaaaattgttcgatgattactgta
gaagttatgtatgcagcaaaagaagctttgtcggcttaa
Photochlorophyllide reductase subunit B (ch1B) amino acid sequence (SEQ ID NO: 30):
MKLAYWMYAGPAHIGVLRVSSSFKNVHAIMHAPLGDDYFNVMRSMLERERDFTPVTASIVDRHVLARGSQEKVVE
NITRKNKEETPDLILLTPTCTSSILQEDLHNFVESALAKPVQIDEHADHKVTQQSALSSVSPLLPLEENTLIVSE
LDKKLSPSSKLHINMPNICIPEGEGEGEQTKNSIFVKSATLTNLSEEELLNQEHHTKTRNHSDVILADVNHYRVN
ELQAADRTLEQIVRYYISQAQKQNCLNITKTAKPSVNIIGIFTLGEHNQHDCRELKRLENDLGIQINEIIPEGGN
VHNLKKLPQAWFNEVPYREIGLMTAMYLKSEFNMPYVAITPMGLIDTAACIRSICKIITTQLLNQTATVQEPSKF
IYPKATSLEQTNILETSQKETILKDNPDSGNTLSTTVEEIETLFNKYIDQQTRFVSQAAWFSRSIDCQNLTGKKA
VVFGDATHSAAMTKLLAREMGIKVSCAGTYCKHDADWFREQVSGFCDQVLITDDHTQVGDMIAQLEPAAIFGTQM
ERHVGKRLDIPCGVISAPVHIQNFPLGYRPFLGYEGTNQIADLVYNSFNLGMEDHLLQIFGGHDSENNSSIATHL
NTNNAINLAPGYLPEGEGSSRTSNVVSTISSEKKAIVWSPEGLAELNKVPGFVRGKVKRNTEKYALQKNCSMITV
EVMYAAKEALSA
Photochlorophyllide reductase subunit L (chIL) nucleic acid sequence (SEQ ID NO:
31):
atgaaattagctgtttacggaaaaggtggtattggaaaatcaacgacaagttgtaatatttcgattgctttacga
aaacgtggtaaaaaagtgttacaaattggttgtgatcctaaacatgatagtacttttacattgacagggttttta
attccaaccattattgatacattaagttctaaagattatcattatgaagatatttggcccgaagatgttatttac
ggaggttatgggggtgtagattgtgttgaagctggaggaccacctgccggtgcggggtgtggtggttatgttgta
ggtgaaacggtaaaacttttaaaagagttaaatgcttttttcgaatacgatgttattttatttgatgttttaggt
gatgttgtttgtggtggctttgctgctccattaaactacgctgattattgtattattgtaactgataatggtttt
gatgctttatttgctgcaaatcgtattgcagcttcagttcgtgaaaaagcacgtacacatccattgcgtttagcg
ggtttaatcggaaatcgtacatcaaaacgtgatttaattgataaatatgtagaagcttgtcctatgccagtatta
gaagttttaccattaattgaagaaattcgtatttcacgtgttaaaggcaaaactttatttgaaatgtcaaataaa
aataatatgacttcggctcatatggatggctctaaaggtgacaattctacagtaggagtgtcagaaactccatcg
gaagattatatttgtaatttttatttaaatattgctgatcaattattaacagaaccagaaggagttattccacgt
gaattagcagataaagaactttttactcttttatcagatttctatcttaaaatttaa
Photochlorophyllide reductase subunit L (chIL) amino acid sequence (SEQ ID NO: 32):
MKLAVYGKGGIGKSTTSCNISIALRKRGKKVLQIGCDPKHDSTFTLTGFLIPTIIDTLSSKDYHYEDIWPEDVIY
GGYGGVDCVEAGGPPAGAGCGGYVVGETVKLLKELNAFFEYDVILFDVLGDVVCGGFAAPLNYADYCIIVTDNGF
DALFAANRIAASVREKARTHPLRLAGLIGNRTSKRDLIDKYVEACPMPVLEVLPLIEEIRISRVKGKTLFEMSNK
NNMTSAHMDGSKGDNSTVGVSETPSEDYICNFYLNIADQLLTEPEGVIPRELADKELFTLLSDFYLKI
Photochlorophyllide reductase subunit N (ch1N) nucleic acid sequence (SEQ ID NO:
33):
atgttagatggtgccacaacgattttaaatttaaatagtttttttgaatgtgaaactggcaattatcatactttt
tgcccgattagctgtgtagcttggttatatcaaaaaatcgaagatagcttttttttagtaattgggacaaaaaca
tgtggttattttttacaaaatgcccttggagttatgatttttgccgaacctaggtatgctatggcagaattagaa
gaaagtgatatttcagcacaattaaacgattataaagaattaaaacgtttatgtttacaaattaaacaagataga
aatcccagcgttattgtttggattggaacttgtacaactgaaattatcaaaatggatttagaagggatggctcca
cgtttagaaactgaaatcggcatacccattgttgtagcacgtgctaatggtttagattatgcttttacacaaggt
gaagacacagttttatcagcaatggccttagcatccttaaaaaaagatgttccttttttagtaggtaatactggg
ttaacaaacaaccagcttctccttgaaaaatcaacttcttcagttaatgggacagacggaaaggaattacttaaa
aaatctcttgtattatttggttccgtaccaagtacagttactacacaattaactttagaattaaaaaaagaaggt
attaatgtatctggatggcttccatctgctaattataaagatttacctacttttaataaagatacacttgtatgt
ggtataaatccttttttaagtcgaacagctaccacgttaatgcgtcgtagtaagtgcacattaatttgtgcaccc
tttccaataggccccgatggcacaagagtttggattgaaaaaatttgtggtgcttttggcattaatcctagtctt
aatccaattactggtaatactaatttatatgatcgtgaacaaaaaattttcaacgggctagaagattatttaaaa
ttattacgtggaaaatctgtattttttatgggtgataatttattagaaatttctttagcacgttttttaacacgt
tgtggtatgattgtttatgaaatcggaattccatatttagataaacgatttcaagcagcagaattagctttatta
gaacaaacttgtaaagaaatgaatgtaccaatgccgcgcattgtagaaaaaccagataattattatcaaattcga
cgtatacgtgaattaaaacctgatttaacgattactggaatggcacatgcaaatccattagaagctcgaggtatt
acaacaaaatggtcagttgaatttacttttgctcaaattcatggatttactaatacacgtgaaattttagaatta
gtaacacagcctcttagacgcaatctaatgtcaaatcaatctgtaaatgctatttcttaa
Photochlorophyllide reductase subunit N (ch1N) amino acid sequence (SEQ ID NO: 34):
MLDGATTILNLNSFFECETGNYHTFCPISCVAWLYQKIEDSFFLVIGTKTCGYFLQNALGVMIFAEPRYAMAELE
ESDISAQLNDYKELKRLCLQIKQDRNPSVIVWIGTCTTEIIKMDLEGMAPRLETEIGIPIVVARANGLDYAFTQG
EDTVLSAMALASLKKDVPFLVGNTGLTNNQLLLEKSTSSVNGTDGKELLKKSLVLFGSVPSTVTTQLTLELKKEG
INVSGWLPSANYKDLPTFNKDTLVCGINPFLSRTATTLMRRSKCTLICAPEPIGPDGTRVWIEKICGAFGINPSL
NPITGNTNLYDREQKIFNGLEDYLKLLRGKSVFFMGDNLLEISLARFLTRCGMIVYEIGIPYLDKRFQAAELALL
EQTCKEMNVPMPRIVEKPDNYYQIRRIRELKPDLTITGMAHANPLEARGITTKWSVEFTFAQIHGETNTREILEL
VTQPLRRNLMSNQSVNAIS
Porphobilinogen deaminase (PBGD1) nucleic acid sequence (SEQ ID NO: 35):
atgcagcagtgcgttggccgctccgtccgcgctccgtccagcagggcggtcgcgcccaaggtcgctggcgctcgt
gtcagccgccgcgtgtgccgcgtctatgcctccgctgttgctaccaagacggtgaagattggcacgcgcggctcg
cccctggctctggcccaggcttacatgactcgcgacctgctgaagaagagcttccctgagctgagcgaggagggt
gctctggagatcgtgatcatcaagaccaccggtgacaaaatcctgaaccagcccctggctgacatcggtggcaag
ggtctgtttaccaaggagatcgatgatgctctgctgagcggcaagattgacatcgccgtgcactccatgaaggac
gtgcccacctacctgcccgagggcaccatcctgccctgcaacctgccccgcgaggatgtgcgcgatgtgttcatc
tcgcctgtcgccaaggacctgagcgagctgcccgccggcgccattgtgggctcggcctcgctgcgccgtcaggcc
cagatcctggccaagtacccccacctcaaggtggagaacttccgcggcaacgtgcagacccgcctgcgcaagctg
aacgagggcgcctgctccgccaccctgctggctctggccggtctgaagcgcctggacatgactgagcacatcacc
aagaccctcagcattgacgagatgctgcccgccgtgagccagggcgccattggcattgcctgccgcaccgacgac
ggcgccagccgcaacctgctggccgccctgaaccacgaggagacccgcatcgccgtggtgtgcgagcgcgccttc
ctgaccgccctggacggctcttgccgcacccccattgccggctacgcgcacaagggcgccgacggcatgctgcac
ttcagcggcctggtggccaccccggacggcaagcagatcatgcgcgctagccgcgtggtgcccttcacggaggcg
gatgccgtcaagtgcggcgaggaggccggcaaggagctcaaggccaacggccccaaggagctgttcatgtactaa
Porphobilinogen deaminase (PBGD1) amino acid sequence (SEQ ID NO: 36):
MQQCVGRSVRAPSSRAVAPKVAGARVSRRVCRVYASAVATKTVKIGTRGSPLALAQAYMTRDLLKKSFPELSEEG
ALEIVIIKTTGDKILNQPLADIGGKGLFTKEIDDALLSGKIDIAVHSMKDVPTYLPEGTILPCNLPREDVRDVFI
SPVAKDLSELPAGAIVGSASLRRQAQILAKYPHLKVENFRGNVQTRLRKLNEGACSATLLALAGLKRLDMTEHIT
KTLSIDEMLPAVSQGAIGIACRTDDGASRNLLAALNHEETRIAVVCERAFLTALDGSCRTPIAGYAHKGADGMLH
FSGLVATPDGKQIMRASRVVPFTEADAVKCGEEAGKELKANGPKELFMY
Porphobilinogen deaminase (PBGD2) nucleic acid sequence (SEQ ID NO: 37):
atgcgatcgtatctgctcaaggctcaagtggcctcatgtcagttttcgcgcacgtcgaaggtctggagactggcg
ccgggttctgacagacgacggtgtcggggcctcactcggacaccgcactgcgcggcccccaccagcgagcccgcc
ccgccatccagcagcggcaagagcgggcaacgaccactcgtgatagccacgcggccatctaagcttgcaaaggag
cagacgcggcaggtgcagcagctgctgctggcggcggcgcagctcaaggacgagcagctgcagctgagcaccctg
gaactggcgtctaggggcgacacgactcagggtgtgtcgctgcgcagtctgggctcgggcgcattcaccgaggag
ctggaccaggctgtgctgtcgggcgctgccgacatgtcggtgcacagcctgaaggactgccccgccgccctggcg
cccgggctgctgctggccgcctgcctgccgcgggccgacccccgggacgtcctcatcgcgcccgaggccacctcg
ctgggcgagctggtgccgggcagccgtgtgggcaccagcagcagccgccgcgcggcgcagatcaagcactccttc
ccccacctgcaggttgtgcagctgcgcggcaatgtggactcgcggctggggcgcatccgcagccgcgacatcggc
gccacagtgctggcggcggcgggcctcaagcggctgggtgtgatgaactcggacgagggtgacactaccgctacg
ggcgccgtgggggtggtgtgcagggcagacgatgagtgggtggtcggcctgctggacgccatctcgcaccgcggc
acggccctggaggtggcggcggagcgggcgtgcctggcagcgctgctgggcggcggcggcgcgtgccagcgttca
gcgttcccggacattgcgtgggcctgccacacgcggcacgaccccgacagcaacacaatggacctggattgcctg
gtggcggacctggagggcaaggagctcttcaggtacacggagttctaccggccggtcattgacgaggtggacgcg
gtgtcgctggggtcgctgtacggcagcctgctgcgcatgatggcgccaccaggcgcggccccctgttggcagcta
ccttcctcgcggcattag
Porphobilinogen deaminase (PBGD2) amino acid sequence (SEQ ID NO: 38):
MRSYLLKAQVASCQFSRTSKVWRLAPGSDRRRCRGLTRTPHCAAPTSEPAPPSSSGKSGQRPLVIATRPSKLAKE
QTRQVQQLLLAAAQLKDEQLQLSTLELASRGDTTQGVSLRSLGSGAFTEELDQAVLSGAADMSVHSLKDCPAALA
PGLLLAACLPRADPRDVLIAPEATSLGELVPGSRVGTSSSRRAAQIKHSFPHLQVVQLRGNVDSRLGRIRSRDIG
ATVLAAAGLKRLGVMNSDEGDTTATGAVGVVCRADDEWVVGLLDAISHRGTALEVAAERACLAALLGGGGACQRS
AFPDIAWACHTRHDPDSNTMDLDCLVADLEGKELFRYTEFYRPVIDEVDAVSLGSLYGSLLRMMAPPGAAPCWQL
PSSRH
Protoporphyrinogen oxidase (PPX1) nucleic acid sequence (SEQ ID NO: 39):
atgatgttgacccagactcctgggaccgccacggcttctagccggcggtcgcagatccgctcggctgcgcacgtc
tccgccaaggtcgcgcctcggcccacgccattctcggtcgcgagccccgcgaccgctgcgagccccgcgaccgcg
gcggcccgccgcacactccaccgcactgctgcggcggccactggtgctcccacggcgtccggagccggcgtcgcc
aagacgctcgacaatgtgtatgacgtgatcgtggtcggtggaggtctctcgggcctggtgaccggccaggccctg
gcggctcagcacaaaattcagaacttccttgttacggaggctcgcgagcgcgtcggcggcaacattacgtccatg
tcgggcgatggctacgtgtgggaggagggcccgaacagcttccagcccaacgatagcatgctgcagattgcggtg
gactctggctgcgagaaggaccttgtgttcggtgaccccacggctccccgcttcgtgtggtgggagggcaagctg
cgccccgtgccctcgggcctggacgccttcaccttcgacctcatgtccatccccggcaagatccgcgccgggctg
ggcgccatcggcctcatcaacggagccatgccctccttcgaggagagtgtggagcagttcatccgccgcaacctg
ggcgatgaggtgttcttccgcctgatcgagcccttctgctccggcgtgtacgcgggcgacccctccaagctgtcc
atgaaggcggccttcaacaggatctggattctggagaagaacggcggcagcctggtgggaggtgccatcaagctg
ttccaggaacgccagtccaacccggccccgccgcgggacccgcgcctgccgcccaagcccaagggccagacggtg
ggctcgttccgcaagggcctgaagatgctgccggacgccattgagcgcaacatccccgacaagatccgcgtgaac
tggaagctggtgtctctgggccgcgaggcggacgggcggtacgggctggtgtacgacacgcccgagggccgtgtc
aaggtgtttgcccgcgccgtggctctgaccgcgcccagctacgtggtggcggacctggtcaaggagcaggcgccc
gccgccgccgaggccctgggctccttcgactacccgccggtgggcgccgtgacgctgtcgtacccgctgagcgcc
gtgcgggaggagcgcaaggcctcggacgggtccgtgccgggcttcggtcagctgcacccgcgcacgcagggcatc
accactctgggcaccatctacagctccagcctgttccccggccgcgcgcccgagggccacatgctgctgctcaac
tacatcggcggcaccaccaaccgcggcatcgtcaaccagaccaccgagcagctggtggagcaggtggacaaggac
ctgcgcaacatggtcatcaagcccgacgcgcccaagccccgtgtggtgggcgtgcgcgtgtggccgcgcgccatc
ccgcagttcaacctgggccacctggagcagctggacaaggcgcgcaaggcgctggacgcggcggggctgcagggc
gtgcacctggggggcaactacgtcagcggtgtggccctgggcaaggtggtggagcacggctacgagtccgcagcc
aacctggccaagagcgtgtccaaggccgcagtcaaggcctaa
Protoporphyrinogen oxidase (PPX1) amino acid sequence (SEQ ID NO: 40):
MMLTQTPGTATASSRRSQIRSAAHVSAKVAPRPTPFSVASPATAASPATAAARRTLHRTAAAATGAPTASGAGVA
KTLDNVYDVIVVGGGLSGLVTGQALAAQHKIQNFLVTEARERVGGNITSMSGDGYVWEEGPNSFQPNDSMLQIAV
DSGCEKDLVEGDPTAPRFVWWEGKLRPVPSGLDAFTFDLMSIPGKIRAGLGAIGLINGAMPSFEESVEQFIRRNL
GDEVFFRLIEPFCSGVYAGDPSKLSMKAAFNRIWILEKNGGSLVGGAIKLFQERQSNPAPPRDPRLPPKPKGQTV
GSFRKGLKMLPDAIERNIPDKIRVNWKLVSLGREADGRYGLVYDTPEGRVKVFARAVALTAPSYVVADLVKEQAP
AAAEALGSFDYPPVGAVTLSYPLSAVREERKASDGSVPGFGQLHPRTQGITTLGTIYSSSLFPGRAPEGHMLLLN
YIGGTTNRGIVNQTTEQLVEQVDKDLRNMVIKPDAPKPRVVGVRVWPRAIPQFNLGHLEQLDKARKALDAAGLQG
VHLGGNYVSGVALGKVVEHGYESAANLAKSVSKAAVKA
Uroporphyrinogen III decarboxylase (UROD1) nucleic acid sequence (SEQ ID NO: 41):
atgcagaccaaggctttcacctctgcgcgcccccagcgggccgctgcgctcaaggcgcagcgcacctcgtcggtg
accgtgcgcgcgaccgcggcccccgccgtggcctctgcccccgccgcctcgggctctgcctctgaccccctgatg
ctgcgcgccatccgcggcgacaaggtggagcgcccgcccgtgtggatgatgcgccaggccggccgctaccagaag
gtgtaccaggacctgtgcaagaagcaccccacgttccgtgagcgctcggagcgcgtggacctggcggtggagatc
tctctgcagccgtggcacgcgttcaagcccgacggcgtcatcctgttcagcgacattctgacccccctgcccggc
atgaacatccccttcgacatggcgcccggccccatcatcatggaccccatccgcaccatggcgcaagtggagaag
gtgacgaagctggacgctgaggccgcctgccccttcgtgggcgagtcgctgcgccagctgcgcacctacatcggc
aaccaggccgcggtcctgggcttcgtgggcgcccccttcaccctggccacctacattgtggagggcggcagctcc
aagaacttcgcgcacatcaagaagatggctttctccacccccgagatcctgcacgccctgctggacaagctggct
gacaacgtggccgactacgtccgctaccaggccgacgccggcgcccaggtggtgcagatcttcgactcgtgggcc
agcgagctgcagccccaggacttcgacgtgttctccggcccctacatcaagaaggtgatcgacagcgtgcgcaag
acccaccccgacctgcccatcatcctctacatcagcggctctggcggcctgctggagcgcatggcctcttgctcg
cccgacatcatctcgctggaccagtcggtggacttcaccgacggcgtcaagcgctgcggcaccaacttcgccttc
cagggcaacatggaccccggcgtcctgttcggctccaaggacttcatcgagaagcgcgtcatggacaccatcaag
gctgcccgcgacgccgacgtgcgccacgtgatgaacctgggccacggcgtgctgcccggcacccccgaggaccac
gtgggccactacttccacgtcgcccgcaccgcccacgagcgcatgtaa
Uroporphyrinogen III decarboxylase (UROD1) amino acid sequence (SEQ ID NO: 42):
MQTKAFTSARPQRAAALKAQRTSSVTVRATAAPAVASAPAASGSASDPLMLRAIRGDKVERPPVWMMRQAGRYQK
VYQDLCKKHPTFRERSERVDLAVEISLQPWHAFKPDGVILFSDILTPLPGMNIPFDMAPGPIIMDPIRTMAQVEK
VTKLDAEAACPFVGESLRQLRTYIGNQAAVLGFVGAPFTLATYIVEGGSSKNFAHIKKMAFSTPEILHALLDKLA
DNVADYVRYQADAGAQVVQIFDSWASELQPQDFDVFSGPYIKKVIDSVRKTHPDLPIILYISGSGGLLERMASCS
PDIISLDQSVDFTDGVKRCGTNFAFQGNMDPGVLFGSKDFIEKRVMDTIKAARDADVRHVMNLGHGVLPGTPEDH
VGHYFHVARTAHERM
Uroporphyrinogen III synthase (HEM4) nucleic acid sequence (SEQ ID NO: 43):
atgtcggccctggacgccgccgccatcccctacgagctagtgccgggtgtgtcctccgctctggccgccccgctg
ttcgccggcgtcccgctcacacacgtcagcctgagcccctcgttcaccgtggtcagcgggcacgacgtggccggc
accgactgggcggcgttccgggggctgcccacgctggtggttctgatggcgggtcgtaacctggggcagatagcc
cggcggcttgtgcaggacgcggggtgggcgcccgatacacctgtaagtcaacctagtggctag
Uroporphyrinogen III synthase (HEM4) amino acid sequence (SEQ ID NO: 44):
MSALDAAAIPYELVPGVSSALAAPLFAGVPLTHVSLSPSFTVVSGHDVAGTDWAAFRGLPTLVVLMAGRNLGQIA
RRLVQDAGWAPDTPVSQPSG
CHLD 5′ untranslated region (regulatory region) (SEQ ID NO: 45):
ggcgtccccacaaccaggacagcctacttcttgaccttattaataagtcgctgcgtgtcgcgactgaccattttg
gcccggacttgcgtgcttgtgatttgtgcttcgactagatccgcgggcaccaagggacgcggacagctgatagtc
aagaactagatcctctgggagcgtctggggctgtccccgctgctcgccaaggaa
CHLD 3′ untranslated region (regulatory region) (SEQ ID NO: 46):
gtgccgagtgactgaggtggcaaggtgcagtggcggcggaggcagttgtgctggggtggcaaggcggacaggcga
agctggtgggttgcgacgaggaggaggtgcacgtgcacgcgtaacataagaagaacagtgggaggacaggtagcg
tgacttgactgggacgaggagcgtactgatgtgtggcgtgtgttggtatgtgagcgttacccctcccctagatag
cggcggtctccactttcaggaggatgagagccatcatgaggctttgagggggcactggttcgtgtgtaggctgag
gctgctgttgaagtcacaaggcagcactgcatgcgcgagtgagtgtggccggatatgcatcgagttgcaggtaca
ctgaaatgaggtgactgcggcgtatatcgctgccagtacaggttgaagcggcgggcacggtgaatggagtactcg
gcctggaacgcttgcgatcagatggtcgagctcaagaagatttggttgagccgttgggtcgtgcgtcatattatg
gcttgcatcttcggggagcggcaagaaacggactccaatgcaggccctcgggcgagaaagattgggcgtgtccgg
gggtgcattctcgccgcgtggggctgcatcgaatttcgcttgagtgccccttcccggggagggggggcggtagtt
caaccccatcatcgtaggggggttgtaaatgccagcccaaactaaa
CHLD Exon 1 (SEQ ID NO: 47):
atgaagtctctctgccatgagctcgctggccccagcgttactgggtgcggccggcgaagcctccggaaggctttc
agcggtgccaagattgcgcaggtctctcgccccgctgtgcttaacagcgtgcagcgccaacagcgtctcgcctgt
tctgccgtggccgagctctccgctgctgagctgcgcg
CHLD Exon 2 (SEQ ID NO: 48):
ccatgaaggtgtctgaggaggactccaagggcttcgatgcggatgtgtcgacccgcctggcccgctcgtaccctc
tggcggccgtggtgggccaggacaacatcaagcaggcgctgctgctgggcgccgtggacaccgggctgggcggca
tcgccatcgccggtcgccgcggtaccgccaagtccatcatggctcgcggcctgcacgctctgctgccgcccattg
aggtggtggagggcagcatctgcaacgccgaccccgaggacccccgctcctgggag
CHLD Exon 3 (SEQ ID NO: 49):
gctggcctggctgagaagtatgcgggcggccctgtgaagaccaagatgcgctcggcgccgtttgtgcagatccct
ctgggtgtgactgaggaccgcttggtgggcactgtggacattgaggcgtccatgaag
CHLD exon 4 (SEQ ID NO: 50):
gagggcaagactgtgttccagcccggcctgctggctgaggcgcaccgcggcatcctgtacgtggacgagatcaac
ctgctggatgacggcattgccaacctgctgctgtccatcctgtcggacggagtcaacgtggtggagcgcgagggc
atctccatcagccaccc
CHLD exon 5 (SEQ ID NO: 51):
ctgccggccgctgctgattgccacctacaaccccgaggagggccctctgcgtgagcacctgctggaccgcatcgc
cattggcctcagcgccgacgtccccagcaccagcgacgagcgcgtcaaggc
cattgacgcagccatccgcttccaggacaagccgcag
CHLD exon 6 (SEQ ID NO: 52):
gacactattgacgacacc gcggagctcaccgacgccctgcgcacctcg
CHLD exon 7 (SEQ ID NO: 53):
gtcatcctggctcgcgagtacctgaaggacgtgaccatcgcgccggagcaggtgacctacattgtggaggaggcg
cgccgcggcggagtccaggggcacc gcgcggagctgtacgcggtcaag
CHLD exon 8 (SEQ ID NO: 54):
tgtgccaaggcgtgtgcggctctggagggccgtgagcgtgtgaacaaggatgacctg
cgccaggccgtgcagctggtcatcctgccgcgcgccaccatcctggaccagcccccgcccgagcaggagcagccc
ccgccgccgcccccgccccctcccccgccgccgccgcag
CHLD exon 9 (SEQ ID NO: 55):
gaccaaatggaggacgaggaccaggaggagaaggaggacgagaaggaggaggaggagaaggagaacgaggaccag
gacgagcccgag
CHLD exon 10 (SEQ ID NO: 56):
atccctcaggagttcatgtttgagtccgagggcgtcatcatggacccctccatcctcatgttcgcgcagcagcag
cagcgcgcgcagggccgctccggccgcgccaagacgctcatcttcagcgacgaccgcggccgctacatcaagccc
atgctgcccaagggtgacaaggtcaagcgcctggcagtggacgccacgcttcgcgccgccgcgcccta
ccagaag
CHLD exon 11 (SEQ ID NO: 57):
attcgccggcagcaggccatcagcgagggcaaggtgcagcgcaaggtgtacgtggacaagccagaca
CHLD exon 12 (SEQ ID NO: 58):
tgcgctccaagaagctggcccgcaaggccggtgcgctggtgatttttgttgtggacgcgtccggctccatggctc
tgaaccgcatgagcgccgccaagggcgcctgcatgcgcctgctggctgagtcgtacaccagccgcgaccaggtgt
gcctcatccccttctacggcgacaaggccgaggtgctgctgccgccctccaagtccatcgccatggcccgccgcc
gcctggactcgctgccctgcggcggcggctcgccccttgcgcacggcctgtccacggcggtacgtgtgggcatgc
aggccagccaggcgggcgaggtgggccgcgtcatgatggtgctcatcacggacggccgcgccaacgtcagcctgg
ccaagtccaacgaggaccccgaggcgctcaagcccgacgcgcccaagcccaccgccgactcgctgaaggacgagg
tgcgcgacatggccaagaaggccgcgtccgccggcatcaacgtgcttgtcattgacacggagaacaagttcgtga
gcaccggctttgcggaggagatctccaaggcagcgcagggcaagtactactacctgcccaacgccagcgacgccg
ccatcgcggcggccgcgtccggcgccatggccgcggccaagggcggctactag
CHLD Intron 1 (SEQ ID NO: 59):
gtgagcgcctactttgatatgtaccaaagataccactgataggtttaggcacggaagatctggacttggaccccg
tttgcgcaagccgggcgatgcacccatttcgcggtcacgccgagcgctggggtgcaatttagcgtgcccgacaag
ctagaaaacagggaattaccatttgtttaattttgttgcgagagatctttgcttgtgtccaccggccgcgcgggg
gaacttccggtgttgcgcaaggttgcgtgcgtgcccaccatcaacacctgtgccaggtctgtgtcacccccaggt
tccaccaccctgcaatcttccaattgtgtctcgtttgctcgttgtctaatagtcgtcctttgctcatccctacct
gcag
CHLD Intron 2 (SEQ ID NO: 60):
gtgaggcagggaaggtgacacaggaggttttgaaagagagacagggaggcaaagatggatggcggggcgggcagt
gactttggggcggcatggagtgggattggtggagtgggattgggcaccatgtatcacagatgttggcaacacagc
gcagggccttgctctgtgcttgtgttgaccgtctagtcccccgtgccctgaaccaagtctttcctcctgacacgg
tcctccatgtcctccttccggcattcccttcctcgtccacag
CHLD Intron 3 (SEQ ID NO: 61):
gtgagccagcaagggaggagaggggaacggccgggtagggcagccggagtttaaccacgccaattcaacggggag
caacggggaagaggaagggccggaagaggacggcaaaagcatttggtgggggcagcggctgtagtcagaagcgca
aaggctgccacagtgtggcccgcaccctcctcaccaccagtttggcatgatcgtttagcatgggctggaatactc
accgccagttctctcctctcccctctcctcccctgtccccgcctgcag
CHLD Intron 4 (SEQ ID NO: 62):
gtgagtgcgcgcgctgggtgtgtttgtgggacggcgcggcattggagcgcaggtgcgggtgctgggccgtgcact
tgtccgttggttcccttggaagcttcgatacacactcttactgcacgctctttaaccgccccccccctccacctc
tgcccgccccgtgcag
CHLD Intron 5 (SEQ ID NO: 63):
gtgggtgggggaaagtgactggatgtcggtgggttttaggtatgtgcgtgtgtacgatgcggggagcagtacgga
agcgggcacgagcggtgagggggcaggattgtggcgcacgctcgggccaagcccgggctcgcgacagagggtggg
cttgtattcgtagtcaagcgcatcaggaagtgcagttgactggattcacctgaaacggcgctgagcgggcggcta
atagaatcccgcttcctgtccgcccctccccttgcccttcaatccgtcag
CHLD Intron 6 (SEQ ID NO: 64):
gtgagtggcgggggccgtgcgtttgtttgttgcgtgggctggctggctggctttgttggatgagggcgctgctca
ccactcatctctttgaatccccacttatccagttgcctgcatgaaaccccgcctgactcactccccaccatcctg
taccgcttttccaaacatccttgcaaccatcccgccatccccacccgcag
CHLD Intron 7 (SEQ ID NO: 65):
gtgaggagttggagggggaaggggcgaggggatgcgacagaagcgagggcgaggggagccggggtgggttgttgc
aagtgtcgtgaattatagaatgaccccaaaagcgccggcccaacagggcctattacttgcgagtcaatccaaccc
ctgatatagggagaatggggtagaggtcgtatcacgacagcaaggatgtacagtgggccttggggttgggaggta
cagggaaaaaggagaggacatggggttgggtaagcggggaataacaaatatacacccagcgtttatggaagtggg
agatggaaacgggggcggacgaacaggaacaggggccggatggaggggctatgggggcatggtgggtgggggtac
ggcgcggggcagagcagggtcttgggtgaatgggcaagatgctgatgcttgggatgaagacactatgagcaaaga
aatggttgttgacgattgccatgatcatcgcagtgggggaggcggggtggcaataccggcagtcaacagttgggg
tgcgatcaagattgattggagtaccagcagtggccgggatctggctgacgtgtctcgagcgagttgctggggtgg
caaggagatgcaggggcagacgacgttgtgcgaccacacttacacacatttccttccccttgcgtgtgtccgtgc
gccctgtgcctccag
CHLD Intron 8 (SEQ ID NO: 66):
gtacgtaaacgtatttgattgctcaggtggttagccttggtgtggctgctgtttgacttgtgcagctgtctttgt
gtacatgttccacaaccctgtactccccatattccgcccccattccag
CHLD Intron 9 (SEQ ID NO: 67):
gtgagaggcggcgcggcggcttgcgggcgaaggcggggggcggggcggaggcaatgcggccgcgcatggccagca
acggaagggctggctatcaacacggcgagcgcacgatattcatataagagtgccatcgtgcaatgctgaatactt
gcgccaaccggatctcgctgctccgcttccaccggactgctttctcatctctccccttcaccctgtgtgtatcca
cag
CHLD Intron 10 (SEQ ID NO: 68):
gtgagtgcccgaggtggtgggtggtgaattggggcacgagggtatgtgggcctaagggagctgaatggggcatgt
tttcttctgagcatcacggtcagagcttgacctgtcctccccgctgtacccccgtgcacggtccgacacag
CHLD Intron 11 (SEQ ID NO: 69):
gtgagtacagcgcatcccggcgcaatcattgggcctagttactgctgcaggactcgtgtgctcttaagggctggc
agctgtcagaagctctactcctcgcactgaccactgtgcctttctctccttcctctctccctccccgcacccctc
ctcccacttcctcaacag
CHLI2 5′ - untranslated region (regulatory region) (SEQ ID NO: 70):
gcagacttccataaagctcttgtaacgctgtaccaactagtaagcggtacaattcgcctgagcccgagcaacgcg
acctttcttgctctgtggatctctgataatctaaccagaccaaaaccttttcactaatctaggcaaca
CHLI2 3′ - untranslated region (regulatory region) (SEQ ID NO: 71):
aaaaggctggtgtaggcctgtcgggtcgtgttaaaggttgctgcgtgaacgtgtaagtgtgacagtgtgccggta
tgtgtgtgtatacatgtgttgcggtgtgcttttgtggcggtacatggtgatgactgagcgggtgggacagagcac
ggttaactgacgagggcagtccgtgcgagacggacgtttttgtagccgaggtgcaaggactgatgacgggctaag
ctgctggagacttggagttgagagtgcaggtggatcgacggtttctctaaggagtatgaataggcaggagggctg
gagacatttggggtgcaaggaggcggtagtatgggagatgtccatgggcggattttggcctctgtaacttcttaa
cgccca
CHLI2 Exon 1 (SEQ ID NO: 72):
atgcagagtctccagggtcagcgcgcgttcactgcggtgcgccagggtcgggcgggtcccctgcggactcgcctg
gtcgtgcgctcgtctgttgccttgccatccacgaaagccgcgaagaagccgaacttcccgttcgtcaagattcag
ggccaggaggagatgaagcttgcactgctgctgaacgtggtcgaccccaacatcggcggagtgcttattatgggt
gaccgcggcactgccaagtcggtcgcg
CHLI2 Exon 2 (SEQ ID NO: 73):
gtccgcgccctggtggatatgcttcccgacattgacgtggttgagggcgacgccttcaacagctcccccaccgac
cccaagttcatgggccccgacaccctgcagcgcttccgcaacggcgagaagctgcccaccgtccgcatgcggacc
cccctg
CHLI2 Exon 3 (SEQ ID NO: 74):
gtggagctgcctctgggcgccaccgaggaccgcatctgcggcaccatcgacatcgagaaggcgctgacgcagggc
atcaaggcctacgagcccggcctgctg
CHLI2 Exon 4 (SEQ ID NO: 75):
gccaaggccaaccgcggcatcctgtatgtggacgaggtgaacctgctggatgatggcctg
CHLI2 Exon 5 (SEQ ID NO: 76):
gttgatgtcgtgctggactcgtcggctagcggcctgaacactgtggagcgtgagggtgtgtccattgtgcaccct
gcccgcttcatcatgattggctcaggcaacccccag
CHLI2 Exon 6 (SEQ ID NO: 77):
gagggtgagctgcgcccgcagctgctggatcgcttcggcatgagcgtcaacgtggccacgctgcaggacaccaag
cagcgcacgcagctggtgctggaccg
CHLI2 Exon 7 (SEQ ID NO: 78):
gcttgcgtacgaggcggaccctgacgcatttgtggactcgtgcaaggccgagcagacggcgctcacggacaagct
ggaggcggcccgccagcgcctgcggtccgtcaagatcagcgaggagctgcag
CHLI2 Exon 8 (SEQ ID NO: 79):
atcctgatctcggacatttgctcgcgcctggatgtggatggcctgcgcggtgacattgtgatcaaccgcgccgcc
aaggcgcttgtggccttcgagggccgcaccgaggtgaccacgaatgacgtggagcgcgtcatctcgggctgcctc
aaccaccg
CHLI2 Exon 9 (SEQ ID NO: 80):
cctgcgcaaggacccgctggaccccattgacaacggcaccaaggtggccatcctgttcaagcgcatgaccgaccc
cgagatcatgaagcgcgaggaggaggccaagaagaagcgcgaggaggcggccgccaaggccaaggcggagggcaa
ggcggaccgccccacgggcgccaaggctggcgcctgggctggcttgccccctcgtcggtaa
CHLI2 Intron 1 (SEQ ID NO: 81):
gtaggtaacacaagcaattatggggcgaagatctaggctccgctgatccgggcgggcaatcggcatcgtcggtgc
aaccgtggggcgtctgtgcaccctttgctggtgccaggttgcctgactcgcctgcattcctgtaccgagccacat
tggctgctttgcagcgtgcatgggacgggtgtaggataagcgctatgtatgcgatagcgcgggtgcaccggcttg
gcatggcaaggttgcggggtgcacatgcgtgccagcgtcccctcagcatcagagtctggatctaagggctcagcg
gcttcctgcgcatgtgggtctttgcgtagtgctacgaagccttataattaaagctcatgtattgagtggtccggg
tttggggcactagtagtgccaggaggcgcgtgccaggttgatatgagcatatcagcacccgttccttgcgaaacg
cttccgttgtgctcccttccccaccacctccccgctcatacccatacatatggctatccgtcctctcattgcttg
cccctacag
CHLI2 Intron 2 (SEQ ID NO: 82):
gtgagcgggcctaccttctgaagacagtcttacgtgttgcactgcagcggtgttgcgcacctctgcttttgcgtg
cgccgggaagcgcggattgcggcctcacagatcaagcccggaaacgcttgttgtttccagcgggtggcacacacg
cgcgcgcgcgcacagtgacaccctcacggccgcgctgccctgcag
CHLI2 Intron 3 (SEQ ID NO: 83):
gtgcgtagtgcatggggagaggggacgaggggaggagggcagggccaataaaccgaaccccaagtcatcgagaca
cagaacccgataatagctcccagatcgccaaggggtgaggcgggaagccaaggatgatgcgttggccgcattgcg
tgttgacgtcaggcttacacagggtctgactggctgtgcttggggtttggcacgcttcttgactggccccgtacg
catgctgcag
CHLI2 Intron 4 (SEQ ID NO: 84):
gtgagtggtggtggtttctgggtcagcagaggacttctgtagtaggtaatgtgggccagggaagtgtggctaaca
tgccaaacacgggggcgcaccagtgcaagctgcattcgctgacgtgcacgggtgcaatgggtgcaaggcgaactg
caatcgcggtgcacagttgccagggctgcgctcacgcttgagtgtctgcacacgcactgcag
CHLI2 Intron 5 (SEQ ID NO: 85):
gtgcgtagcgtgcgcgcatgtacttgtctcccttgtcatgttgggaaaggtcggtccccagcctgcttgcaagat
gcggccggtcagcagctgcggacggtcagcacctacgtgccgaggttgtgtaacatgaatggcgttggggcggcc
gacctgccacaagctgaactgcgaccagcaaggcagctgccagcaacgcacacccgacgtgctacacgcttgtgt
tttgacctcctaaacacacccgcccgctgtctgtcacgtccacag
CHLI2 Intron 6 (SEQ ID NO: 86):
gtaagcggcggcggcgcggggacacggagggacatttcgcgagcatgggttgaggagtcgggaggattcggtggc
tggccggagtcgggagtcggagtcgcgagtcggaagtcaagcttctggcggcttcgtgctgtcgggtgcgctcgc
catgatggcgctgaccggagggcgtcacgctgtgtatgtgggcgcgcag
CHLI2 Intron 7 (SEQ ID NO: 87):
gtacggggcgtacagcgggggcggctgcacggggccagtgaccgacagggcagcacgcggctggcgaagagcgac
aaagtgacagggtgaccaagaccgggtgatgccacgagaggggcgcgggagccgtgcattgggtcgaggagggag
gaatgcaactttacactgatgcctctgtatacggccgccttccgagccctgcaaaccttcgctttcccccgacgc
acgcag
CHLI2 Intron 8 (SEQ ID NO: 88):
gtgagcgcagcgtgcggtggatgcggtgcgcgtgcgggttgccaacttattattttgtacgtggacgcgtggctg
gcgatggcatgtcatggcgcgaatggatattgggcgaatggataccggtaatggtagcacggggcggcagggcct
ggcggtagtggggttgagggggcgaggactccagcgcgcgatacatgccatgttcagcatggccccaactgacag
cgcccgctgccctgtgcgccccgctccctccgcgcacccgctcctcctacacag
CHLH1 5′ - untranslated region (regulatory region) (SEQ ID NO: 89):
ctagtctagagggaactagggaggggcaacagagaa
CHLH1 3′ - untranslated region (regulatory region) (SEQ ID NO: 90):
gcggcctccccttcatggtagcactagttggcgggttgtggttggactaggcggctagggtatatacctagtagc
ggcggctgcggagtggagggctggcgcccagcgcgagggcgtggcctttcctcctggacccgagagcgctccgcg
aggagacggcgagtgagataggcagcagcgagcggagatcgatttgtgaacagttttgtggcgggatcccatagc
ggatgcagagaagaccttagagcagcttcctcggtggagtgaacgagccagagcggagggaaggcgcatgaggga
actgcagggactggaactgcgggagtgcaggtccggtgctaggtccgctaaacagtgcggtctacgcctgtgtgt
gaggtgtgcgtgtgtgtgtgagctgtgcggttttgttgtgcaaagtaggagtgagccgagccgcgcgtactttgt
ggcgtgtttggctgctggcgctgagagccaagagagggtaaacgggtttggtattttatggtgcggggtgaaagc
agccctcgcaggaatggagcgattctgcagcatgatgcacgtgtgcctgcgcgtggatggtggctgttgatatgg
ctctgccactccggcagcaccgctacgatacctagcggtgcctggagtggtctctctgtttggtgcgtgatgttt
gggtttgccgttttgattctttgtttcgtgctgaatggctgaggcggcaagacccctcgtgccagtgtacagagc
ctcacggctccctcggaccccgcgtggggacgtccattcccggtggcggtgtcgcctcggcggtgtaaagcaaaa
aatatttt
CHLH1 Exon 1 (SEQ ID NO: 91):
atgcagacttcctcgcttcttggccggcgcacggcccacccggctgcgggcgcgacgcccaagccg
CHLH1 Exon 2 (SEQ ID NO: 92):
gttgcgccctcgccccgcgtggctagcacccgccag
CHLH1 Exon 3 (SEQ ID NO: 93):
gtcgcgtgcaatgtggcgactggaccccggccgcccatgaccaccttcaccggtggcaacaagggccctgctaag
cagcaggtgtcgctggatctgcgcgacgagg
CHLH1 Exon 4 (SEQ ID NO: 94):
gcgctggcatgttcaccagcaccagcccggagatgcgccgtgtcgtccctgacgatgtgaagggtcgcgttaagg
tgaaggttgtgtacgtggtgctggaggcccagtaccagtcggccatcagcgctgcggtgaagaacatcaacgcca
agaactccaag
CHLH1 Exon 5 (SEQ ID NO: 95):
gtgtgcttcgaggtggtgggctacctgctggaggagctgcgtgaccagaagaacctcgatatgctcaaggaggat
gtggcctctgccaacatcttcatcggctcgctcatcttcattgaggagcttgccgagaag
CHLH1 Exon 6 (SEQ ID NO: 96):
attgtggaggcggtgagccccctgcgcgagaagctggacgcgtgcctgatcttcccgtccatgccggcggtcatg
aagctgaacaagctgggcacgttttcgatggctcagctgggccagtcgaagtcggtgttctcggagttcatcaag
tctgctcgcaag
CHLH1 Exon 7 (SEQ ID NO: 97):
aacaacgacaacttcgaggagggcttgctgaagctggtgcgcaccctgcctaaggtgctgaagtatctgccctcg
gacaaggcgcaggacgccaagaacttcgtgaacagcctgcagtactggctgggcggtaactcggacaacctggag
aacctgctgctgaacaccgtcagcaactacgtgcccgctctgaagggcgtggacttcagcgtggctgagcccacc
gcctaccccgatgtgggtatctggcaccctctggcctcgggcatgtacgaggacctgaaggagtacctgaactg
CHLH1 Exon 8 (SEQ ID NO: 98):
gtacgacacccgcaaggacatggtcttcgccaaggacgcccccgtcattggcctggtgctgcagcgctcgcacct
ggtgactggcgatgagggccactacagcggcgtggtcgctgagctggagagccgcggtgctaaggtcatccccgt
ctttgccg
CHLH1 Exon 9 (SEQ ID NO: 99):
gtggcctggacttctccgcccccgtcaagaagttcttctacgaccccctgggctctggccgcacgttcgtggaca
ccgttgtgtcgctgaccggcttcgcgctggtgggcggccccgcgcgccaggacgcgccgaaggccattgaggcgc
tgaagaacctgaacgtgccctacctggtgtcgctgccgctggtgttccagaccactgaggagtggctggacagcg
agctgggcgtgcaccccgtccaggtggctctgcag
CHLH1 Exon 10 (SEQ ID NO: 100):
gttgccctgcccgagctggatggtgccatggagcccatcgtgttcgctggccgtgactcgaacaccggcaagtcg
cactcgctgcccgaccgcatcgcttcgctgtgcgctcgcgccgtgaactgggccaacctgcgcaagaagcgcaac
gccgagaagaagctggccgtcaccgtgttcagcttcccccctgacaagggcaacgtcggcactgccgcctacctg
aacgtgttcggctccatctaccgcgtgctgaagaacctgcagcgcgagggctacgacgtgggcgccctgccgccc
tcggaggaggatctgatccagtcggtgctgacccagaaggaggccaagttcaactcgaccgacctgcacatcgcc
tacaagatgaaggtggacgagtaccagaagctgtgcccttacgccgaggcgctggaggagaactggggcaagccc
cccggcaccctgaacaccaacggccaggagctgctggtgtacggccgccagtacggcaacgtcttcatcggcgtg
cagcccaccttcggctacgagggcgacccgatgcgcctgctgttctcgaagtcggccagcccccaccacggcttc
gccgcctactacaccttcctggagaagatcttcaaggccgacgccgtgctgcacttcggcacccacggctcgctg
gagttcatgcccggcaagcaggtcggcatgtcgggtgtgtgctaccccgactcgctgatcggcaccatccccaac
ctctactactacgccgccaacaacccgtctgaggccaccatcgccaagcgccgctcgtacgccaacaccatttcg
tacctgacgccgcctgccgagaacgccggcctgtacaagggcctgaaggagctgaaggagctgatcagctcgtac
cagggcatgcgtgagtctggccgcgccgagcagatctgcgccaccatcattgagaccgccaagctgtgcaacctg
gaccgcgacgtgaccctgcccgacgctgacgccaaggacctgaccatggacatgcgcgacagcgttgtgggccag
gtgtaccgcaagctgatggagattgagtcccgcctgctgccctgcggcctgcacgtggtgggctgcccgcccacc
gccgaggaggccgtggccaccctggtcaacatcgctgagctggaccgcccggacaacaacccccccatcaagggc
atgcccggcatcctggcccgcgccattggtcgcgacatcgagtcgatttacagcggcaacaacaagggcgtcctg
gctgacgttgaccagctgcagcgcatcaccgaggcctcccgcacctgcgtgcgcgagttcgtgaaggaccgcacc
ggcctgaacggccgcatcggcaccaactggatcaccaacctgctcaagttcaccggcttctacgtggacccctgg
gtgcgcggcctgcagaacggcgagttcgccagcgccaaccgcgaggagctgatcaccctgttcaactacctggag
ttctgcctgacccag
CHLH1 Exon 11 (SEQ ID NO: 101):
gtggtcaaggacaacgagctgggcgccctggtagaggcgctgaacggccagtacgtcgagcccggccccggcggt
gaccccatccgcaaccccaacgtgctgcccaccggcaagaacatccacgccctggaccctcagtcgattcccact
caggccgcgctgaagagcgcccgcctggtggtggaccgcctgctggaccgcgagcgcgacaacaacggcggcaag
taccccgagaccatcgcgctggtgctgtggggcactgacaacatcaagacctacggcgagtcgctggcccaggtc
atgatgatggtcggtgtcaagcccgtggccgacgccctgggccgcgtgaacaagctggaggtgatccctctggag
gagctgggccgcccccgcgtggacgtggttgtcaactgctcgggtgtgttccgcgacctgttcgtgaaccagatg
ctgctgctggaccgcgccatcaagctggcggccgagcaggacgagcccgatgagatgaacttcgtgcgcaagcac
gccaagcagcaggcggcggagctgggcctgcagagcctgcgcgacgcggccacccgtgtgttctccaacagctcg
ggctcctactcgtccaacgtcaacctggcggtggagaacagcagctggagcgacgagtcgcagctgcaggagatg
tacctgaagcgcaagtcgtacgccttcaactcggaccg
CHLH1 Exon 12 (SEQ ID NO: 102):
ccccggcgccggtggcgagatgcagcgcgacgtgttcgagacggccatgaagaccgtggacgtgaccttccagaa
cctggactcgtccgagatctcgctgaccgatgtgtcgcactacttcgactccgaccccaccaagctggtggcgtc
gctgcgcaacgacggccgcacccccaacgcctacatcgccgacaccaccaccgccaacgcgcaggtccgcactct
gggtgagaccgtgcgcctggacgcccgcaccaagctgctcaaccccaagtggtacgagggcatgcttgcctcggg
ctacgagggcgtgcgcgagatccagaagcgcatgaccaacaccatgggctggtcggccacctcgggcatggtgga
caactgggtgtacgacgaggccaactcgaccttcatcgaggatgcggccatggccgagcgcctgatgaacaccaa
ccccaacagcttccgcaagctggtggccaccttcctggaggccaacggccgcggctactgggacgccaagcccga
gcagctggagcgcctgcgccagctgtacatggacgtggaggacaagattgagggcgtcgaataa
CHLH1 Intron 1 (SEQ ID NO: 103):
gtaggtgtaattagaaggatcaaaacctagcggcctgatctgggactgacggcctcgcgcttcaatcactctgat
gcag
CHLH1 Intron 2 (SEQ ID NO: 104):
gtaggcacggcagaatgctcaatgaacatgcagctacatatgtttgggatcatggctgatctctgtgcgacgggt
ccgcgcag
CHLH1 Intron 3 (SEQ ID NO: 105):
gtgagcagcgcggaccgagcaagcgctggcgatgcagttggatttgttgttcttgggtcaggcgctcgctcgatg
gccagcgcgtgtatttaatgggataagggttgagacaaagcatctcttcgggtaaaaatcttagttttcgacagc
acgttgagaggcatgcaacttgctctttcgcag
CHLH1 Intron 4 (SEQ ID NO: 106):
gtgggtaaggagttgcattatcagtgtggcatggtgttgcgggcgtctggggcgctgcaacagcggcatcgtgcc
gaactgaccgtgccgggctacccgcgtgcag
CHLH1 Intron 5 (SEQ ID NO: 107):
gtgcgctagggttggggtctggagggtgtggattgcgcccaagtgccctgttgcgcttggcggtcgctgtcatga
tgtgagggtgacgtagtgcactcaattgcctgctacgtcaccacctttgatgggctggatctgaggcaggtcagc
tcggttccctgctgcatccagtgtccctgtcgccctgcacgtttgacgctgttcccccttccgcactgtctcgct
ttgcag
CHLH1 Intron 6 (SEQ ID NO: 108):
gtgtgggcacgcgctttgggaagggaggcatacatttttggttgcggttaggctgggcgcggacttggcactcac
acggtcattgcacactcatgtctcaccttcatttacggtcccttgtgccgaactacctacag
CHLH1 Intron 7 (SEQ ID NO: 109):
gtgagcagcatcagggcagagtgcatgaacggattggtggcagtggggaatggaattagacggacacgtctgggc
ggcaatatgttgcgctgcagtttttggggtgtagtgaactagaaaatagggaagagataggccacataacatccg
aaagctcatatttttgcaaccggcgcacctatcacagcccacctgaagggttttgtagtcaacgcgtgcaactga
ctagatgtccccttacctgtctgatttcag
CHLH1 Intron 8 (SEQ ID NO: 110):
gtgaggcggggcggcgctgccctcggtaggggttgcagatggtgatgggtaaccgaatgcatggccaatggggag
tgaaatcaggaaaggaggggtaacacaatgcagggcagcacctgaatcgtgaaggcggagttaggcagggatctg
tcagttcgcctgtcacgtggatgggcgcagctgacctttgtggtgttgtggtgtggcgcag
CHLH1 Intron 9 (SEQ ID NO: 111):
gtgagctcagctgggacatgtaggggctcgggtcgccggagcatcgatgtagaattacgggaggaggggagaggg
gagaggattgcacgaaccgagatgagggcggtggttcgggatttcgggcaaaagctcgtgcggcaagcgttcagt
gactgaagagcagtgtgcttcaactgcccctctgtccctcag
CHLH1 Intron 10 (SEQ ID NO: 112):
gtgcgaccggtgccgctgcgtggccaacagcttggtgccaccttcctgcggtgttgatttacactgtgtgcgtgg
atgtgttggtttttcgcaactttagtctgggctccagctctttgccttcattgatcactcgtcttacctcctgcg
ccatcatttgaatacag
CHLH1 Intron 11 (SEQ ID NO: 113):
gtgagccttaatgcaacacgtgtagccgttcgcatgggtggctgggtcatgctatggttggatcggcgtccgcct
gcttgctactgcctgttcggtaccagcgtttactgaccccgcgtgtgccattcccaccacctaccccctcgcctt
gcag
Ferrochelatase 5′ - Untranslated region (regulatory region) (SEQ ID NO: 114):
gacagtgatatagcaataccgatataataggtttggcgggcttcaccttgtccttacccagaatgtggccctgac
agtcgatttccagcccccttgccactcgctccctgatttcttcaatcaactagttgggtcgttttctcgtaagg
Ferrochelatase 3′ - Untranslated region (regulatory region) (SEQ ID NO: 115):
gggggcgggtggcgagtaaggcgtatggcggagcgaggagatgggctgtggcgtggccggtgttcttttgtgtga
ttggaaacatagacggggtgcggcacgcggcctgactgctgcgcggttggtgtggttgcggggggagcggggtcg
atggggcagcgcgcacgagttggttgaaggaggagggccaggcgctgggctacacccatggtttgaggatgctag
tgagtgatgtgtgcggggggcatggtgtgtaccattcagagtccagatgcacgcacggttgcgtgggagcgttcc
ctgctgtgcatgatgatggcgccttcgatgaatcatctcttgaaggtccaaatgaaacgtctgaagtctgcagag
ggtggtgctggacatgccatccaggcggaagtgggcagctgtgtctgactacaaagtaggtcttgttttgcttgg
atagcgtttggctatgtagcgtgtattctgctcatcaatcacgccaggcgtcagggactacccatgcaagtcggg
agcgtggctggctctggaaaagttgtagctgctaggtggcgttggctggggtgtcatgcatctcggcaggtaggc
ggtagcggtggacgacctctgcagcggagcatgtgcacaagatgtgactgcgcatgcacccgtatatgacggcgt
tggcgtcagttgttgagagtgaacagaggagagacgagcgaagctgccatgcccttagtggctggtgcgagaggg
gaagaaagagagaggaaggactttgcggcagtgccccacgccggagttggggacacggtcatcaacagggcggcg
gagctgggcggagtgggtgtgtgatgggacagggttcaaggcaggttggcgaggtcggagtgggtagaccagtcc
ttcagtgcaagggcattagggcatgatgtaagggctgaagcttg
Ferrochelatase Exon 1 (SEQ ID NO: 116):
atggcgtcgtttggattgatgcaaaggacggtgcactgtccccagcttgtggaggagcggtgttcgccggtcgct
ggctgctctggtcgtggcctgccagttatccagcggcaacg
Ferrochelatase Exon 2 (SEQ ID NO: 117):
gcgtggcgtgtgcagtgccaccaacggtgtccagcgagggcgtgtgctgcgccggacggccgcttcgaccgacgt
ggtctccttcgtggaccccaatgacattagaaaacccgcagcagcagcagctggccctgcggtggataaggtcgg
cgttctgctgttaaaccttggcgggcccgaaaagctcgacgacgtcaagcctttcctgtataacctattcgccga
cccagaaattattcgcctgccagcggcagctcagttcctgcagccgctgctcgcgacgatcatctccacgcttcg
cgccccgaagagcgcggagggctatgaggccattggcggtggtagcccgttgcgtaggattacagacgagcaggc
ggaggcgctggcggagtctctgcgcgccaagggccaacctgcgaacgtgtacgtgggcatgcgctattggcaccc
ctacacggaggaggcgctggagcacattaaggccgacggcgtcacgcgcctggtcatcctcccgctgtaccctca
gttctccatctctaccagcggctccagccttcgactgcttgagtcgctcttcaagagcgacatcgcgctcaagtc
gctgcggcacacggtcatcccgtcctggtaccagcggcggggctacgtgagcgcgatggcggacctgattgtaga
g
Ferrochelatase Exon 3 (SEQ ID NO: 118):
gagctgaagaagttccgggacgtgcccagcgtggagctgtttttctccgcgcacggcgtgcccaagtcctacgtg
gaggaggcgggcgacccatacaaggaggagatggaggagtgcgtgcggctcattacggacgag
Ferrochelatase Exon 4 (SEQ ID NO: 119):
gtcaagcggcgcggcttcgccaacacgcacacgctggcctaccagagccgcgtgggccccgcggaatggctcaag
ccgtacacggatgagtccatcaa
Ferrochelatase Exon 5 (SEQ ID NO: 120):
ggagctgggcaagcgcggcgtcaagtcgctgctggcggtgcccatcagctttgtcagcgagcacattgagacgtt
ggaggagatcgacatggagtaccgcgagctggcggaggagagcg
Ferrochelatase Exon 6 (SEQ ID NO: 121):
gcatccgcaactggggccgcgtgccggcgctgaacaccaacgccgccttcatcgacgacctggcggacgcggtga
tggaggcgctgccctacgtgggctgcctggccgggccgacagactcgctggtgccgctgg
Ferrochelatase Exon 7 (SEQ ID NO: 122):
gcgacctggagatgctgctgcaggcctacgaccgcgagcgccgcacgctgccgtcaccggtggtgatgtgggagt
ggggctggaccaagagcgcggagacgtggaacggccgcattgccatgattgccatcatcatcatcctggcgctgg
aggcagccagcggccagtccatcctcaaaaacctgttcctggcggagtag
Ferrochelatase Intron 1 (SEQ ID NO: 123):
gtgcgataataaatttgcatccttatgaattgctcaatgactaacgagcagcgtccgcgaccacag
Ferrochelatase Intron 2 (SEQ ID NO: 124):
gtgagggtggcattctgtaaagggagttgtggagttgggcagagcgagtgggtttggtcgccagggcgaggatgt
tgcgcgggcgttggcaggaacagggctgctagggcttgcgtggccagcgactagggtttcgactggccagcgccg
ccggggcgcgcttgccgaagctgcacagccccaagcgcttctgtggatcaaatggaaacttgtggcagtgtgtat
gctagcgccttggcgcaagaccaattttagtggtattactgttattactgtggtagcggtgggtattcggcggcg
tggttgttgttgcagccccgtgcgactaagaccgctggcaacgacagcaagccgccgcacccaggcatatacggc
ccaccagcaccaccgtacacaaccacgtgcctttgcactctacgcaccacagcgcgctgctgccgctcccacctc
ccatcccaacggcccctcttacccccacttcacaacccctcctctcacacgccctcctcttccccctcctcttcc
ag
Ferrochelatase Intron 3 (SEQ ID NO: 125):
gtgggccgggcgcagcgggcgggcgggaggggcaggaggggcaggaggggaggaagggaggggaggaagggatgg
aaagctggcgcagcggcagcggcgggacaggtagagggcgctgccccagcggcggcaggtgggcatggtgggcgg
gtaggggcgacgcgtgagggactcgtcaggcatccgcatggcggcgacttgctgctcctcaccgctgacggctgc
atctgctgtgtgcgtaacctggcctggctggcaccgcag
Ferrochelatase Intron 4 (SEQ ID NO: 126):
gtgaggcccgtgggtgggacgcggggagggacgcggggagggggagacgcgggagcgggacaagggtgaggatac
ggggagggaataggagaggccatggggagggatggggacacgggaggatgcacgggcctgggtggagccaggggg
aagtggacgacgagcccggcgggaggagggctgggtagaaggacgcgggaggtggttgggacaggtggacggggc
gtgtggagcatacggcgcaagaagcgggactgagcgggttgcagggatggatgtaatcacggcaagtaagaaccc
cgagtggggctcagcgtgtcagcctgccttatctttcgcgcaagcgctggggttttatttcgctgtacacacgtc
gcgcctttctgccgcag
Ferrochelatase Intron 5 (SEQ ID NO: 127):
gtgaggaggcgccggagttttgggggaaggggtgcggcgtgaagcgagatggcaggggcgaaggaaggagcggat
ggtggctgggtgcaagcggagaggcgacagagagtggaggttttggtggagcggttggggagaggggcgcagcag
ggatgcggccctggggatggcgggacagaagggagcaagtttgccaagtgaagggggggggtgctcaagaggaga
gggcggtggaggttaagacggccgtgctggttatgctggggttgcaaggcgcatgggcgcatggagccgggggag
tttggctgtggatgggcactgcggatgggcacggcttgctactcatgtgcggtcgcggtccgcggtgtgtcagcc
agccaggacccatcccactgggtcttcctgcgtgcctgggactgcttgccgccacccacccattcatcaccacca
ctgcgcagacccaccaacaccgctgccctgaactgctctgactcttggcgctcctcag
Ferrochelatase Intron 6 (SEQ ID NO: 128):
gtgagtcgcgccgtcgcggttggttcgcggatgccggttggcggatgacgttcggcggttggcattgggtttggg
tttgaggggttgttgggtgaggtcgggattggggtcgggattgggggtcgagcgtggggctggcgtggatgatgg
cgtggtctttggaaggggcttggggaggttgcgcgtgtggatgcggacagcatgggcgcgacagtgcgcatgtgc
atgtgctgtgtcaaacgtctggtgcgttcagtgtgtccttgcgtgcctcccaccgtacgcagccatcccgcgcgc
ctggaccgtagagaccgcctacgtgtccgctagcggcctcggcctcagcctaagcgccagtagcgccagcgacac
aagcaacactgtcgctaatggcagcagcggcagcagcagcagtcacgagaatgcccgcggccgggagaaagtgct
cctagccgggggccgccgctagctggtttcctcagcgcgtggacggtggtgccttcatcccgaccaccccaggcg
cgtccccagtcccgtcgagctcgcctgccttgtggcccgccttgaccgccctggcgccacccggtggctcgcata
acgactcgctttccgttctccgcctgacgctgtccgcctgacgctctgcgcttgactctttgcgccttcctcccc
tcttcccccag
Mutant sequenced RedAlgae CHLH DNA (SEQ ID NO: 129):
atgcagacttcctcgcttcttggccggcgcacggcccacccggctgcgggcgcgacgcccaagccggttgcgccc
tcgccccgcgtggctagcacccgccaggtcgcgtgcaatgtggcgactggaccccggccgcccatgaccaccttc
accggtggcaacaagggccctgctaagcagcaggtgtcgctggatctgcgcgacgagggcgctggcatgttcacc
agcaccagcccggagatgcgccgtgtcgtccctgacgatgtgaagggtcgcgttaaggtgaaggttgtgtacgtg
gtgctggaggcccagtaccagtcggccatcagcgctgcggtgaagaacatcaacgccaagaactccaaggtgtgc
ttcgaggtggtgggctacctgctggaggagctgcgtgaccagaagaacctcgatatgctcaaggaggatgtggcc
tctgccaacatcttcatcggctcgctcatcttcattgaggagcttgccgagaagattgtggaggcggtgagcccc
ctgcgcgagaagctggacgcgtgcctgatcttcccgtccatgccggcggtcatgaagctgaacaagctgggcacg
ttttcgatggctcagctgggccagtcgaagtcggtgttctcggagttcatcaagtctgctcgcaagaacaacgac
aacttcgaggagggcttgctgaagctggtgcgcaccctgcctaaggtgctgaagtatctgccctcggacaaggcg
caggacgccaagaacttcgtgaacagcctgcagtactggctgggcggtaactcggacaacctggagaacctgctg
ctgaacaccgtcagcaactacgtgcccgctctgaagggcgtggacttcagcgtggctgagcccaccgcctacccc
gatgtgggtatctggcaccctctggcctcgggcatgtacgaggacctgaaggagtacctgaactggtacgacacc
cgcaaggacatggtcttcgccaaggacgcccccgtcattggcctggtgctgcagcgctcgcacctggtgactggc
gatgagggccactacagcggcgtggtcgctgagctggagagccgcggtgctaaggtcatccccgtctttgccggt
ggcctggacttctccgcccccgtcaagaagttcttctacgaccccctgggctctggccgcacgttcgtggacacc
gttgtgtcgctgaccggcttcgcgctggtgggcggccccgcgcgccaggacgcgccgaaggccattgaggcgctg
aagaacctgaacgtgccctacctggtgtcgctgccgctggtgttccagaccactgaggagtggctggacagcgag
ctgggcgtgcaccccgtccaggtggctctgcaggttgccctgcccgagctggatggtgccatggagcccatcgtg
ttcgctggccgtgactcgaacaccggcaagtcgcactcgctgcccgaccgcatcgcttcgctgtgcgctcgcgcc
gtgaactgggccaacctgcgcaagaagcgcaacgccgagaagaagctggccgtcaccgtgttcagcttcccccct
gacaagggcaacgtcggcactgccgcctacctgaacgtgttcggctccatctaccgcgtgctgaagaacctgcag
cgcgagggctacgacgtgggcgccctgtccgccctcggaggaggatctgatccagtcggtgctgacccagaagga
ggccaagttcaactcgaccgacctgcacatcgcctacaagatgaaggtggacgagtaccagaagctgtgccctta
cgccgaggcgctggaggagaactggggcaagccccccggcaccctgaacaccaacggccaggagctgctggtgta
cggccgccagtacggcaacgtcttcatcggcgtgcagcccaccttcggctacgagggcgacccgatgcgcctgct
gttctcgaagtcggccagcccccaccacggcttcgccgcctactacaccttcctggagaagatcttcaaggccga
cgccgtgctgcacttcggcacccacggctcgctggagttcatgcccggcaagcaggtcggcatgtcgggtgtgtg
ctaccccgactcgctgatcggcaccatccccaacctctactactacgccgccaacaacccgtctgaggccaccat
cgccaagcgccgctcgtacgccaacaccatttcgtacctgacgccgcctgccgagaacgccggcctgtacaaggg
cctgaaggagctgaaggagctgatcagctcgtaccagggcatgcgtgagtctggccgcgccgagcagatctgcgc
caccatcattgagaccgccaagctgtgcaacctggaccgcgacgtgaccctgcccgacgctgacgccaaggacct
gaccatggacatgcgcgacagcgttgtgggccaggtgtaccgcaagctgatggagattgagtcccgcctgctgcc
ctgcggcctgcacgtggtgggctgcccgcccaccgccgaggaggccgtggccaccctggtcaacatcgctgagct
ggaccgcccggacaacaacccccccatcaagggcatgcccggcatcctggcccgcgccattggtcgcgacatcga
gtcgatttacagcggcaacaacaagggcgtcctggctgacgttgaccagctgcagcgcatcaccgaggcctcccg
cacctgcgtgcgcgagttcgtgaaggaccgcaccggcctgaacggccgcatcggcaccaactggatcaccaacct
gctcaagttcaccggcttctacgtggacccctgggtgcgcggcctgcagaacggcgagttcgccagcgccaaccg
cgaggagctgatcaccctgttcaactacctggagttctgcctgacccaggtggtcaaggacaacgagctgggcgc
cctggtagaggcgctgaacggccagtacgtcgagcccggccccggcggtgaccccatccgcaaccccaacgtgct
gcccaccggcaagaacatccacgccctggaccctcagtcgattcccactcaggccgcgctgaagagcgcccgcct
ggtggtggaccgcctgctggaccgcgagcgcgacaacaacggcggcaagtaccccgagaccatcgcgctggtgct
gtggggcactgacaacatcaagacctacggcgagtcgctggcccaggtcatgatgatggtcggtgtcaagcccgt
ggccgacgccctgggccgcgtgaacaagctggaggtgatccctctggaggagctgggccgcccccgcgtggacgt
ggttgtcaactgctcgggtgtgttccgcgacctgttcgtgaaccagatgctgctgctggaccgcgccatcaagct
ggcggccgagcaggacgagcccgatgagatgaacttcgtgcgcaagcacgccaagcagcaggcggcggagctggg
cctgcagagcctgcgcgacgcggccacccgtgtgttctccaacagctcgggctcctactcgtccaacgtcaacct
ggcggtggagaacagcagctggagcgacgagtcgcagctgcaggagatgtacctgaagcgcaagtcgtacgcctt
caactcggaccgccccggcgccggtggcgagatgcagcgcgacgtgttcgagacggccatgaagaccgtggacgt
gaccttccagaacctggactcgtccgagatctcgctgaccgatgtgtcgcactacttcgactccgaccccaccaa
gctggtggcgtcgctgcgcaacgacggccgcacccccaacgcctacatcgccgacaccaccaccgccaacgcgca
ggtccgcactctgggtgagaccgtgcgcctggacgcccgcaccaagctgctcaaccccaagtggtacgagggcat
gcttgcctcgggctacgagggcgtgcgcgagatccagaagcgcatgaccaacaccatgggctggtcggccacctc
gggcatggtggacaactgggtgtacgacgaggccaactcgaccttcatcgaggatgcggccatggccgagcgcct
gatgaacaccaaccccaacagcttccgcaagctggtggccaccttcctggaggccaacggccgcggctactggga
cgccaagcccgagcagctggagcgcctgcgccagctgtacatggacgtggaggacaagattgagggcgtcgaata
a
CHLI1 5′ - untranslated region (regulatory region) (SEQ ID NO: 130):
tcctacagagtaaaggtctaggcgatgcgcgactgaaagactgtgaatcccggcgtcgccgtggtgggatgtggg
ccggtgcgctgtcgcagaggataaattacaggtatcaaacaaggttagggcgttggaaggagcggcgctagggaa
ctgaaatcggatctgcatcggaccctcattccgcgacttgtccttcttttgcctcgccccgcagctcttgagttt
tgttcttgaccctttgacacgaaccaaccgatataaaa
CHLI1 3′ - untranslated region (regulatory region) (SEQ ID NO: 131):
gcggcaggccttcatggtcgtcgttggagcatttgcggaaaggctgatggcagcagatgcagccatgtcagttgt
ggctgaagttgttggctggggcgggagcgggcagcagctgctgcgagcggccgaagcagcggtgctgctttgcgt
atgagaggaagaccagtgccctcgaggaggcgagtgcctgtgtgagtgtcaggacgtgtgacttcggaaactgag
ggcggtgagtagatgtgactggggcttgcaggaagcctactgaccctatcagaaaaggtgagcaggggtatatgg
tctaggagcgttgccggagcgtggctggccagtgctagccgcgcgggctctgttgctcgctggcgcgccgccgcc
ttcacaacagatgccgtagaaatgcagcgatgtgacgaggcgtggcctattctgcaatgtgtgaggcgccaatgg
cgccactgacaaatggaggagtggtcaaagcttgggtacgttttgagagctgcatcgggcagcgaggatcagtgt
gcggtaagaccgacggcagacggattggcaagggaataggagggacgtgggcgtgggcgcccgcgctttgtcgag
gccgcatgagccggccgcttctagacccgtagcccattttgaacaagcgcccacgcgtgctcccgatgggggaca
tcgatcacgggaattgattaaggggcatgtgtggtgtgcaagtgagtgactggtggttccgtccctgtgaggttg
tttcgttggacgtggctgccgggttgcgcgcgggctaagcgggcctgaggcagagcgctggcgtgtagccgcgag
tatcgatctgtaacgtgc
CHLI1 Exon 1 (SEQ ID NO: 132):
atggccctgaacatgcgtgtttcctcttccaaggtcgctgccaagcagcagggccgcatctccgcggtgccggtt
gtgtcgagcaaggtggcctcctccgcccgcgtggcccccttccag
CHLI1 Exon 2 (SEQ ID NO: 133):
ggcgctcccgtggccgcgcagcgcgctgctctgctgg
CHLI1 Exon 3 (SEQ ID NO: 134):
tgcgcgccgctgccgctactgaggtcaaggctgctgagggccgcactgagaaggagctgg
CHLI1 Exon 4 (SEQ ID NO: 135):
gccaggcccgccccatcttccccttcaccgccatcgtgggccaggatgagatgaagctggcgctgattctgaacg
tgatcgaccccaagatcggtggtgtcatgatcatgggcgaccgtggcactggcaagtccaccaccattcgtgccc
tggcggatctgctgcccgagatgcag
CHLI1 Exon 5 (SEQ ID NO: 136):
gtggttgccaacgacccctttaactcggaccccaccgaccccgagctgatgagcgaggaggtgcgcaaccgcgtc
aaggccggcgagcagctgcccgtgtcttccaagaagattcccatggtggacctgcccctgggcgccactgaggac
cgcgtgtgcggcaccatcgacatcgagaaggcgctgaccgagg
CHLI1 Exon 6 (SEQ ID NO: 137):
gtgtcaaggcgttcgagcccggcctgctggccaaggccaaccgcggcatcctgtacgtggatgaggtcaacctgc
tggacgaccacctg
CHLI1 Exon 7 (SEQ ID NO: 138):
gtcgatgtgctgctggactcggccgcctccggctggaacaccgtggagcgcgagggtatctccatcagccacccc
gcccgcttcatcctggtcggctcgg
CHLI1 Exon 8 (SEQ ID NO: 139):
gcaaccccgaggagggtgagctgcgcccccagctgctggatcgcttcggcatgcacgcccagatcggcaccgtca
aggacccccgcctgcgtgtgcagatcgtgtcgcagcgctcgaccttcgacgagaaccccgccgccttccg
CHLI1 Exon 9 (SEQ ID NO: 140):
caaggactacgaggccggccagatggcgctgacccagcgcatcgtggacgcgcgcaagctgctgaagcagggcga
ggtcaactacgacttccgcgtcaagatcagccagatctgctcggacctgaacgtggacggcatccgcggcgacat
cgtgaccaaccgcgccgccaaggccctggccgccttcgagggccgcaccgag
CHLI1 Exon 10 (SEQ ID NO: 141):
gtgacccccgaggacatctaccgtgtcattcccctgtgcctgcgccaccgcctccggaaagaccccctggctgag
atcgacgacggtgaccgcgtgcgtgagatcttcaagcaggtgttcggcatggagtaa
CHLI1 Intron 1 (SEQ ID NO: 142):
gtgtgcagttgcatctaaagaacgtccaattcatggttactgctcgtggatctaagcggttggctcaccagcgtt
ccatggtccccgattcgtgcacgcag
CHLI1 Intron 2 (SEQ ID NO: 143):
gtgagaagccatgatacaaatataaggatttgaagcggtagatctaggacccatcgaacttgagcaccgacttgc
agtccttgccttgtccggcgactgaacttctgcgcttgctttgcag
CHLI1 Intron 3 (SEQ ID NO: 144):
gtaagtgtcgcgcaaagattttctgccgggacgggtctccctcgcaacatctgaacccatggctcgtttttttgc
cccgcag
CHLI1 Intron 4 (SEQ ID NO: 145):
gtgcgcgcctcccccaaccccagtttggcaaatgtgtggttaagcgtcgaaagcgtgaacagaaacaggtgttgc
gggggccgcggaatggctgcaatgggtgctgggggcttcggagggtctgggggcgagtttgggtatacacgggcg
cgcacacttgaaggaacgctcaaggacgacagcggaggcgtggagacagcgccggcccaagcagcctgtacttgt
agctgctggtcagctgaggcatcacgacttgggaccagcacccggcctcacggttgcacaaggccatcaccgcgc
gccaccacccacgcctcttcaaacccatgccggcacctaccgctacccctgtgacacgctccgcacacgccgccc
cgcacaccccaccatgtgacag
CHLI1 Intron 5 (SEQ ID NO: 146):
gtgagagcgaggcgcggggcgtgctctgcaggctagggtgaagatcaggagagccgaagcgggcccgaacagcgc
agagagaggcaagacgacacccctgccgcgttttgatcacaagattcacacccttgctctccccaacgctcccgc
acatag
CHLI1 Intron 6 (SEQ ID NO: 147):
gtgagcaggggcagataggcggtcgggcggctgggcggcaggggctgtgttggctgtgttgggtgtgggctgagg
ctggtgggtgggctggcgggtggcagggatagcggtgaggggatggtgatggggcagaatgggcgggtgggcgga
cacgtggggtcgttgaagggtgtgtggggacggcaactggtatgcgatatgtcggcttggccctggcggggaaag
cattcgcagaatggcgcacgaacgaggccggggagcgagcggggatgggagacgcaacctgcgctgcgaagtgcg
gcgcgcgctccagttgacacgttgcacgaatgtggccagtgttcgcctgagagttatgggttagaccgccagatg
agccggttaagctggtggtcgcggttgatcggctgcttcccttccggttgcacgcctggcaccctaacattaccc
tgtccgctgctgccctttgcccacag
CHLI1 Intron 7 (SEQ ID NO: 148):
gtgagtgcagctgccgctgcggctgctgatggtgacctgtgcgaccacggggctccgcatttctggacgaagcgt
tgtaccatagccgtcttggtccctgatttgggccggctctggtccgaagccttgacatctacagttcaacatggc
cgtataacgatcctgtgcccacccacacgccaccccgccag
CHLI1 Intron 8 (SEQ ID NO: 149):
gtgagcgcgcgctctacgatacggcagacatgtacacactgcggcgcactgtagagcttgcattgcatttcaagg
cctcgaaagagtagggtggtcgttctctggtggtgtccggccacaattatgcaccccggtgttggtgcagcagct
gtgatgtcacaccttgcatcacccccctactgctgccgcctctcctctcttctcgcccgcag
CHLI1 Intron 9 (SEQ ID NO: 150):
gtgagcagagcaatattgcagagggaagggtggcggaagggtgataacggttggggatctagaggggcgagatgg
atgcacacagcgcggggttggttatgcatgcctgcatggacgcgtgcacgcacccctgatctgccggttttccaa
ctggcgatgccgtattatgacctgcagctcaccatcctcatgcttgatttgcctcgctcag
CHLI1 Protein sequence (SEQ ID NO: 151):
MALNMRVSSSKVAAKQQGRISAVPVVSSKVASSARVAPFQGAPVAAQRAALLVRAAAATEVKAAEGRTEKELGQA
RPIFPFTAIVGQDEMKLALILNVIDPKIGGVMIMGDRGTGKSTTIRALADLLPEMQVVANDPFNSDPTDPELMSE
EVRNRVKAGEQLPVSSKKIPMVDLPLGATEDRVCGTIDIEKALTEGVKAFEPGLLAKANRGILYVDEVNLLDDHL
VDVLLDSAASGWNTVEREGISISHPARFILVGSGNPEEGELRPQLLDRFGMHAQIGTVKDPRLRVQIVSQRSTFD
ENPAAFRKDYEAGQMALTQRIVDARKLLKQGEVNYDFRVKISQICSDLNVDGIRGDIVTNRAAKALAAFEGRTEV
TPEDIYRVIPLCLRHRLRKDPLAEIDDGDRVREIFKQVFGME
Mutant protein sequence RedAlgaeCHLH (SEQ ID NO: 152):
MQTSSLLGRRTAHPAAGATPKPVAPSPRVASTRQVACNVATGPRPPMTTFTGGNKGPAKQQVSLDLRDEGAGMFT
STSPEMRRVVPDDVKGRVKVKVVYVVLEAQYQSAISAAVKNINAKNSKVCFEVVGYLLEELRDQKNLDMLKEDVA
SANIFIGSLIFIEELAEKIVEAVSPLREKLDACLIFPSMPAVMKLNKLGTFSMAQLGQSKSVFSEFIKSARKNND
NFEEGLLKLVRTLPKVLKYLPSDKAQDAKNEVNSLQYWLGGNSDNLENLLLNTVSNYVPALKGVDFSVAEPTAYP
DVGIWHPLASGMYEDLKEYLNWYDTRKDMVFAKDAPVIGLVLQRSHLVTGDEGHYSGVVAELESRGAKVIPVFAG
GLDFSAPVKKFFYDPLGSGRTFVDTVVSLTGFALVGGPARQDAPKAIEALKNLNVPYLVSLPLVFQTTEEWLDSE
LGVHPVQVALQVALPELDGAMEPIVFAGRDSNTGKSHSLPDRIASLCARAVNWANLRKKRNAEKKLAVTVFSFPP
DKGNVGTAAYLNVFGSIYRVLKNLQREGYDVGALSALGGGSDPVGADPEGGQVQLDRPAHRLQDEGGRVPEAVPL
RRGAGGELGQAPRHPEHQRPGAAGVRPPVRQRLHRRAAHLRLRGRPDAPAVLEVGQPPPRLRRLLHLPGEDLQGR
RRAALRHPRLAGVHARQAGRHVGCVLPRLADRHHPQPLLLRRQQPV
CHLI1 DNA sequence (SEQ ID NO: 153):
atggccctgaacatgcgtgtttcctcttccaaggtcgctgccaagcagcagggccgcatctccgcggtgccggtt
gtgtcgagcaaggtggcctcctccgcccgcgtggcccccttccagggcgctcccgtggccgcgcagcgcgctgct
ctgctggtgcgcgccgctgccgctactgaggtcaaggctgctgagggccgcactgagaaggagctgggccaggcc
cgccccatcttccccttcaccgccatcgtgggccaggatgagatgaagctggcgctgattctgaacgtgatcgac
cccaagatcggtggtgtcatgatcatgggcgaccgtggcactggcaagtccaccaccattcgtgccctggcggat
ctgctgcccgagatgcaggtggttgccaacgacccctttaactcggaccccaccgaccccgagctgatgagcgag
gaggtgcgcaaccgcgtcaaggccggcgagcagctgcccgtgtcttccaagaagattcccatggtggacctgccc
ctgggcgccactgaggaccgcgtgtgcggcaccatcgacatcgagaaggcgctgaccgagggtgtcaaggcgttc
gagcccggcctgctggccaaggccaaccgcggcatcctgtacgtggatgaggtcaacctgctggacgaccacctg
gtcgatgtgctgctggactcggccgcctccggctggaacaccgtggagcgcgagggtatctccatcagccacccc
gcccgcttcatcctggtcggctcgggcaaccccgaggagggtgagctgcgcccccagctgctggatcgcttcggc
atgcacgcccagatcggcaccgtcaaggacccccgcctgcgtgtgcagatcgtgtcgcagcgctcgaccttcgac
gagaaccccgccgccttccgcaaggactacgaggccggccagatggcgctgacccagcgcatcgtggacgcgcgc
aagctgctgaagcagggcgaggtcaactacgacttccgcgtcaagatcagccagatctgctcggacctgaacgtg
gacggcatccgcggcgacatcgtgaccaaccgcgccgccaaggccctggccgccttcgagggccgcaccgaggtg
acccccgaggacatctaccgtgtcattcccctgtgcctgcgccaccgcctccggaaagaccccctggctgagatc
gacgacggtgaccgcgtgcgtgagatcttcaagcaggtgttcggcatggagtaa

Although the invention has been described with reference to the above example, it will be understood that modifications and variations are encompassed within the spirit and scope of the invention. Accordingly, the invention is limited only by the following claims.

1. An engineered algae having a genetic modification, where the genetic modification results in an accumulation of heme in the algae as compared to an algae lacking the genetic modification.

2. The engineered algae of claim 1, wherein the engineered algae has reduced or absence of chlorophyll production.

3. The engineered algae of claim 1 or claim 2, wherein the algae has red or red-like color.

4. The engineered algae according to any of claims 1-3, wherein the algae is capable of growth on glucose as the sole carbon source.

5. The engineered algae according to any of claims 1-4, wherein the genetic modification comprises a genetic alteration to a chlorophyll synthesis pathway, protoporphyrinogen IX synthesis pathway or heme synthesis pathway.

6. The engineered algae according to any of claims 1-5, wherein the genetic modification is associated with a deficiency in the expression of magnesium chelatase.

7. The engineered algae according to any of claims 1-6, wherein the genetic modification comprises an alteration in one or more of CHLD, CHLI1, CHLI2 or CHLH1.

8. The engineered algae of claim 7, wherein the genetic modification comprises an alteration in an upstream regulatory region, a downstream regulatory region, an exon, an intron or any combination thereof.

9. The engineered algae according to any of claims 5-8, wherein the genetic modification comprises an insertion, a deletion, a point mutation, an inversion, a duplication, a frameshift or any combination thereof.

10. The engineered algae according to any of claims 1-9, wherein the engineered algae has a heme content greater than chlorophyll content.

11. The engineered algae according to any of claims 1-9, wherein the engineered algae has a protoporphyrin IX content greater than chlorophyll content.

12. The engineered algae according to any of claims 1-11, wherein the engineered algae has reduced production of one or more fatty acids.

13. The engineered algae according to any of claims 1-12, wherein the engineered algae further comprises a genetic modification that reduces or eliminates expression of light independent protochlorophyllide oxidoreductase.

14. The engineered algae of claim 13, wherein the genetic modification comprises a mutation or deletion in one or more of ChlB, ChlL or ChlN.

15. The engineered algae according to any of claims 1-14, wherein the engineered algae has upregulated expression of ferrocheletase.

16. The engineered algae according to any of claims 1-15, wherein the engineered algae has upregulated expression of protoporphyrinogen IX oxidase.

17. The engineered algae according to any of claims 1-16, wherein the engineered algae contains a recombinant or heterologous nucleic acid.

18. The engineered algae according to any of claims 1-17, wherein the engineered algae is a Chlamydomonas sp.

19. The engineered algae of claim 18, wherein the Chlamydomonas sp. is Chlamydomonas reinhardtii.

20. An edible composition comprising an algae preparation, wherein the algae preparation comprises an engineered algae of any of claims 1-19 or a portion thereof.

21. The edible composition of claim 20, wherein the edible composition comprises heme derived from the engineered algae.

22. The edible composition of claim 20, wherein the algae preparation comprises algae cells.

23. The edible composition of claim 20, wherein the algae preparation is a fractionated algae preparation.

24. The edible composition according to any of claims 20-23, wherein the algae preparation is red or red-like in color.

25. The edible composition according to any of claims 20-24, wherein the edible composition has a red or red-like color derived from the algae preparation.

26. The edible composition according to any of claims 20-25, wherein the algae preparation confers a meat or meat-like flavor to the edible composition.

27. The edible composition according to any of claims 20-26, wherein the edible composition has a meat or meat-like texture derived from the algae preparation.

28. The edible composition according to claim 27, wherein the meat or meat-like texture is a beef or beef-like texture, a fish or fish-like texture, a chicken or chicken-like texture, a pork or pork-like texture or a texture of a meat replica.

29. The edible composition according to any of claims 20-28, wherein the edible composition is a finished product selected from the group consisting of a beef-like food product, a fish-like product, a chicken-like product, a pork-like product and a meat replica.

30. The edible composition according to any of claims 20-29, wherein the edible composition is vegan, vegetarian or gluten-free.

31. The edible composition according to any of claims 20-30, wherein the edible composition has an appearance of blood derived from the algae preparation.

32. The edible composition according to any of claims 20-31, wherein the algae preparation has a heme content greater than chlorophyll content.

33. The edible composition according to any of claims 20-32, wherein the algae preparation has a protoporphyrin IX content greater than chlorophyll content.

34. The edible composition according to any of claims 20-33, wherein the algae preparation provides at least 5%, 10%, 15%, 20%, 25%, 30%, 35%, 40%, 45%, 50%, 55%, 60%, 65%, 70%, 75%, 80%, 85%, 90%, 95%, 99% or 100% of the total protein content to the edible composition.

35. The edible composition according to any of claims 20-34, wherein the algae preparation provides vitamin A, beta carotene or a combination thereof to the composition.

36. The edible composition of claim 35, wherein the vitamin A, the beta carotene or the combination thereof is at least about 5%, 10%, 15%, 20%, 25%, 30%, 35%, 40%, 45%, 50%, 55%, 60%, 65%, 70%, 75%, 80%, 85%, 90%, 95%, 99% or 100% of the daily recommended requirement.

37. The edible composition according to any of claims 20-36, wherein the algae preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in the edible composition.

38. The edible composition according to any of claims 20-37, wherein the algae preparation provides less than about 0.01%, 0.05%, 0.1%, 0.2%, 0.3%, 0.4%, 0.5%, 0.6%, 0.7%, 0.8%, 0.9%, 1.0%, 1.2%, 1.5%, 2%, 5% or 10% of total saturated fat present in a finished product comprising the edible composition.

39. The edible composition according to any of claims 20-38, wherein the algae preparation provides at least about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 250 mg, 300 mg, 350 mg, 400 mg, 450 mg or 500 mg of omega-3 fatty acids to the edible composition.

40. The edible composition according to any of claims 20-39, wherein the algae preparation has reduced fatty acid content.

41. The edible composition according to any of claims 20-40, wherein the edible product is combined with a protein source, a fat source, a carbohydrate, a starch, a thickener, a vitamin, a mineral, or any combination thereof.

42. The edible composition of claim 41, wherein the protein source is selected from the group consisting of textured wheat protein, textured soy protein and textured pea protein, fungal protein or algal protein.

43. The edible composition of claim 41, wherein the fat source comprises at least one of refined coconut oil or sunflower oil.

44. The edible composition of any of claims 41-43, further comprising at least one of potato starch, methylcellulose, water, and a flavor, wherein the flavor is selected from the group consisting of yeast extract, garlic powder, onion powder, salt, and any combination thereof.

45. The edible composition of any of claims 41-44, wherein the edible product is an ingredient for a burger, a sausage, a kebab, a filet, a fish-alternative, a ground meat-like product or a meatball.

46. The edible composition of claim 45, wherein the burger comprises about 5% of the algae preparation, about 20% textured soy protein and about 20% refined coconut oil.

47. The edible composition of claim 46, further comprising about 3% sunflower oil, about 2% potato starch, about 1% methylcellulose, about 45% water and about 4-9% flavors.

48. The edible composition of claim 46, further comprising about 0.5% Kojac gum, about 0.5% Xanthan gum, about 45% water and about 4-9% flavors.

49. The edible composition of claim 45, wherein the fish-alternative comprises 20% textured soy protein, about 5% of algae preparation, about 65% water and about 10% flavors.

50. The edible composition according to any of claims 20-49, wherein the edible composition is free of animal proteins.

51. The edible composition according to any of claims 20-50, wherein the algae preparation comprises an algae having an increase in protoporphyrinogen IX synthesis or accumulation.

52. The edible composition according to any of claims 20-51, wherein the algae preparation comprises an algae that exhibits a red or red-like color when grown in the dark conditions.

53. The edible composition according to any of claims 20-52, wherein the algae in the algae preparation are recombinant or genetically modified algae.

54. The edible composition according to any of claims 20-53, wherein the algae preparation comprises a Chlamydomonas sp.

55. The edible composition of claim 54, wherein the Chlamydomonas sp. is Chlamydomonas reinhardtii.

56. A method for the production of an edible composition comprising:
(a) culturing an engineered algae according to any of claims 1-19 in a condition where the engineered algae exhibits a red or red-like color and wherein the engineered algae produces heme;
(b) collecting the cultured engineered algae to produce an algae preparation; and
(c) combining the algae preparation with at least one edible ingredient to produce an edible composition.

57. The method of claim 56, wherein the condition comprises a fermentation condition.

58. The method according to any of claims 56-57, wherein the condition comprises acetate as a reduced carbon source for growth of the engineered algae.

59. The method according to any of claims 56-58, wherein the condition comprises sugar as a reduced carbon source for growth of the engineered algae.

60. The method according to any of claims 56-59, wherein the condition comprises dark or limited light conditions.

61. The method according to any of claims 56-60, wherein the method further comprises fractionating the cultured algae to produce the algae preparation.

62. The method according to any of claims 56-61, wherein the algae preparation has a heme content that is greater than chlorophyll content.

63. The method according to any of claims 56-62, wherein the algae preparation has a protoporphyrin IX content that is greater than chlorophyll content.

64. The method according to any of claims 56-63, wherein the condition further comprises iron supplements.

65. The method according to any of claims 56-64, wherein the engineered algae is a Chlamydomonas sp.

66. The method of claim 65, wherein the engineered algae is a Chlamydomonas reinhardtii.

67. The method according to any of claims 56-66, wherein the edible composition has at least one feature selected from the group consisting of a meat or meat-like flavor, a meat or meat-like texture, a blood-like appearance and a meat or meat-like color, wherein the at least one feature is derived from the algae preparation.

68. The method according to any of claims 56-67, wherein the method further comprises producing a finished product comprising the edible composition and wherein the finished product is a beef-like food product, a fish-like product, a chicken-like product, a pork-like product or a meat replica.

69. The method according to any of claims 56-68, wherein the edible composition is free of animal proteins.

70. The method according to any of claims 56-69, wherein the algae preparation is fractionated to remove one or more of starch, protein, PPIX, fatty acids and chlorophyll.

71. A method of making an engineered algae enriched in heme content, comprising:
(a) subjecting an algae strain to a process that produces genetic modification to create a first algae population; and
(b) from the first algae population, selecting a second algae population that is enriched in heme content, and optionally, PPIX content.

72. The method according to claim 71, wherein the process comprises at least one of a random UV mutagenesis, a random chemical mutagenesis, a recombinant genetic engineering, a gene editing, or a gene silencing.

73. The method according to claim 71 or claim 72, further comprising culturing the first algae population in a fermentation condition.

74. The method according to claim 73, wherein the fermentation condition comprises a media having sugar as a sole carbon source.

75. The method according to claim 74, wherein the sugar is selected from the group consisting of glucose, dextrose, fructose, maltose, galactose, sucrose, and ribose.

76. The method according to any of claims 73-75, wherein the fermentation condition comprises a brightness of less than 500 lux.

77. The method of any of claims 73-76, wherein the step of selecting the second algae population comprises sorting or identifying algae cells having a red or red-like color.

78. The method of any of claims 73-77, wherein the selecting is performed by FACS.

79. The method according to any of claims 73-78, wherein the second algae population is selected with its capability to grow in the fermentation condition.

80. The edible composition according to any of claims 20-59, wherein the algae preparation comprises an algae having an increase in protoporphyrinogen IX synthesis or accumulation.

81. The edible composition according to any of claims 20-59, wherein the algae preparation comprises an algae that exhibits a red or red-like color when grown in dark conditions.


SEQUENCE LISTING (153 sequences)

SEQ ID NO: 1 - DNA, 1173 bp, Chlamydomonas reinhardtii
atgcagatgatgcagcgcaacgttgtgggccagcgccccgtcgctggctcccgccgctcgctggtggttgccaacgttgcggaggtgacccgccccgcggtcagcaccaacggcaagcaccggactggtgtgccggagggaactcccatcgtcacccctcaggacctgccctcgcgccctcgccgcaaccgccgcagcgagagcttccgtgcttccgttcgtgaggtgaacgtgtcgcccgccaacttcatcctgccgatcttcatccacgaggagagcaaccagaacgtgcccatcgcctccatgcctggcatcaaccgcctggcgtatggcaagaacgtgattgactacgttgctgaggctcgctcttacggtgtcaaccaggtcgtggttttccccaagacgcccgaccacctgaagacgcaaaccgcggaggaggcgttcaacaagaacggcctcagccagcgcacgatccgcctgctgaaggactctttccctgacctggaggtgtacacggacgtggctctggacccctacaactcggacggccacgacggtatcgtgtcggacgccggtgtgatcctgaacgacgagaccatcgagtacctgtgccgccaggccgtgagccaggccgaggccggtgccgacgtggtgtcgccctctgacatgatggacggccgcgtgggcgccatccgccgcgccctggaccgcgagggcttcaccaacgtgtccatcatgtcctacaccgccaagtacgcctccgcctactacggccccttccgtgacgccctggcgtccgcgcccaagcccggccaggcgcaccgccgcatcccccccaacaagaagacctaccagatggaccccgccaactaccgcgaggccatccgcgaggccaaggccgacgaggccgagggcgctgacatcatgatggtcaagcccggcatgccgtacctggacgtggtacgcctgctgcgtgagaccagcccgctgcccgtggccgtgtaccacgtgtcgggcgagtacgccatgctcaaggcggcggcggagcgcggctggctgaacgagaaggatgccgtgcttgaggccatgacctgcttccgccgcgccggcgctgacctcatcctcacctactacggcattgaggcctccaagtggctggcgggcgagaagtaa

SEQ ID NO: 2 - protein, 390 aa, Chlamydomonas reinhardtii
MQMMQRNVVGQRPVAGSRRSLVVANVAEVTRPAVSTNGKHRTGVPEGTPIVTPQDLPSRPRRNRRSESFRASVREVNVSPANFILPIFIHEESNQNVPIASMPGINRLAYGKNVIDYVAEARSYGVNQVVVFPKTPDHLKTQTAEEAFNKNGLSQRTIRLLKDSFPDLEVYTDVALDPYNSDGHDGIVSDAGVILNDETIEYLCRQAVSQAEAGADVVSPSDMMDGRVGAIRRALDREGFTNVSIMSYTAKYASAYYGPFRDALASAPKPGQAHRRIPPNKKTYQMDPANYREAIREAKADEAEGADIMMVKPGMPYLDVVRLLRETSPLPVAVYHVSGEYAMLKAAAERGWLNEKDAVLEAMTCFRRAGADLILTYYGIEASKWLAGEK

SEQ ID NO: 3 - DNA, 1098 bp, Chlamydomonas reinhardtii
atggcactgcaagcctcaacccgctcgctccagcagcgccgcgccttctcttcggcccagacctccaagcgtgtgtctgtgaccaaggtccgcgcgacggctatcgaggcggagaactatgtgaagcaggctccccagtcgctggtccgcccgggcatcgacactgaggactctatgcgcgctcgcttcgagaaggtgatccgcaacgcccaggactccatctgcaatgctatctccgagatcgatggcaagccgttccaccaggacgcctggacccgccccggcggcggtggcggcatcagccgcgtgctgcaggacggcaacgtgtgggagaaggccggcgtcaacgtgtccgtggtctacggcaccatgccccctgaggcctaccgcgctgccactggcaacgccgagaagctgaagaacaagggtgacggtggccgcgtgcccttcttcgccgccggcatctcgtcggtgatgcacccccgcaacccccactgccccaccatgcacttcaactaccgctacttcgagactgaggagtggaacggcatccccggccagtggtggttcggcggcggcaccgacatcacccccagctatgtggtgcccgaggacatgaagcacttccacggcacctacaaggcggtgtgcgaccgccacgatcccgcttactacgagaagttccgcacctggtgcgatgagtacttcctcatcaagcaccgcggcgagcgccgcggcctgggcggcatcttcttcgatgacctgaacgaccgcaaccccgaggacatcctgaagttctcgaccgacgccgtgaacaacgtggtggaggcatactgccccatcatcaagaagcacatgaacgacccctacacccccgaggagaaggagtggcagcagatccgccgcggccgctacgtggagttcaacctggtctatgaccgcggcaccaccttcggcctgaagaccggcggccgcattgagtcgatcctcatgtccatgccccagaccgcctcatggctgtacgaccaccagcccaaggccggctcgcccgaggccgagctgctcgacgcctgccgcaacccccgcgtctgggtgtaa

SEQ ID NO: 4 - protein, 365 aa, Chlamydomonas reinhardtii
MALQASTRSLQQRRAFSSAQTSKRVSVTKVRATAIEAENYVKQAPQSLVRPGIDTEDSMRARFEKVIRNAQDSICNAISEIDGKPFHQDAWTRPGGGGGISRVLQDGNVWEKAGVNVSVVYGTMPPEAYRAATGNAEKLKNKGDGGRVPFFAAGISSVMHPRNPHCPTMHFNYRYFETEEWNGIPGQWWFGGGTDITPSYVVPEDMKHFHGTYKAVCDRHDPAYYEKFRTWCDEYFLIKHRGERRGLGGIFFDDLNDRNPEDILKFSTDAVNNVVEAYCPIIKKHMNDPYTPEEKEWQQIRRGRYVEFNLVYDRGTTFGLKTGGRIESILMSMPQTASWLYDHQPKAGSPEAELLDACRNPRVWV

SEQ ID NO: 5 - DNA, 1049 bp, Chlamydomonas reinhardtii
atgctgaggaagcagattggtggatctggccagcagcgggcgggcctccgacgggtgaaccaaggacctgcgcgtcggcggttggcaccctgccgcgtggcggcccccgtgcaaacctcgtcctccgtcgccacattcaatggcttcgtggactacattcacggactccagaagaacattctgagcactgctgaggatctggagaacggcgagcggaagtttgttgttgaccgctgggagcgcgacgccagcaaccccaacgccgggtatggcattacgtgcgtgcttgaggacgggaaggtgctggagaaggccgcagccaatatctcagtggtgcgcgggacgctgtcggcgcagcgcgcagtggccatgagctcccgcggccgcagcagcatcgaccccaagggcgggcagccctacgccgcggccgccatgagcctagtgttccacagcgcgcacccgctcatccccacgctgcgcgcgacgtgcggttgttccaggtgggcgatgaggcgtggtacggcggtggctgtgacctgacgcccaactacctagacgtggaggactcgcagtccttccaccgctactggaaggacgtgtgcggcaagtacaagccgggcctgtacaccgagctcaaggagtggtgcgacaggtacttctacatcccggcccgcaaagagcaccgtggcattggcggcctgttctttgatgacatggccactgcggaggcgggctgcgatgtggaggcgtttgtgcgggaagtgggagatggcatcctgccctgctggctgcccatcgtggcgcggcaccgtggccagcccttcacggagcagcagcggcaatggcagctgctgcgccgcggtcgctacatcgagttcaacctgctgtacgaccgcggcatcaagttcggtctggacggcggccgcatcgagagcatcatggtgtcggcgccgccgctgatcgcgtggaagtacaacgtggtgccacagccgggcagccccgaggaggagatgctgaaggtgcttcagcagccccgcgagtgggcctga

SEQ ID NO: 6 - protein, 349 aa, Chlamydomonas reinhardtii
MLRKQIGGSGQQRAGLRRVNQGPARRRLAPCRVAAPVQTSSSVATFNGFVDYIHGLQKNILSTAEDLENGERKFVVDRWERDASNPNAGYGITCVLEDGKVLEKAAANISVVRGTLSAQRAVAMSSRGRSSIDPKGGQPYAAAAMSLVFHSAHPLIPTLRADVRLFQVGDEAWYGGGCDLTPNYLDVEDSQSFHRYWKDVCGKYKPGLYTELKEWCDRYFYIPARKEHRGIGGLFFDDMATAEAGCDVEAFVREVGDGILPCWLPIVARHRGQPFTEQQRQWQLLRRGRYIEFNLLYDRGIKFGLDGGRIESIMVSAPPLIAWKYNVVPQPGSPEEEMLKVLQQPREWA

SEQ ID NO: 7 - DNA, 1482 bp, Chlamydomonas reinhardtii
atggcgtcgtttggattgatgcaaaggacggtgcactgtccccagcttgtggaggagcggtgttcgccggtcgctggctgctctggtcgtggcctgccagttatccagcggcaacggcgtggcgtgtgcagtgccaccaacggtgtccagcgagggcgtgtgctgcgccggacggccgcttcgaccgacgtggtctccttcgtggaccccaatgacattagaaaacccgcagcagcagcagctggccctgcggtggataaggtcggcgttctgctgttaaaccttggcgggcccgaaaagctcgacgacgtcaagcctttcctgtataacctattcgccgacccagaaattattcgcctgccagcggcagctcagttcctgcagccgctgctcgcgacgatcatctccacgcttcgcgccccgaagagcgcggagggctatgaggccattggcggtggtagcccgttgcgtaggattacagacgagcaggcggaggcgctggcggagtctctgcgcgccaagggccaacctgcgaacgtgtacgtgggcatgcgctattggcacccctacacggaggaggcgctggagcacattaaggccgacggcgtcacgcgcctggtcatcctcccgctgtaccctcagttctccatctctaccagcggctccagccttcgactgcttgagtcgctcttcaagagcgacatcgcgctcaagtcgctgcggcacacggtcatcccgtcctggtaccagcggcggggctacgtgagcgcgatggcggacctgattgtagaggagctgaagaagttccgggacgtgcccagcgtggagctgtttttctccgcgcacggcgtgcccaagtcctacgtggaggaggcgggcgacccatacaaggaggagatggaggagtgcgtgcggctcattacggacgaggtcaagcggcgcggcttcgccaacacgcacacgctggcctaccagagccgcgtgggccccgcggaatggctcaagccgtacacggatgagtccatcaaggagctgggcaagcgcggcgtcaagtcgctgctggcggtgcccatcagctttgtcagcgagcacattgagacgttggaggagatcgacatggagtaccgcgagctggcggaggagagcggcatccgcaactggggccgcgtgccggcgctgaacaccaacgccgccttcatcgacgacctggcggacgcggtgatggaggcgctgccctacgtgggctgcctggccgggccgacagactcgctggtgccgctgggcgacctggagatgctgctgcaggcctacgaccgcgagcgccgcacgctgccgtcaccggtggtgatgtgggagtggggctggaccaagagcgcggagacgtggaacggccgcattgccatgattgccatcatcatcatcctggcgctggaggcagccagcggccagtccatcctcaaaaacctgttcctggcggagtag

SEQ ID NO: 8 - protein, 492 aa, Chlamydomonas reinhardtii
MASFGLMQRTVHCPQLVEERCSPVAGCSGRGLPVIQRQRRGVCSATNGVQRGRVLRRTAASTDVVSFVDPNDIRKPAAAAAGPAVDKVGVLLLNLGGPEKLDDVKPFLYNLFADPEIIRLPAAAQFLQPLLATIISTLRAPKSAEGYEAIGGGSPLRRITDEQAEALAESLRAKGQPANVYVGMRYWHPYTEEALEHIKADGVTRLVILPLYPQFSISTSGSSLRLLESLFKSDIALKSLRHTVIPSWYQRRGYVSAMADLIVEELKKFRDVPSVELFFSAHGVPKSYVEEAGDPYKEEMEECVRLITDEVKRRGFANTHTLAYQSRVGPAEWLKPYTDESIKELGKRGVKSLLAVPISFVSEHIETLEEIDMEYRELAEESGIRNWGRVPALNTNAAFIDDLADAVMEALPYVGCLAGPTDSLVPLGDLEMLLQAYDRERRTLPSPVVWEWGWTKSAETWNGRIAMIAIIIILALEAASGQSILKNLFLAE

SEQ ID NO: 9 - DNA, 1392 bp, Chlamydomonas reinhardtii
atgcagatgcagctgaacgccaagaccgtgcagggcgccttcaaggcgcagcgccctcgctctgtccgcggcaacgtggcggtgcgcgcagtggccgctccccctaagctggtcaccaagcgctccgaggagatcttcaaggaggctcaggagctgctgcccggtggcgtgaactcgcccgtgcgcgctttccgctcggttggtggcggccccatcgtcttcgacagggtcaagggtgcctactgctgggacgtcgatggcaacaagtacatcgactacgttggctcttggggccctgccatttgcggccacggcaacgacgaggtcaacaacgccctgaaggcgcagatcgacaagggcacctcgttcggtgctccctgcgagctggagaacgtgctggccaagatggtgattgaccgcgtgccctcggtggagatggtgcgcttcgtgtcctcgggcactgaggcgtgcctgtcggtgctgcgcctgatgcgcgcatacaccggccgcgagaaggtgctgaagttcaccggctgctaccacggccacgccgactccttcctggtgaaggccggctccggtgtgatcaccctgggcctgcccgactcgcccggtgtgcccaagagcaccgccgccgccaccctgaccgccacctacaacaacctggactccgtgcgcgagctgttcgccgccaacaagggcgagattgccggtgtgatcctggagcccgtggtcggcaacagcggcttcattgtgcccaccaaggagttcctgcagggcctgcgcgagatctgcacggctgagggcgccgtgctgtgcttcgatgaggtcatgaccggcttccgcattgccaagggctgcgcccaggagcacttcggtatcacccccgacctgaccaccatgggcaaggtcattggtggcggcatgcctgtgggcgcctacggcggcaagaaggagatcatgaagatggtcgcccccgccggccccatgtaccaggccggcaccctttcgggcaaccccatggccatgactgccggcatcaagacgctggagatcctgggccgccccggcgcctacgagcacctggagaaggtgaccaagcgcctgatcgacggcatcatggccgccgccaaggagcacagccacgagatcaccggcggcaacatcagcggcatgtttggcttcttcttctgcaagggccctgtgacctgcttcgaggacgccctggcggccgacactgccaagttcgcgcgcttccaccgcggcatgctggaggagggcgtctacctggctccctcgcagttcgaggccggcttcacctctctggcccactccgaggcggacgtggatgccacgatcgccgccgctcgccgcgtgttcgcccgcatctaa

SEQ ID NO: 10 - protein, 463 aa, Chlamydomonas reinhardtii
MQMQLNAKTVQGAFKAQRPRSVRGNVAVRAVAAPPKLVTKRSEEIFKEAQELLPGGVNSPVRAFRSVGGGPIVFDRVKGAYCWDVDGNKYIDYVGSWGPAICGHGNDEVNNALKAQIDKGTSFGAPCELENVLAKMVIDRVPSVEMVRFVSSGTEACLSVLRLMRAYTGREKVLKFTGCYHGHADSFLVKAGSGVITLGLPDSPGVPKSTAAATLTATYNNLDSVRELFAANKGEIAGVILEPVVGNSGFIVPTKEFLQGLREICTAEGAVLCFDEVMTGFRIAKGCAQEHFGITPDLTTMGKVIGGGMPVGAYGGKKEIMKMVAPAGPMYQAGTLSGNPMAMTAGIKTLEILGRPGAYEHLEKVTKRLIDGIMAAAKEHSHEITGGNISGMFGFFFCKGPVTCFEDALAADTAKFARFHRGMLEEGVYLAPSQFEAGFTSLAHSEADVDATIAAARRVFARI

SEQ ID NO: 11 - DNA, 1569 bp, Chlamydomonas reinhardtii
atgcagaccactatgcagcagcgtctccagggccgtaacgtggccgggcggagcgtcgctccctcggtccctgcccatcgctccttccactcacaccgggctgccactcaaaccgctacgatcagcgctgctgctagctcaaccaccaagctgccagcttcgcatctggagagcagcaagaaggcgctggattcgctgaagcagcaggccgtcaatcgctacgcgggtgacaagaagagctccattattgccattggtctcaccattcacaacgcacccgtggagctgcgcgagaagctggctgtgcctgaggctgaatggccgcgtgctattgaggagctctgccagttcccgcacatcgaggaggccgcggtgctgtcgacgtgcaatcgcatggagctctacgttgtcggtctgtcgtggcaccgcggcgttcgcgaggtggaggagtggctgtctcgcaccagcggcgtgcctctggatgagctgcgcccctacctgttcctgctgcgcgaccgcgacgccacgcaccacctgatgcgcgtgtcgggtggccttgactcgctggttatgggcgagggccagattctcgcccaagtgcgccaggtctacaaggtcggccagaactgccccggcttcggtcgccacctgaacggcctgttcaagcaggctatcaccgctggcaagcgcgtgcgtgccgagacctccatctccaccggctccgtctccgtctcatccgccgccgtcgagctggcgcagctcaagctccccacccacaactggtccgacgctaaggtctgcatcatcggcgctggcaagatgtctacgctgctggtgaagcacctgcagagcaagggctgcaaggaggtgacggtgctcaaccgctctctgccgcgcgcccaggcgctggcggaggagttccctgaggtcaagttcaacatccacctgatgcccgacctgctgcagtgcgtggaggccagcgacgtcatcttcgccgcctccggctctgaggagatcctcatccacaaggagcatgtcgaggccatgtccaagccatcggacgttgttggctccaagcgccgcttcgtcgacatctccgtgccccgcaacatcgcccccgccatcaacgagctggagcacggcatcgtctacaacgtcgacgacctgaaggaggttgtggccgccaacaaggagggccgcgcgcaggcggccgccgaggccgaggtgctgatccgcgaggagcagcgcgcgttcgaggcctggcgtgactctctggagaccgtgcccaccatcaaggcgctgcgctccaaggccgagaccatccgcgccgccgagtttgagaaggccgtgtctcgcctgggcgaggggctatccaagaagcagctcaaggcggtggaggagctcagcaagggcatcgtcaacaagctgctgcacgggcccatgacggcactgcgctgcgacggcaccgatccggatgccgtgggccagaccctcgcgaacatggaggccctggagcgcatgttccagctctcggaggtggacgtggccgcgctggcgggcaagcagtaa

SEQ ID NO: 12 - protein, 522 aa, Chlamydomonas reinhardtii
MQTTMQQRLQGRNVAGRSVAPSVPAHRSFHSHRAATQTATISAAASSTTKLPASHLESSKKALDSLKQQAVNRYAGDKKSSIIAIGLTIHNAPVELREKLAVPEAEWPRAIEELCQFPHIEEAAVLSTCNRMELYVVGLSWHRGVREVEEWLSRTSGVPLDELRPYLFLLRDRDATHHLMRVSGGLDSLVMGEGQILAQVRQVYKVGQNCPGFGRHLNGLFKQAITAGKRVRAETSISTGSVSVSSAAVELAQLKLPTHNWSDAKVCIIGAGKMSTLLVKHLQSKGCKEVTVLNRSLPRAQALAEEFPEVKFNIHLMPDLLQCVEASDVIFAASGSEEILIHKEHVEAMSKPSDVVGSKRRFVDISVPRNIAPAINELEHGIVYNVDDLKEVVAANKEGRAQAAAEAEVLIREEQRAFEAWRDSLETVPTIKALRSKAETIRAAEFEKAVSRLGEGLSKKQLKAVEELSKGIVNKLLHGPMTALRCDGTDPDAVGQTLANMEALERMFQLSEVDVAALAGKQ

SEQ ID NO: 13 - DNA, 1779 bp, Chlamydomonas reinhardtii
atgttatactcacaatttaaacattcggtgcctttaggccgtaagtctccccttctttcagggggccccccttctgggggtcgcccaacaacggctgcctcaggcctaggtcgcaacgtggccgtaagaattgggaccccgttgggctttgcccttcgggcccaggtaattatggcagctgcgggcaatactagcggtgcgccgcaccccgtaggggagtcccagcctgcgttgtcccaggtggattctcaacttgtaattgagtgtgaaacaggaaattaccatactttttgcccaattagttgtgtttcttggttataccaaaaaattgaagatagttttttcttagttattggtacaaaaacgtgtgggtattttttacaaaatgctttaggggttatgatttttgccgaacctcgttacgctatggcggaattagaagaaagcgatatttcggcgcaattaaatgattacaaagaattaaaacgtctatgtttacaaattaaacaagaccgtaacccaagtgttattgtgtggattggcacatgcacaaccgaaattattaaaatggatttagaaggtatggcaccgaaactagaagctgaaatcggtattccaattgtggtagcacgcgcaaatggacttgattatgcttttacacaaggtgaagatactgttttagctgcgatggtccaaaaatgcccggaattaggcgctattccagctattgtacctcagattccttctgactctcgtacacttagccaactatctgtagcggcttcggtacccgaaaacagtgcgtctgggccagaaggggagccttcactagcccagaagggaatggattctaagttaacaaacaactctccatgccgagtagattctgtctcagaatctaccccggcgtttcctggacgtgctccgcacgtcgggaaaagtactcctcaaaatttagttttatttggttcattacctagcacgatggcaaatcaactggagtttgaattaaaacgccaaggtattaatgttactgggtggttacctgcggctcgctattcatctttacctgcattaggtgaaaacgtgtatgtttgtgggattaatccatttttaagtcgaactgctacttctttaatgcgtcgtcgtaaatgcaaattaatttcagctcctttcccaattggtccagatggtacaaaagcttgggtcgaaaaaatttgtaatgttttcggtgttacaccaactggtttagaagatcgtgaacgtcttgtttgggaaggtttaaaagattatttaaatttcgtaaaagggaaatctgttttctttatgggtgataatctgttagaaatttcattagcccgttttttaattcgctgtggtatgaccgtttatgaaatcggtattccgtacatggaccaacgatttcaagctggggaattagaattattaaaaaaaacatgcatggaaatgaacgtgcccctaccgcgtattgttgaaaaacctgataattactatcaaattcaacgtattaaagaattacaaccagatttagttattaccggcatggcccatgcaaacccactggaagcgcgcggcattactacgaaatggtccgttgaatttacgtttgcgcaaattcatgggtttggcaacgcacgtgatatcttagaattagttacaaaaccgttacgtcgtaataaaaatctatctaaatatcaatttccgttagatagctgggacaagcctgcttccgtaggcgctcacgaactgtcggcctaa

SEQ ID NO: 14 - protein, 592 aa, Chlamydomonas reinhardtii
MLYSQFKHSVPLGRKSPLLSGGPPSGGRPTTAASGLGRNVAVRIGTPLGFALRAQVIMAAAGNTSGAPHPVGESQPALSQVDSQLVIECETGNYHTFCPISCVSWLYQKIEDSFFLVIGTKTCGYFLQNALGVMIFAEPRYAMAELEESDISAQLNDYKELKRLCLQIKQDRNPSVIVWIGTCTTEIIKMDLEGMAPKLEAEIGIPIVVARANGLDYAFTQGEDTVLAAMVQKCPELGAIPAIVPQIPSDSRTLSQLSVAASVPENSASGPEGEPSLAQKGMDSKLTNNSPCRVDSVSESTPAFPGRAPHVGKSTPQNLVLFGSLPSTMANQLEFELKRQGINVTGWLPAARYSSLPALGENVYVCGINPFLSRTATSLMRRRKCKLISAPFPIGPDGTKAWVEKICNVFGVTPTGLEDRERLVWEGLKDYLNFVKGKSVFFMGDNLLEISLARFLIRCGMTVYEIGIPYMDQRFQAGELELLKKTCMEMNVPLPRIVEKPDNYYQIQRIKELQPDLVITGMAHANPLEARGITTKWSVEFTFAQIHGFGNARDILELVTKPLRRNKNLSKYQFPLDSWDKPASVGAHELSA

SEQ ID NO: 15 - DNA, 1644 bp, Chlamydomonas reinhardtii
atgaaattagcgtattggatgtatgcgggaccggctcatattggaacattacgagttgcaagctcgtttcgaaatgtgcatgctattatgcatgctcccttaggcgatgattattttaacgtaatgcgttcaatgttagaacgtgaacgtgattttacgccagtgacggcaagtattgttgatcgtcatgttttagctcgtggttcacaagaaaaagttgttgaaaacattcaacgaaaagataaagaagaatgtccggatttaattttattaacaccaacatgtacctcaagtattttgcaagaagatttacaaaattttgtaaatcgcgcggccgaagtagcaaagcgttcggatgttttattagctgacgttaaccattaccgagtgaatgaattacaagcggctgaccgtacgttagagcaaattgtacgcttttatttagaaaaagaagtaaataaacttcacgcggagttaggcggccttaaaaaaccgcttcgctttgcccagcgtacccaaaagccgtctgccaatattttaggcatgtttacactaggtttccataatcaacatgactgtcgtgaattaaaacgtttattaaatgatttaggtatcgaagtcaatgaagtgattcctgaaggtagttttgtacatggattaaaaaatttaccaaaagcgtggtttaacatcgtcccgtatcgtgaagttggtttaatgacggcaatttatttagaaaaagaatttggcatgccttatacctcaatcacgccaatgggcattattgacaccgcggcgtttattcgtgaaattgcggccatttgtagtcaaattagcacttcacaggcatctacaaactcaactgaaggactccagaggggagaaaatgtcagtttaactgaaactaattcgattatttttaataaagcaaaatatgaacaatacattaatcaacaaacgcattttgtttctcaagcagcttggttttcacgttctattgactgtcaaaatttaaccggtaaaaaaaccgttgtgtttggtgatgcaactcacgcggcaagtatgacgaaaattcttgtgcgcgaaatgggtattcatgttgtttgcgcgggcacgtattgtaaacatgatgcagattggtttagagagcaagtttcaggtttttgtgatcaagttttaattacagatgatcacagccaaattgcggaaatcattgctcaaattgaacctgcagccatttttggtacacaaatggaacgtcatgttgggaaaaggttagatattccttgtggggttatttctgcaccggtacatattcaaaacttcccactaggctttagaccgtttttagggtatgaaggtactaatcaaatttccgatttagtttataattcgtttagtttaggtatggaagatcacttactagaaattttcaacggtcatgacaataaagaagttattacacgttcgtattcttcagaaactgatttagaatggacaaaagaagcattagatgaactagctcgtgttcctggttttgttcgttcaaaagttaaacgtaatactgaaaaatttgcgcgtacaaataaaaatcaagttattactattgaagttatgtacgcagctaaagaagcggtatcagcgtaa

SEQ ID NO: 16 - protein, 547 aa, Chlamydomonas reinhardtii
MKLAYWMYAGPAHIGTLRVASSFRNVHAIMHAPLGDDYFNVMRSMLERERDFTPVTASIVDRHVLARGSQEKVVENIQRKDKEECPDLILLTPTCTSSILQEDLQNFVNRAAEVAKRSDVLLADVNHYRVNELQAADRTLEQIVRFYLEKEVNKLHAELGGLKKPLRFAQRTQKPSANILGMFTLGFHNQHDCRELKRLLNDLGIEVNEVIPEGSFVHGLKNLPKAWFNIVPYREVGLMTAIYLEKEFGMPYTSITPMGIIDTAAFIREIAAICSQISTSQASTNSTEGLQRGENVSLTETNSIIFNKAKYEQYINQQTHFVSQAAWFSRSIDCQNLTGKKTVVFGDATHAASMTKILVREMGIHVVCAGTYCKHDADWFREQVSGFCDQVLITDDHSQIAEIIAQIEPAAIFGTQMERHVGKRLDIPCGVISAPVHIQNFPLGFRPFLGYEGTNQISDLVYNSFSLGMEDHLLEIFNGHDNKEVITRSYSSETDLEWTKEALDELARVPGFVRSKVKRNTEKFARTNKNQVITIEVMYAAKEAVSA

SEQ ID NO: 17 - DNA, 957 bp, Chlamydomonas reinhardtii
atgaaattagcagtttatggcaaaggtggtattggtaaatccacaacaagttgtaacatttcaattgcattagcaaaacgtggcaaaaaagtattacaaattggttgtgatccaaaacacgatagtacttttacattaaccggttttttaattccaacaattattgatactttacaaagtaaagattatcattacgaagatgtttggccggaagatgttatttaccaaggctacgggagtgtggattgtgttgaagcaggtggcccgccagccggcgccggctgtggtgggtatgttgttggtgaaacagttaaattattaaaagaattaaatgcattttatgaatatgatgttattctgtttgatgttttaggggatgttgtatgtggtgggtttgctgcacctttaaattacgccgactattgcattattgtcacagataatggctttgatgcgttatttgccgcaaaccgtattgctgcttcagtgcgcgaaaaagcgcgcattcacccattacgtttagctgggttaattgggaatcgtacagccaaacgcgatttaatcgataaatacgttgaagcgtgcccgatgccagtcttagaggtattaccgttaattgaagacattcgtgtgtcacgcgtaaaaggtaaaacattatttgaaatggcagaacatgattcatcattacactacatttgtgacttttatttaaatattgcggatcaattattaactgaaccagaaggtgttgttccgcgcgaattagcagaccgtgaattatttactctattatcagatttctatttaaacgctgggactcctagccctagtggatctgagttcggctcaggcgcccttagcggaacgagcggcgaaacagctcccggtaatatgggtcagcacatgagtaacgcagtaaaaacaaacgaacaggaaatgaatttctttcttgtgtaa

SEQ ID NO: 18 - protein, 318 aa, Chlamydomonas reinhardtii
MKLAVYGKGGIGKSTTSCNISIALAKRGKKVLQIGCDPKHDSTFTLTGFLIPTIIDTLQSKDYHYEDVWPEDVIYQGYGSVDCVEAGGPPAGAGCGGYVVGETVKLLKELNAFYEYDVILFDVLGDVVCGGFAAPLNYADYCIIVTDNGFDALFAANRIAASVREKARIHPLRLAGLIGNRTAKRDLIDKYVEACPMPVLEVLPLIEDIRVSRVKGKTLFEMAEHDSSLHYICDFYLNIADQLLTEPEGVVPRELADRELFTLLSDFYLNAGTPSPSGSEFGSGALSGTSGETAPGNMGQHMSNAVKTNEQEMNFFLV

SEQ ID NO: 19 - DNA, 3762 bp, Chlamydomonas reinhardtii
atgcggattgtgctggtcagcggcttcgagagctttaacgtgggcctgtacaaggatgcggcggagctgctgaagcgctccatgcccaacgtcacactccaggtgttctccgaccgcgacctggcctccgacgccacccgctcccggctggaggcggctctggggcgcgccgacatcttcttcggatcactgctgttcgactacgaccaggtggagtggctacgggcccggctggagcgggtgcctgtgcggctagtgtttgagtcggcgttggagctcatgagctgcaacaaggtggggtcgttcatgatgggcggcggcggtcccggcggcggcccgcccggcaaggcgcccggcccgccgcccgcggtgaagaaggttctctccatgtttggaagcggtcgcgaggaggacaagatgggcggctcctccaatgtggtggccatgttcagttacctggtggagaccctgatggagccaacgggtgggttatttggtagttggtggttgtgttatggttggccgtttcggttgggtgatctgggctggtatctacaacccccctcaaccctcacgcctccaggctacgtgccgccgcctgtggtggagactcccgcactgggctgcctccacccctccgcgcccggccgctacttcgagtcccccgccgagtacatgaagtggtacgccagggagggcccgctgcgcggcacgggcgccccggtggttggcgtgctgctgtaccgcaagcatgtgatcaccgaccagccgtacatcccgcagctggtcagccagctggaggcggaggggctgctgcccgtgcccatcttcatcaacggcgtggaggcgcacaccgtggttcgcgacctgctgacctccgtgcacgagcaggatctgcttgcacgcggcgagacgggcgccatcagccccaccctgaagcgggacgcggtcaaggtggacgcggtggtgagcaccattggcttcccgctggtgggcggccccgccggcaccatggagggcgggcggcaggcggaggtggccaaggccatcctgggcgccaaggacgtgccgtacacggtggcggcgccgctgcttattcaggacatggagagctggagcagggacggcgtggcgggtctccagagtgtggtgctgtactcgctgccggagctggacggcgcagtggacacggtgccactgggggggctggtgggggacgacatctacctggtgccggagcgggtgaagaagctggcggggcggctcaagtcgtggcgtacgacacgcactaagcatgcctctgtttgtgacgtccagcccctcccccccccgtctcccctctccaccctccctctcccttcctctcccttcctctcactctccaccctcttccccctccgcccaaacataacgaggcgggggctgctgggcgcaagcgggccctggagtacccgctgcgacctagctagtccaactccacccatcccccaatgccgcaatagctttccggagatgagcacacacacacacacacacacacacacacacacacacacacacacacacacacacacacgccacccacgcacacacacacacacacacgctccccccgctcgccacacccccatcccaccccacccgcaggagctgctgacgtaccccgcggactggggcccggccgagtggggcccgctgccctacctgcccgaccccgacgtgctggttcgccgcatggaggcgcagtggggcgagctgcgagcctaccgcggcctcaacacctcggcgcgcggcatgttccaggagtacggggctgacgtggtcctgcacttcggcatgcacggcaccgtggagtggttgcctggggcgccgctggggaacaacggcctcagctggagcgacgtgctgctcggcgagctgccaaacgtgtacgtgtacgctgccaacaacccctccgagtccatcgtggcaaagcggcgcggctacggcaccatcgtcagccacaacgtgccgccgtacgggcgggcgggtctgtacaagcagctttccagcctcaaggagacgcttcaggagtaccgcgaggccgcgcaggccgcacgtgcccgagcaggagccagcagcagcagcggcagtagcagcagtagcagtagcagcggcagtggcagtagcagcagcagtgtggagctgcgggcggcgttggcaccggtgttcgacgcctacactgaccgcctgtatgcctacctgcagctgctggaggggcggctgttcagcgaggggctacacgtactgggagcgccgccggcgccgccgcaggtgggtggttttcccgcgagcttccaacggtaccgtaaactgcccaactgcccaacttctccccaaacacaggaggctgtcaagatccggaacctgctcatgcagaacacgcaggagctggacgggctgctcaagggcctgggtgggcgttacgtgcttcccgaggcgggcggcgacctgctgcgggacgggtcgggcgtgctgcccaccggccgcaacatccacgcactggacccctaccgcatgccctcccccgccgccatggcccgtggggcggcggtggcggcggccattcttgagcagcaccgggcggctaacagcggggcgtggcccgagacctgcgccgtcaacctgtgggggctggactccatcaagagcaagggcgagagtgtgggggtggtgctggcgctggtgggggcggtgccggtgcgcgagggtacgggccgcgtcgcgcgcttccaactggtgccgctgtcagagttgggccggccgcgtgtggacgtgctttgtaacatgagcggcatcttccgcgactccttccagaacgtggtggagctgctcgacgacctgtttgcaagggccgccgccgccgctgacgagccagatgacatgaacttcatcgccaaacacgcccgagccatggagaagcagggcctgtccgccacctcggcccgcctgttctccaacccggctggcgactacgggtcgatggtcaacgagcgagtggggcagggcagctgggccaacggcgacgagctgggtgacacgtgggcggcccgcaacgccttcagctacggccgaggcaaggagcgaggcacggcgcggcccgaggtgctgcaggcgctgctcaagaccacggaccggatcgtgcagcagatcgacagtgtggagtacggcctgacagacatccaggagtactacgccaacacgggcgccctcaagagagccgccgaggtggccaaaggcgacccgggccccggtggccggcggccgcgcgtggggtgttccattgtggaggcctttggcggcgcgggcgcgggcgcgggcggcgccggtggagcgggcgtgccgccgcctcgcgagctggaggaggtgctgcgcctggagtaccgctcgaagctgctcaaccccaagtgggcccgggccatggcggcgcagggcagcggcggcgcctacgagatcagtcagcgcatgacggcgttggtgggctggggcgccaccaccgatttcagggagggctgggtgtgggacccaggcgccatggacacgtatgtgggcgatgaggagatggccagcaagctcaagaagaacaacccgcaggcctttgccaacgtgctgcggcgcatgctggaggcggcgggccgcggcatgtggagccccaacaaggaccagctggcacagctcaagtcgctgtacagcgagatggacgaccagctggagggggtgacg

SEQ ID NO: 20 - protein, 1254 aa, Chlamydomonas reinhardtii
MRIVLVSGFESFNVGLYKDAAELLKRSMPNVTLQVFSDRDLASDATRSRLEAALGRADIFFGSLLFDYDQVEWLRARLERVPVRLVFESALELMSCNKVGSFMMGGGGPGGGPPGKAPGPPPAVKKVLSMFGSGREEDKMGGSSNVVAMFSYLVETLMEPTGGLFGSWWLCYGWPFRLGDLGWYLQPPSTLTPPGYVPPPVVETPALGCLHPSAPGRYFESPAEYMKWYAREGPLRGTGAPVVGVLLYRKHVITDQPYIPQLVSQLEAEGLLPVPIFINGVEAHTVVRDLLTSVHEQDLLARGETGAISPTLKRDAVKVDAVVSTIGFPLVGGPAGTMEGGRQAEVAKAILGAKDVPYTVAAPLLIQDMESWSRDGVAGLQSVVLYSLPELDGAVDTVPLGGLVGDDIYLVPERVKKLAGRLKSWRTTRTKHASVCDVQPLPPPSPLSTLPLPSSPFLSLSTLFPLRPNITRRGLLGASGPWSTRCDLASPTPPIPQCRNSFPEMSTHTHTHTHTHTHTHTHTHTRHPRTHTHTHAPPARHTPIPPHPQELLTYPADWGPAEWGPLPYLPDPDVLVRRMEAQWGELRAYRGLNTSARGMFQEYGADVVLHFGMHGTVEWLPGAPLGNNGLSWSDVLLGELPNVYVYAANNPSESIVAKRRGYGTIVSHNVPPYGRAGLYKQLSSLKETLQEYREAAQAARARAGASSSSGSSSSSSSSGSGSSSSSVELRAALAPVFDAYTDRLYAYLQLLEGRLFSEGLHVLGAPPAPPQVGGFPASFQRYRKLPNCPTSPQTQEAVKIRNLLMQNTQELDGLLKGLGGRYVLPEAGGDLLRDGSGVLPTGRNIHALDPYRMPSPAAMARGAAVAAAILEQHRAANSGAWPETCAVNLWGLDSIKSKGESVGVVLALVGAVPVREGTGRVARFQLVPLSELGRPRVDVLCNMSGIFRDSFQNVVELLDDLFARAAAAADEPDDMNFIAKHARAMEKQGLSATSARLFSNPAGDYGSMVNERVGQGSWANGDELGDTWAARNAFSYGRGKERGTARPEVLQALLKTTDRIVQQIDSVEYGLTDIQEYYANTGALKRAAEVAKGDPGPGGRRPRVGCSIVEAFGGAGAGAGGAGGAGVPPPRELEEVLRLEYRSKLLNPKWARAMAAQGSGGAYEISQRMTALVGWGATTDFREGWVWDPGAMDTYVGDEEMASKLKKNNPQAFANVLRRMLEAAGRGMWSPNKDQLAQLKSLYSEMDDQLEGVT

SEQ ID NO: 21 - DNA, 1254 bp, Chlamydomonas reinhardtii
atggccctgaacatgcgtgtttcctcttccaaggtcgctgccaagcagcagggccgcatctccgcggtgccggttgtgtcgagcaaggtggcctcctccgcccgcgtggcccccttccagggcgctcccgtggccgcgcagcgcgctgctctgctggtgcgcgccgctgccgctactgaggtcaaggctgctgagggccgcactgagaaggagctgggccaggcccgccccatcttccccttcaccgccatcgtgggccaggatgagatgaagctggcgctgattctgaacgtgatcgaccccaagatcggtggtgtcatgatcatgggcgaccgtggcactggcaagtccaccaccattcgtgccctggcggatctgctgcccgagatgcaggtggttgccaacgacccctttaactcggaccccaccgaccccgagctgatgagcgaggaggtgcgcaaccgcgtcaaggccggcgagcagctgcccgtgtcttccaagaagattcccatggtggacctgcccctgggcgccactgaggaccgcgtgtgcggcaccatcgacatcgagaaggcgctgaccgagggtgtcaaggcgttcgagcccggcctgctggccaaggccaaccgcggcatcctgtacgtggatgaggtcaacctgctggacgaccacctggtcgatgtgctgctggactcggccgcctccggctggaacaccgtggagcgcgagggtatctccatcagccaccccgcccgcttcatcctggtcggctcgggcaaccccgaggagggtgagctgcgcccccagctgctggatcgcttcggcatgcacgcccagatcggcaccgtcaaggacccccgcctgcgtgtgcagatcgtgtcgcagcgctcgaccttcgacgagaaccccgccgccttccgcaaggactacgaggccggccagatggcgctgacccagcgcatcgtggacgcgcgcaagctgctgaagcagggcgaggtcaactacgacttccgcgtcaagatcagccagatctgctcggacctgaacgtggacggcatccgcggcgacatcgtgaccaaccgcgccgccaaggccctggccgccttcgagggccgcaccgaggtgacccccgaggacatctaccgtgtcattcccctgtgcctgcgccaccgcctccggaaagaccccctggctgagatcgacgacggtgaccgcgtgcgtgagatcttcaagcaggtgttcggcatggagtaa

SEQ ID NO: 22 - protein, 417 aa, Chlamydomonas reinhardtii
MALNMRVSSSKVAAKQQGRISAVPVVSSKVASSARVAPFQGAPVAAQRAALLVRAAAATEVKAAEGRTEKELGQARPIFPFTAIVGQDEMKLALILNVIDPKIGGVMIMGDRGTGKSTTIRALADLLPEMQVVANDPFNSDPTDPELMSEEVRNRVKAGEQLPVSSKKIPMVDLPLGATEDRVCGTIDIEKALTEGVKAFEPGLLAKANRGILYVDEVNLLDDHLVDVLLDSAASGWNTVEREGISISHPARFILVGSGNPEEGELRPQLLDRFGMHAQIGTVKDPRLRVQIVSQRSTFDENPAAFRKDYEAGQMALTQRIVDARKLLKQGEVNYDFRVKISQICSDLNVDGIRGDIVTNRAAKALAAFEGRTEVTPEDIYRVIPLCLRHRLRKDPLAEIDDGDRVREIFKQVFGME

SEQ ID NO: 23 - DNA, 1278 bp, Chlamydomonas reinhardtii
atgcagagtctccagggtcagcgcgcgttcactgcggtgcgccagggtcgggcgggtcccctgcggactcgcctggtcgtgcgctcgtctgttgccttgccatccacgaaagccgcgaagaagccgaacttcccgttcgtcaagattcagggccaggaggagatgaagcttgcactgctgctgaacgtggtcgaccccaacatcggcggagtgcttattatgggtgaccgcggcactgccaagtcggtcgcggtccgcgccctggtggatatgcttcccgacattgacgtggttgagggcgacgccttcaacagctcccccaccgaccccaagttcatgggccccgacaccctgcagcgcttccgcaacggcgagaagctgcccaccgtccgcatgcggacccccctggtggagctgcctctgggcgccaccgaggaccgcatctgcggcaccatcgacatcgagaaggcgctgacgcagggcatcaaggcctacgagcccggcctgctggccaaggccaaccgcggcatcctgtatgtggacgaggtgaacctgctggatgatggcctggttgatgtcgtgctggactcgtcggctagcggcctgaacactgtggagcgtgagggtgtgtccattgtgcaccctgcccgcttcatcatgattggctcaggcaacccccaggagggtgagctgcgcccgcagctgctggatcgcttcggcatgagcgtcaacgtggccacgctgcaggacaccaagcagcgcacgcagctggtgctggaccggcttgcgtacgaggcggaccctgacgcatttgtggactcgtgcaaggccgagcagacggcgctcacggacaagctggaggcggcccgccagcgcctgcggtccgtcaagatcagcgaggagctgcagatcctgatctcggacatttgctcgcgcctggatgtggatggcctgcgcggtgacattgtgatcaaccgcgccgccaaggcgcttgtggccttcgagggccgcaccgaggtgaccacgaatgacgtggagcgcgtcatctcgggctgcctcaaccaccgcctgcgcaaggacccgctggaccccattgacaacggcaccaaggtggccatcctgttcaagcgcatgaccgaccccgagatcatgaagcgcgaggaggaggccaagaagaagcgcgaggaggcggccgccaaggccaaggcggagggcaaggcggaccgccccacgggcgccaaggctggcgcctgggctggcttgccccctcgtcggtaa

SEQ ID NO: 24 - protein, 425 aa, Chlamydomonas reinhardtii
MQSLQGQRAFTAVRQGRAGPLRTRLVVRSSVALPSTKAAKKPNFPFVKIQGQEEMKLALLLNVVDPNIGGVLIMGDRGTAKSVAVRALVDMLPDIDVVEGDAFNSSPTDPKFMGPDTLQRFRNGEKLPTVRMRTPLVELPLGATEDRICGTIDIEKALTQGIKAYEPGLLAKANRGILYVDEVNLLDDGLVDVVLDSSASGLNTVEREGVSIVHPARFIMIGSGNPQEGELRPQLLDRFGMSVNVATLQDTKQRTQLVLDRLAYEADPDAFVDSCKAEQTALTDKLEAARQRLRSVKISEELQILISDICSRLDVDGLRGDIVINRAAKALVAFEGRTEVTTNDVERVISGCLNHRLRKDPLDPIDNGTKVAILFKRMTDPEIMKREEEAKKKREEAAAKAKAEGKADRPTGAKAGAWAGLPPRR

SEQ ID NO: 25 - DNA, 2304 bp, Chlamydomonas reinhardtii
atgaagtctctctgccatgagctcgctggccccagcgttactgggtgcggccggcgaagcctccggaaggctttcagcggtgccaagattgcgcaggtctctcgccccgctgtgcttaacagcgtgcagcgccaacagcgtctcgcctgttctgccgtggccgagctctccgctgctgagctgcgcgccatgaaggtgtctgaggaggactccaagggcttcgatgcggatgtgtcgacccgcctggcccgctcgtaccctctggcggccgtggtgggccaggacaacatcaagcaggcgctgctgctgggcgccgtggacaccgggctgggcggcatcgccatcgccggtcgccgcggtaccgccaagtccatcatggctcgcggcctgcacgctctgctgccgcccattgaggtggtggagggcagcatctgcaacgccgaccccgaggacccccgctcctgggaggctggcctggctgagaagtatgcgggcggccctgtgaagaccaagatgcgctcggcgccgtttgtgcagatccctctgggtgtgactgaggaccgcttggtgggcactgtggacattgaggcgtccatgaaggagggcaagactgtgttccagcccggcctgctggctgaggcgcaccgcggcatcctgtacgtggacgagatcaacctgctggatgacggcattgccaacctgctgctgtccatcctgtcggacggagtcaacgtggtggagcgcgagggcatctccatcagccacccctgccggccgctgctgattgccacctacaaccccgaggagggccctctgcgtgagcacctgctggaccgcatcgccattggcctcagcgccgacgtccccagcaccagcgacgagcgcgtcaaggccattgacgcagccatccgcttccaggacaagccgcaggacactattgacgacaccgcggagctcaccgacgccctgcgcacctcggtcatcctggctcgcgagtacctgaaggacgtgaccatcgcgccggagcaggtgacctacattgtggaggaggcgcgccgcggcggagtccaggggcaccgcgcggagctgtacgcggtcaagtgtgccaaggcgtgtgcggctctggagggccgtgagcgtgtgaacaaggatgacctgcgccaggccgtgcagctggtcatcctgccgcgcgccaccatcctggaccagcccccgcccgagcaggagcagcccccgccgccgcccccgccccctcccccgccgccgccgcaggaccaaatggaggacgaggaccaggaggagaaggaggacgagaaggaggaggaggagaaggagaacgaggaccaggacgagcccgagatccctcaggagttcatgtttgagtccgagggcgtcatcatggacccctccatcctcatgttcgcgcagcagcagcagcgcgcgcagggccgctccggccgcgccaagacgctcatcttcagcgacgaccgcggccgctacatcaagcccatgctgcccaagggtgacaaggtcaagcgcctggcagtggacgccacgcttcgcgccgccgcgccctaccagaagattcgccggcagcaggccatcagcgagggcaaggtgcagcgcaaggtgtacgtggacaagccagacatgcgctccaagaagctggcccgcaaggccggtgcgctggtgatttttgttgtggacgcgtccggctccatggctctgaaccgcatgagcgccgccaagggcgcctgcatgcgcctgctggctgagtcgtacaccagccgcgaccaggtgtgcctcatccccttctacggcgacaaggccgaggtgctgctgccgccctccaagtccatcgccatggcccgccgccgcctggactcgctgccctgcggcggcggctcgccccttgcgcacggcctgtccacggcggtacgtgtgggcatgcaggccagccaggcgggcgaggtgggccgcgtcatgatggtgctcatcacggacggccgcgccaacgtcagcctggccaagtccaacgaggaccccgaggcgctcaagcccgacgcgcccaagcccaccgccgactcgctgaaggacgaggtgcgcgacatggccaagaaggccgcgtccgccggcatcaacgtgcttgtcattgacacggagaacaagttcgtgagcaccggctttgcggaggagatctccaaggcagcgcagggcaagtactactacctgcccaacgccagcgacgccgccatcgcggcggccgcgtccggcgccatggccgcggccaagggcggctactag

SEQ ID NO: 26 - protein, 767 aa, Chlamydomonas reinhardtii
MKSLCHELAGPSVTGCGRRSLRKAFSGAKIAQVSRPAVLNSVQRQQRLACSAVAELSAAELRAMKVSEEDSKGFDADVSTRLARSYPLAAVVGQDNIKQALLLGAVDTGLGGIAIAGRRGTAKSIMARGLHALLPPIEVVEGSICNADPEDPRSWEAGLAEKYAGGPVKTKMRSAPFVQIPLGVTEDRLVGTVDIEASMKEGKTVFQPGLLAEAHRGILYVDEINLLDDGIANLLLSILSDGVNVVEREGISISHPCRPLLIATYNPEEGPLREHLLDRIAIGLSADVPSTSDERVKAIDAAIRFQDKPQDTIDDTAELTDALRTSVILAREYLKDVTIAPEQVTYIVEEARRGGVQGHRAELYAVKCAKACAALEGRERVNKDDLRQAVQLVILPRATILDQPPPEQEQPPPPPPPPPPPPPQDQMEDEDQEEKEDEKEEEEKENEDQDEPEIPQEFMFESEGVIMDPSILMFAQQQQRAQGRSGRAKTLIFSDDRGRYIKPMLPKGDKVKRLAVDATLRAAAPYQKIRRQQAISEGKVQRKVYVDKPDMRSKKLARKAGALVIFVVDASGSMALNRMSAAKGACMRLLAESYTSRDQVCLIPFYGDKAEVLLPPSKSIAMARRRLDSLPCGGGSPLAHGLSTAVRVGMQASQAGEVGRVMMVLITDGRANVSLAKSNEDPEALKPDAPKPTADSLKDEVRDMAKKAASAGINVLVIDTENKFVSTGFAEEISKAAQGKYYYLPNASDAAIAAAASGAMAAAKGGY

SEQ ID NO: 27 - DNA, 4200 bp, Chlamydomonas reinhardtii
atgcagacttcctcgcttcttggccggcgcacggcccacccggctgcgggcgcgacgcccaagccggttgcgccctcgccccgcgtggctagcacccgccaggtcgcgtgcaatgtggcgactggaccccggccgcccatgaccaccttcaccggtggcaacaagggccctgctaagcagcaggtgtcgctggatctgcgcgacgagggcgctggcatgttcaccagcaccagcccggagatgcgccgtgtcgtccctgacgatgtgaagggtcgcgttaaggtgaaggttgtgtacgtggtgctggaggcccagtaccagtcggccatcagcgctgcggtgaagaacatcaacgccaagaactccaaggtgtgcttcgaggtggtgggctacctgctggaggagctgcgtgaccagaagaacctcgatatgctcaaggaggatgtggcctctgccaacatcttcatcggctcgctcatcttcattgaggagcttgccgagaagattgtggaggcggtgagccccctgcgcgagaagctggacgcgtgcctgatcttcccgtccatgccggcggtcatgaagctgaacaagctgggcacgttttcgatggctcagctgggccagtcgaagtcggtgttctcggagttcatcaagtctgctcgcaagaacaacgacaacttcgaggagggcttgctgaagctggtgcgcaccctgcctaaggtgctgaagtatctgccctcggacaaggcgcaggacgccaagaacttcgtgaacagcctgcagtactggctgggcggtaactcggacaacctggagaacctgctgctgaacaccgtcagcaactacgtgcccgctctgaagggcgtggacttcagcgtggctgagcccaccgcctaccccgatgtgggtatctggcaccctctggcctcgggcatgtacgaggacctgaaggagtacctgaactggtacgacacccgcaaggacatggtcttcgccaaggacgcccccgtcattggcctggtgctgcagcgctcgcacctggtgactggcgatgagggccactacagcggcgtggtcgctgagctggagagccgcggtgctaaggtcatccccgtctttgccggtggcctggacttctccgcccccgtcaagaagttcttctacgaccccctgggctctggccgcacgttcgtggacaccgttgtgtcgctgaccggcttcgcgctggtgggcggccccgcgcgccaggacgcgccgaaggccattgaggcgctgaagaacctgaacgtgccctacctggtgtcgctgccgctggtgttccagaccactgaggagtggctggacagcgagctgggcgtgcaccccgtccaggtggctctgcaggttgccctgcccgagctggatggtgccatggagcccatcgtgttcgctggccgtgactcgaacaccggcaagtcgcactcgctgcccgaccgcatcgcttcgctgtgcgctcgcgccgtgaactgggccaacctgcgcaagaagcgcaacgccgagaagaagctggccgtcaccgtgttcagcttcccccctgacaagggcaacgtcggcactgccgcctacctgaacgtgttcggctccatctaccgcgtgctgaagaacctgcagcgcgagggctacgacgtgggcgccctgccgccctcggaggaggatctgatccagtcggtgctgacccagaaggaggccaagttcaactcgaccgacctgcacatcgcctacaagatgaaggtggacgagtaccagaagctgtgcccttacgccgaggcgctggaggagaactggggcaagccccccggcaccctgaacaccaacggccaggagctgctggtgtacggccgccagtacggcaacgtcttcatcggcgtgcagcccaccttcggctacgagggcgacccgatgcgcctgctgttctcgaagtcggccagcccccaccacggcttcgccgcctactacaccttcctggagaagatcttcaaggccgacgccgtgctgcacttcggcacccacggctcgctggagttcatgcccggcaagcaggtcggcatgtcgggtgtgtgctaccccgactcgctgatcggcaccatccccaacctctactactacgccgccaacaacccgtctgaggccaccatcgccaagcgccgctcgtacgccaacaccatttcgtacctgacgccgcctgccgagaacgccggcctgtacaagggcctgaaggagctgaaggagctgatcagctcgtaccagggcatgcgtgagtctggccgcgccgagcagatctgcgccaccatcattgagaccgccaagctgtgcaacctggaccgcgacgtgaccctgcccgacgctgacgccaaggacctgaccatggacatgcgcgacagcgttgtgggccaggtgtaccgcaagctgatggagattgagtcccgcctgctgccctgcggcctgcacgtggtgggctgcccgcccaccgccgaggaggccgtggccaccctggtcaacatcgctgagctggaccgcccggacaacaacccccccatcaagggcatgcccggcatcctggcccgcgccattggtcgcgacatcgagtcgatttacagcggcaacaacaagggcgtcctggctgacgttgaccagctgcagcgcatcaccgaggcctcccgcacctgcgtgcgcgagttcgtgaaggaccgcaccggcctgaacggccgcatcggcaccaactggatcaccaacctgctcaagttcaccggcttctacgtggacccctgggtgcgcggcctgcagaacggcgagttcgccagcgccaaccgcgaggagctgatcaccctgttcaactacctggagttctgcctgacccaggtggtcaaggacaacgagctgggcgccctggtagaggcgctgaacggccagtacgtcgagcccggccccggcggtgaccccatccgcaaccccaacgtgctgcccaccggcaagaacatccacgccctggaccctcagtcgattcccactcaggccgcgctgaagagcgcccgcctggtggtggaccgcctgctggaccgcgagcgcgacaacaacggcggcaagtaccccgagaccatcgcgctggtgctgtggggcactgacaacatcaagacctacggcgagtcgctggcccaggtcatgatgatggtcggtgtcaagcccgtggccgacgccctgggccgcgtgaacaagctggaggtgatccctctggaggagctgggccgcccccgcgtggacgtggttgtcaactgctcgggtgtgttccgcgacctgttcgtgaaccagatgctgctgctggaccgcgccatcaagctggcggccgagcaggacgagcccgatgagatgaacttcgtgcgcaagcacgccaagcagcaggcggcggagctgggcctgcagagcctgcgcgacgcggccacccgtgtgttctccaacagctcgggctcctactcgtccaacgtcaacctggcggtggagaacagcagctggagcgacgagtcgcagctgcaggagatgtacctgaagcgcaagtcgtacgccttcaactcggaccgccccggcgccggtggcgagatgcagcgcgacgtgttcgagacggccatgaagaccgtggacgtgaccttccagaacctggactcgtccgagatctcgctgaccgatgtgtcgcactacttcgactccgaccccaccaagctggtggcgtcgctgcgcaacgacggccgcacccccaacgcctacatcgccgacaccaccaccgccaacgcgcaggtccgcactctgggtgagaccgtgcgcctggacgcccgcaccaagctgctcaaccccaagtggtacgagggcatgcttgcctcgggctacgagggcgtgcgcgagatccagaagcgcatgaccaacaccatgggctggtcggccacctcgggcatggtggacaactgggtgtacgacgaggccaactcgaccttcatcgaggatgcggccatggccgagcgcctgatgaacaccaaccccaacagcttccgcaagctggtggccaccttcctggaggccaacggccgcggctactgggacgccaagcccgagcagctggagcgcctgcgccagctgtacatggacgtggaggacaagattgagggcgtcgaataa

SEQ ID NO: 28 - protein, 1399 aa, Chlamydomonas reinhardtii
MQTSSLLGRRTAHPAAGATPKPVAPSPRVASTRQVACNVATGPRPPMTTFTGGNKGPAKQQVSLDLRDEGAGMFTSTSPEMRRVVPDDVKGRVKVKVVYVVLEAQYQSAISAAVKNINAKNSKVCFEVVGYLLEELRDQKNLDMLKEDVASANIFIGSLIFIEELAEKIVEAVSPLREKLDACLIFPSMPAVMKLNKLGTFSMAQLGQSKSVFSEFIKSARKNNDNFEEGLLKLVRTLPKVLKYLPSDKAQDAKNFVNSLQYWLGGNSDNLENLLLNTVSNYVPALKGVDFSVAEPTAYPDVGIWHPLASGMYEDLKEYLNWYDTRKDMVFAKDAPVIGLVLQRSHLVTGDEGHYSGVVAELESRGAKVIPVFAGGLDFSAPVKKFFYDPLGSGRTFVDTVVSLTGFALVGGPARQDAPKAIEALKNLNVPYLVSLPLVFQTTEEWLDSELGVHPVQVALQVALPELDGAMEPIVFAGRDSNTGKSHSLPDRIASLCARAVNWANLRKKRNAEKKLAVTVFSFPPDKGNVGTAAYLNVFGSIYRVLKNLQREGYDVGALPPSEEDLIQSVLTQKEAKFNSTDLHIAYKMKVDEYQKLCPYAEALEENWGKPPGTLNTNGQELLVYGRQYGNVFIGVQPTFGYEGDPMRLLFSKSASPHHGFAAYYTFLEKIFKADAVLHFGTHGSLEFMPGKQVGMSGVCYPDSLIGTIPNLYYYAANNPSEATIAKRRSYANTISYLTPPAENAGLYKGLKELKELISSYQGMRESGRAEQICATIIETAKLCNLDRDVTLPDADAKDLTMDMRDSVVGQVYRKLMEIESRLLPCGLHVVGCPPTAEEAVATLVNIAELDRPDNNPPIKGMPGILARAIGRDIESIYSGNNKGVLADVDQLQRITEASRTCVREFVKDRTGLNGRIGTNWITNLLKFTGFYVDPWVRGLQNGEFASANREELITLFNYLEFCLTQVVKDNELGALVEALNGQYVEPGPGGDPIRNPNVLPTGKNIHALDPQSIPTQAALKSARLVVDRLLDRERDNNGGKYPETIALVLWGTDNIKTYGESLAQVMMMVGVKPVADALGRVNKLEVIPLEELGRPRVDVVVNCSGVFRDLFVNQMLLLDRAIKLAAEQDEPDEMNFVRKHAKQQAAELGLQSLRDAATRVFSNSSGSYSSNVNLAVENSSWSDESQLQEMYLKRKSYAFNSDRPGAGGEMQRDVFETAMKTVDVTFQNLDSSEISLTDVSHYFDSDPTKLVASLRNDGRTPNAYIADTTTANAQVRTLGETVRLDARTKLLNPKWYEGMLASGYEGVREIQKRMTNTMGWSATSGMVDNWVYDEANSTFIEDAAMAERLMNTNPNSFRKLVATFLEANGRGYWDAKPEQLERLRQLYMDVEDKIEGVE

SEQ ID NO: 29 - DNA, 2064 bp, Chlamydomonas reinhardtii
atgaaattagcttattggatgtacgcaggtcccgctcatatcggtgtgttgcgtgttagcagctcttttaaaaatgtacatgccattatgcatgctcctttaggagatgattattttaatgtaatgcgttccatgttagaacgtgaacgtgattttacaccagtaacagccagtattgtagatcgtcatgttttagcaagaggatcgcaagaaaaagtggttgaaaatattacgcgaaaaaataaagaagaaactcctgatttaattttattaactcctacttgtacgtcaagcattttacaagaagatttacacaattttgttgaatcggcattagctaaaccagtacaaatagatgaacatgcagaccataaagtaactcaacaaagtgcactttcaagtgtatcccctttactaccgcttgaagaaaatacattaatagtaagtgaactagataagaagcttagcccgtctagcaagttgcatattaatatgcccaatatttgtattcccgaaggagaaggggaaggggagcagactaaaaattcaatttttgttaaatctgcaactttaacaaatttgtcagaagaggaactattaaatcaagaacatcataccaaaacaagaaatcactctgacgttattttagctgatgtaaaccattatcgtgtaaatgaattacaagctgcagatcgtactcttgaacaaattgtacgttattatatttctcaagcacaaaaacaaaattgtttaaacattactaaaacagccaaaccatctgtaaatattattggtatttttactttgggttttcataatcaacatgattgtcgtgaattaaaacgtttatttaatgatttaggtattcaaatcaatgaaatcatacctgaaggcggaaatgtacacaacttaaaaaaattaccccaagcttggtttaattttgtgccctaccgtgaaattggcttaatgactgctatgtatttaaaatccgagtttaatatgccttacgtcgcaattactcctatgggattaattgatacggctgcttgtattcgttcaatttgtaaaatcattacaactcaattattaaatcagacggctacagtgcaggagccatcaaaatttatttacccgaaggcgacgtcattagaacaaaccaatattctcgaaacctctcaaaaagaaactattcttaaagacaatccagatagcggaaataccctttctacaactgtagaagaaattgaaactttatttaataaatatatcgatcaacaaactcgttttgtttcccaagcagcctggttttcacgttctattgactgtcaaaatttaacaggtaaaaaagccgtagttttcggagatgctacacattcagctgccatgacaaaattattagcacgtgaaatgggtattaaggtttcatgcgctggaacttattgcaaacacgatgcggattggtttagagagcaagttagtgggttttgtgatcaagttttaattaccgatgatcacacacaagtaggggatatgattgcacaattagaacctgcagccatttttgggacacaaatggaacgtcacgttggtaaacgtttagatattccatgtggtgttatatctgctcctgtgcatattcaaaactttccgttaggttatcgaccttttttaggttatgaaggtacaaatcaaatagctgatttagtgtataattcatttaatcttggaatggaagaccatttattacaaatttttggaggtcatgattcagaaaacaattcgtcaattgcaacgcatttgaatacaaataacgcaataaatttagcgccaggatatttacctgagggagaaggcagtagtagaacttcaaatgtagtgtctacaatttctagtgaaaaaaaagccattgtatggtctccagaaggtttagcagaattaaataaagtcccaggatttgttcgaggaaaagttaaacgtaatacggaaaaatatgctttacaaaaaaattgttcgatgattactgtagaagttatgtatgcagcaaaagaagctttgtcggcttaa

SEQ ID NO: 30 - protein, 687 aa, Chlamydomonas reinhardtii
MKLAYWMYAGPAHIGVLRVSSSFKNVHAIMHAPLGDDYFNVMRSMLERERDFTPVTASIVDRHVLARGSQEKVVENITRKNKEETPDLILLTPTCTSSILQEDLHNFVESALAKPVQIDEHADHKVTQQSALSSVSPLLPLEENTLIVSELDKKLSPSSKLHINMPNICIPEGEGEGEQTKNSIFVKSATLTNLSEEELLNQEHHTKTRNHSDVILADVNHYRVNELQAADRTLEQIVRYYISQAQKQNCLNITKTAKPSVNIIGIFTLGFHNQHDCRELKRLFNDLGIQINEIIPEGGNVHNLKKLPQAWFNFVPYREIGLMTAMYLKSEFNMPYVAITPMGLIDTAACIRSICKIITTQLLNQTATVQEPSKFIYPKATSLEQTNILETSQKETILKDNPDSGNTLSTTVEEIETLFNKYIDQQTRFVSQAAWFSRSIDCQNLTGKKAVVFGDATHSAAMTKLLAREMGIKVSCAGTYCKHDADWFREQVSGFCDQVLITDDHTQVGDMIAQLEPAAIFGTQMERHVGKRLDIPCGVISAPVHIQNFPLGYRPFLGYEGTNQIADLVYNSFNLGMEDHLLQIFGGHDSENNSSIATHLNTNNAINLAPGYLPEGEGSSRTSNVVSTISSEKKAIVWSPEGLAELNKVPGFVRGKVKRNTEKYALQKNCSMITVEVMYAAKEALSA

SEQ ID NO: 31 - DNA, 882 bp, Chlamydomonas reinhardtii
atgaaattagctgtttacggaaaaggtggtattggaaaatcaacgacaagttgtaatatttcgattgctttacgaaaacgtggtaaaaaagtgttacaaattggttgtgatcctaaacatgatagtacttttacattgacagggtttttaattccaaccattattgatacattaagttctaaagattatcattatgaagatatttggcccgaagatgttatttacggaggttatgggggtgtagattgtgttgaagctggaggaccacctgccggtgcggggtgtggtggttatgttgtaggtgaaacggtaaaacttttaaaagagttaaatgcttttttcgaatacgatgttattttatttgatgttttaggtgatgttgtttgtggtggctttgctgctccattaaactacgctgattattgtattattgtaactgataatggttttgatgctttatttgctgcaaatcgtattgcagcttcagttcgtgaaaaagcacgtacacatccattgcgtttagcgggtttaatcggaaatcgtacatcaaaacgtgatttaattgataaatatgtagaagcttgtcctatgccagtattagaagttttaccattaattgaagaaattcgtatttcacgtgttaaaggcaaaactttatttgaaatgtcaaataaaaataatatgacttcggctcatatggatggctctaaaggtgacaattctacagtaggagtgtcagaaactccatcggaagattatatttgtaatttttatttaaatattgctgatcaattattaacagaaccagaaggagttattccacgtgaattagcagataaagaactttttactcttttatcagatttctatcttaaaatttaa

SEQ ID NO: 32 - protein, 293 aa, Chlamydomonas reinhardtii
MKLAVYGKGGIGKSTTSCNISIALRKRGKKVLQIGCDPKHDSTFTLTGFLIPTIIDTLSSKDYHYEDIWPEDVIYGGYGGVDCVEAGGPPAGAGCGGYVVGETVKLLKELNAFFEYDVILFDVLGDVVCGGFAAPLNYADYCIIVTDNGFDALFAANRIAASVREKARTHPLRLAGLIGNRTSKRDLIDKYVEACPMPVLEVLPLIEEIRISRVKGKTLFEMSNKNNMTSAHMDGSKGDNSTVGVSETPSEDYICNFYLNIADQLLTEPEGVIPRELADKELFTLLSDFYLKI

SEQ ID NO: 33 - DNA, 1410 bp, Chlamydomonas reinhardtii
atgttagatggtgccacaacgattttaaatttaaatagtttttttgaatgtgaaactggcaattatcatactttttgcccgattagctgtgtagcttggttatatcaaaaaatcgaagatagcttttttttagtaattgggacaaaaacatgtggttattttttacaaaatgcccttggagttatgatttttgccgaacctaggtatgctatggcagaattagaagaaagtgatatttcagcacaattaaacgattataaagaattaaaacgtttatgtttacaaattaaacaagatagaaatcccagcgttattgtttggattggaacttgtacaactgaaattatcaaaatggatttagaagggatggctccacgtttagaaactgaaatcggcatacccattgttgtagcacgtgctaatggtttagattatgcttttacacaaggtgaagacacagttttatcagcaatggccttagcatccttaaaaaaagatgttccttttttagtaggtaatactgggttaacaaacaaccagcttctccttgaaaaatcaacttcttcagttaatgggacagacggaaaggaattacttaaaaaatctcttgtattatttggttccgtaccaagtacagttactacacaattaactttagaattaaaaaaagaaggtattaatgtatctggatggcttccatctgctaattataaagatttacctacttttaataaagatacacttgtatgtggtataaatccttttttaagtcgaacagctaccacgttaatgcgtcgtagtaagtgcacattaatttgtgcaccctttccaataggccccgatggcacaagagtttggattgaaaaaatttgtggtgcttttggcattaatcctagtcttaatccaattactggtaatactaatttatatgatcgtgaacaaaaaattttcaacgggctagaagattatttaaaattattacgtggaaaatctgtattttttatgggtgataatttattagaaatttctttagcacgttttttaacacgttgtggtatgattgtttatgaaatcggaattccatatttagataaacgatttcaagcagcagaattagctttattagaacaaacttgtaaagaaatgaatgtaccaatgccgcgcattgtagaaaaaccagataattattatcaaattcgacgtatacgtgaattaaaacctgatttaacgattactggaatggcacatgcaaatccattagaagctcgaggtattacaacaaaatggtcagttgaatttacttttgctcaaattcatggatttactaatacacgtgaaattttagaattagtaacacagcctcttagacgcaatctaatgtcaaatcaatctgtaaatgctatttcttaa

SEQ ID NO: 34 - protein, 469 aa, Chlamydomonas reinhardtii
MLDGATTILNLNSFFECETGNYHTFCPISCVAWLYQKIEDSFFLVIGTKTCGYFLQNALGVMIFAEPRYAMAELEESDISAQLNDYKELKRLCLQIKQDRNPSVIVWIGTCTTEIIKMDLEGMAPRLETEIGIPIVVARANGLDYAFTQGEDTVLSAMALASLKKDVPFLVGNTGLTNNQLLLEKSTSSVNGTDGKELLKKSLVLFGSVPSTVTTQLTLELKKEGINVSGWLPSANYKDLPTFNKDTLVCGINPFLSRTATTLMRRSKCTLICAPFPIGPDGTRVWIEKICGAFGINPSLNPITGNTNLYDREQKIFNGLEDYLKLLRGKSVFFMGDNLLEISLARFLTRCGMIVYEIGIPYLDKRFQAAELALLEQTCKEMNVPMPRIVEKPDNYYQIRRIRELKPDLTITGMAHANPLEARGITTKWSVEFTFAQIHGFTNTREILELVTQPLRRNLMSNQSVNAIS

SEQ ID NO: 35 - DNA, 1050 bp, Chlamydomonas reinhardtii
atgcagcagtgcgttggccgctccgtccgcgctccgtccagcagggcggtcgcgcccaaggtcgctggcgctcgtgtcagccgccgcgtgtgccgcgtctatgcctccgctgttgctaccaagacggtgaagattggcacgcgcggctcgcccctggctctggcccaggcttacatgactcgcgacctgctgaagaagagcttccctgagctgagcgaggagggtgctctggagatcgtgatcatcaagaccaccggtgacaaaatcctgaaccagcccctggctgacatcggtggcaagggtctgtttaccaaggagatcgatgatgctctgctgagcggcaagattgacatcgccgtgcactccatgaaggacgtgcccacctacctgcccgagggcaccatcctgccctgcaacctgccccgcgaggatgtgcgcgatgtgttcatctcgcctgtcgccaaggacctgagcgagctgcccgccggcgccattgtgggctcggcctcgctgcgccgtcaggcccagatcctggccaagtacccccacctcaaggtggagaacttccgcggcaacgtgcagacccgcctgcgcaagctgaacgagggcgcctgctccgccaccctgctggctctggccggtctgaagcgcctggacatgactgagcacatcaccaagaccctcagcattgacgagatgctgcccgccgtgagccagggcgccattggcattgcctgccgcaccgacgacggcgccagccgcaacctgctggccgccctgaaccacgaggagacccgcatcgccgtggtgtgcgagcgcgccttcctgaccgccctggacggctcttgccgcacccccattgccggctacgcgcacaagggcgccgacggcatgctgcacttcagcggcctggtggccaccccggacggcaagcagatcatgcgcgctagccgcgtggtgcccttcacggaggcggatgccgtcaagtgcggcgaggaggccggcaaggagctcaaggccaacggccccaaggagctgttcatgtactaa

SEQ ID NO: 36 - protein, 349 aa, Chlamydomonas reinhardtii
MQQCVGRSVRAPSSRAVAPKVAGARVSRRVCRVYASAVATKTVKIGTRGSPLALAQAYMTRDLLKKSFPELSEEGALEIVIIKTTGDKILNQPLADIGGKGLFTKEIDDALLSGKIDIAVHSMKDVPTYLPEGTILPCNLPREDVRDVFISPVAKDLSELPAGAIVGSASLRRQAQILAKYPHLKVENFRGNVQTRLRKLNEGACSATLLALAGLKRLDMTEHITKTLSIDEMLPAVSQGAIGIACRTDDGASRNLLAALNHEETRIAVVCERAFLTALDGSCRTPIAGYAHKGADGMLHFSGLVATPDGKQIMRASRVVPFTEADAVKCGEEAGKELKANGPKELFMY

SEQ ID NO: 37 - DNA, 1143 bp, Chlamydomonas reinhardtii
atgcgatcgtatctgctcaaggctcaagtggcctcatgtcagttttcgcgcacgtcgaaggtctggagactggcgccgggttctgacagacgacggtgtcggggcctcactcggacaccgcactgcgcggcccccaccagcgagcccgccccgccatccagcagcggcaagagcgggcaacgaccactcgtgatagccacgcggccatctaagcttgcaaaggagcagacgcggcaggtgcagcagctgctgctggcggcggcgcagctcaaggacgagcagctgcagctgagcaccctggaactggcgtctaggggcgacacgactcagggtgtgtcgctgcgcagtctgggctcgggcgcattcaccgaggagctggaccaggctgtgctgtcgggcgctgccgacatgtcggtgcacagcctgaaggactgccccgccgccctggcgcccgggctgctgctggccgcctgcctgccgcgggccgacccccgggacgtcctcatcgcgcccgaggccacctcgctgggcgagctggtgccgggcagccgtgtgggcaccagcagcagccgccgcgcggcgcagatcaagcactccttcccccacctgcaggttgtgcagctgcgcggcaatgtggactcgcggctggggcgcatccgcagccgcgacatcggcgccacagtgctggcggcggcgggcctcaagcggctgggtgtgatgaactcggacgagggtgacactaccgctacgggcgccgtgggggtggtgtgcagggcagacgatgagtgggtggtcggcctgctggacgccatctcgcaccgcggcacggccctggaggtggcggcggagcgggcgtgcctggcagcgctgctgggcggcggcggcgcgtgccagcgttcagcgttcccggacattgcgtgggcctgccacacgcggcacgaccccgacagcaacacaatggacctggattgcctggtggcggacctggagggcaaggagctcttcaggtacacggagttctaccggccggtcattgacgaggtggacgcggtgtcgctggggtcgctgtacggcagcctgctgcgcatgatggcgccaccaggcgcggccccctgttggcagctaccttcctcgcggcattag

SEQ ID NO: 38 - protein, 380 aa, Chlamydomonas reinhardtii
MRSYLLKAQVASCQFSRTSKVWRLAPGSDRRRCRGLTRTPHCAAPTSEPAPPSSSGKSGQRPLVIATRPSKLAKEQTRQVQQLLLAAAQLKDEQLQLSTLELASRGDTTQGVSLRSLGSGAFTEELDQAVLSGAADMSVHSLKDCPAALAPGLLLAACLPRADPRDVLIAPEATSLGELVPGSRVGTSSSRRAAQIKHSFPHLQVVQLRGNVDSRLGRIRSRDIGATVLAAAGLKRLGVMNSDEGDTTATGAVGVVCRADDEWVVGLLDAISHRGTALEVAAERACLAALLGGGGACQRSAFPDIAWACHTRHDPDSNTMDLDCLVADLEGKELFRYTEFYRPVIDEVDAVSLGSLYGSLLRMMAPPGAAPCWQLPSSRH

SEQ ID NO: 39 - DNA, 1692 bp, Chlamydomonas reinhardtii
atgatgttgacccagactcctgggaccgccacggcttctagccggcggtcgcagatccgctcggctgcgcacgtctccgccaaggtcgcgcctcggcccacgccattctcggtcgcgagccccgcgaccgctgcgagccccgcgaccgcggcggcccgccgcacactccaccgcactgctgcggcggccactggtgctcccacggcgtccggagccggcgtcgccaagacgctcgacaatgtgtatgacgtgatcgtggtcggtggaggtctctcgggcctggtgaccggccaggccctggcggctcagcacaaaattcagaacttccttgttacggaggctcgcgagcgcgtcggcggcaacattacgtccatgtcgggcgatggctacgtgtgggaggagggcccgaacagcttccagcccaacgatagcatgctgcagattgcggtggactctggctgcgagaaggaccttgtgttcggtgaccccacggctccccgcttcgtgtggtgggagggcaagctgcgccccgtgccctcgggcctggacgccttcaccttcgacctcatgtccatccccggcaagatccgcgccgggctgggcgccatcggcctcatcaacggagccatgccctccttcgaggagagtgtggagcagttcatccgccgcaacctgggcgatgaggtgttcttccgcctgatcgagcccttctgctccggcgtgtacgcgggcgacccctccaagctgtccatgaaggcggccttcaacaggatctggattctggagaagaacggcggcagcctggtgggaggtgccatcaagctgttccaggaacgccagtccaacccggccccgccgcgggacccgcgcctgccgcccaagcccaagggccagacggtgggctcgttccgcaagggcctgaagatgctgccggacgccattgagcgcaacatccccgacaagatccgcgtgaactggaagctggtgtctctgggccgcgaggcggacgggcggtacgggctggtgtacgacacgcccgagggccgtgtcaaggtgtttgcccgcgccgtggctctgaccgcgcccagctacgtggtggcggacctggtcaaggagcaggcgcccgccgccgccgaggccctgggctccttcgactacccgccggtgggcgccgtgacgctgtcgtacccgctgagcgccgtgcgggaggagcgcaaggcctcggacgggtccgtgccgggcttcggtcagctgcacccgcgcacgcagggcatcaccactctgggcaccatctacagctccagcctgttccccggccgcgcgcccgagggccacatgctgctgctcaactacatcggcggcaccaccaaccgcggcatcgtcaaccagaccaccgagcagctggtggagcaggtggacaaggacctgcgcaacatggtcatcaagcccgacgcgcccaagccccgtgtggtgggcgtgcgcgtgtggccgcgcgccatcccgcagttcaacctgggccacctggagcagctggacaaggcgcgcaaggcgctggacgcggcggggctgcagggcgtgcacctggggggcaactacgtcagcggtgtggccctgggcaaggtggtggagcacggctacgagtccgcagccaacctggccaagagcgtgtccaaggccgcagtcaaggcctaa

SEQ ID NO: 40 - protein, 563 aa, Chlamydomonas reinhardtii
MMLTQTPGTATASSRRSQIRSAAHVSAKVAPRPTPFSVASPATAASPATAAARRTLHRTAAAATGAPTASGAGVAKTLDNVYDVIVVGGGLSGLVTGQALAAQHKIQNFLVTEARERVGGNITSMSGDGYVWEEGPNSFQPNDSMLQIAVDSGCEKDLVFGDPTAPRFVWWEGKLRPVPSGLDAFTFDLMSIPGKIRAGLGAIGLINGAMPSFEESVEQFIRRNLGDEVFFRLIEPFCSGVYAGDPSKLSMKAAFNRIWILEKNGGSLVGGAIKLFQERQSNPAPPRDPRLPPKPKGQTVGSFRKGLKMLPDAIERNIPDKIRVNWKLVSLGREADGRYGLVYDTPEGRVKVFARAVALTAPSYVVADLVKEQAPAAAEALGSFDYPPVGAVTLSYPLSAVREERKASDGSVPGFGQLHPRTQGITTLGTIYSSSLFPGRAPEGHMLLLNYIGGTTNRGIVNQTTEQLVEQVDKDLRNMVIKPDAPKPRVVGVRVWPRAIPQFNLGHLEQLDKARKALDAAGLQGVHLGGNYVSGVALGKVVEHGYESAANLAKSVSKAAVKA

SEQ ID NO: 41 - DNA, 1173 bp, Chlamydomonas reinhardtii
atgcagaccaaggctttcacctctgcgcgcccccagcgggccgctgcgctcaaggcgcagcgcacctcgtcggtgaccgtgcgcgcgaccgcggcccccgccgtggcctctgcccccgccgcctcgggctctgcctctgaccccctgatgctgcgcgccatccgcggcgacaaggtggagcgcccgcccgtgtggatgatgcgccaggccggccgctaccagaaggtgtaccaggacctgtgcaagaagcaccccacgttccgtgagcgctcggagcgcgtggacctggcggtggagatctctctgcagccgtggcacgcgttcaagcccgacggcgtcatcctgttcagcgacattctgacccccctgcccggcatgaacatccccttcgacatggcgcccggccccatcatcatggaccccatccgcaccatggcgcaagtggagaaggtgacgaagctggacgctgaggccgcctgccccttcgtgggcgagtcgctgcgccagctgcgcacctacatcggcaaccaggccgcggtcctgggcttcgtgggcgcccccttcaccctggccacctacattgtggagggcggcagctccaagaacttcgcgcacatcaagaagatggctttctccacccccgagatcctgcacgccctgctggacaagctggctgacaacgtggccgactacgtccgctaccaggccgacgccggcgcccaggtggtgcagatcttcgactcgtgggccagcgagctgcagccccaggacttcgacgtgttctccggcccctacatcaagaaggtgatcgacagcgtgcgcaagacccaccccgacctgcccatcatcctctacatcagcggctctggcggcctgctggagcgcatggcctcttgctcgcccgacatcatctcgctggaccagtcggtggacttcaccgacggcgtcaagcgctgcggcaccaacttcgccttccagggcaacatggaccccggcgtcctgttcggctccaaggacttcatcgagaagcgcgtcatggacaccatcaaggctgcccgcgacgccgacgtgcgccacgtgatgaacctgggccacggcgtgctgcccggcacccccgaggaccacgtgggccactacttccacgtcgcccgcaccgcccacgagcgcatgtaa

SEQ ID NO: 42 - protein, 390 aa, Chlamydomonas reinhardtii
MQTKAFTSARPQRAAALKAQRTSSVTVRATAAPAVASAPAASGSASDPLMLRAIRGDKVERPPVWMMRQAGRYQKVYQDLCKKHPTFRERSERVDLAVEISLQPWHAFKPDGVILFSDILTPLPGMNIPFDMAPGPIIMDPIRTMAQVEKVTKLDAEAACPFVGESLRQLRTYIGNQAAVLGFVGAPFTLATYIVEGGSSKNFAHIKKMAFSTPEILHALLDKLADNVADYVRYQADAGAQVVQIFDSWASELQPQDFDVFSGPYIKKVIDSVRKTHPDLPIILYISGSGGLLERMASCSPDIISLDQSVDFTDGVKRCGTNFAFQGNMDPGVLFGSKDFIEKRVMDTIKAARDADVRHVMNLGHGVLPGTPEDHVGHYFHVARTAHERM

SEQ ID NO: 43 - DNA, 288 bp, Chlamydomonas reinhardtii
atgtcggccctggacgccgccgccatcccctacgagctagtgccgggtgtgtcctccgctctggccgccccgctgttcgccggcgtcccgctcacacacgtcagcctgagcccctcgttcaccgtggtcagcgggcacgacgtggccggcaccgactgggcggcgttccgggggctgcccacgctggtggttctgatggcgggtcgtaacctggggcagatagcccggcggcttgtgcaggacgcggggtgggcgcccgatacacctgtaagtcaacctagtggctag

SEQ ID NO: 44 - protein, 95 aa, Chlamydomonas reinhardtii
MSALDAAAIPYELVPGVSSALAAPLFAGVPLTHVSLSPSFTVVSGHDVAGTDWAAFRGLPTLVVLMAGRNLGQIARRLVQDAGWAPDTPVSQPSG

SEQ ID NO: 45 - DNA, 204 bp, Chlamydomonas reinhardtii
ggcgtccccacaaccaggacagcctacttcttgaccttattaataagtcgctgcgtgtcgcgactgaccattttggcccggacttgcgtgcttgtgatttgtgcttcgactagatccgcgggcaccaagggacgcggacagctgatagtcaagaactagatcctctgggagcgtctggggctgtccccgctgctcgccaaggaa

SEQ ID NO: 46 - DNA, 721 bp, Chlamydomonas reinhardtii
gtgccgagtgactgaggtggcaaggtgcagtggcggcggaggcagttgtgctggggtggcaaggcggacaggcgaagctggtgggttgcgacgaggaggaggtgcacgtgcacgcgtaacataagaagaacagtgggaggacaggtagcgtgacttgactgggacgaggagcgtactgatgtgtggcgtgtgttggtatgtgagcgttacccctcccctagatagcggcggtctccactttcaggaggatgagagccatcatgaggctttgagggggcactggttcgtgtgtaggctgaggctgctgttgaagtcacaaggcagcactgcatgcgcgagtgagtgtggccggatatgcatcgagttgcaggtacactgaaatgaggtgactgcggcgtatatcgctgccagtacaggttgaagcggcgggcacggtgaatggagtactcggcctggaacgcttgcgatcagatggtcgagctcaagaagatttggttgagccgttgggtcgtgcgtcatattatggcttgcatcttcggggagcggcaagaaacggactccaatgcaggccctcgggcgagaaagattgggcgtgtccgggggtgcattctcgccgcgtggggctgcatcgaatttcgcttgagtgccccttcccggggagggggggcggtagttcaaccccatcatcgtaggggggttgtaaatgccagcccaaactaaa

SEQ ID NO: 47 - DNA, 187 bp, Chlamydomonas reinhardtii
atgaagtctctctgccatgagctcgctggccccagcgttactgggtgcggccggcgaagcctccggaaggctttcagcggtgccaagattgcgcaggtctctcgccccgctgtgcttaacagcgtgcagcgccaacagcgtctcgcctgttctgccgtggccgagctctccgctgctgagctgcgcg

SEQ ID NO: 48 - DNA, 281 bp, Chlamydomonas reinhardtii
ccatgaaggtgtctgaggaggactccaagggcttcgatgcggatgtgtcgacccgcctggcccgctcgtaccctctggcggccgtggtgggccaggacaacatcaagcaggcgctgctgctgggcgccgtggacaccgggctgggcggcatcgccatcgccggtcgccgcggtaccgccaagtccatcatggctcgcggcctgcacgctctgctgccgcccattgaggtggtggagggcagcatctgcaacgccgaccccgaggacccccgctcctgggag

SEQ ID NO: 49 - DNA, 132 bp, Chlamydomonas reinhardtii
gctggcctggctgagaagtatgcgggcggccctgtgaagaccaagatgcgctcggcgccgtttgtgcagatccctctgggtgtgactgaggaccgcttggtgggcactgtggacattgaggcgtccatgaag

SEQ ID NO: 50 - DNA, 167 bp, Chlamydomonas reinhardtii
gagggcaagactgtgttccagcccggcctgctggctgaggcgcaccgcggcatcctgtacgtggacgagatcaacctgctggatgacggcattgccaacctgctgctgtccatcctgtcggacggagtcaacgtggtggagcgcgagggcatctccatcagccaccc

SEQ ID NO: 51 - DNA, 163 bp, Chlamydomonas reinhardtii
ctgccggccgctgctgattgccacctacaaccccgaggagggccctctgcgtgagcacctgctggaccgcatcgccattggcctcagcgccgacgtccccagcaccagcgacgagcgcgtcaaggccattgacgcagccatccgcttccaggacaagccgcag

SEQ ID NO: 52 - DNA, 48 bp, Chlamydomonas reinhardtii
gacactattgacgacaccgcggagctcaccgacgccctgcgcacctcg

SEQ ID NO: 53 - DNA, 123 bp, Chlamydomonas reinhardtii
gtcatcctggctcgcgagtacctgaaggacgtgaccatcgcgccggagcaggtgacctacattgtggaggaggcgcgccgcggcggagtccaggggcaccgcgcggagctgtacgcggtcaag

SEQ ID NO: 54 - DNA, 171 bp, Chlamydomonas reinhardtii
tgtgccaaggcgtgtgcggctctggagggccgtgagcgtgtgaacaaggatgacctgcgccaggccgtgcagctggtcatcctgccgcgcgccaccatcctggaccagcccccgcccgagcaggagcagcccccgccgccgcccccgccccctcccccgccgccgccgcag

SEQ ID NO: 55 - DNA, 87 bp, Chlamydomonas reinhardtii
gaccaaatggaggacgaggaccaggaggagaaggaggacgagaaggaggaggaggagaaggagaacgaggaccaggacgagcccgag

SEQ ID NO: 56 - DNA, 225 bp, Chlamydomonas reinhardtii
atccctcaggagttcatgtttgagtccgagggcgtcatcatggacccctccatcctcatgttcgcgcagcagcagcagcgcgcgcagggccgctccggccgcgccaagacgctcatcttcagcgacgaccgcggccgctacatcaagcccatgctgcccaagggtgacaaggtcaagcgcctggcagtggacgccacgcttcgcgccgccgcgccctaccagaag

SEQ ID NO: 57 - DNA, 67 bp, Chlamydomonas reinhardtii
attcgccggcagcaggccatcagcgagggcaaggtgcagcgcaaggtgtacgtggacaagccagaca

SEQ ID NO: 58 - DNA, 653 bp, Chlamydomonas reinhardtii
tgcgctccaagaagctggcccgcaaggccggtgcgctggtgatttttgttgtggacgcgtccggctccatggctctgaaccgcatgagcgccgccaagggcgcctgcatgcgcctgctggctgagtcgtacaccagccgcgaccaggtgtgcctcatccccttctacggcgacaaggccgaggtgctgctgccgccctccaagtccatcgccatggcccgccgccgcctggactcgctgccctgcggcggcggctcgccccttgcgcacggcctgtccacggcggtacgtgtgggcatgcaggccagccaggcgggcgaggtgggccgcgtcatgatggtgctcatcacggacggccgcgccaacgtcagcctggccaagtccaacgaggaccccgaggcgctcaagcccgacgcgcccaagcccaccgccgactcgctgaaggacgaggtgcgcgacatggccaagaaggccgcgtccgccggcatcaacgtgcttgtcattgacacggagaacaagttcgtgagcaccggctttgcggaggagatctccaaggcagcgcagggcaagtactactacctgcccaacgccagcgacgccgccatcgcggcggccgcgtccggcgccatggccgcggccaagggcggctactag

SEQ ID NO: 59 - DNA, 379 bp, Chlamydomonas reinhardtii
gtgagcgcctactttgatatgtaccaaagataccactgataggtttaggcacggaagatctggacttggaccccgtttgcgcaagccgggcgatgcacccatttcgcggtcacgccgagcgctggggtgcaatttagcgtgcccgacaagctagaaaacagggaattaccatttgtttaattttgttgcgagagatctttgcttgtgtccaccggccgcgcgggggaacttccggtgttgcgcaaggttgcgtgcgtgcccaccatcaacacctgtgccaggtctgtgtcacccccaggttccaccaccctgcaatcttccaattgtgtctcgtttgctcgttgtctaatagtcgtcctttgctcatccctacctgcag

SEQ ID NO: 60 - DNA, 267 bp, Chlamydomonas reinhardtii
gtgaggcagggaaggtgacacaggaggttttgaaagagagacagggaggcaaagatggatggcggggcgggcagtgactttggggcggcatggagtgggattggtggagtgggattgggcaccatgtatcacagatgttggcaacacagcgcagggccttgctctgtgcttgtgttgaccgtctagtcccccgtgccctgaaccaagtctttcctcctgacacggtcctccatgtcctccttccggcattcccttcctcgtccacag

SEQ ID NO: 61 - DNA, 273 bp, Chlamydomonas reinhardtii
gtgagccagcaagggaggagaggggaacggccgggtagggcagccggagtttaaccacgccaattcaacggggagcaacggggaagaggaagggccggaagaggacggcaaaagcatttggtgggggcagcggctgtagtcagaagcgcaaaggctgccacagtgtggcccgcaccctcctcaccaccagtttggcatgatcgtttagcatgggctggaatactcaccgccagttctctcctctcccctctcctcccctgtccccgcctgcag

SEQ ID NO: 62 - DNA, 166 bp, Chlamydomonas reinhardtii
gtgagtgcgcgcgctgggtgtgtttgtgggacggcgcggcattggagcgcaggtgcgggtgctgggccgtgcacttgtccgttggttcccttggaagcttcgatacacactcttactgcacgctctttaaccgccccccccctccacctctgcccgccccgtgcag

SEQ ID NO: 63 - DNA, 275 bp, Chlamydomonas reinhardtii
gtgggtgggggaaagtgactggatgtcggtgggttttaggtatgtgcgtgtgtacgatgcggggagcagtacggaagcgggcacgagcggtgagggggcaggattgtggcgcacgctcgggccaagcccgggctcgcgacagagggtgggcttgtattcgtagtcaagcgcatcaggaagtgcagttgactggattcacctgaaacggcgctgagcgggcggctaatagaatcccgcttcctgtccgcccctccccttgcccttcaatccgtcag

SEQ ID NO: 64 - DNA, 200 bp, Chlamydomonas reinhardtii
gtgagtggcgggggccgtgcgtttgtttgttgcgtgggctggctggctggctttgttggatgagggcgctgctcaccactcatctctttgaatccccacttatccagttgcctgcatgaaaccccgcctgactcactccccaccatcctgtaccgcttttccaaacatccttgcaaccatcccgccatccccacccgcag

SEQ ID NO: 65 - DNA, 690 bp, Chlamydomonas reinhardtii
gtgaggagttggagggggaaggggcgaggggatgcgacagaagcgagggcgaggggagccggggtgggttgttgcaagtgtcgtgaattatagaatgaccccaaaagcgccggcccaacagggcctattacttgcgagtcaatccaacccctgatatagggagaatggggtagaggtcgtatcacgacagcaaggatgtacagtgggccttggggttgggaggtacagggaaaaaggagaggacatggggttgggtaagcggggaataacaaatatacacccagcgtttatggaagtgggagatggaaacgggggcggacgaacaggaacaggggccggatggaggggctatgggggcatggtgggtgggggtacggcgcggggcagagcagggtcttgggtgaatgggcaagatgctgatgcttgggatgaagacactatgagcaaagaaatggttgttgacgattgccatgatcatcgcagtgggggaggcggggtggcaataccggcagtcaacagttggggtgcgatcaagattgattggagtaccagcagtggccgggatctggctgacgtgtctcgagcgagttgctggggtggcaaggagatgcaggggcagacgacgttgtgcgaccacacttacacacatttccttccccttgcgtgtgtccgtgcgccctgtgcctccag

SEQ ID NO: 66 - DNA, 123 bp, Chlamydomonas reinhardtii
gtacgtaaacgtatttgattgctcaggtggttagccttggtgtggctgctgtttgacttgtgcagctgtctttgtgtacatgttccacaaccctgtactccccatattccgcccccattccag

SEQ ID NO: 67 - DNA, 228 bp, Chlamydomonas reinhardtii
gtgagaggcggcgcggcggcttgcgggcgaaggcggggggcggggcggaggcaatgcggccgcgcatggccagcaacggaagggctggctatcaacacggcgagcgcacgatattcatataagagtgccatcgtgcaatgctgaatacttgcgccaaccggatctcgctgctccgcttccaccggactgctttctcatctctccccttcaccctgtgtgtatccacag

SEQ ID NO: 68 - DNA, 146 bp, Chlamydomonas reinhardtii
gtgagtgcccgaggtggtgggtggtgaattggggcacgagggtatgtgggcctaagggagctgaatggggcatgttttcttctgagcatcacggtcagagcttgacctgtcctccccgctgtacccccgtgcacggtccgacacag

SEQ ID NO: 69 - DNA, 168 bp, Chlamydomonas reinhardtii
gtgagtacagcgcatcccggcgcaatcattgggcctagttactgctgcaggactcgtgtgctcttaagggctggcagctgtcagaagctctactcctcgcactgaccactgtgcctttctctccttcctctctccctccccgcacccctcctcccacttcctcaacag

SEQ ID NO: 70 - DNA, 143 bp, Chlamydomonas reinhardtii
gcagacttccataaagctcttgtaacgctgtaccaactagtaagcggtacaattcgcctgagcccgagcaacgcgacctttcttgctctgtggatctctgataatctaaccagaccaaaaccttttcactaatctaggcaaca

SEQ ID NO: 71 - DNA, 381 bp, Chlamydomonas reinhardtii
aaaaggctggtgtaggcctgtcgggtcgtgttaaaggttgctgcgtgaacgtgtaagtgtgacagtgtgccggtatgtgtgtgtatacatgtgttgcggtgtgcttttgtggcggtacatggtgatgactgagcgggtgggacagagcacggttaactgacgagggcagtccgtgcgagacggacgtttttgtagccgaggtgcaaggactgatgacgggctaagctgctggagacttggagttgagagtgcaggtggatcgacggtttctctaaggagtatgaataggcaggagggctggagacatttggggtgcaaggaggcggtagtatgggagatgtccatgggcggattttggcctctgtaacttcttaacgccca

SEQ ID NO: 72 - DNA, 252 bp, Chlamydomonas reinhardtii
atgcagagtctccagggtcagcgcgcgttcactgcggtgcgccagggtcgggcgggtcccctgcggactcgcctggtcgtgcgctcgtctgttgccttgccatccacgaaagccgcgaagaagccgaacttcccgttcgtcaagattcagggccaggaggagatgaagcttgcactgctgctgaacgtggtcgaccccaacatcggcggagtgcttattatgggtgaccgcggcactgccaagtcggtcgcg

SEQ ID NO: 73 - DNA, 156 bp, Chlamydomonas reinhardtii
gtccgcgccctggtggatatgcttcccgacattgacgtggttgagggcgacgccttcaacagctcccccaccgaccccaagttcatgggccccgacaccctgcagcgcttccgcaacggcgagaagctgcccaccgtccgcatgcggacccccctg

SEQ ID NO: 74 - DNA, 102 bp, Chlamydomonas reinhardtii
gtggagctgcctctgggcgccaccgaggaccgcatctgcggcaccatcgacatcgagaaggcgctgacgcagggcatcaaggcctacgagcccggcctgctg

SEQ ID NO: 75 - DNA, 60 bp, Chlamydomonas reinhardtii
gccaaggccaaccgcggcatcctgtatgtggacgaggtgaacctgctggatgatggcctg

SEQ ID NO: 76 - DNA, 111 bp, Chlamydomonas reinhardtii
gttgatgtcgtgctggactcgtcggctagcggcctgaacactgtggagcgtgagggtgtgtccattgtgcaccctgcccgcttcatcatgattggctcaggcaacccccag

SEQ ID NO: 77 - DNA, 101 bp, Chlamydomonas reinhardtii
gagggtgagctgcgcccgcagctgctggatcgcttcggcatgagcgtcaacgtggccacgctgcaggacaccaagcagcgcacgcagctggtgctggaccg

SEQ ID NO: 78 - DNA, 127 bp, Chlamydomonas reinhardtii
gcttgcgtacgaggcggaccctgacgcatttgtggactcgtgcaaggccgagcagacggcgctcacggacaagctggaggcggcccgccagcgcctgcggtccgtcaagatcagcgaggagctgcag

SEQ ID NO: 79 - DNA, 158 bp, Chlamydomonas reinhardtii
atcctgatctcggacatttgctcgcgcctggatgtggatggcctgcgcggtgacattgtgatcaaccgcgccgccaaggcgcttgtggccttcgagggccgcaccgaggtgaccacgaatgacgtggagcgcgtcatctcgggctgcctcaaccaccg

SEQ ID NO: 80 - DNA, 211 bp, Chlamydomonas reinhardtii
cctgcgcaaggacccgctggaccccattgacaacggcaccaaggtggccatcctgttcaagcgcatgaccgaccccgagatcatgaagcgcgaggaggaggccaagaagaagcgcgaggaggcggccgccaaggccaaggcggagggcaaggcggaccgccccacgggcgccaaggctggcgcctgggctggcttgccccctcgtcggtaa

SEQ ID NO: 81 - DNA, 534 bp, Chlamydomonas reinhardtii
gtaggtaacacaagcaattatggggcgaagatctaggctccgctgatccgggcgggcaatcggcatcgtcggtgcaaccgtggggcgtctgtgcaccctttgctggtgccaggttgcctgactcgcctgcattcctgtaccgagccacattggctgctttgcagcgtgcatgggacgggtgtaggataagcgctatgtatgcgatagcgcgggtgcaccggcttggcatggcaaggttgcggggtgcacatgcgtgccagcgtcccctcagcatcagagtctggatctaagggctcagcggcttcctgcgcatgtgggtctttgcgtagtgctacgaagccttataattaaagctcatgtattgagtggtccgggtttggggcactagtagtgccaggaggcgcgtgccaggttgatatgagcatatcagcacccgttccttgcgaaacgcttccgttgtgctcccttccccaccacctccccgctcatacccatacatatggctatccgtcctctcattgcttgcccctacag

SEQ ID NO: 82 - DNA, 195 bp, Chlamydomonas reinhardtii
gtgagcgggcctaccttctgaagacagtcttacgtgttgcactgcagcggtgttgcgcacctctgcttttgcgtgcgccgggaagcgcggattgcggcctcacagatcaagcccggaaacgcttgttgtttccagcgggtggcacacacgcgcgcgcgcgcacagtgacaccctcacggccgcgctgccctgcag

SEQ ID NO: 83 - DNA, 235 bp, Chlamydomonas reinhardtii
gtgcgtagtgcatggggagaggggacgaggggaggagggcagggccaataaaccgaaccccaagtcatcgagacacagaacccgataatagctcccagatcgccaaggggtgaggcgggaagccaaggatgatgcgttggccgcattgcgtgttgacgtcaggcttacacagggtctgactggctgtgcttggggtttggcacgcttcttgactggccccgtacgcatgctgcag

SEQ ID NO: 84 - DNA, 212 bp, Chlamydomonas reinhardtii
gtgagtggtggtggtttctgggtcagcagaggacttctgtagtaggtaatgtgggccagggaagtgtggctaacatgccaaacacgggggcgcaccagtgcaagctgcattcgctgacgtgcacgggtgcaatgggtgcaaggcgaactgcaatcgcggtgcacagttgccagggctgcgctcacgcttgagtgtctgcacacgcactgcag

SEQ ID NO: 85 - DNA, 270 bp, Chlamydomonas reinhardtii
gtgcgtagcgtgcgcgcatgtacttgtctcccttgtcatgttgggaaaggtcggtccccagcctgcttgcaagatgcggccggtcagcagctgcggacggtcagcacctacgtgccgaggttgtgtaacatgaatggcgttggggcggccgacctgccacaagctgaactgcgaccagcaaggcagctgccagcaacgcacacccgacgtgctacacgcttgtgttttgacctcctaaacacacccgcccgctgtctgtcacgtccacag

SEQ ID NO: 86 - DNA, 199 bp, Chlamydomonas reinhardtii
gtaagcggcggcggcgcggggacacggagggacatttcgcgagcatgggttgaggagtcgggaggattcggtggctggccggagtcgggagtcggagtcgcgagtcggaagtcaagcttctggcggcttcgtgctgtcgggtgcgctcgccatgatggcgctgaccggagggcgtcacgctgtgtatgtgggcgcgcag

SEQ ID NO: 87 - DNA, 231 bp, Chlamydomonas reinhardtii
gtacggggcgtacagcgggggcggctgcacggggccagtgaccgacagggcagcacgcggctggcgaagagcgacaaagtgacagggtgaccaagaccgggtgatgccacgagaggggcgcgggagccgtgcattgggtcgaggagggaggaatgcaactttacactgatgcctctgtatacggccgccttccgagccctgcaaaccttcgctttcccccgacgcacgcag

SEQ ID NO: 88 - DNA, 279 bp, Chlamydomonas reinhardtii
gtgagcgcagcgtgcggtggatgcggtgcgcgtgcgggttgccaacttattattttgtacgtggacgcgtggctggcgatggcatgtcatggcgcgaatggatattgggcgaatggataccggtaatggtagcacggggcggcagggcctggcggtagtggggttgagggggcgaggactccagcgcgcgatacatgccatgttcagcatggccccaactgacagcgcccgctgccctgtgcgccccgctccctccgcgcacccgctcctcctacacag

SEQ ID NO: 89 - DNA, 36 bp, Chlamydomonas reinhardtii
ctagtctagagggaactagggaggggcaacagagaa

SEQ ID NO: 90 - DNA, 833 bp, Chlamydomonas reinhardtii
gcggcctccccttcatggtagcactagttggcgggttgtggttggactaggcggctagggtatatacctagtagcggcggctgcggagtggagggctggcgcccagcgcgagggcgtggcctttcctcctggacccgagagcgctccgcgaggagacggcgagtgagataggcagcagcgagcggagatcgatttgtgaacagttttgtggcgggatcccatagcggatgcagagaagaccttagagcagcttcctcggtggagtgaacgagccagagcggagggaaggcgcatgagggaactgcagggactggaactgcgggagtgcaggtccggtgctaggtccgctaaacagtgcggtctacgcctgtgtgtgaggtgtgcgtgtgtgtgtgagctgtgcggttttgttgtgcaaagtaggagtgagccgagccgcgcgtactttgtggcgtgtttggctgctggcgctgagagccaagagagggtaaacgggtttggtattttatggtgcggggtgaaagcagccctcgcaggaatggagcgattctgcagcatgatgcacgtgtgcctgcgcgtggatggtggctgttgatatggctctgccactccggcagcaccgctacgatacctagcggtgcctggagtggtctctctgtttggtgcgtgatgtttgggtttgccgttttgattctttgtttcgtgctgaatggctgaggcggcaagacccctcgtgccagtgtacagagcctcacggctccctcggaccccgcgtggggacgtccattcccggtggcggtgtcgcctcggcggtgtaaagcaaaaaatatttt

SEQ ID NO: 91 - DNA, 66 bp, Chlamydomonas reinhardtii
atgcagacttcctcgcttcttggccggcgcacggcccacccggctgcgggcgcgacgcccaagccg

SEQ ID NO: 92 - DNA, 36 bp, Chlamydomonas reinhardtii
gttgcgccctcgccccgcgtggctagcacccgccag

SEQ ID NO: 93 - DNA, 106 bp, Chlamydomonas reinhardtii
gtcgcgtgcaatgtggcgactggaccccggccgcccatgaccaccttcaccggtggcaacaagggccctgctaagcagcaggtgtcgctggatctgcgcgacgagg

SEQ ID NO: 94 - DNA, 161 bp, Chlamydomonas reinhardtii
gcgctggcatgttcaccagcaccagcccggagatgcgccgtgtcgtccctgacgatgtgaagggtcgcgttaaggtgaaggttgtgtacgtggtgctggaggcccagtaccagtcggccatcagcgctgcggtgaagaacatcaacgccaagaactccaag

SEQ ID NO: 95 - DNA, 135 bp, Chlamydomonas reinhardtii
gtgtgcttcgaggtggtgggctacctgctggaggagctgcgtgaccagaagaacctcgatatgctcaaggaggatgtggcctctgccaacatcttcatcggctcgctcatcttcattgaggagcttgccgagaag

SEQ ID NO: 96 - DNA, 162 bp, Chlamydomonas reinhardtii
attgtggaggcggtgagccccctgcgcgagaagctggacgcgtgcctgatcttcccgtccatgccggcggtcatgaagctgaacaagctgggcacgttttcgatggctcagctgggccagtcgaagtcggtgttctcggagttcatcaagtctgctcgcaag

SEQ ID NO: 97 - DNA, 299 bp, Chlamydomonas reinhardtii
aacaacgacaacttcgaggagggcttgctgaagctggtgcgcaccctgcctaaggtgctgaagtatctgccctcggacaaggcgcaggacgccaagaacttcgtgaacagcctgcagtactggctgggcggtaactcggacaacctggagaacctgctgctgaacaccgtcagcaactacgtgcccgctctgaagggcgtggacttcagcgtggctgagcccaccgcctaccccgatgtgggtatctggcaccctctggcctcgggcatgtacgaggacctgaaggagtacctgaactg

SEQ ID NO: 98 - DNA, 158 bp, Chlamydomonas reinhardtii
gtacgacacccgcaaggacatggtcttcgccaaggacgcccccgtcattggcctggtgctgcagcgctcgcacctggtgactggcgatgagggccactacagcggcgtggtcgctgagctggagagccgcggtgctaaggtcatccccgtctttgccg

SEQ ID NO: 99 - DNA, 260 bp, Chlamydomonas reinhardtii
gtggcctggacttctccgcccccgtcaagaagttcttctacgaccccctgggctctggccgcacgttcgtggacaccgttgtgtcgctgaccggcttcgcgctggtgggcggccccgcgcgccaggacgcgccgaaggccattgaggcgctgaagaacctgaacgtgccctacctggtgtcgctgccgctggtgttccagaccactgaggagtggctggacagcgagctgggcgtgcaccccgtccaggtggctctgcag

SEQ ID NO: 100 - DNA, 1515 bp, Chlamydomonas reinhardtii
gttgccctgcccgagctggatggtgccatggagcccatcgtgttcgctggccgtgactcgaacaccggcaagtcgcactcgctgcccgaccgcatcgcttcgctgtgcgctcgcgccgtgaactgggccaacctgcgcaagaagcgcaacgccgagaagaagctggccgtcaccgtgttcagcttcccccctgacaagggcaacgtcggcactgccgcctacctgaacgtgttcggctccatctaccgcgtgctgaagaacctgcagcgcgagggctacgacgtgggcgccctgccgccctcggaggaggatctgatccagtcggtgctgacccagaaggaggccaagttcaactcgaccgacctgcacatcgcctacaagatgaaggtggacgagtaccagaagctgtgcccttacgccgaggcgctggaggagaactggggcaagccccccggcaccctgaacaccaacggccaggagctgctggtgtacggccgccagtacggcaacgtcttcatcggcgtgcagcccaccttcggctacgagggcgacccgatgcgcctgctgttctcgaagtcggccagcccccaccacggcttcgccgcctactacaccttcctggagaagatcttcaaggccgacgccgtgctgcacttcggcacccacggctcgctggagttcatgcccggcaagcaggtcggcatgtcgggtgtgtgctaccccgactcgctgatcggcaccatccccaacctctactactacgccgccaacaacccgtctgaggccaccatcgccaagcgccgctcgtacgccaacaccatttcgtacctgacgccgcctgccgagaacgccggcctgtacaagggcctgaaggagctgaaggagctgatcagctcgtaccagggcatgcgtgagtctggccgcgccgagcagatctgcgccaccatcattgagaccgccaagctgtgcaacctggaccgcgacgtgaccctgcccgacgctgacgccaaggacctgaccatggacatgcgcgacagcgttgtgggccaggtgtaccgcaagctgatggagattgagtcccgcctgctgccctgcggcctgcacgtggtgggctgcccgcccaccgccgaggaggccgtggccaccctggtcaacatcgctgagctggaccgcccggacaacaacccccccatcaagggcatgcccggcatcctggcccgcgccattggtcgcgacatcgagtcgatttacagcggcaacaacaagggcgtcctggctgacgttgaccagctgcagcgcatcaccgaggcctcccgcacctgcgtgcgcgagttcgtgaaggaccgcaccggcctgaacggccgcatcggcaccaactggatcaccaacctgctcaagttcaccggcttctacgtggacccctgggtgcgcggcctgcagaacggcgagttcgccagcgccaaccgcgaggagctgatcaccctgttcaactacctggagttctgcctgacccag

SEQ ID NO: 101 - DNA, 713 bp, Chlamydomonas reinhardtii
gtggtcaaggacaacgagctgggcgccctggtagaggcgctgaacggccagtacgtcgagcccggccccggcggtgaccccatccgcaaccccaacgtgctgcccaccggcaagaacatccacgccctggaccctcagtcgattcccactcaggccgcgctgaagagcgcccgcctggtggtggaccgcctgctggaccgcgagcgcgacaacaacggcggcaagtaccccgagaccatcgcgctggtgctgtggggcactgacaacatcaagacctacggcgagtcgctggcccaggtcatgatgatggtcggtgtcaagcccgtggccgacgccctgggccgcgtgaacaagctggaggtgatccctctggaggagctgggccgcccccgcgtggacgtggttgtcaactgctcgggtgtgttccgcgacctgttcgtgaaccagatgctgctgctggaccgcgccatcaagctggcggccgagcaggacgagcccgatgagatgaacttcgtgcgcaagcacgccaagcagcaggcggcggagctgggcctgcagagcctgcgcgacgcggccacccgtgtgttctccaacagctcgggctcctactcgtccaacgtcaacctggcggtggagaacagcagctggagcgacgagtcgcagctgcaggagatgtacctgaagcgcaagtcgtacgccttcaactcggaccg

SEQ ID NO: 102 - DNA, 589 bp, Chlamydomonas reinhardtii
ccccggcgccggtggcgagatgcagcgcgacgtgttcgagacggccatgaagaccgtggacgtgaccttccagaacctggactcgtccgagatctcgctgaccgatgtgtcgcactacttcgactccgaccccaccaagctggtggcgtcgctgcgcaacgacggccgcacccccaacgcctacatcgccgacaccaccaccgccaacgcgcaggtccgcactctgggtgagaccgtgcgcctggacgcccgcaccaagctgctcaaccccaagtggtacgagggcatgcttgcctcgggctacgagggcgtgcgcgagatccagaagcgcatgaccaacaccatgggctggtcggccacctcgggcatggtggacaactgggtgtacgacgaggccaactcgaccttcatcgaggatgcggccatggccgagcgcctgatgaacaccaaccccaacagcttccgcaagctggtggccaccttcctggaggccaacggccgcggctactgggacgccaagcccgagcagctggagcgcctgcgccagctgtacatggacgtggaggacaagattgagggcgtcgaataa

SEQ ID NO: 103 - DNA, 79 bp, Chlamydomonas reinhardtii
gtaggtgtaattagaaggatcaaaacctagcggcctgatctgggactgacggcctcgcgcttcaatcactctgatgcag

SEQ ID NO: 104 - DNA, 83 bp, Chlamydomonas reinhardtii
gtaggcacggcagaatgctcaatgaacatgcagctacatatgtttgggatcatggctgatctctgtgcgacgggtccgcgcag

SEQ ID NO: 105 - DNA, 183 bp, Chlamydomonas reinhardtii
gtgagcagcgcggaccgagcaagcgctggcgatgcagttggatttgttgttcttgggtcaggcgctcgctcgatggccagcgcgtgtatttaatgggataagggttgagacaaagcatctcttcgggtaaaaatcttagttttcgacagcacgttgagaggcatgcaacttgctctttcgcag

SEQ ID NO: 106 - DNA, 106 bp, Chlamydomonas reinhardtii
gtgggtaaggagttgcattatcagtgtggcatggtgttgcgggcgtctggggcgctgcaacagcggcatcgtgccgaactgaccgtgccgggctacccgcgtgcag

SEQ ID NO: 107 - DNA, 231 bp, Chlamydomonas reinhardtii
gtgcgctagggttggggtctggagggtgtggattgcgcccaagtgccctgttgcgcttggcggtcgctgtcatgatgtgagggtgacgtagtgcactcaattgcctgctacgtcaccacctttgatgggctggatctgaggcaggtcagctcggttccctgctgcatccagtgtccctgtcgccctgcacgtttgacgctgttcccccttccgcactgtctcgctttgcag

SEQ ID NO: 108 - DNA, 137 bp, Chlamydomonas reinhardtii
gtgtgggcacgcgctttgggaagggaggcatacatttttggttgcggttaggctgggcgcggacttggcactcacacggtcattgcacactcatgtctcaccttcatttacggtcccttgtgccgaactacctacag

SEQ ID NO: 109 - DNA, 255 bp, Chlamydomonas reinhardtii
gtgagcagcatcagggcagagtgcatgaacggattggtggcagtggggaatggaattagacggacacgtctgggcggcaatatgttgcgctgcagtttttggggtgtagtgaactagaaaatagggaagagataggccacataacatccgaaagctcatatttttgcaaccggcgcacctatcacagcccacctgaagggttttgtagtcaacgcgtgcaactgactagatgtccccttacctgtctgatttcag

SEQ ID NO: 110 - DNA, 211 bp, Chlamydomonas reinhardtii
gtgaggcggggcggcgctgccctcggtaggggttgcagatggtgatgggtaaccgaatgcatggccaatggggagtgaaatcaggaaaggaggggtaacacaatgcagggcagcacctgaatcgtgaaggcggagttaggcagggatctgtcagttcgcctgtcacgtggatgggcgcagctgacctttgtggtgttgtggtgtggcgcag

SEQ ID NO: 111 - DNA, 192 bp, Chlamydomonas reinhardtii
gtgagctcagctgggacatgtaggggctcgggtcgccggagcatcgatgtagaattacgggaggaggggagaggggagaggattgcacgaaccgagatgagggcggtggttcgggatttcgggcaaaagctcgtgcggcaagcgttcagtgactgaagagcagtgtgcttcaactgcccctctgtccctcag

SEQ ID NO: 112 - DNA, 167 bp, Chlamydomonas reinhardtii
gtgcgaccggtgccgctgcgtggccaacagcttggtgccaccttcctgcggtgttgatttacactgtgtgcgtggatgtgttggtttttcgcaactttagtctgggctccagctctttgccttcattgatcactcgtcttacctcctgcgccatcatttgaatacag

SEQ ID NO: 113 - DNA, 154 bp, Chlamydomonas reinhardtii
gtgagccttaatgcaacacgtgtagccgttcgcatgggtggctgggtcatgctatggttggatcggcgtccgcctgcttgctactgcctgttcggtaccagcgtttactgaccccgcgtgtgccattcccaccacctaccccctcgccttgcag

SEQ ID NO: 114 - DNA, 149 bp, Chlamydomonas reinhardtii
gacagtgatatagcaataccgatataataggtttggcgggcttcaccttgtccttacccagaatgtggccctgacagtcgatttccagcccccttgccactcgctccctgatttcttcaatcaactagttgggtcgttttctcgtaagg

SEQ ID NO: 115 - DNA, 944 bp, Chlamydomonas reinhardtii
gggggcgggtggcgagtaaggcgtatggcggagcgaggagatgggctgtggcgtggccggtgttcttttgtgtgattggaaacatagacggggtgcggcacgcggcctgactgctgcgcggttggtgtggttgcggggggagcggggtcgatggggcagcgcgcacgagttggttgaaggaggagggccaggcgctgggctacacccatggtttgaggatgctagtgagtgatgtgtgcggggggcatggtgtgtaccattcagagtccagatgcacgcacggttgcgtgggagcgttccctgctgtgcatgatgatggcgccttcgatgaatcatctcttgaaggtccaaatgaaacgtctgaagtctgcagagggtggtgctggacatgccatccaggcggaagtgggcagctgtgtctgactacaaagtaggtcttgttttgcttggatagcgtttggctatgtagcgtgtattctgctcatcaatcacgccaggcgtcagggactacccatgcaagtcgggagcgtggctggctctggaaaagttgtagctgctaggtggcgttggctggggtgtcatgcatctcggcaggtaggcggtagcggtggacgacctctgcagcggagcatgtgcacaagatgtgactgcgcatgcacccgtatatgacggcgttggcgtcagttgttgagagtgaacagaggagagacgagcgaagctgccatgcccttagtggctggtgcgagaggggaagaaagagagaggaaggactttgcggcagtgccccacgccggagttggggacacggtcatcaacagggcggcggagctgggcggagtgggtgtgtgatgggacagggttcaaggcaggttggcgaggtcggagtgggtagaccagtccttcagtgcaagggcattagggcatgatgtaagggctgaagcttg

SEQ ID NO: 116 - DNA, 116 bp, Chlamydomonas reinhardtii
atggcgtcgtttggattgatgcaaaggacggtgcactgtccccagcttgtggaggagcggtgttcgccggtcgctggctgctctggtcgtggcctgccagttatccagcggcaacg

SEQ ID NO: 117 - DNA, 676 bp, Chlamydomonas reinhardtii
gcgtggcgtgtgcagtgccaccaacggtgtccagcgagggcgtgtgctgcgccggacggccgcttcgaccgacgtggtctccttcgtggaccccaatgacattagaaaacccgcagcagcagcagctggccctgcggtggataaggtcggcgttctgctgttaaaccttggcgggcccgaaaagctcgacgacgtcaagcctttcctgtataacctattcgccgacccagaaattattcgcctgccagcggcagctcagttcctgcagccgctgctcgcgacgatcatctccacgcttcgcgccccgaagagcgcggagggctatgaggccattggcggtggtagcccgttgcgtaggattacagacgagcaggcggaggcgctggcggagtctctgcgcgccaagggccaacctgcgaacgtgtacgtgggcatgcgctattggcacccctacacggaggaggcgctggagcacattaaggccgacggcgtcacgcgcctggtcatcctcccgctgtaccctcagttctccatctctaccagcggctccagccttcgactgcttgagtcgctcttcaagagcgacatcgcgctcaagtcgctgcggcacacggtcatcccgtcctggtaccagcggcggggctacgtgagcgcgatggcggacctgattgtagag

SEQ ID NO: 118 - DNA, 138 bp, Chlamydomonas reinhardtii
gagctgaagaagttccgggacgtgcccagcgtggagctgtttttctccgcgcacggcgtgcccaagtcctacgtggaggaggcgggcgacccatacaaggaggagatggaggagtgcgtgcggctcattacggacgag

SEQ ID NO: 119 - DNA, 98 bp, Chlamydomonas reinhardtii
gtcaagcggcgcggcttcgccaacacgcacacgctggcctaccagagccgcgtgggccccgcggaatggctcaagccgtacacggatgagtccatcaa

SEQ ID NO: 120 - DNA, 119 bp, Chlamydomonas reinhardtii
ggagctgggcaagcgcggcgtcaagtcgctgctggcggtgcccatcagctttgtcagcgagcacattgagacgttggaggagatcgacatggagtaccgcgagctggcggaggagagcg

SEQ ID NO: 121 - DNA, 135 bp, Chlamydomonas reinhardtii
gcatccgcaactggggccgcgtgccggcgctgaacaccaacgccgccttcatcgacgacctggcggacgcggtgatggaggcgctgccctacgtgggctgcctggccgggccgacagactcgctggtgccgctgg

SEQ ID NO: 122 - DNA, 200 bp, Chlamydomonas reinhardtii
gcgacctggagatgctgctgcaggcctacgaccgcgagcgccgcacgctgccgtcaccggtggtgatgtgggagtggggctggaccaagagcgcggagacgtggaacggccgcattgccatgattgccatcatcatcatcctggcgctggaggcagccagcggccagtccatcctcaaaaacctgttcctggcggagtag

SEQ ID NO: 123 - DNA, 66 bp, Chlamydomonas reinhardtii
gtgcgataataaatttgcatccttatgaattgctcaatgactaacgagcagcgtccgcgaccacag

SEQ ID NO: 124 - DNA, 527 bp, Chlamydomonas reinhardtii
gtgagggtggcattctgtaaagggagttgtggagttgggcagagcgagtgggtttggtcgccagggcgaggatgttgcgcgggcgttggcaggaacagggctgctagggcttgcgtggccagcgactagggtttcgactggccagcgccgccggggcgcgcttgccgaagctgcacagccccaagcgcttctgtggatcaaatggaaacttgtggcagtgtgtatgctagcgccttggcgcaagaccaattttagtggtattactgttattactgtggtagcggtgggtattcggcggcgtggttgttgttgcagccccgtgcgactaagaccgctggcaacgacagcaagccgccgcacccaggcatatacggcccaccagcaccaccgtacacaaccacgtgcctttgcactctacgcaccacagcgcgctgctgccgctcccacctcccatcccaacggcccctcttacccccacttcacaacccctcctctcacacgccctcctcttccccctcctcttccag

SEQ ID NO: 125 - DNA, 264 bp, Chlamydomonas reinhardtii
gtgggccgggcgcagcgggcgggcgggaggggcaggaggggcaggaggggaggaagggaggggaggaagggatggaaagctggcgcagcggcagcggcgggacaggtagagggcgctgccccagcggcggcaggtgggcatggtgggcgggtaggggcgacgcgtgagggactcgtcaggcatccgcatggcggcgacttgctgctcctcaccgctgacggctgcatctgctgtgtgcgtaacctggcctggctggcaccgcag

SEQ ID NO: 126 - DNA, 392 bp, Chlamydomonas reinhardtii
gtgaggcccgtgggtgggacgcggggagggacgcggggagggggagacgcgggagcgggacaagggtgaggatacggggagggaataggagaggccatggggagggatggggacacgggaggatgcacgggcctgggtggagccagggggaagtggacgacgagcccggcgggaggagggctgggtagaaggacgcgggaggtggttgggacaggtggacggggcgtgtggagcatacggcgcaagaagcgggactgagcgggttgcagggatggatgtaatcacggcaagtaagaaccccgagtggggctcagcgtgtcagcctgccttatctttcgcgcaagcgctggggttttatttcgctgtacacacgtcgcgcctttctgccgcag

SEQ ID NO: 127 - DNA, 508 bp, Chlamydomonas reinhardtii
gtgaggaggcgccggagttttgggggaaggggtgcggcgtgaagcgagatggcaggggcgaaggaaggagcggatggtggctgggtgcaagcggagaggcgacagagagtggaggttttggtggagcggttggggagaggggcgcagcagggatgcggccctggggatggcgggacagaagggagcaagtttgccaagtgaagggggggggtgctcaagaggagagggcggtggaggttaagacggccgtgctggttatgctggggttgcaaggcgcatgggcgcatggagccgggggagtttggctgtggatgggcactgcggatgggcacggcttgctactcatgtgcggtcgcggtccgcggtgtgtcagccagccaggacccatcccactgggtcttcctgcgtgcctgggactgcttgccgccacccacccattcatcaccaccactgcgcagacccaccaacaccgctgccctgaactgctctgactcttggcgctcctcag

SEQ ID NO: 128 - DNA, 686 bp, Chlamydomonas reinhardtii
gtgagtcgcgccgtcgcggttggttcgcggatgccggttggcggatgacgttcggcggttggcattgggtttgggtttgaggggttgttgggtgaggtcgggattggggtcgggattgggggtcgagcgtggggctggcgtggatgatggcgtggtctttggaaggggcttggggaggttgcgcgtgtggatgcggacagcatgggcgcgacagtgcgcatgtgcatgtgctgtgtcaaacgtctggtgcgttcagtgtgtccttgcgtgcctcccaccgtacgcagccatcccgcgcgcctggaccgtagagaccgcctacgtgtccgctagcggcctcggcctcagcctaagcgccagtagcgccagcgacacaagcaacactgtcgctaatggcagcagcggcagcagcagcagtcacgagaatgcccgcggccgggagaaagtgctcctagccgggggccgccgctagctggtttcctcagcgcgtggacggtggtgccttcatcccgaccaccccaggcgcgtccccagtcccgtcgagctcgcctgccttgtggcccgccttgaccgccctggcgccacccggtggctcgcataacgactcgctttccgttctccgcctgacgctgtccgcctgacgctctgcgcttgactctttgcgccttcctcccctcttcccccag

SEQ ID NO: 129 - DNA, 4201 bp, Artificial Sequence
  RedAlgae CHLH DNA
atgcagacttcctcgcttcttggccggcgcacggcccacccggctgcgggcgcgacgcccaagccggttgcgccctcgccccgcgtggctagcacccgccaggtcgcgtgcaatgtggcgactggaccccggccgcccatgaccaccttcaccggtggcaacaagggccctgctaagcagcaggtgtcgctggatctgcgcgacgagggcgctggcatgttcaccagcaccagcccggagatgcgccgtgtcgtccctgacgatgtgaagggtcgcgttaaggtgaaggttgtgtacgtggtgctggaggcccagtaccagtcggccatcagcgctgcggtgaagaacatcaacgccaagaactccaaggtgtgcttcgaggtggtgggctacctgctggaggagctgcgtgaccagaagaacctcgatatgctcaaggaggatgtggcctctgccaacatcttcatcggctcgctcatcttcattgaggagcttgccgagaagattgtggaggcggtgagccccctgcgcgagaagctggacgcgtgcctgatcttcccgtccatgccggcggtcatgaagctgaacaagctgggcacgttttcgatggctcagctgggccagtcgaagtcggtgttctcggagttcatcaagtctgctcgcaagaacaacgacaacttcgaggagggcttgctgaagctggtgcgcaccctgcctaaggtgctgaagtatctgccctcggacaaggcgcaggacgccaagaacttcgtgaacagcctgcagtactggctgggcggtaactcggacaacctggagaacctgctgctgaacaccgtcagcaactacgtgcccgctctgaagggcgtggacttcagcgtggctgagcccaccgcctaccccgatgtgggtatctggcaccctctggcctcgggcatgtacgaggacctgaaggagtacctgaactggtacgacacccgcaaggacatggtcttcgccaaggacgcccccgtcattggcctggtgctgcagcgctcgcacctggtgactggcgatgagggccactacagcggcgtggtcgctgagctggagagccgcggtgctaaggtcatccccgtctttgccggtggcctggacttctccgcccccgtcaagaagttcttctacgaccccctgggctctggccgcacgttcgtggacaccgttgtgtcgctgaccggcttcgcgctggtgggcggccccgcgcgccaggacgcgccgaaggccattgaggcgctgaagaacctgaacgtgccctacctggtgtcgctgccgctggtgttccagaccactgaggagtggctggacagcgagctgggcgtgcaccccgtccaggtggctctgcaggttgccctgcccgagctggatggtgccatggagcccatcgtgttcgctggccgtgactcgaacaccggcaagtcgcactcgctgcccgaccgcatcgcttcgctgtgcgctcgcgccgtgaactgggccaacctgcgcaagaagcgcaacgccgagaagaagctggccgtcaccgtgttcagcttcccccctgacaagggcaacgtcggcactgccgcctacctgaacgtgttcggctccatctaccgcgtgctgaagaacctgcagcgcgagggctacgacgtgggcgccctgtccgccctcggaggaggatctgatccagtcggtgctgacccagaaggaggccaagttcaactcgaccgacctgcacatcgcctacaagatgaaggtggacgagtaccagaagctgtgcccttacgccgaggcgctggaggagaactggggcaagccccccggcaccctgaacaccaacggccaggagctgctggtgtacggccgccagtacggcaacgtcttcatcggcgtgcagcccaccttcggctacgagggcgacccgatgcgcctgctgttctcgaagtcggccagcccccaccacggcttcgccgcctactacaccttcctggagaagatcttcaaggccgacgccgtgctgcacttcggcacccacggctcgctggagttcatgcccggcaagcaggtcggcatgtcgggtgtgtgctaccccgactcgctgatcggcaccatccccaacctctactactacgccgccaacaacccgtctgaggccaccatcgccaagcgccgctcgtacgccaacaccatttcgtacctgacgccgcctgccgagaacgccggcctgtacaagggcctgaaggagctgaaggagctgatcagctcgtaccagggcatgcgtgagtctggccgcgccgagcagatctgcgccaccatcattgagaccgccaagctgtgcaacctggaccgcgacgtgaccctgcccgacgctgacgccaaggacctgaccatggacatgcgcgacagcgttgtgggccaggtgtaccgcaagctgatggagattgagtcccgcctgctgccctgcggcctgcacgtggtgggctgcccgcccaccgccgaggaggccgtggccaccctggtcaacatcgctgagctggaccgcccggacaacaacccccccatcaagggcatgcccggcatcctggcccgcgccattggtcgcgacatcgagtcgatttacagcggcaacaacaagggcgtcctggctgacgttgaccagctgcagcgcatcaccgaggcctcccgcacctgcgtgcgcgagttcgtgaaggaccgcaccggcctgaacggccgcatcggcaccaactggatcaccaacctgctcaagttcaccggcttctacgtggacccctgggtgcgcggcctgcagaacggcgagttcgccagcgccaaccgcgaggagctgatcaccctgttcaactacctggagttctgcctgacccaggtggtcaaggacaacgagctgggcgccctggtagaggcgctgaacggccagtacgtcgagcccggccccggcggtgaccccatccgcaaccccaacgtgctgcccaccggcaagaacatccacgccctggaccctcagtcgattcccactcaggccgcgctgaagagcgcccgcctggtggtggaccgcctgctggaccgcgagcgcgacaacaacggcggcaagtaccccgagaccatcgcgctggtgctgtggggcactgacaacatcaagacctacggcgagtcgctggcccaggtcatgatgatggtcggtgtcaagcccgtggccgacgccctgggccgcgtgaacaagctggaggtgatccctctggaggagctgggccgcccccgcgtggacgtggttgtcaactgctcgggtgtgttccgcgacctgttcgtgaaccagatgctgctgctggaccgcgccatcaagctggcggccgagcaggacgagcccgatgagatgaacttcgtgcgcaagcacgccaagcagcaggcggcggagctgggcctgcagagcctgcgcgacgcggccacccgtgtgttctccaacagctcgggctcctactcgtccaacgtcaacctggcggtggagaacagcagctggagcgacgagtcgcagctgcaggagatgtacctgaagcgcaagtcgtacgccttcaactcggaccgccccggcgccggtggcgagatgcagcgcgacgtgttcgagacggccatgaagaccgtggacgtgaccttccagaacctggactcgtccgagatctcgctgaccgatgtgtcgcactacttcgactccgaccccaccaagctggtggcgtcgctgcgcaacgacggccgcacccccaacgcctacatcgccgacaccaccaccgccaacgcgcaggtccgcactctgggtgagaccgtgcgcctggacgcccgcaccaagctgctcaaccccaagtggtacgagggcatgcttgcctcgggctacgagggcgtgcgcgagatccagaagcgcatgaccaacaccatgggctggtcggccacctcgggcatggtggacaactgggtgtacgacgaggccaactcgaccttcatcgaggatgcggccatggccgagcgcctgatgaacaccaaccccaacagcttccgcaagctggtggccaccttcctggaggccaacggccgcggctactgggacgccaagcccgagcagctggagcgcctgcgccagctgtacatggacgtggaggacaagattgagggcgtcgaataa

SEQ ID NO: 130 - DNA, 263 bp, Chlamydomonas reinhardtii
tcctacagagtaaaggtctaggcgatgcgcgactgaaagactgtgaatcccggcgtcgccgtggtgggatgtgggccggtgcgctgtcgcagaggataaattacaggtatcaaacaaggttagggcgttggaaggagcggcgctagggaactgaaatcggatctgcatcggaccctcattccgcgacttgtccttcttttgcctcgccccgcagctcttgagttttgttcttgaccctttgacacgaaccaaccgatataaaa

SEQ ID NO: 131 - DNA, 843 bp, Chlamydomonas reinhardtii
gcggcaggccttcatggtcgtcgttggagcatttgcggaaaggctgatggcagcagatgcagccatgtcagttgtggctgaagttgttggctggggcgggagcgggcagcagctgctgcgagcggccgaagcagcggtgctgctttgcgtatgagaggaagaccagtgccctcgaggaggcgagtgcctgtgtgagtgtcaggacgtgtgacttcggaaactgagggcggtgagtagatgtgactggggcttgcaggaagcctactgaccctatcagaaaaggtgagcaggggtatatggtctaggagcgttgccggagcgtggctggccagtgctagccgcgcgggctctgttgctcgctggcgcgccgccgccttcacaacagatgccgtagaaatgcagcgatgtgacgaggcgtggcctattctgcaatgtgtgaggcgccaatggcgccactgacaaatggaggagtggtcaaagcttgggtacgttttgagagctgcatcgggcagcgaggatcagtgtgcggtaagaccgacggcagacggattggcaagggaataggagggacgtgggcgtgggcgcccgcgctttgtcgaggccgcatgagccggccgcttctagacccgtagcccattttgaacaagcgcccacgcgtgctcccgatgggggacatcgatcacgggaattgattaaggggcatgtgtggtgtgcaagtgagtgactggtggttccgtccctgtgaggttgtttcgttggacgtggctgccgggttgcgcgcgggctaagcgggcctgaggcagagcgctggcgtgtagccgcgagtatcgatctgtaacgtgc

SEQ ID NO: 132 - DNA, 120 bp, Chlamydomonas reinhardtii
atggccctgaacatgcgtgtttcctcttccaaggtcgctgccaagcagcagggccgcatctccgcggtgccggttgtgtcgagcaaggtggcctcctccgcccgcgtggcccccttccag

SEQ ID NO: 133 - DNA, 37 bp, Chlamydomonas reinhardtii
ggcgctcccgtggccgcgcagcgcgctgctctgctgg

SEQ ID NO: 134 - DNA, 60 bp, Chlamydomonas reinhardtii
tgcgcgccgctgccgctactgaggtcaaggctgctgagggccgcactgagaaggagctgg

SEQ ID NO: 135 - DNA, 176 bp, Chlamydomonas reinhardtii
gccaggcccgccccatcttccccttcaccgccatcgtgggccaggatgagatgaagctggcgctgattctgaacgtgatcgaccccaagatcggtggtgtcatgatcatgggcgaccgtggcactggcaagtccaccaccattcgtgccctggcggatctgctgcccgagatgcag

SEQ ID NO: 136 - DNA, 193 bp, Chlamydomonas reinhardtii
gtggttgccaacgacccctttaactcggaccccaccgaccccgagctgatgagcgaggaggtgcgcaaccgcgtcaaggccggcgagcagctgcccgtgtcttccaagaagattcccatggtggacctgcccctgggcgccactgaggaccgcgtgtgcggcaccatcgacatcgagaaggcgctgaccgagg

SEQ ID NO: 137 - DNA, 89 bp, Chlamydomonas reinhardtii
gtgtcaaggcgttcgagcccggcctgctggccaaggccaaccgcggcatcctgtacgtggatgaggtcaacctgctggacgaccacctg

SEQ ID NO: 138 - DNA, 100 bp, Chlamydomonas reinhardtii
gtcgatgtgctgctggactcggccgcctccggctggaacaccgtggagcgcgagggtatctccatcagccaccccgcccgcttcatcctggtcggctcgg

SEQ ID NO: 139 - DNA, 145 bp, Chlamydomonas reinhardtii
gcaaccccgaggagggtgagctgcgcccccagctgctggatcgcttcggcatgcacgcccagatcggcaccgtcaaggacccccgcctgcgtgtgcagatcgtgtcgcagcgctcgaccttcgacgagaaccccgccgccttccg

SEQ ID NO: 140 - DNA, 202 bp, Chlamydomonas reinhardtii
caaggactacgaggccggccagatggcgctgacccagcgcatcgtggacgcgcgcaagctgctgaagcagggcgaggtcaactacgacttccgcgtcaagatcagccagatctgctcggacctgaacgtggacggcatccgcggcgacatcgtgaccaaccgcgccgccaaggccctggccgccttcgagggccgcaccgag

SEQ ID NO: 141 - DNA, 132 bp, Chlamydomonas reinhardtii
gtgacccccgaggacatctaccgtgtcattcccctgtgcctgcgccaccgcctccggaaagaccccctggctgagatcgacgacggtgaccgcgtgcgtgagatcttcaagcaggtgttcggcatggagtaa

SEQ ID NO: 142 - DNA, 101 bp, Chlamydomonas reinhardtii
gtgtgcagttgcatctaaagaacgtccaattcatggttactgctcgtggatctaagcggttggctcaccagcgttccatggtccccgattcgtgcacgcag

SEQ ID NO: 143 - DNA, 121 bp, Chlamydomonas reinhardtii
gtgagaagccatgatacaaatataaggatttgaagcggtagatctaggacccatcgaacttgagcaccgacttgcagtccttgccttgtccggcgactgaacttctgcgcttgctttgcag

SEQ ID NO: 144 - DNA, 82 bp, Chlamydomonas reinhardtii
gtaagtgtcgcgcaaagattttctgccgggacgggtctccctcgcaacatctgaacccatggctcgtttttttgccccgcag

SEQ ID NO: 145 - DNA, 397 bp, Chlamydomonas reinhardtii
gtgcgcgcctcccccaaccccagtttggcaaatgtgtggttaagcgtcgaaagcgtgaacagaaacaggtgttgcgggggccgcggaatggctgcaatgggtgctgggggcttcggagggtctgggggcgagtttgggtatacacgggcgcgcacacttgaaggaacgctcaaggacgacagcggaggcgtggagacagcgccggcccaagcagcctgtacttgtagctgctggtcagctgaggcatcacgacttgggaccagcacccggcctcacggttgcacaaggccatcaccgcgcgccaccacccacgcctcttcaaacccatgccggcacctaccgctacccctgtgacacgctccgcacacgccgccccgcacaccccaccatgtgacag

SEQ ID NO: 146 - DNA, 156 bp, Chlamydomonas reinhardtii
gtgagagcgaggcgcggggcgtgctctgcaggctagggtgaagatcaggagagccgaagcgggcccgaacagcgcagagagaggcaagacgacacccctgccgcgttttgatcacaagattcacacccttgctctccccaacgctcccgcacatag

SEQ ID NO: 147 - DNA, 476 bp, Chlamydomonas reinhardtii
gtgagcaggggcagataggcggtcgggcggctgggcggcaggggctgtgttggctgtgttgggtgtgggctgaggctggtgggtgggctggcgggtggcagggatagcggtgaggggatggtgatggggcagaatgggcgggtgggcggacacgtggggtcgttgaagggtgtgtggggacggcaactggtatgcgatatgtcggcttggccctggcggggaaagcattcgcagaatggcgcacgaacgaggccggggagcgagcggggatgggagacgcaacctgcgctgcgaagtgcggcgcgcgctccagttgacacgttgcacgaatgtggccagtgttcgcctgagagttatgggttagaccgccagatgagccggttaagctggtggtcgcggttgatcggctgcttcccttccggttgcacgcctggcaccctaacattaccctgtccgctgctgccctttgcccacag

SEQ ID NO: 148 - DNA, 191 bp, Chlamydomonas reinhardtii
gtgagtgcagctgccgctgcggctgctgatggtgacctgtgcgaccacggggctccgcatttctggacgaagcgttgtaccatagccgtcttggtccctgatttgggccggctctggtccgaagccttgacatctacagttcaacatggccgtataacgatcctgtgcccacccacacgccaccccgccag

SEQ ID NO: 149 - DNA, 212 bp, Chlamydomonas reinhardtii
gtgagcgcgcgctctacgatacggcagacatgtacacactgcggcgcactgtagagcttgcattgcatttcaaggcctcgaaagagtagggtggtcgttctctggtggtgtccggccacaattatgcaccccggtgttggtgcagcagctgtgatgtcacaccttgcatcacccccctactgctgccgcctctcctctcttctcgcccgcag

SEQ ID NO: 150 - DNA, 211 bp, Chlamydomonas reinhardtii
gtgagcagagcaatattgcagagggaagggtggcggaagggtgataacggttggggatctagaggggcgagatggatgcacacagcgcggggttggttatgcatgcctgcatggacgcgtgcacgcacccctgatctgccggttttccaactggcgatgccgtattatgacctgcagctcaccatcctcatgcttgatttgcctcgctcag

SEQ ID NO: 151 - protein, 417 aa, Chlamydomonas reinhardtii; identical to SEQ ID NO: 22

SEQ ID NO: 152 - protein, 721 aa, Artificial Sequence
  Mutant protein sequence RedAlgaeCHLH
MQTSSLLGRRTAHPAAGATPKPVAPSPRVASTRQVACNVATGPRPPMTTFTGGNKGPAKQQVSLDLRDEGAGMFTSTSPEMRRVVPDDVKGRVKVKVVYVVLEAQYQSAISAAVKNINAKNSKVCFEVVGYLLEELRDQKNLDMLKEDVASANIFIGSLIFIEELAEKIVEAVSPLREKLDACLIFPSMPAVMKLNKLGTFSMAQLGQSKSVFSEFIKSARKNNDNFEEGLLKLVRTLPKVLKYLPSDKAQDAKNFVNSLQYWLGGNSDNLENLLLNTVSNYVPALKGVDFSVAEPTAYPDVGIWHPLASGMYEDLKEYLNWYDTRKDMVFAKDAPVIGLVLQRSHLVTGDEGHYSGVVAELESRGAKVIPVFAGGLDFSAPVKKFFYDPLGSGRTFVDTVVSLTGFALVGGPARQDAPKAIEALKNLNVPYLVSLPLVFQTTEEWLDSELGVHPVQVALQVALPELDGAMEPIVFAGRDSNTGKSHSLPDRIASLCARAVNWANLRKKRNAEKKLAVTVFSFPPDKGNVGTAAYLNVFGSIYRVLKNLQREGYDVGALSALGGGSDPVGADPEGGQVQLDRPAHRLQDEGGRVPEAVPLRRGAGGELGQAPRHPEHQRPGAAGVRPPVRQRLHRRAAHLRLRGRPDAPAVLEVGQPPPRLRRLLHLPGEDLQGRRRAALRHPRLAGVHARQAGRHVGCVLPRLADRHHPQPLLLRRQQPV

SEQ ID NO: 153 - DNA, 1254 bp, Chlamydomonas reinhardtii; identical to SEQ ID NO: 21